SEQUENCE LISTING (21 sequences)

SEQ ID NO: 1 - protein, 23 aa, Homo sapiens
AEPRQEFEVMEDHAGTYGLGDRK

SEQ ID NO: 2 - protein, 5 aa, synthetic construct
SYGMS

SEQ ID NO: 3 - protein, 17 aa, synthetic construct
TINSGGTYTYYPDSVKG

SEQ ID NO: 4 - protein, 8 aa, synthetic construct
SYSGAMDY

SEQ ID NO: 5 - protein, 117 aa, synthetic construct
EVQLVESGGGLVQPGGSLRLSCAASGLIFRSYGMSWVRQAPGKGLEWVATINSGGTYTYYPDSVKGRFTISRDNSKNTLYLQMNSLRAEDTAVYYCANSYSGAMDYWGQGTLVTVSS

SEQ ID NO: 6 - protein, 16 aa, synthetic construct
RSSQSIVHSNGNTYLE

SEQ ID NO: 7 - protein, 7 aa, synthetic construct
KVSNRFS

SEQ ID NO: 8 - protein, 9 aa, synthetic construct
FQGSLVPWT

SEQ ID NO: 9 - protein, 112 aa, synthetic construct
DDVLTQTPLSLPVTPGQPASISCRSSQSIVHSNGNTYLEWYLQKPGQSPQLLIYKVSNRFSGVPDRFSGSGSGTDFTLKISRVEAEDVGVYYCFQGSLVPWTFGQGTKVEIK

SEQ ID NO: 10 - protein, 23 aa, Macaca fascicularis
AEPRQEFDVMEDHAGTYGLGDRK

SEQ ID NO: 11 - protein, 444 aa, synthetic construct
EVQLVESGGGLVQPGGSLRLSCAASGLIFRSYGMSWVRQAPGKGLEWVATINSGGTYTYYPDSVKGRFTISRDNSKNTLYLQMNSLRAEDTAVYYCANSYSGAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 443 aa, synthetic construct
EVQLVESGGGLVQPGGSLRLSCAASGLIFRSYGMSWVRQAPGKGLEWVATINSGGTYTYYPDSVKGRFTISRDNSKNTLYLQMNSLRAEDTAVYYCANSYSGAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 13 - protein, 219 aa, synthetic construct
DDVLTQTPLSLPVTPGQPASISCRSSQSIVHSNGNTYLEWYLQKPGQSPQLLIYKVSNRFSGVPDRFSGSGSGTDFTLKISRVEAEDVGVYYCFQGSLVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 117 aa, synthetic construct
EVQLVESGGDLAKPGGSLKLSCTASGLIFRSYGMSWVRQTPDKRLEWVATINSGGTYTYYPDSVKGRFTISRDNAKNTLYLQMSSLKSEDTAMYYCANSYSGAMDYWGQGTSVTVSS

SEQ ID NO: 15 - protein, 112 aa, synthetic construct
DDLLTQTPLSLPVSLGDPASISCRSSQSIVHSNGNTYFEWYLQKPGQSPKLLIYKVSNRFSGVPDRFSGSGSGTDFTLKISRVEAEDLGVYYCFQGSLVPWTFGGGTKLEIK

SEQ ID NO: 16 - protein, 5 aa, synthetic construct
SYGMS

SEQ ID NO: 17 - protein, 17 aa, synthetic construct
TINSGGTYTYYPDSVKG

SEQ ID NO: 18 - protein, 8 aa, synthetic construct
SYSGAMDY

SEQ ID NO: 19 - protein, 16 aa, synthetic construct
RSSQSIVHSNGNTYFE

SEQ ID NO: 20 - protein, 7 aa, synthetic construct
KVSNRFS

SEQ ID NO: 21 - protein, 9 aa, synthetic construct
FQGSLVPWT

(19) United States
(12) Patent Application Publication
(10) Pub. No.: US 2024/0383972 A1
(43) Pub. Date: Nov. 21, 2024

(54) METHODS OF TREATING TAU PATHOLOGIES

(71) Applicant: Genentech, Inc., South San Francisco, CA (US)

(72) Inventors: Edmond Huatung Teng, South San Francisco, CA (US); Balazs Toth, South San Francisco, CA (US); Paul Manser, South San Francisco, CA (US); Michael Keeley, South San Francisco, CA (US)

(21) Appl. No.: 18/587,251

(22) Filed: Feb. 26, 2024

Related U.S. Application Data
(63) Continuation of application No. PCT/US2022/075528, filed on Aug. 26, 2022.
(60) Provisional application No. 63/389,269, filed on Jul. 14, 2022.
(60) Provisional application No. 63/275,884, filed on Nov. 4, 2021.
(60) Provisional application No. 63/238,737, filed on Aug. 30, 2021.
(60) Provisional application No. 63/238,052, filed on Aug. 27, 2021.

(51) Int. Cl. C07K 16/18; A61K 39/00; A61P 25/28

(52) U.S. Cl. CPC C07K 16/18; A61P 25/28; A61K 2039/505; C07K 2317/24; C07K 2317/76

(57) ABSTRACT

The present disclosure relates to methods of slowing cognitive decline in mild-to-moderate and moderate Alzheimer's disease, and other Tau pathologies, using anti-Tau antibodies. The disclosure provides first-in-class immunotherapy for use in reducing clinical decline in mild-to-moderate AD and moderate AD, in particular, significantly reducing rate of decline in cognitive capacity to a clinically meaningful extent, and significantly retaining memory. The disclosure also relates to use of anti-Tau antibodies to intercept cell-to-cell spread of pathological Tau in the brains of patients with AD or related Tauopathies.

### CROSS-REFERENCE TO RELATED APPLICATIONS

This application claims priority to and benefit of U.S. Provisional Application No. 63/238,052, filed Aug. 27, 2021, U.S. Provisional Application No. 63/238,737, filed Aug. 30, 2021, U.S. Provisional Application No. 63/275,884, filed Nov. 4, 2021, and U.S. Provisional Application No. 63/389,269, filed Jul. 14, 2022, the contents of which are hereby incorporated by reference in their entireties.

### TECHNICAL FIELD

The present disclosure relates to methods of slowing cognitive decline in mild-to-moderate Alzheimer's disease and other Tau pathologies using anti-Tau antibodies.

### SEQUENCE LISTING

The instant application contains a Sequence Listing which has been submitted electronically in XML format and is hereby incorporated by reference in its entirety. Said XML copy, created on Aug. 26, 2022, is named 000218-0061-WO1_SL.xml and is 15,329 bytes in size.

### BACKGROUND

Alzheimer's Disease (AD) is the most common cause of dementia, affecting an estimated 5.7 million individuals in the United States (Alzheimer's Association 2018). It is estimated that 46.8 million people have AD or other dementias (Prince et al., World Alzheimer Report 2015: The Global Impact of Dementia, Alzheimer's Disease International, 2015 (available at www.alz.co.uk/research/WorldAlzheimerReport2015.pdf.). The disease is characterized pathologically by the accumulation of plaques containing extracellular P amyloid (AP) peptide and intracellular neurofibrillary tangles containing aggregates of the microtubule-associated protein Tau in the brain neocortex. Diagnosis is made through the clinical assessment of the neurologic and neuropsychiatric signs and symptoms of AD and the exclusion of other causes of cognitive dysfunction. AD is commonly classified into preclinical, prodromal, mild, moderate, and severe stages by the presence and severity of clinically relevant functional and/or cognitive decline, categorization often being facilitated by global measures, such as the Clinical Dementia Rating (CDR) scale (Morris, Neurology 1993; 43:241-4), or the Mini-Mental State Examination (“MMSE;” Folstein et al. J Psychiatr Res 1975; 12:189-98). Approved medical therapies that inhibit acetylcholinesterase (“AChE”) activity or antagonize N-methyl-D-aspartate receptors in the brain may temporarily improve the symptoms of AD in some patients but do not modify the progression of the disease (Cummings, N. Engl. J. Med. 2004; 351:56-67)

The deposition of extracellular amyloid plaques and intracellular Tau aggregates in the brain are the hallmark pathologic findings in AD, first reported by Alois Alzheimer in 1906. Intracellular neurofibrillary tangles are composed of aggregated and abnormally phosphorylated Tau protein. Tau, encoded by the MAPT gene and expressed in the human brain as six alternatively spliced isoforms, having a length of 352-441 amino acids. The six isoforms comprise combinations of three 29-residue near-amino-terminal inserts(0N, TN, and 2N) and two carboxy-terminal repeat domains (3R and 4R) (Wang and Mandelkow, Nat. Rev. Neurosci. 2016 January; 17(1):5-21). While the intracellular aggregates are found as neurofibrillary tangles in the neuronal soma and as neuropil threads in the dendritic compartment, it is believed that the spread of Tau pathology through the brain is mediated by soluble Tau in the extracellular brain environment (Braak et al., Brain 2015; 138:2814-33; Wang and Mandelkow, Nat. Rev. Neurosci. 2016 January; 17(1):5-21).

The spatial distribution of Tau pathology in patients with AD correlates with decline in the cognitive domains sub-served by the affected cortical networks (Ossenkoppele et al., Brain 2016; 139(Pt 5):1551-67). Knockout of the Tau gene in an AD transgenic mouse model is protective against cognitive deficits (Roberson et al., Science 2007; 316:750-4). It has been thought that therapies that reduce the spread of Tau in the brain may alleviate cognitive dysfunction and block further synaptic loss, axon degeneration, and neuronal cell death.

Nonetheless, significant failures have marked the development of therapeutic anti-Tau antibodies for the treatment of AD and other tauopathies, such as progressive supranuclear palsy (PSP). For example, a Phase 2 trial of C2N-8E12, a humanized IgG4 antibody that recognizes an aggregated form of Tau, was discontinued when administration of the drug to PSP patients failed to show any therapeutic beneficial effects (ALZFORUM Networking for a Cure, “AbbVie's Tau Antibody Flops in Progressive Supranuclear Palsy,” Jul. 26, 2019, available at https://www.alzforum.org/news/research-news/abbvies-Tau-antibody-flops-progressive-supranuclear-palsy), as well as being more recently discontinued for use in AD patients (ALZFORUM Networking for a Cure, Therapeutics, “Tilavonemab,” available at https://www.alzforum.org/therapeutics/tilavonemab, updated Jan. 17, 2022). Similarly, a Phase 2 trial with Gosuranemab, a humanized monoclonal antibody against an N-terminal fragment of Tau, was discontinued when administration to PSP patients failed to show efficacy in the interim analysis (Sandusky-Beltran, et al., 2020; Neuropharmacology 175:108104).

Moreover, in a recent phase 2 trial with Gosuranemab for AD, the drug failed to meet its primary efficacy endpoint in patients with mild cognitive impairment (MCI) and mild AD dementia, and no therapeutic benefits were seen on various exploratory endpoints, despite the study's reported lowering of N-terminal Tau in patients' cerebrospinal fluid (CSF) (Biogen Inc., “Biogen Announces Topline Results from Phase 2 Study of Gosuranemab, an Anti-Tau Antibody, for Alzheimer's Disease,” Jun. 16, 2021, available at https://www.globenewswire.com/news-release/2021/06/16/2248550/0/en/Biogen-Announces-Topline-Results-From-Phase-2-Study-of-Gosuranemab-an-Anti-Tau-Antibody-for-Alzheimer-s-Disease.html). Similarly, zagotenemab for AD also has been discontinued, after missing its primary endpoint in a Phase 2 clinical trial (ALZFORUM Networking for a Cure, Therapeutics, “Zagotenemab,” available at https://www.alzforum.org/therapeutics/zagotenemab, updated Oct. 29, 2021). A Phase 2 trial of the anti-Tau antibody, semorinemab, in early (prodromal to mild) AD, also did not meet its primary efficacy and secondary endpoints (AC Immune, “AC Immune Reports Top Line Results from TAURIEL Phase 2 Trial Evaluating Semorinemab in Early Alzheimer's Disease,” Sep. 23, 2020, available at https://ir.acimmune.com/news-releases/news-release-details/ac-immune-reports-top-line-results-Tauriel-phase-2-trial).

It is estimated that one in nine people over the age of 65 have AD—the aggregated yearly costs for health care, long-term care and hospice care by and on behalf of individuals afflicted with AD are over $200 billion in 2013, and are estimated to rise to $1.2 trillion by 2050 (Alzheimer's Association, “2013 Alzheimer's Disease Facts and Figures,” Alzheimer's and Dementia 9:208-245, 2013). AD is the sixth-leading cause of death in the United States as of 2013 (id.).

Existing therapies for AD provide only modest symptomatic benefit and fail to slow progression of the underlying neurodegenerative process. There is a tremendous unmet medical need for a disease-modifying therapeutic for AD, particularly among patients with mild-to-moderate AD and moderate AD, as well as for patients with other Tau pathologies.

### SUMMARY OF THE DISCLOSURE

In one aspect, the present disclosure provides a method of slowing decline in cognitive capacity in a patient diagnosed with mild-to-moderate Alzheimer's disease (AD), comprising administering to said patient a 4500 mg dose of humanized monoclonal anti-Tau antibody, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

In another aspect, the present disclosure provides a method of maintaining cognitive capacity within 5 points of an Alzheimer's disease Assessment Scale, Cognitive Subscale, 11-item version (ADAS-Cog11) score of a patient diagnosed with mild-to-moderate AD, comprising administering to said patient a 4500 mg dose of humanized monoclonal anti-Tau antibody, wherein the ADAS-Cog11 score of the patient assessed after administration of 12 to 17 doses of said antibody is no more than 2.5, no more than 3, no more than 3.5, no more than 4, no more than 4.5, or no more than 5 points higher than an ADAS-Cog11 score of the patient assessed before administration of said antibody, thereby maintaining cognitive capacity within 5 points of the ADAS-Cog11 score of the patient, and wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

In another aspect, the present disclosure provides a method of slowing decline in cognitive capacity in a patient diagnosed with moderate AD, comprising administering to said patient a 4500 mg dose of humanized monoclonal anti-Tau antibody, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

In another aspect, the present disclosure provides a method of maintaining cognitive capacity within 5 points of an Alzheimer's disease Assessment Scale, Cognitive Subscale, 11-item version (ADAS-Cog11) score of a patient diagnosed with moderate AD, comprising administering to said patient a 4500 mg dose of humanized monoclonal anti-Tau antibody, wherein the ADAS-Cog11 score of the patient assessed after administration of 12 to 17 doses of said antibody is no more than 2.5, no more than 3, no more than 3.5, no more than 4, no more than 4.5, or no more than 5 points higher than an ADAS-Cog11 score of the patient assessed before administration of said antibody, thereby maintaining cognitive capacity within 5 points of the ADAS-Cog11 score of the patient, and wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

In another aspect, the present disclosure provides a method of slowing memory decline in a patient diagnosed with mild-to-moderate AD, comprising administering to said patient a 4500 mg dose of humanized monoclonal anti-Tau antibody, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

In another aspect, the present disclosure provides a method of maintaining memory within 2.5 points of an Alzheimer's disease Assessment Scale, Cognitive Subscale, 11-item version (ADAS-Cog11) memory domain score of a patient diagnosed with mild-to-moderate AD, comprising administering to said patient a 4500 mg dose of humanized monoclonal anti-Tau antibody, wherein the ADAS-Cog11 memory domain score of the patient assessed after administration of 12 to 17 doses of said antibody is no more than 1, no more than 1.5, no more than 1.7, no more than 2, no more than 2.3, or no more than 2.5 points higher than an ADAS-Cog11 memory domain score of the patient assessed before administration of said antibody, thereby maintaining memory within 2.5 points of the ADAS-Cog11 memory domain score of the patient, and wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

In another aspect, the present disclosure provides a method of slowing memory decline in a patient diagnosed with moderate AD, comprising administering to said patient a 4500 mg dose of humanized monoclonal anti-Tau antibody to slow the decline in memory in the patient, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

In another aspect, the present disclosure provides a method of maintaining memory within 2.5 points of an Alzheimer's disease Assessment Scale, Cognitive Subscale, 11-item version (ADAS-Cog11) memory domain score of a patient diagnosed with moderate AD, comprising administering to said patient a 4500 mg dose of humanized monoclonal anti-Tau antibody, wherein the ADAS-Cog11 memory domain score of the patient assessed after administration of 12 to 17 doses of said antibody is no more than 1, no more than 1.5, no more than 1.7, no more than 2, no more than 2.3, or no more than 2.5 points higher than an ADAS-Cog11 memory domain score of the patient assessed before administration of said antibody, thereby maintaining memory within 2.5 points of the ADAS-Cog11 memory domain score of the patient, and wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

In another aspect, the present disclosure provides a method of slowing decline in language capacity in a patient diagnosed with mild-to-moderate AD, comprising administering to said patient a 4500 mg dose of humanized monoclonal anti-Tau antibody, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

In another aspect, the present disclosure provides a method of slowing decline in language capacity in a patient diagnosed with moderate AD, comprising administering to said patient a 4500 mg dose of humanized monoclonal anti-Tau antibody, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

In another aspect, the present disclosure provides a method of slowing decline in praxis capacity in a patient diagnosed with mild-to-moderate AD, comprising administering to said patient a 4500 mg dose of humanized monoclonal anti-Tau antibody, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

In another aspect, the present disclosure provides a method of slowing decline in praxis capacity in a patient diagnosed with moderate AD, comprising administering to said patient a 4500 mg dose of humanized monoclonal anti-Tau antibody, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

In another aspect, the present disclosure provides a method of treating a patient diagnosed with mild-to-moderate AD without increased risk of an adverse event, comprising administering to said patient a 4500 mg dose of humanized monoclonal anti-Tau antibody, without increasing (or without significantly increasing) the risk of a treatment emergent adverse event, optionally wherein the dose is repeated for 12 to 17 doses, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

In another aspect, the present disclosure provides a method of treating a patient diagnosed with moderate AD without increased risk of an adverse event, comprising administering to said patient a 4500 mg dose of humanized monoclonal anti-Tau antibody, without increasing (or without significantly increasing) the risk of a treatment emergent adverse event, optionally wherein the dose is repeated for 12 to 17 doses, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

In another aspect, the present disclosure provides a humanized monoclonal anti-Tau antibody provided at a dose of 4500 mg for use in slowing decline in cognitive capacity in a patient diagnosed with mild-to-moderate AD, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

In another aspect, the present disclosure provides a humanized monoclonal anti-Tau antibody provided at a dose of 4500 mg for use in maintaining cognitive capacity no more than 5 points higher than an Alzheimer's disease Assessment Scale, Cognitive Subscale, 11-item version (ADAS-Cog11) score of a patient diagnosed with mild-to-moderate AD, after administration of 12 to 17 doses, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

In another aspect, the present disclosure provides a humanized monoclonal anti-Tau antibody provided at a dose of 4500 mg for use in slowing decline in cognitive capacity in a patient diagnosed with moderate AD, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

In another aspect, the present disclosure provides a humanized monoclonal anti-Tau antibody provided at a dose of 4500 mg for use in maintaining cognitive capacity no more than 5 points higher than an Alzheimer's disease Assessment Scale, Cognitive Subscale, 11-item version (ADAS-Cog11) score of a patient diagnosed with moderate AD, after administration of 12 to 17 doses, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

In another aspect, the present disclosure provides a humanized monoclonal anti-Tau antibody provided at a dose of 4500 mg for use in slowing memory decline in a patient diagnosed with mild-to-moderate AD, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

In another aspect, the present disclosure provides a humanized monoclonal anti-Tau antibody provided at a dose of 4500 mg for use in maintaining memory within 2.5 points of an Alzheimer's disease Assessment Scale, Cognitive Subscale, 11-item version (ADAS-Cog11) memory domain score of a patient diagnosed with mild-to-moderate AD, after administration of 12 to 17 doses, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

In another aspect, the present disclosure provides a humanized monoclonal anti-Tau antibody provided at a dose of 4500 mg for use in slowing memory decline in a patient diagnosed with moderate AD, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

In another aspect, the present disclosure provides a humanized monoclonal anti-Tau antibody provided at a dose of 4500 mg for use in maintaining memory within 2.5 points of an Alzheimer's disease Assessment Scale, Cognitive Subscale, 11-item version (ADAS-Cog11) memory domain score of a patient diagnosed with moderate AD, after administration of 12 to 17 doses, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

In another aspect, the present disclosure provides a humanized monoclonal anti-Tau antibody provided at a dose of 4500 mg for use in slowing decline in language capacity in a patient diagnosed with mild-to-moderate AD, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

In another aspect, the present disclosure provides a humanized monoclonal anti-Tau antibody provided at a dose of 4500 mg for use in slowing decline in language capacity in a patient diagnosed with moderate AD, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

In another aspect, the present disclosure provides a humanized monoclonal anti-Tau antibody provided at a dose of 4500 mg for use in slowing decline in praxis capacity in a patient diagnosed with mild-to-moderate AD, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

In another aspect, the present disclosure provides a humanized monoclonal anti-Tau antibody provided at a dose of 4500 mg for use in slowing decline in praxis capacity in a patient diagnosed with moderate AD, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

In another aspect, the present disclosure provides a humanized monoclonal anti-Tau antibody provided at a dose of 4500 mg for use in treating a patient diagnosed with mild-to-moderate AD without increased risk of an adverse event, optionally wherein the dose is repeated for 12 to 17 doses, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

In another aspect, the present disclosure provides a humanized monoclonal anti-Tau antibody provided at a dose of 4500 mg for use in treating a patient diagnosed with moderate AD without increased risk of an adverse event, optionally wherein the dose is repeated for 12 to 17 doses, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

In another aspect, the present disclosure provides use of a humanized monoclonal anti-Tau antibody for the manufacture of a medicament for slowing decline in cognitive capacity in a patient diagnosed with mild-to-moderate AD, wherein the anti-Tau antibody is formulated to be provided at a dose of 4500 mg and comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

In another aspect, the present disclosure provides use of a humanized monoclonal anti-Tau antibody for the manufacture of a medicament for maintaining cognitive capacity no more than 5 points higher than an Alzheimer's disease Assessment Scale, Cognitive Subscale, 11-item version (ADAS-Cog11) score of a patient diagnosed with mild-to-moderate AD, following administration of 12 to 17 doses, wherein the anti-Tau antibody is formulated to be provided at a dose of 4500 mg and comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

In another aspect, the present disclosure provides use of a humanized monoclonal anti-Tau antibody for the manufacture of a medicament for slowing decline in cognitive capacity in a patient diagnosed with moderate AD, wherein the anti-Tau antibody is formulated to be provided at a dose of 4500 mg and comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

In another aspect, the present disclosure provides use of a humanized monoclonal anti-Tau antibody for the manufacture of a medicament for maintaining cognitive capacity no more than 5 points higher than an Alzheimer's disease Assessment Scale, Cognitive Subscale, 11-item version (ADAS-Cog11) score of a patient diagnosed with moderate AD, following administration of 12 to 17 doses, wherein the anti-Tau antibody is formulated to be provided at a dose of 4500 mg and comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

In another aspect, the present disclosure provides use of a humanized monoclonal anti-Tau antibody for the manufacture of a medicament for slowing memory decline in a patient diagnosed with mild-to-moderate AD, wherein the antibody is formulated to be provided at a dose of 4500 mg and comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

In another aspect, the present disclosure provides use of a humanized monoclonal anti-Tau antibody for the manufacture of a medicament for maintaining memory within 2.5 points of an Alzheimer's disease Assessment Scale, Cognitive Subscale, 11-item version (ADAS-Cog11) memory domain score of a patient diagnosed with mild-to-moderate AD, following administration of 12 to 17 doses, wherein the anti-Tau antibody is formulated to be provided at a dose of 4500 mg and comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

In another aspect, the present disclosure provides use of a humanized monoclonal anti-Tau antibody for the manufacture of a medicament for slowing memory decline in a patient diagnosed with moderate AD, wherein the antibody is formulated to be provided at a dose of 4500 mg and comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

In another aspect, the present disclosure provides use of a humanized monoclonal anti-Tau antibody for the manufacture of a medicament for maintaining memory within 2.5 points of an Alzheimer's disease Assessment Scale, Cognitive Subscale, 11-item version (ADAS-Cog11) memory domain score of a patient diagnosed with moderate AD, following administration of 12 to 17 doses, wherein the anti-Tau antibody is formulated to be provided at a dose of 4500 mg and comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

In another aspect, the present disclosure provides use of a humanized monoclonal anti-Tau antibody for the manufacture of a medicament for slowing decline in language capacity in a patient diagnosed with mild-to-moderate AD, wherein the antibody is formulated to be provided at a dose of 4500 mg and comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

In another aspect, the present disclosure provides use of a humanized monoclonal anti-Tau antibody for the manufacture of a medicament for slowing decline in language capacity in a patient diagnosed with moderate AD, wherein the antibody is formulated to be provided at a dose of 4500 mg and comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

In another aspect, the present disclosure provides use of a humanized monoclonal anti-Tau antibody for the manufacture of a medicament for slowing decline in praxis capacity in a patient diagnosed with mild-to-moderate AD, wherein the antibody is formulated to be provided at a dose of 4500 mg and comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

In another aspect, the present disclosure provides use of a humanized monoclonal anti-Tau antibody for the manufacture of a medicament for slowing decline in praxis capacity in a patient diagnosed with moderate AD, wherein the antibody is formulated to be provided at a dose of 4500 mg and comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

In another aspect, the present disclosure provides use of a humanized monoclonal anti-Tau antibody for the manufacture of a medicament for treating a patient diagnosed with mild-to-moderate AD without increased risk of an adverse event, wherein the anti-Tau antibody is formulated to be provided at a dose of 4500 mg and comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

In another aspect, the present disclosure provides use of a humanized monoclonal anti-Tau antibody for the manufacture of a medicament for treating a patient diagnosed with moderate AD without increased risk of an adverse event, wherein the anti-Tau antibody is formulated to be provided at a dose of 4500 mg and comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

In some embodiments, the adverse event of the method, the anti-Tau antibody for use, or the use of this disclosure is at least one or more selected from the group consisting of: an infusion-related reaction, a neuroimaging abnormality, immunogenicity; suicide ideation, headache, worsening cognitive function, alteration of consciousness, seizures, unsteadiness, vomiting, a fall, urinary tract infection, anxiety, headache, agitation, depression, dizziness, diarrhea, hypertension, nasopharyngitis, arthralgia, constipation, COVID-19, insomnia, upper respiratory tract infection, abdominal pain, back pain, cough, hematuria, nausea, extremity pain, anemia, confused state, and hallucination.

In some embodiments, the Tau PET tracer of the method, the anti-Tau antibody for use, or the use of this disclosure is administered to the patient before and/or after administration of the antibody, does not increase the risk of an adverse event.

In some embodiments, the patient of the method, the anti-Tau antibody for use, or the use of this disclosure has a Mini-Mental State Exam (MMSE) score of 16-19, inclusive, before administration of said antibody, optionally an MMSE of 16-18, inclusive, before administration of said antibody.

In some embodiments, the patient of the method, the anti-Tau antibody for use, or the use of this disclosure has a Clinical Dementia Rating Global Score (CDR-GS) of 1 or 2 before administration of said antibody.

In some embodiments, the dose of the method, the anti-Tau antibody for use, or the use of this disclosure is repeated at least 5 times, at least 8 times, or at least 10 times, or the dose is repeated for 5-17 doses, 10-17 doses, or 12-17 doses. In some embodiments, the dose of the method, the anti-Tau antibody for use, or the use of this disclosure is repeated for 13-15 doses, 13-14 doses, 14-15 doses, or 14 doses. In some embodiments, the dose of the method, the anti-Tau antibody for use, or the use of this disclosure is repeated for 12-16 doses. In some embodiments, the dose of the method, the anti-Tau antibody for use, or the use of this disclosure is repeated for 14-17 doses

In some embodiments, the antibody of the method, the anti-Tau antibody for use, or the use of this disclosure is administered for at least 24 weeks, optionally at least once every 4 weeks (or monthly). In some embodiments, the antibody of the method, the anti-Tau antibody for use, or the use of this disclosure is administered for at least 36 weeks, optionally at least once every 4 weeks (or monthly).

In some embodiments, the antibody of the method, the anti-Tau antibody for use, or the use of this disclosure is administered for at least 40, 44, 48, 52, 56, 60, 64, 68, 72, 76, 80, 84, 88, 92, 96, 100, 104, 108, 112, 116, 120, 124, 128, 132, 136, 140, 144, 148, 152, 156, 160, 164, or 168 weeks, optionally at least once every 4 weeks (or monthly). In some embodiments, the antibody of the method, the anti-Tau antibody for use, or the use of this disclosure is administered for at least 40, 44, 48, 52, 56, or 60 weeks, optionally at least once every 4 weeks (or monthly).

In some embodiments, the dose of the method, the anti-Tau antibody for use, or the use of this disclosure the antibody is administered at least once every 4 weeks (or monthly) for at least 48 weeks.

In some embodiments, the ADAS-Cog11 score of the method, the anti-Tau antibody for use, or the use of this disclosure is assessed after administration of said antibody to the patient and is no more than 2.5, no more than 3, no more than 3.5, no more than 4, no more than 4.5, or no more than 5 points higher than an ADAS-Cog11 score of the patient assessed before administration of said antibody. In some embodiments, the ADAS-Cog11 score of the method, the anti-Tau antibody for use, or the use of this disclosure is assessed after administration of said antibody to the patient and is no more than 4 points higher than an ADAS-Cog11 score of the patient assessed before administration of said antibody. In some embodiments, the ADAS-Cog11 score of the method, the anti-Tau antibody for use, or the use of this disclosure is assessed after administration of said antibody to the patient and is 2-4 points higher than an ADAS-Cog11 score of the patient assessed before administration of said antibody. In some embodiments, the ADAS-Cog11 score of the method, the anti-Tau antibody for use, or the use of this disclosure is assessed after administration of said antibody to the patient and is 3-4 points higher than an ADAS-Cog11 score of the patient assessed before administration of said antibody.

In some embodiments, the ADAS-Cog11 memory domain score of the method, the anti-Tau antibody for use, or the use of this disclosure, is assessed after administration of said antibody to the patient and is no more than 1, no more than 1.5, no more than 1.7, no more than 2, no more than 2.3, or no more than 2.5 points higher than an ADAS-Cog11 memory domain score of the patient assessed before administration of said antibody. In some embodiments, the ADAS-Cog11 memory domain score of the method, the anti-Tau antibody for use, or the use of this disclosure is assessed after administration of said antibody to the patient and is no more than 2 points higher than an ADAS-Cog11 memory domain score of the patient assessed before administration of said antibody. In some embodiments, the ADAS-Cog11 memory domain score of the method, the anti-Tau antibody for use, or the use of this disclosure is assessed after administration of said antibody to the patient and is 1-2 points higher than an ADAS-Cog11 memory domain score of the patient assessed before administration of said antibody. In some embodiments, the ADAS-Cog11 memory domain score of the method, the anti-Tau antibody for use, or the use of this disclosure is assessed after administration of said antibody to the patient and is 1.5-2.5 points higher than an ADAS-Cog11 memory domain score of the patient assessed before administration of said antibody.

In some embodiments, the antibody of the method, the anti-Tau antibody for use, or the use of this disclosure is administered at least once every 4 weeks (or monthly) for at least 48 weeks.

In some embodiments, the ADAS-Cog11 score (of the patient) of the method, the anti-Tau antibody for use, or the use of this disclosure is assessed after administration of said antibody is reduced by at least 25%, at least 30%, at least 35%, at least 40%, at least 45%, or at least 50% compared to that expected without administration of the antibody. In some embodiments, the ADAS-Cog11 score of the method, the anti-Tau antibody for use, or the use of this disclosure is assessed after administration of said antibody to the patient and is reduced by at least 40% compared to that expected without administration of said antibody. In some embodiments, the ADAS-Cog11 score of the method, the anti-Tau antibody for use, or the use of this disclosure is assessed after administration of said antibody to the patient and is reduced by 25-50% compared to that expected without administration of said antibody. In some embodiments, the ADAS-Cog11 score of the method, the anti-Tau antibody for use, or the use of this disclosure is assessed after administration of said antibody to the patient and is reduced by 40-50% compared to that expected without administration of said antibody.

In some embodiments, the antibody of the method, the anti-Tau antibody for use, or the use of this disclosure is administered to the patient at least once every 4 weeks (or monthly) for at least 48 weeks.

In some embodiments, the method, the anti-Tau antibody for use, or the use of this disclosure comprises administering the antibody once every two weeks, every three weeks, or every four weeks. In some embodiments, the method, the anti-Tau antibody for use, or the use of this disclosure comprises administering the antibody once every two weeks for one to five doses, and then once every four weeks (or once monthly). In some embodiments, the method, the anti-Tau antibody for use, or the use of this disclosure comprises administering the antibody once every two weeks for three doses, and then once every four weeks (or once monthly).

In some embodiments, the method, the anti-Tau antibody for use, or the use of this disclosure comprises administering the antibody intravenously.

In some embodiments, the administration of the method, the anti-Tau antibody for use, or the use of this disclosure occurs at an infusion rate of 0.5 to 3.0 mL/minute. In some embodiments, the administration of the method, the anti-Tau antibody for use, or the use of this disclosure occurs at an infusion rate of 0.5-3.0 mL/minute, every four weeks (or monthly).

In some embodiments, the infusion rate of the method, the anti-Tau antibody for use, or the use of this disclosure is 0.5-1 mL/min, optionally for 10-120 minutes of a first infusion; and 3 mL/minute thereafter.

In some embodiments, the antibody of the method, the anti-Tau antibody for use, or the use of this disclosure is an IgG4 antibody.

In some embodiments, the antibody of the method, the anti-Tau antibody for use, or the use of this disclosure comprises M252Y, S254T, and T256E mutations, according to EU numbering.

In some embodiments, the antibody of the method, the anti-Tau antibody for use, or the use of this disclosure comprises an S228P mutation, according to EU numbering.

In some embodiments, the antibody of the method, the anti-Tau antibody for use, or the use of this disclosure comprises a heavy chain variable region comprising an amino acid sequence that is at least 95% identical to the sequence of SEQ ID NO: 5; and/or a light chain variable region comprising an amino acid sequence that is at least 95% identical to the sequence of SEQ ID NO: 9. In some embodiments, the antibody of the method, the anti-Tau antibody for use, or the use of this disclosure comprises a heavy chain variable region having the amino acid sequence of SEQ ID NO: 5 and/or a light chain variable region having the amino acid sequence of SEQ ID NO: 9.

In some embodiments, the antibody of the method, the anti-Tau antibody for use, or the use of this disclosure is semorinemab.

In some embodiments, the patient of the method, the anti-Tau antibody for use, or the use of this disclosure is Apoε4 positive.

In some embodiments, the patient of the method, the anti-Tau antibody for use, or the use of this disclosure is Apoε4 negative.

In some embodiments, the patient of the method, the anti-Tau antibody for use, or the use of this disclosure has an MMSE score of 19-21 before administration of the antibody.

In some embodiments, the patient of the method, the anti-Tau antibody for use, or the use of this disclosure has an MMSE score of 16-19, inclusive, before administration of the antibody, optionally MMSE score of 16-18, inclusive, before administration of the antibody.

In some embodiments, the patient of the method, the anti-Tau antibody for use, or the use of this disclosure is Tau positive and/or amyloid beta (Abeta) positive, optionally wherein the patient is determined to be Tau positive by administering to the patient a positron emission tomography (PET) tracer that binds to Tau and optionally wherein the patient is determined to be Abeta positive by administering to the patient a PET tracer that binds to Abeta.

In some embodiments, the level of Tau of the method, the anti-Tau antibody for use, or the use of this disclosure is measured by a standardized uptake value ratio (SUVR) measurement of a scan that shows distribution of the PET tracer in the patient's brain.

In some embodiments, the patient of the method, the anti-Tau antibody for use, or the use of this disclosure has a high level of Tau, wherein the high level of Tau corresponds to one or more of:
(i) an intracerebral Tau level above or equal to median Genentech Tau Probe 1 (GTP1) whole cortical gray (WCG) (top medium split);
(ii) an SUVR measurement from the temporal region that is equal to or greater than 1.325; and
(iii) an SUVR measurement from the whole cortical gray (WCG) region that is equal to or greater than 1.245.

In some embodiments, the patient of the method, the anti-Tau antibody for use, or the use of this disclosure has a low level of Tau, wherein the low level of Tau corresponds to one or more of:
(i) an intracerebral Tau level below median GTP1 WCG (bottom medium split);
(ii) an SUVR measurement from the temporal region that is less than 1.325; and
(iii) an SUVR measurement from the WCG that is less than 1.245.

In some embodiments, the PET tracer that binds to Tau of the method, the anti-Tau antibody for use, or the use of this disclosure is at least one selected from the group consisting of [¹⁸F] Genentech Tau Probe 1 ([¹⁸F]GTP1), RO-948, AV-1451 (Flortaucipir), PI-2014, PI-2620, MK-6240, and T-808 and the PET tracer that binds to Abeta of the method, the anti-Tau antibody for use, or the use of this disclosure is at least one selected from the group consisting of florbetapir, florebetaben, and flutemetamol.

In some embodiments, the Tau of the method, the anti-Tau antibody for use, or the use of this disclosure is measured in a CSF sample or a plasma sample taken from the patient.

In some embodiments, the patient of the method, the anti-Tau antibody for use, or the use of this disclosure is co-administered one or more additional agents.

In some embodiments, the one or more additional agents of the method, the anti-Tau antibody for use, or the use of this disclosure are selected from the group consisting of: a symptomatic medication, a neurological drug, a corticosteroid, an antibiotic, an antiviral agent, an additional anti-Tau antibody, a Tau inhibitor, an anti-amyloid beta antibody, a beta-amyloid aggregation inhibitor, an anti-BACE1 antibody, a BACE1 inhibitor; a cholinesterase inhibitor; an NMDA receptor antagonist; a monoamine depletor; an ergoloid mesylate; an anticholinergic antiparkinsonism agent; a dopaminergic antiparkinsonism agent; a tetrabenazine; an anti-inflammatory agent; a hormone; a vitamin; a dimebolin; a homoTaurine; a serotonin receptor activity modulator; an interferon, and a glucocorticoid.

In some embodiments, the symptomatic medication of the method, the anti-Tau antibody for use, or the use of this disclosure is selected from the group consisting of a cholinesterase inhibitor, galantamine, rivastigmine, donepezil, an N-methyl-D-aspartate receptor antagonist, memantine, and a food supplement (optionally wherein the food supplement is Souvenaid®).

In some embodiments, the anti-amyloid beta antibody of the method, the anti-Tau antibody for use, or the use of this disclosure is aducanemab, lecanemab, or donanemab.

In some embodiments, the anti-amyloid beta antibody of the method, the anti-Tau antibody for use, or the use of this disclosure is crenezumab or gantenerumab.

In some embodiments, the additional anti-Tau antibody of the method, the anti-Tau antibody for use, or the use of this disclosure is selected from the group consisting of a different N-terminal binder, a mid-domain binder, and a fibrillar Tau binder.

In some embodiments, the additional anti-Tau antibody of the method, the anti-Tau antibody for use, or the use of this disclosure is selected from the group consisting of Gosuranemab, Tilavonemab, Bepranemab, and Zagotenemab.

In some embodiments, the one or more additional agents of the method, the anti-Tau antibody for use, or the use of this disclosure comprises a therapeutic agent that specifically binds to a target selected from the group consisting of beta secretase, Tau, presenilin, amyloid precursor protein or portions thereof, amyloid beta peptide or oligomers or fibrils thereof, death receptor 6 (DR6), receptor for advanced glycation end-products (RAGE), parkin, and huntingtin.

In some embodiments, the monoamine depletor of the method, the anti-Tau antibody for use, or the use of this disclosure is tetrabenazine.

In some embodiments, the anticholinergic antiparkinsonism agent o of the method, the anti-Tau antibody for use, or the use of this disclosure is selected from the group consisting of procyclidine, diphenhydramine, trihexylphenidyl, benztropine, biperiden and trihexyphenidyl.

In some embodiments, the dopaminergic antiparkinsonism agent of the method, the anti-Tau antibody for use, or the use of this disclosure is selected from the group consisting of: entacapone, selegiline, pramipexole, bromocriptine, rotigotine, selegiline, ropinirole, rasagiline, apomorphine, carbidopa, levodopa, pergolide, tolcapone and amantadine.

In some embodiments, the anti-inflammatory agent of the method, the anti-Tau antibody for use, or the use of this disclosure is selected from the group consisting of a nonsteroidal anti-inflammatory drug and indomethacin.

In some embodiments, the hormone of the method, the anti-Tau antibody for use, or the use of this disclosure is selected from the group consisting of estrogen, progesterone, and leuprolide.

In some embodiments, the vitamin of the method, the anti-Tau antibody for use, or the use of this disclosure is selected from the group consisting of folate and nicotinamide.

In some embodiments, the homoTaurine of the method, the anti-Tau antibody for use, or the use of this disclosure is 3-aminopropanesulfonic acid or 3APS.

In some embodiments, the serotonin receptor activity modulator of the method, the anti-Tau antibody for use, or the use of this disclosure is xaliproden.

In some embodiments, the administration of the antibody of the method, the anti-Tau antibody for use, or the use of this disclosure does not increase the risk of an adverse event.

In some embodiments, the adverse event of the method, the anti-Tau antibody for use, or the use of this disclosure is at least one selected from the group consisting of: an infusion-related reaction, a neuroimaging abnormality, immunogenicity; suicide ideation, headache, worsening cognitive function, alteration of consciousness, seizures, unsteadiness, vomiting, a fall, urinary tract infection, anxiety, headache, agitation, depression, dizziness, diarrhea, hypertension, nasopharyngitis, arthralgia, constipation, COVID-19, insomnia, upper respiratory tract infection, abdominal pain, back pain, cough, hematuria, nausea, extremity pain, anemia, confused state, and hallucination.

In some embodiments, the patient of the method, the anti-Tau antibody for use, or the use of this disclosure is Black or Hispanic or has a non-European ethnic origin.

In another aspect, the present disclosure provides a method of slowing decline in cognitive capacity in a patient diagnosed with mild-to-moderate AD, the method comprising intravenously administering to the patient a 4500 mg dose of semorinemab at a frequency of Q2W for 3 doses and subsequently at a frequency of Q4W.

In another aspect, the present disclosure provides a method of maintaining cognitive capacity within 5 points of an ADAS-Cog11 score of a patient diagnosed with mild-to-moderate AD, the method comprising intravenously administering to the patient a 4500 mg dose of semorinemab at a frequency of Q2W for 3 doses and subsequently at a frequency of Q4W.

In another aspect, the present disclosure provides a method of treating a patient diagnosed with mild-to-moderate AD without increased risk of an adverse event, the method comprising intravenously administering to the patient a 4500 mg dose of semorinemab at a frequency of Q2W for 3 doses and subsequently at a frequency of Q4W.

In another aspect, the present disclosure provides a method of slowing memory decline in a patient diagnosed with mild-to-moderate AD, the method comprising intravenously administering to the patient a 4500 mg dose of semorinemab at a frequency of Q2W for 3 doses and subsequently at a frequency of Q4W.

In another aspect, the present disclosure provides a method of maintaining cognitive capacity within 2.5 points of an ADAS-Cog11 score of a patient diagnosed with mild-to-moderate AD, the method comprising intravenously administering to the patient a 4500 mg dose of semorinemab at a frequency of Q2W for 3 doses and subsequently at a frequency of Q4W.

In another aspect, the present disclosure provides a method of slowing decline in language capacity in a patient diagnosed with mild-to-moderate AD, the method comprising intravenously administering to the patient a 4500 mg dose of semorinemab at a frequency of Q2W for 3 doses and subsequently at a frequency of Q4W.

In another aspect, the present disclosure provides a method of slowing decline in praxis capacity in a patient diagnosed with mild-to-moderate AD, the method comprising intravenously administering to the patient a 4500 mg dose of semorinemab at a frequency of Q2W for 3 doses and subsequently at a frequency of Q4W.

In another aspect, the present disclosure provides a method of slowing decline in cognitive capacity in a patient diagnosed with moderate AD, the method comprising intravenously administering to the patient a 4500 mg dose of semorinemab at a frequency of Q2W for 3 doses and subsequently at a frequency of Q4W.

In another aspect, the present disclosure provides a method of maintaining cognitive capacity within 5 points of an ADAS-Cog11 score of a patient diagnosed with moderate AD, the method comprising intravenously administering to the patient a 4500 mg dose of semorinemab at a frequency of Q2W for 3 doses and subsequently at a frequency of Q4W.

In another aspect, the present disclosure provides a method of treating a patient diagnosed with moderate AD without increased risk of an adverse event, the method comprising intravenously administering to the patient a 4500 mg dose of semorinemab at a frequency of Q2W for 3 doses and subsequently at a frequency of Q4W.

In another aspect, the present disclosure provides a method of slowing memory decline in a patient diagnosed with moderate AD, the method comprising intravenously administering to the patient a 4500 mg dose of semorinemab at a frequency of Q2W for 3 doses and subsequently at a frequency of Q4W.

In another aspect, the present disclosure provides a method of maintaining cognitive capacity within 2.5 points of an ADAS-Cog11 score of a patient diagnosed with moderate AD, the method comprising intravenously administering to the patient a 4500 mg dose of semorinemab at a frequency of Q2W for 3 doses and subsequently at a frequency of Q4W.

In another aspect, the present disclosure provides a method of slowing decline in language capacity in a patient diagnosed with moderate AD, the method comprising intravenously administering to the patient a 4500 mg dose of semorinemab at a frequency of Q2W for 3 doses and subsequently at a frequency of Q4W.

In another aspect, the present disclosure provides a method of slowing decline in praxis capacity in a patient diagnosed with moderate AD, the method comprising intravenously administering to the patient a 4500 mg dose of semorinemab at a frequency of Q2W for 3 doses and subsequently at a frequency of Q4W.

In another aspect, the present disclosure provides semorinemab at a dose of 4500 mg for use in slowing decline in cognitive capacity in a patient diagnosed with mild-to-moderate AD, wherein the semorinemab is intravenously administered at a frequency of Q2W for 3 doses and subsequently at a frequency of Q4W.

In another aspect, the present disclosure provides semorinemab at a dose of 4500 mg for use in maintaining cognitive capacity no more than 5 points higher than an ADAS-Cog11 score of a patient diagnosed with mild-to-moderate AD, wherein the semorinemab is intravenously administered at a frequency of Q2W for 3 doses and subsequently at a frequency of Q4W.

In another aspect, the present disclosure provides semorinemab at a dose of 4500 mg for use in treating a patient diagnosed with mild-to-moderate AD without increased (or without significantly increased) risk of an adverse event, wherein the semorinemab is intravenously administered at a frequency of Q2W for 3 doses and subsequently at a frequency of Q4W.

In another aspect, the present disclosure provides semorinemab at a dose of 4500 mg for use in slowing memory decline in a patient diagnosed with mild-to-moderate AD, wherein the semorinemab is intravenously administered at a frequency of Q2W for 3 doses and subsequently at a frequency of Q4W.

In another aspect, the present disclosure provides semorinemab at a dose of 4500 mg for use in maintaining cognitive capacity no more than 2.5 points higher than an ADAS-Cog11 score of a patient diagnosed with mild-to-moderate AD, wherein the semorinemab is intravenously administered at a frequency of Q2W for 3 doses and subsequently at a frequency of Q4W.

In another aspect, the present disclosure provides semorinemab at a dose of 4500 mg for use in slowing decline in language capacity in a patient diagnosed with mild-to-moderate AD, wherein the semorinemab is intravenously administered at a frequency of Q2W for 3 doses and subsequently at a frequency of Q4W.

In another aspect, the present disclosure provides semorinemab at a dose of 4500 mg for use in slowing decline in praxis capacity in a patient diagnosed with mild-to-moderate AD, wherein the semorinemab is intravenously administered at a frequency of Q2W for 3 doses and subsequently at a frequency of Q4W.

In another aspect, the present disclosure provides semorinemab at a dose of 4500 mg for use in slowing decline in cognitive capacity in a patient diagnosed with moderate AD, wherein the semorinemab is intravenously administered at a frequency of Q2W for 3 doses and subsequently at a frequency of Q4W.

In another aspect, the present disclosure provides semorinemab at a dose of 4500 mg for use in maintaining cognitive capacity no more than 5 points higher than an ADAS-Cog11 score of a patient diagnosed with moderate AD, wherein the semorinemab is intravenously administered at a frequency of Q2W for 3 doses and subsequently at a frequency of Q4W.

In another aspect, the present disclosure provides semorinemab at a dose of 4500 mg for use in treating a patient diagnosed with moderate AD without increased (or without significantly increased) risk of an adverse event, wherein the semorinemab is intravenously administered at a frequency of Q2W for 3 doses and subsequently at a frequency of Q4W.

In another aspect, the present disclosure provides semorinemab at a dose of 4500 mg for use in slowing memory decline in a patient diagnosed with moderate AD, wherein the semorinemab is intravenously administered at a frequency of Q2W for 3 doses and subsequently at a frequency of Q4W.

In another aspect, the present disclosure provides semorinemab at a dose of 4500 mg for use in maintaining cognitive capacity no more than 2.5 points higher than an ADAS-Cog11 score of a patient diagnosed with moderate AD, wherein the semorinemab is intravenously administered at a frequency of Q2W for 3 doses and subsequently at a frequency of Q4W.

In another aspect, the present disclosure provides semorinemab at a dose of 4500 mg for use in slowing decline in language capacity in a patient diagnosed with moderate AD, wherein the semorinemab is intravenously administered at a frequency of Q2W for 3 doses and subsequently at a frequency of Q4W.

In another aspect, the present disclosure provides semorinemab at a dose of 4500 mg for use in slowing decline in praxis capacity in a patient diagnosed with moderate AD, wherein the semorinemab is intravenously administered at a frequency of Q2W for 3 doses and subsequently at a frequency of Q4W.

In another aspect, the present disclosure provides use of semorinemab for the manufacture of a medicament for slowing decline in cognitive capacity in a patient diagnosed with mild-to-moderate AD, wherein the semorinemab is formulated for intravenous administration at a dose of 4500 mg, wherein the medicament is administered at a frequency of Q2W for 3 doses and subsequently at a frequency of Q4W.

In another aspect, the present disclosure provides use of semorinemab for the manufacture of a medicament for maintaining cognitive capacity no more than 5 points higher than an ADAS-Cog11 score of a patient diagnosed with mild-to-moderate AD, wherein the semorinemab is formulated for intravenous administration at a dose of 4500 mg, wherein the medicament is formulated to be administered at a frequency of Q2W for 3 doses and subsequently at a frequency of Q4W.

In another aspect, the present disclosure provides use of semorinemab for the manufacture of a medicament for treating a patient diagnosed with mild-to-moderate AD without increased (or without significantly increased) risk of an adverse event, wherein the semorinemab is formulated for intravenous administration at a dose of 4500 mg, wherein the medicament is formulated to be administered at a frequency of Q2W for 3 doses and subsequently at a frequency of Q4W.

In another aspect, the present disclosure provides use of semorinemab for the manufacture of a medicament for slowing memory decline in a patient diagnosed with mild-to-moderate AD, wherein the semorinemab is formulated for intravenous administration at a dose of 4500 mg, wherein the medicament is formulated to be administered at a frequency of Q2W for 3 doses and subsequently at a frequency of Q4W.

In another aspect, the present disclosure provides use of semorinemab for the manufacture of a medicament for maintaining cognitive capacity no more than 2.5 points higher than an ADAS-Cog11 score of a patient diagnosed with mild-to-moderate AD, wherein the semorinemab is formulated for intravenous administration at a dose of 4500 mg, wherein the medicament is formulated to be administered at a frequency of Q2W for 3 doses and subsequently at a frequency of Q4W.

In another aspect, the present disclosure provides use of semorinemab for the manufacture of a medicament for slowing decline in language capacity in a patient diagnosed with mild-to-moderate AD, wherein the semorinemab is formulated for intravenous administration at a dose of 4500 mg, wherein the medicament is formulated to be administered at a frequency of Q2W for 3 doses and subsequently at a frequency of Q4W.

In another aspect, the present disclosure provides use of semorinemab for the manufacture of a medicament for slowing decline in praxis capacity in a patient diagnosed with mild-to-moderate AD, wherein the semorinemab is formulated for intravenous administration at a dose of 4500 mg, wherein the medicament is formulated to be administered at a frequency of Q2W for 3 doses and subsequently at a frequency of Q4W.

In another aspect, the present disclosure provides use of semorinemab for the manufacture of a medicament for slowing decline in cognitive capacity in a patient diagnosed with moderate AD, wherein the semorinemab is formulated for intravenous administration at a dose of 4500 mg, wherein the medicament is administered at a frequency of Q2W for 3 doses and subsequently at a frequency of Q4W.

In another aspect, the present disclosure provides use of semorinemab for the manufacture of a medicament for maintaining cognitive capacity no more than 5 points higher than an ADAS-Cog11 score of a patient diagnosed with moderate AD, wherein the semorinemab is formulated for intravenous administration at a dose of 4500 mg, wherein the medicament is formulated to be administered at a frequency of Q2W for 3 doses and subsequently at a frequency of Q4W.

In another aspect, the present disclosure provides use of semorinemab for the manufacture of a medicament for treating a patient diagnosed with moderate AD without increased (or without significantly increased) risk of an adverse event, wherein the semorinemab is formulated for intravenous administration at a dose of 4500 mg, wherein the medicament is formulated to be administered at a frequency of Q2W for 3 doses and subsequently at a frequency of Q4W.

In another aspect, the present disclosure provides use of semorinemab for the manufacture of a medicament for slowing memory decline in a patient diagnosed with moderate AD, wherein the semorinemab is formulated for intravenous administration at a dose of 4500 mg, wherein the medicament is formulated to be administered at a frequency of Q2W for 3 doses and subsequently at a frequency of Q4W.

In another aspect, the present disclosure provides use of semorinemab for the manufacture of a medicament for maintaining cognitive capacity no more than 2.5 points higher than an ADAS-Cog11 score of a patient diagnosed with moderate AD, wherein the semorinemab is formulated for intravenous administration at a dose of 4500 mg, wherein the medicament is formulated to be administered at a frequency of Q2W for 3 doses and subsequently at a frequency of Q4W.

In another aspect, the present disclosure provides use of semorinemab for the manufacture of a medicament for slowing decline in language capacity in a patient diagnosed with moderate AD, wherein the semorinemab is formulated for intravenous administration at a dose of 4500 mg, wherein the medicament is formulated to be administered at a frequency of Q2W for 3 doses and subsequently at a frequency of Q4W.

In another aspect, the present disclosure provides use of semorinemab for the manufacture of a medicament for slowing decline in praxis capacity in a patient diagnosed with moderate AD, wherein the semorinemab is formulated for intravenous administration at a dose of 4500 mg, wherein the medicament is formulated to be administered at a frequency of Q2W for 3 doses and subsequently at a frequency of Q4W.

In some embodiments, the semorinemab of the method, the semorinemab for use, or the use of the present disclosure is administered at a frequency of Q4W for at least 10 doses. In some embodiments, the semorinemab of the method, the semorinemab for use, or the use of the present disclosure is administered at a frequency of Q4W for at least 13 doses. In some embodiments, the semorinemab of the method, the semorinemab for use, or the use of the present disclosure is administered at a frequency of Q4W for at least 16 doses.

In some embodiments, the semorinemab of the method, the semorinemab for use, or the use of the present disclosure is administered at an infusion rate of 0.5 mL/min to 3.0 mL/min.

In some embodiments, the infusion rate of the method, the semorinemab for use, or the use of the present disclosure is 0.5 mL/min to 1 mL/min, optionally for 10-120 minutes of a first infusion; and 3 mL/minute thereafter.

In some embodiments, the method, the semorinemab for use, or the use of the present disclosure further comprises intravenously administering to the patient a 4500 mg dose of semorinemab at a frequency of Q4W for 96 weeks.

### BRIEF DESCRIPTION OF THE DRAWINGS

This patent or patent application file contains at least one drawing executed in color. Copies of this patent or patent application publication with color drawings will be provided by the Office upon request and payment of the necessary fee.

FIGS. 1A and 1B provide schematics, showing the overall study design, including a screening period, a double-blind treatment period, an optional open-label extension (OLE) period, and a safety follow up period. Due to COVID-19, blinded period was extended to 60 weeks for participants who missed one or more dose of study drug; participants were given a supplemental Q2W dose if there were ≥2 consecutive missed study drug infusions. Q2W refers to every 2 weeks and Q4W refers to every 4 weeks. ^{a}refers to extensions to the 8-week screening period may be granted on a case-by-case basis by contacting the Medical Monitor. ^{b}refers to extensions to the 15-day baseline visit period may be granted on a case-by-case basis by contacting the Medical Monitor. ° refers to for Cohort 2 and Cohort 3, if two or more consecutive study drug infusions are missed during the double-blind treatment period, study drug administration will be re-initiated with Q2W dosing for the next three doses, followed by Q4W dosing thereafter.

FIG. 2 depicts study cohort assignment. DBP refers to double-blind period. OLE refers to open-label extension. V3 refers to Version 3.

FIG. 3 is a schematic, showing randomization of study participants.

FIGS. 4A-4B is a schematic showing the dosing and frequency for Cohort 1 (FIG. 4A) and Cohort 2 (FIG. 4B) during the double-blind treatment period.

FIG. 5A depicts amino acid sequences of the 6 HVR's of semorinemab; FIG. 5B depicts amino acid sequences of the VH and VL domains of semorinemab.

FIG. 6 depicts adjusted mean change (with 95% CI) in ADAS-Cog11 over time for patients with ADAS-Cog11 at baseline and at least one post-baseline value who missed a maximum of one semorinemab dose. MITT population is defined using ADAS-Cog11 for all endpoints. Estimates are from analysis based on mixed-effect model of repeated measures (MMRM) using an unstructured covariance matrix: change=baseline+age+Apoε+DS results+Baseline concomitant medication+Analysis visit+Treatment+Treatment*Analysis Visit+Analysis Visit*Baseline (repeated values over Analysis baseline correspond to disease worsening). Descriptive statistics at baseline include patients with ADAS-Cog11 at baseline and at least one post-baseline value. mITT population is defined using ADAS-Cog11 for all endpoints. Estimates are from analysis based on mixed-effect model of repeated measures (MMRM) using an unstructured covariance matrix: Change=Baseline+Age+Apoε4+DS result+Baseline conmed+Analysis Visit+Treatment+Treatment*Analysis Visit+Analysis Visit*Baseline (repeated value over Analysis Visit). Unadjusted p-values for pairwise comparisons with the control group are presented. An increase in the endpoint from baseline corresponds to disease worsening. Rave data extract: Aug. 2, 2021. Clinical data cut-off is: Jun. 12, 2021.

FIGS. 7A-7B depict change in ADAS-Cog11 over time in two different populations. An increase in the endpoint from baseline corresponds to disease worsening. Rave data extract: Aug. 2, 2021. Clinical data cut-off is: Jun. 12, 2021. FIG. 7A depicts the adjusted mean plot (with 95% CI) from the Mixed-Effect repeated measures model that analyzes the change from baseline of ADAS-Cog11 in the Double Blind Period. Patients are Modified Intent to Treat (MITT) with max one missed dose and who completed their week 49 ADAS-Cog assessments. Max 1 missed dose, week 49 completers. Adjusted Mean Plot (with 95% CI) from Mixed-Effect repeated measures model analysis of change from baseline, ADAS-Cog11, Double-Blind Period, Max one dose missed flag, MITT Patients, Week 49 Completers. FIG. 7B depicts the adjusted mean plot (with 95% CI) from the Mixed-Effect repeated measures model that analyzes the change from baseline of ADAS-Cog11 in the Double Blind Period, among the Modified Intent to Treat (MITT) Patients. Adjusted Mean Plot (with 95% CI) from Mixed-Effect repeated measures model analysis of change from baseline, ADAS-Cog11, Double-Blind Period, MITT Patients.

FIGS. 8A-8B depict adjusted mean change (with 95% CI) in ADAS-Cog11 overtime in study cohorts. An increase in the endpoint from baseline corresponds to disease worsening. Rave data extract: Aug. 2, 2021. Clinical data cut-off is: Jun. 12, 2021. FIG. 8A depicts the adjusted mean plot (with 95% CI) from the Mixed-Effect repeated measures model that analyzes the change from baseline of ADAS-Cog11, in the Double-Blind Period, for Modified Intent to Treat (MITT) Patients, who were assigned to the Actual study cohort 1. Adjusted Mean Plot (with 95% CI) from Mixed-Effect repeated measures model analysis of change from baseline, ADAS-Cog11, Double-Blind Period, MITT Patients, Actual study cohort 1. FIG. 8B depicts the adjusted mean plot (with 95% CI) from the Mixed-Effect repeated measures model that analyzes the change from baseline of ADAS-Cog11, in the Double-Blind Period, for Modified Intent to Treat (MITT) Patients, who were assigned to the Actual study cohort 2. Adjusted Mean Plot (with 95% CI) from Mixed-Effect repeated measures model analysis of change from baseline, ADAS-Cog11, Double-Blind Period, MITT Patients, Actual study cohort 2.

FIG. 9 depicts the cumulative probability of a certain level of change from baseline to week 49 of ADAS-Cog11, in the semorinemab and the placebo arms, for the MITT population.

FIGS. 10A-10B depict consistent differences between subgroups of semorinemab versus placebo treated patients. FIG. 10A depicts a forest plot showing the differences of adjusted means from the Mixed-Effect repeated measures model that analyzes the change from baseline to week 49 of ADAS-Cog11 for Modified Intent to Treat (MITT) subjects who missed maximum one dose. Pre-specified subgroups included high or low GTP1 (where high GTP1 is defined as above or equal to median GTP1 WCG and low GTP1 is defined as below median GTP1 WCG); MMSE 16-18 or MMSE 19-21; and Apoε4 positive or Apoε4 negative. Difference in Adjusted Means versus Placebo: Forest Plot from Mixed-Effect repeated measures model analysis from change from baseline. ADAS-Cog11, Double-Blind Period, Max one dose missed flag, MITT Patients. An increase in the endpoint from baseline corresponds to disease worsening. Rave data extract: Aug. 2, 2021. Clinical data cut-off is: Jun. 12, 2021.

FIG. 10B compares ADAS-Cog11 results with ADCS-ADL results, further depicting forest plots showing the differences of adjusted means from the Mixed-Effect repeated measures model that analyzes the change from baseline to week 49 of ADAS-Cog11, or ADCS-ADL, for MITT subjects who missed maximum one dose, for the different pre-specified subgroups.

FIGS. 11A-11B depict primary end point results for the mITT population. FIG. 11A depicts ADAS-Cog11 adjusted change from baseline over time in placebo (grey circles) and Semorinemab (green circles) arms. FIG. 11B depicts ADCS-ADL adjusted change from baseline over time in placebo (grey circles) and Semorinemab (green circles) arms. The graphs are depicted as slowing downwards from baseline values, for ease of comparison.

FIGS. 12A-12C depict treatment effects in different cognitive domains (i.e., memory, language, praxis) within the ADAS-Cog11 (as defined as by Verma et al., Alzheimer's Research and Therapy, 2015) for the mITT population over time (weeks post-baseline), that is, depicting ADAS-Cog11 cognitive domain analysis, including memory, language and praxis domains. The results show a primarily memory domain-driven treatment effect in ADAS-Cog11 for the mITT population over time (weeks post-baseline), as well as a small effect in the language domain. FIG. 12A depicts unadjusted change from baseline for the ADAS-Cog11 memory domain over time; FIG. 12B depicts unadjusted change from baseline for the ADAS-Cog11 language domain over time; FIG. 12C depicts unadjusted change from baseline for the ADAS-Cog11 praxis domain over time.

FIGS. 13A-13D depict secondary endpoint results in mITT (MMSE and CDR-SB). FIG. 13A depicts MMSE adjusted change from baseline over time (weeks post-baseline) in the placebo (grey circles) and Semorinemab (green circles) arms. FIG. 13B depicts the Forest plot from the Mixed-Effect repeated measures model that analyzes the change from baseline in MMSE for different pre-specified subgroups (high or low GTP1 (where high GTP1 is defined as above or equal to median GTP1 WCG and low GTP1 is defined as below median GTP1 WCG); MMSE 16-18 or MMSE 19-21; and Apoε4 positive or Apoε4 negative). FIG. 13C depicts CDR-SB (inverse) adjusted change from baseline over time (weeks post-baseline) in the placebo (grey circles) and Semorinemab (green circles) arms. FIG. 13D depicts the Forest plot from the Mixed-Effect repeated measures model that analyzes the change from baseline in CDR-SB for the different pre-specified subgroups (high or low GTP1 (where high GTP1 is defined as above or equal to median GTP1 WCG and low GTP1 is defined as below median GTP1 WCG); MMSE 16-18 or MMSE 19-21; and Apoε4 positive or Apoε4 negative).

FIGS. 14A-14G depict no significant effect in Tau accumulation in mild-to-moderate AD and no significant differences in regional analysis. The y-axis is “annualized” so both week 49 and week 61 time-points are used. FIG. 14A depicts unadjusted annualized change from baseline in [¹⁸F]GTP1 tau PET signal of the Whole Cortical Grey region for the placebo (grey) or Semorinemab (green) arms. FIG. 14B shows whole cortical gray region in red, illustrating the location of the whole cortical gray region. FIG. 14C depicts unadjusted annualized change from baseline in [¹⁸F]GTP1 tau PET signal of the frontal region for the placebo (grey) or Semorinemab (green) arms. FIG. 14D depicts unadjusted annualized change from baseline in [¹⁸F]GTP1 tau PET signal of the temporal region for the placebo (grey) or Semorinemab (green) arms. FIG. 14E depicts the unadjusted annualized change from baseline in [¹⁸F]GTP1 tau PET signal of the parietal region for the placebo (grey) or Semorinemab (green) arms. FIG. 14F depicts unadjusted annualized change from baseline in [¹⁸F]GTP1 tau PET signal of the occipital region for the placebo (grey) or Semorinemab (green) arms. FIG. 14G illustrates ROI based on anatomical atlas Hammers template.

FIGS. 15A-15B depict serum pharmacokinetics and plasma pharmacodynamics. FIG. 15A depicts Semorinemab concentration in serum (μg/mL) over time (days), giving a CSF/serum ratio in line with that observed of other monoclonal antibodies. FIG. 15B depicts plasma Tau concentration (pg/mL) over time (days) in the placebo (grey circles) and Semorinemab (green circles) arms, showing support for semorinemab engagement with tau peripherally.

### DETAILED DESCRIPTION OF THE DISCLOSURE

The results of Phase 2 clinical studies in AD patients disclosed herein demonstrate that semorinemab, an N-terminal binding Tau antibody, slows clinical decline and disease progression in patients with mild-to-moderate AD or moderate AD, in particular, slowing the decline in cognitive capacity. Furthermore, the effect is seen in patients having Tau pathology typical of patients diagnosed with mild-to-moderate or moderate AD. Additionally, the results demonstrate that these effects occur without significant incidence of adverse events such as neuroimaging abnormalities or suicide ideation.

The trial used semorinemab, also referred to herein as MTAU9937A or RO7105705, a pan Tau IgG4 monoclonal antibody that is designed to bind and intercept extracellular isoforms of Tau and potentially slow cell-to cell-spread and propagation of Tau pathology throughout cortical and sub cortical networks. Semorinemab targets all currently-known isoforms of full-length tau, with or without post-translational modifications (e.g., phosphorylation).

The IgG4 backbone of semorinemab has reduced Fc-gamma receptor binding affinity compared with the human IgG1 subclass, and thus a reduced immune effector response. Semorinemab has also been engineered to contain three mutations (M249Y, S251T, and T253E [YTE]) in the fragment crystallizable (Fc) region of the heavy chain that enhance binding to the neonatal Fc receptor (FcRn) and have been shown to slow peripheral antibody clearance in humans, potentially augmenting exposure levels (Robbie et al. Antimicrob Agents Chemother 2013; 57:6147-53). In some embodiments, the antibody comprises an IgG4 heavy chain comprising S228P, M252Y, S254T, and T256E mutations, according to EU numbering, and lacking the C-terminal lysine. In some embodiments, the humanized monoclonal anti-Tau antibody of this disclosure is an IgG4 antibody. In some embodiments, the humanized monoclonal anti-Tau antibody of this disclosure comprises M252Y, S254T, and T256E mutations, according to EU numbering. In some embodiments, the humanized monoclonal anti-Tau antibody of this disclosure comprises an S228P mutation, according to EU numbering.

This disclosure provides methods for treating and monitoring patients diagnosed with mild-to-moderate or moderate AD, including Apoε4 positive patients, patients with MMSE scores of 16-21, inclusive, and patients having Tau pathology typically seen in the brains of patients diagnosed with mild-to-moderate or moderate AD. As exemplified herein, a humanized monoclonal anti-Tau antibody has been shown to be effective to significantly reduce decline in cognitive capacity in mild-to-moderate AD, without an increased incidence of adverse events, including neuroimaging abnormalities or suicide ideation. The results demonstrate statistically significant and clinically meaningful reduction in rate of cognitive decline, compared to that expected without the antibody, for AD patients, including patients beyond the early or mild stages of the disease. The present disclosure also demonstrates that 4500 mg is an effective dose and provides a therapeutic benefit after a course of repeat doses.

Accordingly, this disclosure provides therapeutic agents for modulating the progression of AD and improved methods of using the same.

The present disclosure provides methods of treating patients suffering from mild-to-moderate or moderate AD, as well as other related Tau pathologies, comprising administering a humanized monoclonal anti-Tau antibody, or antigen-binding fragment thereof. In some embodiments, the tau pathology is a primary tauopathy. In some embodiments, the tau pathology is a neurodegenerative tauopathy. In some embodiments, the tauopathy is selected from mild-to-moderate AD, moderate AD, amyotrophic lateral sclerosis, Parkinson's disease, Creutzfeldt-Jacob disease, dementia pugilistica, Down's syndrome, Gerstmann-Straussler-Scheinker disease, inclusion-body myositis, prion protein cerebral amyloid angiopathy, traumatic brain injury, amyotrophic lateral sclerosis/parkinsonism-dementia complex of Guam, non-Guamanian motor neuron disease with neurofibrillary tangles, argyrophilic grain dementia, corticobasal degeneration, diffuse neurofibrillary tangles with calcification, frontotemporal dementia, frontotemporal dementia with parkinsonism linked to chromosome 17, Hallevorden-Spatz disease, multiple system atrophy, Niemann-Pick disease type C, pallido-ponto-nigral degeneration, Pick's disease, progressive subcortical gliosis, progressive supranuclear palsy, subacute sclerosing panencephalitis, tangle only dementia, postencephalitic parkinsonism, and myotonic dystrophy. In some embodiments, the tauopathy is progressive supranuclear palsy. In some embodiments, the tauopathy is mild-to-moderate AD. In some embodiments, the tauopathy is moderate AD. In some embodiments, the tauopathy is mild-to-moderate or moderate AD.

In some embodiments, the antibody, or antigen-binding fragment thereof, is capable of binding monomeric, oligomeric, non-phosphorylated, and phosphorylated forms of Tau, for example with a K_{D} of less than 100 nM, less than 75 nM, or less than 50 nM. In some embodiments, the antibody binds an epitope within the N-terminal region of Tau, for example an epitope within amino acid residues 2 to 24 of mature human Tau (e.g., amino acid residues 2 to 24 as set forth in SEQ ID NO:1) and/or an epitope within or spanning amino acid residues 6 to 23 of mature human Tau (e.g., amino acid residues 6 to 23 as set forth in SEQ ID NO:1). In some embodiments, the antibody is a monoclonal antibody. In some embodiments, the antibody is a human, humanized, or chimeric antibody. In some embodiments, the antibody is a humanized antibody. In some embodiments, the antibody is an antibody fragment that binds human Tau. In some embodiments, the human Tau comprises the sequence of SEQ ID NO: 1. In some embodiments, the antibody binds cynomolgus monkey Tau (SEQ ID NO: 10).

In some embodiments, the antibody is an IgG4 antibody. In particular embodiments, the antibody, or antigen-binding fragment thereof, comprises six hypervariable regions (HVRs) wherein HVR-H1 has the amino acid sequence of SEQ ID NO:2, HVR-H2 has the amino acid sequence of SEQ ID NO:3, HVR-H3 has the amino acid sequence of SEQ ID NO:4, HVR-L1 has the amino acid sequence of SEQ ID NO:6, HVR-L2 has the amino acid sequence of SEQ ID NO:7, and HVR-L3 has the amino acid sequence of SEQ ID NO:8. In some embodiments, the antibody, or antigen-binding fragment thereof, comprises a heavy chain variable region (VH) having the amino acid sequence of SEQ ID NO:5, and a light chain variable region (VL) having the amino acid sequence of SEQ ID NO:9. In some embodiments, the antibody, or antigen-binding fragment thereof, comprises a VH having the amino acid sequence of SEQ ID NO: 5. In some embodiments, the antibody, or antigen-binding fragment thereof, comprises a VL having the amino acid sequence of SEQ ID NO: 9. In some embodiments, the antibody is semorinemab.

The methods of treatment provided herein can be applied to patients suffering from AD or other Tau pathologies, as described further herein. Suitable patients include patients suffering from mild-to-moderate AD, patients suffering from moderate AD, Apoε4 positive patients suffering from mild-to-moderate AD, Apoε4 positive patients suffering from moderate AD, Apoε4 negative patients suffering from mild-to-moderate AD, Apoε4 negative patients suffering from moderate AD, patients with an MMSE score of 16-21, particularly those with a 16-19 MMSE score or 16-18 MMSE score (e.g., 16-17, 17-18, 17-19, or 18-19), and/or with a CDR-GS of 1 or 2, and Tau positive patients, in particular, patients having Tau pathophysiology consistent with that seen in patients diagnosed with mild-to-moderate AD. In some embodiments, treatment reduces the Tau load in the extracellular space between neuronal cells in the patient's brain, particularly, aggregate Tau.

In some aspects, the methods provided herein are methods of reducing decline due to AD in patients suffering from mild-to-moderate AD. In some aspects, the methods provided herein are methods of reducing decline due to AD in patients suffering from moderate AD. In some embodiments, the decline is one or more of: clinical decline, cognitive decline, and functional decline. In some embodiments, the decline is clinical decline. In some embodiments, the decline is a decline in cognitive capacity or cognitive decline. Various tests and scales have been developed to measure cognitive capacity (including memory) and/or function. In various embodiments, one or more scale is used to measure clinical, functional, or cognitive decline.

A standard measurement of cognitive capacity is the Alzheimer's disease Assessment Scale, Cognitive Subscale, for example, the 11-item version (ADAS-Cog11). Thus, in some embodiments, the reduction or slowing in decline in cognitive capacity (or cognitive decline) in patients treated with an anti-Tau antibody is determined using the ADAS-Cog11 scale. An increase in ADAS-Cog11 score is indicative of worsening in a patient's condition.

A standard measurement of functional capacity is the Alzheimer's Disease Cooperative Study Activities of Daily Living Inventory (ADCS-ADL). Thus, in some embodiments, reduction or slowing in functional decline (or decline in functional ability or functional decline) in patients treated with an anti-Tau antibody is determined using the ADCS-ADL scale. A decrease in ADCS-ADL score is indicative of worsening in a patient's condition.

In some embodiments, the reduction or slowing in cognitive and/or functional decline (or decline in cognitive capacity and/or functional ability) in patients treated with an anti-Tau antibody is determined by a Clinical Dementia Rating Scale-Sum of Boxes (CDR-SOB or CDR-SB) score and/or a Mini Mental State Examination (MMSE). In some embodiments, the reduction or slowing in clinical decline in patients treated with an anti-Tau antibody is determined by a Neuropsychiatric Inventory (NPI) and/or a Caregiver Global Impression Scales for Alzheimer's disease (CaGI-Alz). In some embodiments, reducing or slowing cognitive decline comprises one or more of slowing memory loss, retaining memory capacity, increasing memory capacity, increasing memory function, or increasing cognitive function.

In some embodiments, decline of one or more types is assessed and one or more of the foregoing tests or scales is used to measure slowed disease progression. In some embodiments, the measurement or score (from one or more tests) is compared to the respective score at baseline, prior to administration of the antibody. In some embodiments, the slowing in decline is seen at least 13 weeks, at least 24 weeks, at least 25 weeks, at least 37 weeks, at least 49 weeks, at least 61 weeks, at least 69 weeks, or at least 73 weeks after the beginning of treatment with the antibody. In some embodiments, slowing in decline is seen after administration of the antibody for at least 40, 45, 47, 49, 51, 53, 55, 57, or 60 weeks, optionally at least once every 4 weeks (or monthly). In some embodiments, slowing in decline is seen after administration of the antibody for at least 40, 45, 47, 49, 51, 53, 55, 57, 60, 64, 68, 72, 76, 80, 84, 88, 92, 96, 100, 104, 108, 112, 116, 120, 124, 128, 132, 136, 140, 144, 148, 152, 156, 160, 164, or 168 weeks, optionally at least once every 4 weeks (or monthly).

In certain embodiments, the disclosure provides a method of maintaining cognitive capacity within 5 points of (i.e. no more than 5 points higher than) an ADAS-Cog11 score of a patient diagnosed with mild-to-moderate AD or moderate AD, for example, where ADAS-Cog11 scores are assessed for the patient before and after administration of the antibody, e.g., after repeat administration of a certain number of doses of said antibody. In some embodiments, the patient's ADAS-Cog11 score after administration of the antibody is no more than 2.5, no more than 3, no more than 3.5, no more than 4, no more than 4.5, or no more than 5 points higher than that before administration of the antibody. In some embodiments, the patient's ADAS-Cog11 score after administration of the antibody is 2-4, 3-4, or 4 points higher than that before administration of the antibody. In particular embodiments, the ADAS-Cog11 score is assessed after administration of 5-15 doses (that is, repeat administration of 5-15 4500 mg-doses) of the antibody, e.g., 10-15 doses, 12-15 doses, 13-15 doses, 13-14 doses, 14-15 doses, or 14 doses of the antibody. In some embodiments, the antibody is semorinemab. In some embodiments, the antibody is administered at least once every 4 weeks (or monthly) for at least 49 weeks. In some embodiments, a humanized monoclonal anti-Tau antibody is administered at least once every 4 weeks (or monthly) for at least 48 weeks.

In some embodiments, the patient's ADAS-Cog11 after administration of the antibody is reduced by at least 25%, at least 30%, at least 35%, at least 40%, at least 45%, or at least 50% compared to that expected without administration of the antibody (e.g., compared to a comparable placebo arm of a clinical study). Typically, a change in 3 points or more is considered clinically meaningful. In certain embodiments, the patient's ADAS-Cog11 score after administration of the antibody is reduced by 25-50%, 40-50%, or 40% compared to that expected without administration of said antibody (e.g., compared to a comparable placebo arm of a clinical study). In some embodiments, the antibody is semorinemab. In a particular embodiment, the antibody is administered at least once every 4 weeks (or monthly) for at least 49 weeks. In some embodiments, semorinemab is administered at least once every 4 weeks (or monthly) for at least 48 weeks.

In some embodiments, an ADAS-Cog11 score of the patient assessed after administration of a humanized monoclonal anti-Tau antibody is reduced by at least 25%, at least 30%, at least 35%, at least 40%, at least 45%, or at least 50% compared to that expected without administration of the antibody. In some embodiments, an ADAS-Cog11 score of the patient assessed after administration of a humanized monoclonal anti-Tau antibody is reduced by at least 25% compared to that expected without administration of the antibody. In some embodiments, an ADAS-Cog11 score of the patient assessed after administration of a humanized monoclonal anti-Tau antibody is reduced by at least 30% compared to that expected without administration of the antibody. In some embodiments, an ADAS-Cog11 score of the patient assessed after administration of a humanized monoclonal anti-Tau antibody is reduced by at least 35% compared to that expected without administration of the antibody. In some embodiments, an ADAS-Cog11 score of the patient assessed after administration of a humanized monoclonal anti-Tau antibody is reduced by at least 40% compared to that expected without administration of the antibody. In some embodiments, an ADAS-Cog11 score of the patient assessed after administration of a humanized monoclonal anti-Tau antibody is reduced by at least 45% compared to that expected without administration of the antibody. In some embodiments, an ADAS-Cog11 score of the patient assessed after administration of a humanized monoclonal anti-Tau antibody is reduced by at least 50% compared to that expected without administration of the antibody. In some embodiments, an ADAS-Cog11 score of the patient assessed after administration of a humanized monoclonal anti-Tau antibody is reduced by 25-50% compared to that expected without administration of the antibody. In some embodiments, an ADAS-Cog11 score of the patient assessed after administration of a humanized monoclonal anti-Tau antibody is reduced by 40-50% compared to that expected without administration of the antibody. In some embodiments, an ADAS-Cog11 score of the patient assessed after administration of a humanized monoclonal anti-Tau antibody is reduced by 25% compared to that expected without administration of the antibody. In some embodiments, an ADAS-Cog11 score of the patient assessed after administration of a humanized monoclonal anti-Tau antibody is reduced by 30% compared to that expected without administration of the antibody. In some embodiments, an ADAS-Cog11 score of the patient assessed after administration of a humanized monoclonal anti-Tau antibody is reduced by 35% compared to that expected without administration of the antibody. In some embodiments, an ADAS-Cog11 score of the patient assessed after administration of a humanized monoclonal anti-Tau antibody is reduced by 40% compared to that expected without administration of the antibody. In some embodiments, an ADAS-Cog11 score of the patient assessed after administration of a humanized monoclonal anti-Tau antibody is reduced by 45% compared to that expected without administration of the antibody. In some embodiments, an ADAS-Cog11 score of the patient assessed after administration of a humanized monoclonal anti-Tau antibody is reduced by 50% compared to that expected without administration of the antibody.

An antibody, or antigen-binding fragment thereof, of the disclosure is administered at a dose that is effective to treat the AD or other Tau pathology, as described herein. Suitable doses are described herein and can range from about 200 mg to about 20,000 mg, e.g., about 225 mg, about 675 mg, about 1200 mg, about 1500 mg, about 2100 mg, about 4200 mg, about 4500 mg, about 8100 mg, about 8400 mg, or about 16800 mg. In some embodiments, the dose is 225 mg, 675 mg, 1200 mg, 1500 mg, 2100 mg, 4200 mg, 4500 mg, 8100 mg, 8400 mg, or 16800 mg. In some embodiments, the dose is about 4000 mg to about 5000 mg, about 4000 mg to about 4500 mg, or about 4500 mg to about 5000 mg. In some embodiments, the dose is 4000 mg to 5000 mg, 4000 mg to 4500 mg, or 4500 mg to 5000 mg. In some embodiments, the dose is about 4500 mg. In some embodiments, the dose is 4500 mg. In some embodiments, the dose is 4500 mg administered intravenously at an infusion rate of 0.5-3.0 mL/min, e.g., 0.5 mL/min, 1 m/min, 1.5, mL/min, or 3 mL/min. In some embodiments, the dose used is 30 mg/kg to 60 mg/kg, 40 mg/kg to 50 mg/kg, 50 mg/kg to 60 mg/kg, or 50 mg/kg.

In the methods provided herein, a variety of dosage regimens are contemplated including dosage regimens in which the antibody is administered repeatedly, e.g., on a weekly or monthly schedule, over an extended period of time, e.g., months to years. In some embodiments, the antibody is administered once every 2, 3, 4, 5, 6, 7, or 8 weeks. In some instances, the antibody is administered every 2 weeks for the first 2, 3, or 4 doses, and every 4 weeks (or every month) thereafter.

The humanized monoclonal anti-Tau antibodies of the present disclosure provides a further benefit in that they do not increase the incidence of adverse events such as neuroimaging abnormalities or suicidal ideation. As shown herein, there was no statistically significant increase in these adverse events in the treatment arm relative to the placebo arm. Thus, the present disclosure further provides methods of treating patients suffering from mild-to-moderate or moderate AD without increasing (without significantly increasing) the incidence of adverse events such as neuroimaging abnormalities, suicidal ideation, headache, worsening cognitive function, alteration of consciousness, seizures, unsteadiness, and vomiting.

The present disclosure further provides pharmaceutical formulations suitable for use in the methods of treatment disclosed herein. The pharmaceuticals can be formulated for any convenient route of administration, e.g., parenteral or intravenous injection, and will typically include, in addition to the anti-Tau antibody described herein, one or more acceptable carriers, excipients, and/or diluents suited to the desired mode of administration. In some embodiments, the anti-Tau antibody may be formulated for intravenous administration. Further embodiments are described herein. The pharmaceutical formulations can be package in unit dosage forms for ease of use.

Use of the anti-Tau antibodies for treatment of AD or other Tau pathologies, as described herein, can be combined with other therapies, including one or more anti-amyloid beta (Abeta) antibodies, or one or more anti-Tau antibodies other than semorinemab. Non-limiting examples of other therapies include a neurological drug, a corticosteroid, an antibiotic, an antiviral agent, an additional anti-Tau antibody, a Tau inhibitor, an anti-amyloid beta antibody, an beta-amyloid aggregation inhibitor, an anti-BACE1 antibody, a BACE1 inhibitor, a therapeutic agent that specifically binds a target, a cholinesterase inhibitor, an NMDA receptor antagonist, a monoamine depletory, an ergoloid mesylate, an anticholinergic antiparkinsonism agent, a dopaminergic antiparkinsonism agent, a tetrabenazine, an anti-inflammatory agent, a hormone, a vitamin, a dimebolin, a homoTaurine, a serotonin receptor activity modulator, an interferon, and a glucocorticoid.

In some embodiments, the patient is being treated with a concomitant medication, e.g., a symptomatic medication. In some embodiments, the symptomatic medication is selected from the group consisting of a cholinesterase inhibitor, galantamine, rivastigmine, donepezil, an N-methyl-D-aspartate receptor antagonist, memantine, and a food supplement (optionally wherein the food supplement is Souvenaid®). The symptomatic medication, in some embodiments, is a cholinesterase inhibitor, such as galantamine, rivastigmine, and/or donepezil. In some embodiments, the symptomatic medication is an N-methyl-D-aspartate receptor antagonist. In some embodiments, the symptomatic medication is memantine. In some embodiments, the symptomatic medication is a food supplement, such as the food supplement, Souvenaid®.

The disclosure relates, in part, to the surprising finding that anti-Tau monoclonal antibodies can reduce the rate of clinical decline in cognitive capacity in patients suffering from mild-to-moderate AD or moderate AD to a statistically significant extent, as demonstrated by a Phase 2 study, in particular, reducing memory decline. The disclosure provides first-in-class immunotherapy for use in reducing cognitive clinical decline, particularly reducing memory decline, associated with pathological Tau in the brains of AD patients and patients with other Tauopathies, such as progressive supranuclear palsy (PSP).

The clinical trial met a primary endpoint in a first ever Tau antibody Phase 2 trial in mild-to-moderate AD and moderate AD, reducing the rate of cognitive clinical decline from baseline compared to placebo, as measured by ADAS-Cog11, and marking the first-ever clinical proof of concept for an anti-Tau-specific approach in treating AD or related Tau pathologies. Furthermore, as shown in the Examples herein, antibodies that bind to an N-terminal region of Tau reduce the rate of cognitive clinical decline in patients diagnosed with mild-to-moderate AD or moderate AD and, in particular, by reducing the rate of loss in memory function.

The Phase 2 clinical trial disclosed herein was a multicenter, randomized, double-blind, placebo-controlled, parallel-group clinical trial designed to evaluate the clinical efficacy, safety, pharmacokinetics, and pharmacodynamics of semorinemab in patients with mild-to-moderate AD (MMSE 16-21, CDR-GS 1 or 2). The study involved a screening period, a double-blind treatment period of 48 weeks (Weeks 1-49 for participants in Cohort 1) or 60 weeks (Weeks 1-61 for participants in Cohort 2), an optional open-label extension (OLE) period, and a safety follow-up period, with ADAS-Cog11 and ADCS-ADL tools as the co-primary endpoints, and CDR-SB, MMSE, and safety as secondary endpoints. The study followed 272 participants across 43 study centers globally.

The clinical trial disclosed herein provides the first evidence that a Tau-targeted therapy reduces the rate of clinical decline in a neurodegenerative disease, involving Tau pathologies (tauopathies). Top line data from the Phase 2 study demonstrated that semorinemab:
slowed decline relative to placebo on one primary endpoint: ADAS-Cog11—a measure of cognition, meeting one of the co-primary clinical endpoints of reducing the rate of cognitive decline from baseline compared to placebo, as measured by ADAS-Cog11; and
was safe and well tolerated, with an acceptable safety profile and no unanticipated signals.

The study also demonstrated an efficacious dose of anti-Tau antibody to slow the decline in cognitive capacity in patients diagnosed with mild-to-moderate AD, in particular, slowing memory decline. The 4500 mg Q4W dosing regimen in this study took into consideration the safety profiles from the nonclinical toxicology studies, safety and PK profiles from the Phase I study (GN39058), and results from the target engagement modeling exercise. In the target engagement modeling analysis, simulations were conducted to predict percent target (i.e., Tau) engagement by semorinemab in the interstitial fluid of the brain. Results demonstrated that high target engagement (i.e., >80%) was possible for the 4500 mg dose under various scenarios (e.g., several plasma:brain partitioning ratios and semorinemab binding affinities). Nonetheless, the extent of target engagement required for clinical efficacy was unknown before the results of this trial.

The study also surprisingly found clinical response in patients at a particular stage of disease, that is, mild-to-moderate severity of AD. Patients in this study met standard research criteria for AD (according to the NIA AA Diagnostic Criteria and Guidelines for AD), with mild-to-moderate disease severity (overall, the population had an MMSE of 16-21 points, inclusive, and a CDR GS of 1 or 2).

Without being bound to theory, Tau pathology appears to continue increasing at this stage of the disease (Jack et al. Lancet Neurol. 2013; 12:207-16). Targeting the spread of Tau in the mild-to-moderate stages of AD proved more efficacious, in some embodiments, than prior interventions targeting Aβ at this stage of the disease but that failed to ameliorate further clinical decline (Doody et al. N Engl J Med 2013; Doody et al. N Engl J Med 2014; 370:311-21369:341-5; Salloway et al. N Engl J Med 2014; 370:322-33; Egan et al. N Engl J Med 2018; 378:1691-1703). Whereas most of the accumulation of Aβ pathology may have already occurred by the time patients with AD reach the moderate stages of disease, Tau pathology appears to continue to increase (Jack et al. Lancet Neurol. 2013; 12:207-16). Further, at different stages of AD, tau pathology may manifest in different primary configurations, which may play different roles in both the further spread of tau pathology and the relative contribution of tau pathology to clinical decline. While the exact nature of the spreading species of tau in AD remains uncertain, both the structure of tau neurofibrillary tangles in the brain parenchyma and the relative abundance of different soluble phospho-tau species in CSF may evolve with increasing disease severity, such that different anti-tau therapeutics may have differential efficacy at different stages of the disease. Nonetheless, for over 30 years, studies have suggested continued progression of Tau pathology in patients with moderate AD without ever before successfully demonstrating use of an anti-Tau approach to reduce the rate of clinical decline. Consider, e.g., studies examining Tau indices via neuropathology (Nelson et al. J Neuropathol Exp Neurol. 2012; 71:362-81), CSF Tau levels (Kanai et al. Ann Neurol 1998; 44:17-26) and PET imaging (Ishiki et al. PLoS One 2015; 10:e0140311).

The Phase 2 clinical trial disclosed herein also demonstrated that the anti-Tau antibody approach treated patients diagnosed with mild-to-moderate AD or moderate AD, without increased risk of an adverse event. Unlike Aβ, Tau is not known to deposit in vascular structures, and administration of an antibody against Tau may not cause vasogenic edema or microhemorrhage as seen in some anti-AΔ therapies. Tau pathology occurs primarily intracellularly in the cytoplasm of diseased neurons (Braak et al. Acta Neuropathol 2006; 112:389-404), and soluble Tau from the extracellular space is found in the CSF (Blennow and Zetterberg, J Alzheimers Dis. 2009; 18:413-7). Further, semorinemab has an IgG4 backbone associated with reduced effector function.

On the other hand, the occurrence of imaging abnormalities believed to represent cerebral vasogenic edema and microhemorrhage have been reported in association with the investigational use of immunotherapy targeting the Aβ peptide, possibly by interacting with Aβ deposited in or around blood vessels and eliciting an immune response. Symptoms, when present in association with such imaging abnormalities, have been reported to include headache, worsening cognitive function, alteration of consciousness, seizures, unsteadiness, and vomiting (Salloway et al. Neurology 2009; 73:2061-70.; Sperling et al. Lancet Neurol. 2012; 11:241-9). The present disclosure provides treatment advantages in not eliciting said adverse effects. In some embodiments, no imaging abnormalities occur following administration of the anti-Tau antibody. In some embodiments, treatment is achieved without headache, worsening cognitive function, alteration of consciousness, seizures, unsteadiness, and/or vomiting.

### General

Practice of the methods, as well as preparation and use of the compositions disclosed herein employ, unless otherwise indicated, conventional techniques in molecular biology, biochemistry, chromatin structure and analysis, computational chemistry, cell culture, recombinant DNA and related fields as are within the skill of the art. These techniques are fully explained in the literature.

The term “herein” means the entire disclosure.

It should be understood that any of the embodiments described herein, including those described under different aspects of the disclosure and different parts of the specification (including embodiments described only in the Examples) can be combined with one or more other embodiments disclosed herein, unless explicitly disclaimed or improper. Combination of embodiments are not limited to those specific combinations claimed via the multiple dependent claims.

Any publications, patents and published patent applications referred to in this disclosure are specifically incorporated by reference herein. In case of conflict, the present specification, including its specific definitions, will control.

Throughout this specification, the word “comprise” or variations such as “comprises” or “comprising,” which is synonymous with “including,” “containing,” or “characterized by,” is inclusive or open-ended and does not exclude additional, unrecited elements or method steps.

Throughout the specification, where compositions are described as having, including, or comprising (or variations thereof), specific components, it is contemplated that compositions also may consist essentially of, or consist of, the recited components. Similarly, where methods or processes are described as having, including, or comprising specific process steps, the processes also may consist essentially of, or consist of, the recited processing steps. Further, it should be understood that the order of steps or order for performing certain actions is immaterial so long as the compositions and methods described herein remains operable. Moreover, two or more steps or actions can be conducted simultaneously.

The term “consisting of” excludes any element, step, or ingredient not specifically recited.

The term “consisting essentially of” limits the scope of a disclosure to the specified materials or steps and those that do not materially affect the basic and novel characteristic(s) of the disclosure.

Any example(s) following the term “e.g.” or “for example” is not meant to be exhaustive or limiting.

The articles “a,” “an” and “the” are used herein to refer to one or to more than one (i.e., to at least one) of the grammatical object of the article. By way of example, “an element” means one element or more than one element.

As used herein, the term “about” modifying the quantity of an ingredient, parameter, calculation, or measurement in the compositions employed in the methods of the disclosure refers to the variation in the numerical quantity that can occur, for example, through typical measuring and liquid handling procedures used for making isolated polypeptides or pharmaceutical compositions in the real world; through inadvertent error in these procedures; through differences in the manufacture, source, or purity of the ingredients employed to make the compositions or carry out the methods; and the like without having a substantial effect on the chemical or physical attributes of the compositions or methods of the disclosure. Such variation can be typically within 10%, more typically still within 5%, of a given value or range. The term “about” also encompasses amounts that differ due to different equilibrium conditions for a composition resulting from a particular initial mixture. Whether or not modified by the term “about,” the paragraphs include equivalents to the quantities. Reference to “about” a value or parameter herein includes (and describes) embodiments that are directed to that value or parameter per se. For example, description referring to “about X” includes description of “X.” Numeric ranges are inclusive of the numbers defining the range.

The term “or” as used herein should be understood to mean “and/or,” unless the context clearly indicates otherwise.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g., 1 to 6.1, and ending with a maximum value of 10 or less, e.g., 5.5 to 10. The disclosure of a range should also be considered as disclosure of the endpoints of that range.

Exemplary methods and materials are described herein, although methods and materials similar or equivalent to those described herein can also be used in the practice or testing of the present disclosure. The materials, methods, and examples are illustrative only and not intended to be limiting.

### Certain Definitions and Abbreviations

Unless defined otherwise, technical and scientific terms used herein have the same meaning as commonly understood by one of ordinary skill in the art to which this disclosure belongs. Singleton et al., Dictionary of Microbiology and Molecular Biology 2nd ed., J. Wiley & Sons (New York, N.Y. 1994), and March, Advanced Organic Chemistry Reactions, Mechanisms and Structure 4th ed., John Wiley & Sons (New York, N.Y. 1992), provide one skilled in the art with a general guide to many of the terms used in the present disclosure.

For purposes of interpreting this specification, the following definitions will apply and whenever appropriate. In the event that any definition set forth below conflicts with any document incorporated herein by reference, the definition set forth below shall control.

Ranges provided in the specification and appended claims include both end-points and all points between the end-points. Thus, for example, a range of 2.0 to 3.0 includes 2.0, 3.0, and all points between 2.0 and 3.0.

The following terms, unless otherwise indicated, shall be understood to have the following meanings:

“Administering” or “administration of” a substance, a compound or an agent to a subject refers to the contact of that substance, compound or agent to the subject or a cell, tissue, organ or bodily fluid of the subject. For example, a compound or an agent can be administered intravenously or subcutaneously. Administering can also be performed, for example, once, a plurality of times, and/or over one or more extended periods. In some embodiments, the administration includes both direct administration, including self-administration, and indirect administration, including the act of prescribing a drug. For example, as used herein, a physician who instructs a subject to self-administer a drug, or to have the drug administered by another and/or who provides a subject with a prescription for a drug is administering the drug to the subject. The terms “administering,” “administration of,” and “administered” are used interchangeably with “provided” and its different forms.

The phrase “substantially similar,” or “substantially the same,” as used herein, denotes a sufficiently high degree of similarity between two numeric values, such that one of skill in the art would consider the difference between the two values to be of little or no biological and/or statistical significance within the context of the biological characteristic measured by said values (e.g., Kd values). The difference between said two values is less than about 50%, less than about 40%, less than about 30%, less than about 20%, less than about 10%, less than about 5%, less than about 1%, less than about 0.5%, or less than about 0.1%, as a function of the value.

The terms “antibody” and “immunoglobulin” (“Ig”) are used interchangeably in the broadest sense and refers to an immunoglobulin molecule (e.g., complete antibodies, antibody fragment or modified antibodies) capable of recognizing and binding to a specific target or antigen, such as a carbohydrate, polynucleotide, lipid, polypeptide, etc., through at least one antigen recognition site, located in the variable region of the immunoglobulin molecule. The terms include, but are not limited to, monoclonal antibodies (for example, full length or intact monoclonal antibodies), polyclonal antibodies, multivalent antibodies (e.g., bispecific antibodies), antibodies with polyepitopic specificity, single chain antibodies, multi-specific antibodies (for example, bispecific antibodies, trispecific antibodies, tetraspecific antibodies), and fragments of antibodies, provided they exhibit the desired biological activity. Such antibodies can be chimeric, humanized, human, synthetic, and/or affinity matured. In some embodiments, “antibody” and/or “immunoglobulin” (Ig) refers to a polypeptide comprising at least two heavy (H) chains (about 50-70 kDa) and two light (L) chains (about 25 kDa), optionally inter-connected by disulfide bonds. There are two types of light chain: λ and κ. In humans, λ and κ light chains are similar, but only one type is present in each antibody. Heavy chains are classified as mu, delta, gamma, alpha, or epsilon, and define the antibody's isotype as IgM, IgD, IgG, IgA, and IgE, respectively. See generally, Fundamental Immunology Ch. 7 (Paul, W., ed., 2nd ed. Raven Press, N.Y. (1989)) (incorporated by reference in its entirety). The methods, uses, and compositions-for-use disclosed herein utilize IgG antibodies.

As used herein, the terms “amyloid beta,” “amyloid β,” “Abeta,” and “Aβ” are used interchangeably and refer to peptides of 36-43 amino acid residues that are the main component of the amyloid plaques found in the brains of people with Alzheimer's disease.

As used herein, the terms “additional anti-Tau antibody” and “different anti-Tau antibody” are used interchangeably and refer to an anti-Tau antibody that does not have the same six CDRs of semorinemab.

As used herein, the term “antigen-binding fragment” refers to a portion (or fragment) of an antibody that retains the antibodies binding specificity. Accordingly, as used herein, an antigen-binding fragment retains the six CDRs of the reference antibody.

The terms “full length antibody,” “intact antibody,” and “whole antibody” are used herein interchangeably to refer to an antibody having a structure substantially similar to a native antibody structure or having heavy chains that contain an Fc region as defined herein.

“Antibody fragments” comprise only a portion of an intact antibody, wherein the portion preferably retains at least one, and typically most or all, of the functions normally associated with that portion when present in an intact antibody. In some embodiments, an antibody fragment comprises an antigen-binding site of the intact antibody and thus retains the ability to bind antigen. In some embodiments, an antibody fragment, for example one that comprises the Fc region, retains at least one of the biological functions normally associated with the Fc region when present in an intact antibody, such as FcRn binding, antibody half-life modulation, ADCC function and complement binding. In some embodiments, an antibody fragment is a monovalent antibody that has an in vivo half-life substantially similar to an intact antibody. For example, such an antibody fragment may comprise an antigen-binding arm linked to an Fc sequence capable of conferring in vivo stability to the fragment. Examples of antibody fragments include but are not limited to Fv, Fab, Fab′, Fab′-SH, F(ab′)₂, diabodies, linear antibodies, single-chain antibody molecules (e.g., scFv), and multispecific antibodies formed from antibody fragments.

As used herein, the terms “Fe,” “Fe region” or “Fc domain” are used interchangeably herein and refer a C-terminal region of an immunoglobulin heavy chain that contains at least a portion of the constant region. The term includes native sequence Fc regions and variant Fc regions. In some embodiments, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991. The skilled artisan would recognize that EU numbering may differ from the residue numbering of a sequence disclosed herein. For example, the skilled artisan would recognize that S225P, M249Y, S251T and T253E mutations based on residue numbering in SEQ ID NO: 11 would be S228P, M252Y, S254T, and T256E mutations according to EU numbering. For IgG, the Fc domain comprises immunoglobulin domains Cγ2 and Cγ3 and the lower hinge region between Cγ1 and Cγ2.

As used herein, the term “Fe variant” or “variant Fe” refers to a protein comprising an amino acid modification in an Fc domain.

The terms “Fe gamma receptor,” “Fc-gamma receptor,” “FcγR” and “FcgammaR,” as used herein, are used interchangeably and refer to any member of the family of proteins that bind the IgG antibody Fe region and is encoded by an FcγR gene. An FcγR may be from any organism. In some embodiments, the FcγR is a human FcγR. In humans this family includes but is not limited to FcγRI (CD64), including isoforms FcγRIa, FcγRIb, and FcγRIc; FcγRII (CD32), including isoforms FcγRIIa (including allotypes H131 and R131), FcγRIIb (including FcγRIIb-1 and FcγRIIb-2), and FcγRIIc; and FcγRIII (CD16), including isoforms FcγRIIIa (including allotypes V158 and F158) and FcγRIIIb (including allotypes FcγRIIb-NA1 and FcγRIIb-NA2) (Jefferis et al., 2002, Immunol Lett 82:57-65, entirely incorporated by reference), as well as any undiscovered human FcγRs or FcγR isoforms or allotypes.

The term “FcRn” or “neonatal Fe Receptor,” as used herein, refers to a protein that binds the IgG antibody Fc region and is encoded at least in part by an FcRn gene. The FcRn may be from any organism. In some embodiments, the FcRn is a human FcRn. As is known in the art, the functional FcRn protein comprises two polypeptides, often referred to as the heavy chain and light chain. The light chain is beta-2-microglobulin and the heavy chain is encoded by the FcRn gene. Unless otherwise noted herein, FcRn or an FcRn protein refers to the complex of FcRn heavy chain with beta-2-microglobulin. A variety of FcRn variants can be used to increase binding to the FcRn receptor, and in some cases, to increase serum half-life. In general, unless otherwise noted, the Fc monomers disclosed herein retain binding to the FcRn receptor (and, as noted below, can include amino acid variants to increase binding to the FcRn receptor).

As used herein, the term “effector function” refers to those biological activities attributable to the Fc region of an antibody, which vary with the antibody isotype, and result from the interaction of an antibody Fc region with an Fc receptor or another effector molecule (e.g., Fc receptor-Like (FcRL) molecules, complement component Clq, and Tripartite motif-containing protein 21 (TRIM21)). Examples of antibody effector functions include: Clq binding and complement dependent cytotoxicity (CDC); Fc receptor binding; antibody-dependent cell-mediated cytotoxicity (ADCC); phagocytosis; down regulation of cell surface receptors (e.g., B cell receptor); and B cell activation. It is known in the art that wild-type IgG4 antibodies have less effector function than wild-type IgG1 antibodies. Effector functions include, but are not limited to, antibody dependent cell-mediated cytotoxicity (ADCC), antibody dependent cell-mediated phagocytosis (ADCP) and complement-dependent cellular cytotoxicity (CDC). The term “ADCC” or “antibody dependent cell-mediated cytotoxicity,” as used herein, refers to the cell-mediated reaction wherein nonspecific cytotoxic cells that express FcγRs recognize bound antibody on a target cell and subsequently cause lysis of the target cell. ADCC is correlated with binding to FcγRIIIa; increased binding to FcγRIIIa leads to an increase in ADCC activity. As is discussed herein, many embodiments of the present disclosure ablate ADCC activity entirely. The term “ADCP” or “antibody dependent cell-mediated phagocytosis,” as used herein, refers to the cell-mediated reaction wherein nonspecific cytotoxic cells that express FcγRs recognize bound antibody on a target cell and subsequently cause phagocytosis of the target cell. The term “CDC” or “complement-dependent cellular cytotoxicity,” as used herein, refers to an effector function which leads to the activation of the classical complement pathway, which is triggered by the binding of an antibody to an antigen on the target cell, which activates a series of cascades containing complement-related protein groups in blood.

An antibody that “binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more.

As used herein, the term “affinity” or “binding affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity, which reflects a 1:1 interaction between members of a binding pair (e.g., antibody binding arm and antigen). The affinity of a molecule X for its partner Y can generally be represented by the dissociation constant (Kd). Affinity can be measured by common methods known in the art, including those described herein, any of which can be used for purposes of the present disclosure. A Kd binding affinity constant can be measured by surface plasmon resonance, for example using the BIACORE® system (Pharmacia Biosensor AB, Uppsala, Sweden and Piscataway, N.J.) See also, Jonsson et al., Ann. Biol. Clin. 51:19 26 (1993); Jonsson et al., Biotechniques 11:620 627 (1991); Jonsson et al., J. Mol. Recognit. 8:125 131 (1995); Johnsson et al., Anal. Biochem. 198:268 277 (1991); Hearty S et al., Methods Mol Biol. 907:411-42 (2012), each incorporated herein by reference. The KD may also be measured using a KinExA® system (Sapidyne Instruments, Hanover, Germany and Boise, ID).

The terms “amino acid” and “amino acid identity,” as used herein, refer to one of the 20 naturally occurring amino acids that are coded for by DNA and RNA.

The term “amino acid substitution” or “substitution,” as used herein, refers to the replacement of an amino acid at a particular position in a parent polypeptide sequence with a different amino acid. In particular, in some embodiments, the substitution is to an amino acid that is not naturally occurring at the particular position, either not naturally occurring within the organism or in any organism. For example, the substitution E272Y refers to a variant polypeptide, in this case an Fc variant, in which the glutamic acid at position 272 is replaced with tyrosine. For clarity, a protein which has been engineered to change the nucleic acid coding sequence but not change the starting amino acid (for example exchanging CGG (encoding arginine) to CGA (still encoding arginine) to increase host organism expression levels) is not an “amino acid substitution”; that is, despite the creation of a new gene encoding the same protein, if the protein has the same amino acid at the particular position that it started with, it is not considered an amino acid substitution.

The terms “amino acid insertion,” “amino acid addition” or “addition” or “insertion,” as used herein, refer to the addition of an amino acid sequence at a particular position in a parent polypeptide sequence. For example, —233E, _233E or 233E designates an insertion of glutamic acid after position 233 and before position 234. Additionally, —233ADE, _233ADE or 233ADE designates an insertion of AlaAspGlu after position 233 and before position 234.

The term “amino acid deletion” or “deletion,” as used herein, refers to the removal of an amino acid sequence at a particular position in a parent polypeptide sequence. For example, E233- or E233 #, E233( ), E233_or E233del designates a deletion of glutamic acid at position 233. Additionally, EDA233-, EDA233_or EDA233 #designates a deletion of the sequence GluAspAla that begins at position 233.

As used herein, the terms “polypeptide,” “peptide” and “protein” are used interchangeably to refer to a polymer of amino acid residues.

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

“Native antibodies” refer to naturally occurring immunoglobulin molecules with varying structures. For example, native IgG antibodies are heterotetrameric glycoproteins of about 150,000 Daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, California, or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:
100 times the fraction (X/Y)
where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “monoclonal antibody” as used herein refers to an antibody obtained from a population of substantially homogeneous antibodies, i.e., the individual antibodies comprising the population are identical and/or bind the same epitope, except for possible naturally occurring mutations, or arising during production of a monoclonal antibody preparation, that may be present in minor amounts. Monoclonal antibodies are highly specific, being directed against a single antigen. Furthermore, in contrast to polyclonal antibody preparations that typically include different antibodies directed against different determinants (epitopes), each monoclonal antibody is directed against a single determinant on the antigen. The modifier “monoclonal” indicates the character of the antibody as being obtained from a substantially homogeneous population of antibodies and is not to be construed as requiring production of the antibody by any particular method. For example, the monoclonal antibodies to be used in accordance with the present disclosure may be made by a variety of techniques, including but not limited to the hybridoma method, recombinant DNA methods, phage-display methods, and methods utilizing transgenic animals containing all or part of the human immunoglobulin loci.

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al, Proc. Natl. Acad. Sci. USA 81:6851-6855 (1984)).

The “class” of an antibody refers to the type of constant domain or constant region possessed by its heavy chain. There are five major classes of antibodies: IgA, IgD, IgE, IgG, and IgM, and several of these may be further divided into subclasses (or “isotypes”), e.g., IgG1, IgG2, IgG3, IgG4, IgA1, and IgA2. The heavy chain constant domains that correspond to the different classes of immunoglobulins are called α, δ, ε, γ, and μ, respectively.

“Humanized” forms of non-human (e.g., murine) antibodies are chimeric antibodies that contain minimal sequence derived from non-human immunoglobulin. A “humanized antibody” refers to a chimeric antibody comprising amino acid residues from non-human HVRs and amino acid residues from human FRs. For the most part, humanized antibodies are human immunoglobulins (recipient antibody) in which residues from a hypervariable region of the recipient are replaced by residues from a hypervariable region of a non-human species (donor antibody), such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all the FRs are those of a human immunoglobulin sequence. The humanized antibody optionally will also comprise at least a portion of an immunoglobulin constant region (Fc), typically that of a human immunoglobulin. For further details, see Jones et al, Nature 321:522-525 (1986); Riechmann et al, Nature 332:323-329 (1988); and Presta, Curr. Op. Struct. Biol. 2:593-596 (1992). See also the following review articles and references cited therein: Vaswani and Hamilton, Ann. Allergy, Asthma & Immunol. 1: 105-115 (1998); Harris, Biochem. Soc. Transactions 23: 1035-1038 (1995); Hurle and Gross, Curr. Op. Biotech. 5:428-433 (1994).

A “human antibody” is one that comprises an amino acid sequence corresponding to that of an antibody produced by a human or a human cell and/or has been derived from a non-human source that utilizes human antibody repertoires or other human antibody-encoding sequences, for example, made using any of the techniques for making human antibodies as disclosed herein. Such techniques include, but are not limited to, screening human-derived combinatorial libraries, such as phage display libraries (see, e.g., Marks et al, J. Mol. Biol, 222: 581-597 (1991) and Hoogenboom et al, Nucl Acids Res., 19: 4133-4137 (1991)); using human myeloma and mouse-human heteromyeloma cell lines for the production of human monoclonal antibodies (see, e.g., Kozbor J. Immunol., 133: 3001 (1984); Brodeur et al, Monoclonal Antibody Production Techniques and Applications, pp. 55-93 (Marcel Dekker, Inc., New York, 1987); and Boemer et al., J. Immunol., 147: 86 (1991)); and generating monoclonal antibodies in transgenic animals (e.g., mice) that are capable of producing a full repertoire of human antibodies in the absence of endogenous immunoglobulin production (see, e.g., Jakobovits et al, Proc. Natl. Acad. Sci USA, 90: 2551 (1993); Jakobovits et al., Nature, 362: 255 (1993); Bruggermann et al., Year in Immunol, 7: 33 (1993)). This definition of a human antibody specifically excludes a humanized antibody comprising antigen-binding residues from a non-human animal.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6th ed., W.H. Freeman and Co., page 91 (2007)). A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively (see, e.g., Portolano et al, J. Immunol. 150:880-887 (1993); Clarkson et al, Nature 352:624-628 (1991)).

The term “hypervariable region,” “HVR,” or “HV,” when used herein refers to the regions of an antibody variable domain that are hypervariable in sequence and/or form structurally defined loops and/or contain the antigen-contacting residues (“antigen contacts”). Generally, antibodies comprise six hypervariable regions: three in the VH (H1, H2, H3) and three in the VL (L1, L2, L3). A number of hypervariable region delineations are in use and are encompassed herein. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1991)). Chothia refers instead to the location of the structural loops (Chothia and Lesk J. Mol. Biol. 196:901-917 (1987)). The AbM hypervariable regions represent a compromise between the Kabat CDRs and Chothia structural loops and are used by Oxford Molecular's AbM antibody modeling software. The “contact” hypervariable regions are based on an analysis of the available complex crystal structures.

Exemplary HVRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991));
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)); and
(d) combinations of (a), (b), and/or (c), including HVR amino acid residues 46-56 (L2), 47-56 (L2), 48-56 (L2), 49-56 (L2), 26-35 (H1), 26-35b (H1), 49-65 (H2), 93-102 (H3), and 94-102 (H3).
   Unless otherwise indicated, HVR residues and other residues in the variable domain (e.g., FR residues) are numbered herein according to Kabat et al., supra.

“Framework” or “FR” residues are those variable domain residues other than the hypervariable region residues as herein defined. The FR of a variable domain generally includes four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence.

A “human consensus framework” is a framework that represents the most commonly occurring amino acid residue in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al. Sequences of Proteins of Immunological Interest, Fifth Edition, NIH Publication 91-3242, Bethesda MD (1991), vols. 1-3. In some embodiments, for the VL, the subgroup is subgroup kappa I. In some embodiments, for the VH, the subgroup is subgroup III as in Kabat et al.

An “immunoconjugate” is an antibody conjugated to one or more heterologous molecule(s), including but not limited to an additional therapy (an additional therapeutic agent).

A “naked antibody” refers to an antibody that is not conjugated to a heterologous moiety (e.g., a further therapeutic moiety) or radiolabel. The naked antibody may be present in a pharmaceutical formulation.

An “isolated antibody” is one that has been identified and separated and/or recovered from a component of its natural environment. Contaminant components of its natural environment are materials that would interfere with diagnostic or therapeutic uses for the antibody, and may include enzymes, hormones, and other proteinaceous or nonproteinaceous solutes. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al, J. Chromatogr. B 848:79-87 (2007).

The terms “nucleic acid,” “polynucleotide” and “oligonucleotide” are used interchangeably and refer to a deoxyribonucleotide or ribonucleotide polymer, in linear or circular conformation, and in either single- or double-stranded form. For the purposes of the present disclosure, these terms are not to be construed as limiting with respect to the length of a polymer.

The term “position,” as used herein, refers to a location in the sequence of a protein or a polynucleotide. Positions may be numbered sequentially, or according to an established format, for example the EU index for antibody numbering. A position may be defined relative to a reference sequence. In such cases, the reference sequence is provided for comparison purposes.

The term “residue,” as used herein, refers to a position in a protein and its associated amino acid identity. For example, Asparagine 297 (also referred to as Asn297 or N297) is a residue at position 297 in a specific protein.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell but may contain mutations. Mutant progeny that have the same function or biological activity as screened or selected for in the originally transformed cell are included herein.

An “isolated nucleic acid” refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

An “isolated nucleic acid encoding an anti-Tau antibody” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The term “target,” as used herein, refers to any native molecule from any vertebrate source, including mammals such as primates (e.g., humans) and rodents (e.g., mice and rats), unless otherwise indicated. The term encompasses “full-length,” unprocessed target as well as any form of target that results from processing in the cell. The term also encompasses naturally occurring variants of targets, e.g., splice variants or allelic variants.

The term “anti-target antibody” or “an antibody that binds to a target” refers to an antibody that is capable of binding the target with sufficient affinity such that the antibody is useful as a diagnostic and/or therapeutic agent in targeting the target. In some embodiments, the extent of binding of an anti-target antibody to an unrelated, non-target protein is less than about 10% of the binding of the antibody to target as measured, e.g., by a radioimmunoassay (RIA). In certain embodiments, an anti-target antibody binds to an epitope of a target that is conserved among different species.

The term “Tau,” as used herein, refers to any native Tau protein from any vertebrate source, including mammals such as primates (e.g., humans) and rodents (e.g., mice and rats), unless otherwise indicated. The term encompasses full-length, unprocessed Tau as well as any form of Tau that results from processing in the cell. The term also encompasses naturally occurring variants of Tau, e.g., splice variants or allelic variants. Tau is known to exist in different forms. The structure and sequences of Tau are well known to one of ordinary skill in the art and methods of producing said peptides or of extracting them from brain and other tissues also are known. Tau and Tau peptides are commercially available in various forms.

The term “pTau,” as used herein, refers to Tau in which a serine, a threonine or a tyrosine residue is phosphorylated by a protein kinase by the addition of a covalently bound phosphate group. In some embodiments, pTau is phosphorylated on a serine or on a threonine residue. In some embodiments, pTau is phosphorylated on serine at position 409 and/or serine at position 404. Additional phosphorylation sites on tau include, without limitation Thr181, Ser199, Ser202, Thr205, Thr217, Thr231, Ser235, Ser422, and Thr534.

The terms “soluble Tau” or “soluble Tau protein,” as used herein, refer to solubilized Tau protein/peptide monomers, Tau-like peptides/proteins, modified or truncated Tau peptides/proteins, and/or other derivatives of Tau peptides/proteins monomers, wherein any of these are soluble, substantially soluble, or completely soluble. “Soluble Tau” also refers to Tau protein oligomers that are soluble, substantially soluble, or completely soluble. Conversely, “soluble Tau” excludes neurofibrillary tangles (NFT).

The term “insoluble Tau,” as used herein, refers to multiple aggregated monomers of Tau peptides or proteins, of Tau-like peptides/proteins, of modified or truncated Tau peptides/proteins, and/or of other derivatives of Tau peptides/proteins, wherein any of these are insoluble in the mammalian or human body, more particularly in the brain. Insoluble Tau generally forms oligomeric or polymeric structures that are insoluble both in vitro in aqueous medium and in vivo in the mammalian or human body, more particularly in the brain. “Insoluble Tau” particularly includes neurofibrillary tangles (NFT).

The terms “monomeric Tau,” “Tau monomer,” and “a monomeric form of Tau,” as used herein, refer to completely (or substantially) solubilized Tau proteins without aggregated complexes in aqueous medium.

The terms “aggregated Tau,” “oligomeric Tau,” “Tau oligomer,” and “oligomeric form of Tau,” as used herein, refer to multiple aggregated monomers of Tau peptides or proteins, of Tau-like peptides/proteins, of modified or truncated Tau peptides/proteins, and/or of other derivatives of Tau peptides/proteins forming oligomeric or polymeric structures that are insoluble in the mammalian or human body, more particularly in the brain. Generally, aggregated Tau is insoluble (completely insoluble, or substantially insoluble) both in vitro in aqueous medium and in vivo in the mammalian or human body, more particularly in the brain.

The terms “pTau PHF,” “PHF,” and “paired helical filaments,” as used herein, refer to pairs of filaments wound into helices with a periodicity of 160 nm visible on electron microscopy. Width varies between 10 and 22 nm. PHF are the predominant structures in neurofibrillary tangles of AD and neuropil threads. PHF may also be seen in some but not all dystrophic neurites associated with neuritic plaques. The major component of PHF is a hyper-phosphorylated form of microtubule-associated protein Tau. PHF may be partially composed of disulfide-linked antiparallel hyper-phosphorylated Tau proteins. PHF Tau may be truncated of its C-terminal 20 amino acid residues. The mechanisms underlying PHF formation are uncertain but hyper-phosphorylation of Tau may disengage it from microtubules, increasing the soluble pool of Tau from which PHF can be formed inside neurons.

The terms “tau pathology,” “tauopathy,” “tau protein-associated disease,” or “tau-associated disease” are used interchangeably herein, and refer to a group of diseases and disorders caused by or associated with Tau aggregates in the extracellular space of a patient's brain, including those caused by or associated with the formation of neurofibrillary or neuropil threads. Such diseases include, but are not limited to, neurological disorders, such as AD, and diseases or conditions characterized by a loss of cognitive capacity. Non-limiting examples of tau pathologies include amyotrophic lateral sclerosis, Parkinson's disease, Creutzfeldt-Jacob disease, dementia pugilistica, Down's syndrome, Gerstmann-Straussler-Scheinker disease, inclusion-body myositis, prion protein cerebral amyloid angiopathy, traumatic brain injury, amyotrophic lateral sclerosis/parkinsonism-dementia complex of Guam, non-Guamanian motor neuron disease with neurofibrillary tangles, argyrophilic grain dementia, corticobasal degeneration, diffuse neurofibrillary tangles with calcification, frontotemporal dementia, frontotemporal dementia with parkinsonism linked to chromosome 17, Hallevorden-Spatz disease, multiple system atrophy, Niemann-Pick disease type C, pallido-ponto-nigral degeneration, Pick's disease, progressive subcortical gliosis, progressive supranuclear palsy, subacute sclerosing panencephalitis, tangle only dementia, postencephalitic parkinsonism, and myotonic dystrophy. In some embodiments, the tauopathy is progressive supranuclear palsy. In some embodiments, the tau pathology is characterized by Tau pathology in the extracellular space of the patient's brain substantially similar to that found in mild-to-moderate AD, and such Tau pathologies are referred to herein as a tau pathology related to mild-to-moderate AD or a “related Tau pathology.” In some embodiments, the tau pathology is AD. In some embodiments, the tau pathology is mild-to-moderate AD. In some embodiments, the tau pathology is moderate AD. In some embodiments, the tau pathology is mild-to-moderate or moderate AD.

A “Tau positive” individual refers to a patient having brain Tau isoforms and/or levels that are typical of Tau pathologies, e.g., a patient having a positive Tau positron emission tomography (PET) scan (e.g., a positive [¹⁸F]GTP1 scan); and/or having a Tau serum or plasma detection characteristic of a tauopathy; and/or having a Tau CSF detection characteristic of a tauopathy. In some embodiments, the Tau load is consistent with that seen in patients diagnosed with mild-to-moderate AD. Current tau PET tracers only reliably detect AD tau deposits, and do not consistently label/detect PSP, FTD, or CTE tau deposits. It follows that elevated ptau181, ptau217, and ptau 231 are relatively specific to AD and are not generally found elevated in other tauopathies.

Alzheimer's Disease (“AD”) generally is diagnosed based on clinical history, clinical examination, and established imaging modalities.

Abeta positivity may be used in AD diagnosis. In some embodiments, the AD patient is determined to be amyloid positive (McKhann et al. Alzheimers Dement 2011; 7:263-9; Dubois et al. Lancet Neurol. 2014; 13:614-29). Biomarker evidence of Abeta deposition can be assessed by decreased CSF Abetal-42 levels (e.g., using a pre-specified cutoff point and the Roche Diagnostics Elecsys® Amyloid [1-42] immunoassay (below cutoff; ≤1,000 μg/mL)) and/or a centralized visual assessment of the brain by amyloid PET imaging. Both approaches have been shown to correlate with the “gold standard” of Aβ pathology at autopsy (Shaw et al. Ann Neurol. 2009:65; 403-13; Clark et al. JAMA 2011; 305:275-83; Le Bastard et al. J Alzheimer's Dis 2013; 33:117-31) (see Example 1, using this approach for enrollment in the clinical study).

This approach is in line with emerging evidence that indicates consistency between amyloid PET imaging and CSF biomarkers. Low CSF Aβ 1-42 shows an inverse relationship with in vivo Aβ cortical load as measured with Pittsburgh Compound B amyloid PET imaging (Fagan et al. Ann Neurol. 2006; 59:512-9; Forsberg et al. Neurobiol Aging 2008; 29:1456-65; Tolboom et al. J Nucl Med 2009; 50:1464-70). There is concordance on the information obtained via amyloid PET imaging and low CSF Aβ 1-42 in broad populations across a range of severity of AD (pre dementia through mild to moderate AD; Jagust et al. Neurology 2009; 73:1193-9; Fagan et al. Arch Neurol. 2011; 68:1137-44; Landau et al. Ann Neurol. 2013; 74:826-36; Zwan et al. J Alzheimer's Dis 2014; 41:801-7).

Further, both the NIA AA Diagnostic Criteria and Guidelines for AD, as well as the Qualification Opinion from the European Medicines Agency's Committee for Medicinal Products for Human Use have encouraged the use of CSF biomarkers and/or PET amyloid imaging for enrichment of trials in mild to moderate AD dementia (2012), and the U.S. Food and Drug Administration (FDA) draft guidance has done so for early AD (2018). Although the FDA guidance refers to the early stage of AD in which individuals present with mild cognitive impairment, biomarkers of amyloid pathology are also expected to add value to patient selection in moderate AD. Additional details for diagnosing AD is provided in Table 1, below.

TABLE 1

| | |
|---|---|
| Probable AD | Probable AD dementia with documented decline |
| dementia with | In persons who meet the core clinical criteria |
| increased | for probable AD dementia, documented cognitive |
| level of | decline increases the certainty that the condition |
| certainty | represents an active, evolving pathologic process, |
| | but it does not specifically increase the certainty |
| | that the process is that of AD pathophysiology. |
| | Probable AD dementia with documented decline is |
| | defined as follows: evidence of progressive cognitive |
| | decline on subsequent evaluations based on information |
| | from informants and cognitive testing in the context |
| | of either formal neuropsychological evaluation |
| | or standardized mental status examinations. |
| | Probable AD dementia in a carrier of a causative |
| | AD genetic mutation |
| | In persons who meet the core clinical criteria for |
| | probable AD dementia, evidence of a causative genetic |
| | mutation (in APP, PSEN1, or PSEN2), increases the |
| | certainty that the condition is caused by AD pathology. |
| | The workgroup noted that carriage of the ε4 allele |
| | of the apolipoprotein E gene was not sufficiently |
| | specific to be considered in this category. |
| Probable AD | AD dementia is part of a continuum of clinical and |
| dementia with | biological phenomena. AD dementia is fundamentally |
| evidence of | a clinical diagnosis. To make a diagnosis of AD |
| the AD | dementia with biomarker support, the core clinical |
| pathophys- | diagnosis of AD dementia must first be satisfied. |
| iological | Relevant biomarkers include amyloid-β(CSF or PET); |
| process | Tau (CSF or PET) |

AD = Alzheimer's disease; CSF = cerebrospinal fluid; NIA-AA = National Institute on Aging/Alzheimer's Association; PET = positron emission tomography.

The term “early Alzheimer's disease” ˜ or “early AD” as used herein (e.g., a “patient diagnose with early AD” or a “patient suffering from early AD”) includes prodromal-to-mild AD severity.

AD patients may be identified as having one or more AD biomarkers, for example, amyloid positive patients or patients having a positive Tau PET scan. One or more commercial amyloid tracers may be used, e.g., florbetapir, florbetaben, flutemetamol, and the like. One or more tau PET tracers may be used, e.g., Tau tracer is [18F] Genentech Tau Probe 1 ([18F]GTP1), as described in U.S. Pat. No. 10,076,581. In some embodiments, other Tau probes can be used. Examples of such tracer molecules include but are not limited to: RO-948 (F. Hoffmann-La Roche A G); AV-1451 (“Flortaucipir”, Avid, Inc.); PI-2014, and PI-2620 (AC Immune); MK-6240 (Merck Sharp & Dohme); and T-808 (Eli Lilly & Co.). Methods of quantifying the Tau distribution in a patient's brain, based on imaging a radio-labeled tracer, include “Standardized Uptake Value Ratio” (SUVR) (see, e.g., J. Nucl. Med., S. Sanabria Bohorquez et al., 58(1), (2017), incorporated herein by reference).

In some embodiments, the AD patient is identified using a PET tracer. In some embodiments, the PET tracer binds to Tau. In some embodiments, the PET tracer binds to Abeta. In some embodiments, the PET tracer binds to Tau or Abeta. In some embodiments, the PET tracer that binds to Tau is at least one selected from the group consisting of [¹⁸F] Genentech Tau Probe 1 ([¹⁸F]GTP1), RO-948, AV-1451 (Flortaucipir), PI-2014, PI-2620, MK-6240, and T-808 and the PET tracer that binds to Abeta is at least one selected from the group consisting of florbetapir, florebetaben, and flutemetamol. In some embodiments, the PET tracer that binds to Tau is at least one selected from the group consisting of [¹⁸F] Genentech Tau Probe 1 ([¹⁸F]GTP1), RO-948, AV-1451 (Flortaucipir), PI-2014, PI-2620, MK-6240, and T-808. In some embodiments, the PET tracer that binds to Abeta is at least one selected from the group consisting of florbetapir, florebetaben, and flutemetamol. In some embodiments, the PET tracer is [¹⁸F] Genentech Tau Probe 1 ([¹⁸F]GTP1). In some embodiments, the PET tracer is RO-948. In some embodiments, the PET tracer is AV-1451 (Flortaucipir). In some embodiments, the PET tracer is PI-2014. In some embodiments, the PET tracer is PI-2620. In some embodiments, the PET tracer is MK-6240. In some embodiments, the PET tracer is T-808. In some embodiments, the PET tracer is florbetapir. In some embodiments, the PET tracer is florebetaben. In some embodiments, the PET tracer is flutemetamol.

In some embodiments, prodromal AD refers to AD characterized by a CDR score of 0.5. AD disease progresses from prodromal to mild, mild to moderate, and moderate to severe.

In some embodiments, the term “mild Alzheimer's disease” or “mild AD” can refer to AD characterized by an MMSE score of 20 to 26. In some embodiments, mild AD refers to AD characterized by a CDR score of 1.

The term “mild-to-moderate Alzheimer's disease” or “mild-to-moderate AD” as used herein encompasses both mild and moderate AD. As used herein, mild-to-moderate AD is characterized by an MMSE score of 16-to-21. Within the group of patients having scores of 16 to 21, those with MMSE scores of 18 and below may be considered to have moderate AD and those with scores of 19 and above may be considered to have mild AD

The term “moderate Alzheimer's disease” or “moderate AD” as used herein (e.g., a “patient diagnosed with moderate AD”) generally refers to a stage of AD characterized by lower MMSE scores, such as a MMSE score of 10-19. In some embodiments, moderate AD refers to AD characterized by a CDR score of 2. Patients with moderate AD can be distinguished from those having mild cognitive impairment or mild dementia stage of disease.

Mild-to-moderate AD can be assessed by one of more of the following: (1) The Mini Mental State Examination; (2) The Clinical Dementia Rating Scale; (3) Clinical Dementia Rating-Sum of Boxes; (4) The Alzheimer's Disease Assessment Scale-Cognitive Subscale; (5) ADAS-Cog12; (6) ADAS-Cog1l: (7) The Alzheimer's Disease Cooperative Study Group-Activities of Daily Living Inventory or the Alzheimer's Disease Cooperative Study Group-Activities of Daily Living Scale; (8) The Neuropsychiatric Inventory; (9) The Caregiver Global Impression Scales for Alzheimer's Disease; (10) Instrumental Activities of Daily Living scale; and (11) Amsterdam Activities of Daily Living Questionnaire.

### (1) The Mini Mental State Examination

The Mini Mental State Examination (“MMSE”) is a brief clinical cognitive examination commonly used to screen for dementia and other cognitive deficits (Folstein et al. J Psychiatr Res 1975; 12:189-98). The MMSE provides a total score of 0-30. Scores of 26 and lower are generally considered to indicate a deficit. The lower the numerical score on the MMSE, the greater the tested patient's deficit or impairment relative to another individual with a higher score. An increase in MMSE score may be indicative of improvement in the patient's condition, whereas a decrease in MMSE score may denote worsening in the patient's condition. In some embodiments, a stable MMSE score may be indicative of a slowing, delay, or halt of the progression of AD, or a lack of appearance of new clinical, functional, or cognitive symptoms or impairments, or an overall stabilization of disease.

### (2) The Clinical Dementia Rating Scale

The Clinical Dementia Rating Scale (“CDR”) (Morris Neurology 1993; 43:2412-4) is a semi structured interview that yields five degrees of impairment in performance for each of six categories of cognitively based functioning: memory, orientation, judgment and problem solving, community affairs, home and hobbies, and personal care. The CDR was originally designed with a global score: 0—no dementia; 0.5—questionable dementia, 1—mild dementia, 2—moderate dementia, 3—severe dementia.

### (3) Clinical Dementia Rating-Sum of Boxes

A complete CDR-SB score is based on the sum of the scores across all 6 boxes. Subscores can be obtained for each of the boxes or components individually as well, e.g., CDR/Memory or CDR/Judgment and Problem solving. As used herein, a “decline in CDR-SB performance” or an “increase in CDR-SB score” indicates a worsening in the patient's condition and may reflect progression of AD.

The term “CDR-SB” refers to the Clinical Dementia Rating-Sum of Boxes, which provides a score between 0 and 18 (O'Bryant et al., 2008, Arch Neurol 65: 1091-1095). CDR-SB score is based on semi-structured interviews of patients and caregiver informants and yields five degrees of impairment in performance for each of six categories of cognitively-based functioning: memory, orientation, judgment/problem solving, community affairs, home and hobbies, and personal care. The test is administered to both the patient and the caregiver and each component (or each “box”) is scored on a scale of 0 to 3 (the five degrees are 0 (no impairment, 0.5 (questionable impairment), 1 (mild impairment), 2 (moderate impairment), and 3 (severe impairment)). The sum of the score for the six categories is the CDR-SB score. A decrease in CDR-SB score may be indicative of improvement in the patient's condition, whereas an increase in CDR-SB score may be indicative of worsening of the patient's condition. In some embodiments, a stable CDR-SB score may be indicative of a slowing, delay, or halt of the progression of AD, or a lack of appearance of new clinical, functional, or cognitive symptoms or impairments, or an overall stabilization of disease.

### (4) The Alzheimer's Disease Assessment Scale-Cognitive Subscale

The Alzheimer's Disease Assessment Scale-Cognitive Subscale (“ADAS Cog”) is a frequently used scale to assess cognition in clinical trials for mild-to-moderate AD (Rozzini et al. Int J Geriatr Psychiatry 2007; 22:1217-22.; Connor and Sabbagh, J Alzheimers Dis. 2008; 15:461-4; Ihl et al. Int J Geriatr Psychiatry 2012; 27:15-21). The ADAS-Cog is an examiner-administered battery that assesses multiple cognitive domains, including memory, comprehension praxis, orientation, and spontaneous speech (Rosen et al. 1984, Am J Psychiatr(r 141:1356-64; Mohs et al. 1997, Alzheimer Dis Assoc Disord 11(S2):S13-S21). The ADAS-Cog is a standard primary endpoint in AD treatment trials (Mani 2004, Stat Med 23:305-14). The higher the numerical score on the ADAS-Cog, the greater the tested patient's deficit or impairment relative to another individual with a lower score. The ADAS-Cog may be used to assess whether a treatment for AD is therapeutically effective. An increase in ADAS-Cog score is indicative of worsening in the patient's condition, whereas a decrease in ADAS-Cog score denotes improvement in the patient's condition. In some embodiments, a stable ADAS-Cog score may be indicative of a slowing, delay, or halt of the progression of AD, or a lack of appearance of new clinical or cognitive symptoms or impairments, or an overall stabilization of disease.

### (5) ADAS-Cog12

The ADAS-Cog12 is the 70-point version of the ADAS-Cog plus a 10-point Delayed Word Recall item assessing recall of a learned word list. The ADAS-Cog11 is another version, with a range from 0-70. Other ADAS-Cog scales include the ADAS-CoL13 and ADAS-Cog14

### (6) ADAS-Cog11

A decrease in ADAS-Cog11 score may be indicative of improvement in the patient's condition, whereas an increase in ADAS-Cog11 score may be indicative of worsening of the patient's condition. In some embodiments, a stable ADAS-Cog11 score may be indicative of a slowing, delay, or halt of the progression of AD, or a reduction in the progression of clinical or cognitive decline, or a lack of appearance of new clinical or cognitive symptoms or impairments, or an overall stabilization of disease.

The component subtests of the ADAS-Cog11 can be grouped into three cognitive domains: memory, language, and praxis (Verma et al. Alzheimer's Research & Therapy 2015). This “breakdown” can improve sensitivity in measuring decline in cognitive capacity, e.g., when focused in the mild-to-moderate AD stage (Verma, 2015). Thus ADAS-Cog11 scores can be analyzed for changes on each of three cognitive domains: a memory domain, a language domain, and a praxis domain. A memory domain value of an ADAS-Cog11 score may be referred to herein as an “ADAS-Cog11 memory domain score” or simply “memory domain.” Slowing memory decline may refer to reducing rate of loss in memory capacity and/faculty, retaining memory, and/or reducing memory loss. Slowing memory decline can be evidenced, e.g., by smaller (or less negative) scores on the ADAS-Cog11 memory domain (see, e.g., Table 12).

Similarly, a language domain value of an ADAS-Cog11 score may be referred to herein as an “ADAS-Cog11 language domain score” or simply “language domain;” and a praxis domain value of an ADAS-Cog11 score may be referred to herein as an “ADAS-Cog11 praxis domain score” or simply “praxis domain.” Praxis can refer to the planning and/or execution of simple tasks and/or praxis can refer to the ability to conceptualize, plan, and execute a complex sequence of motor actions, as well as copy drawings or three-dimensional constructions, and following commands.

The memory domain score is further divided into components including scores reflecting a subject's ability to recognize and/or recall words, thereby assessing capabilities in “word recognition” or “word recall.” A word recognition assessment of an ADAS-Cog11 memory domain score may be referred to herein as an “ADAS-Cog11 word recognition score” or simply “word recognition score.” For example, equivalent alternate forms of subtests for word recall and word recognition can be used in successive test administrations for a given patient. In some embodiments, slowing memory decline may refer particularly to slowing decline in word recognition capability, e.g., reducing rate of word recognition loss, retaining memory in terms of recognizing words, and/or reducing memory loss in recalling a list of words. Slowing memory decline can be evidenced, e.g., by smaller (or less negative) scores on the word recognition component of the ADAS-Cog11 memory domain.

### (7) The Alzheimer's Disease Cooperative Study Group-Activities of Daily Living Inventory or the Alzheimer's Disease Cooperative Study Group-Activities of Daily Living Scale

The Alzheimer's Disease Cooperative Study Group-Activities of Daily Living Inventory or the Alzheimer's Disease Cooperative Study Group-Activities of Daily Living Scale (“ADCS-ADL;” Galasko et al. Alzheimer Dis Assoc Disord 1997; 11(Suppl 2):S33-9) is the most widely used scale for assessing functional outcomes in patients with AD (Vellas et al. Lancet Neurol. 2008; 7:436-50). Scores range from 0 to 78, with higher scores indicating better ADL function. The ADCS-ADL is administered to caregivers and covers both basic ADL (e.g., eating and toileting) and more complex ADL or instrumental ADL (e.g., using the telephone, managing finances, preparing a meal) (Galasko et al. Alzheimer Disease and Associated Disorders, 1997 11(Supp12), S33-S39).

### (8) The Neuropsychiatric Inventory

The Neuropsychiatric Inventory (“NPI”) (Cummings et al. Neurology 1994; 44:2308-14) is a widely used scale that assesses the behavioral symptoms in AD, including their frequency, severity, and associated distress. Individual symptom scores range from 0 to 12 and total NPI scores range from 0 to 144. NPI is administered to caregivers and refers to the behavior of the patient over the preceding month.

### (9) The Caregiver Global Impression Scales for Alzheimer's Disease

The Caregiver Global Impression Scales for Alzheimer's Disease (“CaGI-Alz”) is a novel scale used in clinical studies described herein, and is comprised of four items to assess the caregiver's perception of the patient's change in disease severity. All items are rated on a 7-point Likert type scale from 1 (very much improved since treatment started/previous CaGI Alz assessment) to 7 (very much worsened since treatment started/previous CaGI Alz assessment).

### (10) Instrumental Activities of Daily Living Scale

The term “iADL” refers to the Instrumental Activities of Daily Living scale (Lawton, M. P., and Brody. E. M., 1969, Gerontologist 9: 179-186). This scale measures the ability to perform typical daily activities such as housekeeping, laundry, operating a telephone, shopping, preparing meals, etc. The lower the score, the more impaired the individual is in conducting activities of daily living

### (11) Amsterdam Acclivities of Daily Living Questionnaire

Another scale that may be used is the “Amsterdam Activities of Daily Living Questionnaire (A-IADL-Q).”

In some embodiments, “mild-to-moderate AD” refers to AD dementia of moderate severity, as defined by a screening MMSE score of 16-21 points, inclusive, and a CDR GS of 1 or 2, and where AD is diagnosed based on standard research criteria according to the National Institute on Aging-Alzheimer's Association (NIA AA) Diagnostic Criteria and Guidelines for AD, outlined above in Table 1. In Example 1, e.g., these scores together provide evidence of mild-to-moderate AD (e.g., with mild AD correlating with an MMSE score of 19-21 and/or a CDR GS of 1; and moderate AD correlating with an MMSE score of 16-18 and/or a CDR GS of 2).

“Amyloidoses” refers to a group of diseases and disorders associated with amyloid or amyloid-like protein such as the amyloid β protein involved in AD.

“Anti-Tau immunoglobulin,” “anti-Tau antibody,” and “antibody that binds Tau” are used interchangeably herein, and refer to an antibody that is capable of binding Tau (e.g., human Tau) with sufficient affinity such that the antibody is useful as a diagnostic and/or therapeutic agent in targeting Tau. In some embodiments, the extent of binding of an anti-Tau antibody to an unrelated, non-Tau protein is less than about 10% of the binding of the antibody to Tau as measured, e.g., by a radioimmunoassay (RIA). In some embodiments, an antibody that binds to Tau has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M). In some embodiments, an anti-Tau antibody binds to an epitope of Tau that is conserved among Tau from different species. In some cases, the antibody binds monomeric Tau, oligomeric Tau, and/or phosphorylated Tau. In some embodiments, the anti-Tau antibody binds to monomeric Tau, oligomeric Tau, non-phosphorylated Tau, and phosphorylated Tau with comparable affinities, such as with affinities that differ by no more than 50-fold from one another. In some embodiments, an antibody that binds monomeric Tau, oligomeric Tau, non-phosphorylated Tau, and phosphorylated Tau is referred to as a “pan-Tau antibody.” A non-limiting example of an anti-Tau antibody is semorinemab.

An “N-terminal binding anti-Tau antibody” or “N-terminal binder” refers to an antibody that binds Tau towards its N-terminal end, e.g., binding to an epitope within amino acids 2 to 24 of mature human Tau, such as, in some embodiments, binding to an epitope within/spanning amino acids 6 to 23 of mature human Tau.

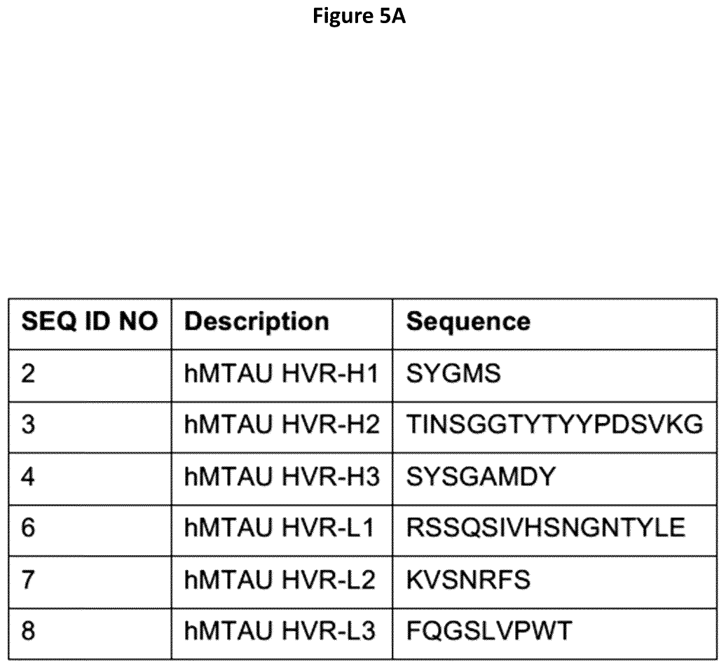

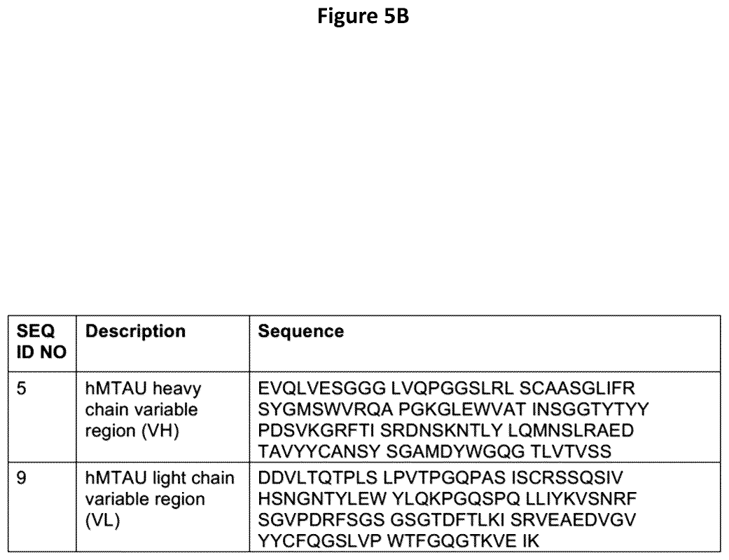

The terms “semorinemab,” “MTAU9937A,” and “RO7105705” are used interchangeably herein, and refer to a specific anti-Tau antibody that binds to monomeric, oligomeric, non-phosphorylated, and phosphorylated forms of Tau. In some embodiments, the anti-Tau antibody binds to an N-terminal region of Tau, for example, an epitope within residues 2 to 24, such as an epitope within/spanning residues 6 to 23. In some embodiments, the anti-Tau antibody comprises HVR sequences set forth in FIG. 5A. In some embodiments, the anti-Tau antibody comprises: an HVR-H1 comprising the amino acid sequence SEQ ID NO: 2; an HVR-H2 sequence comprising the amino acid sequence SEQ ID NO: 3; an HVR-H3 sequence comprising the amino acid sequence SEQ ID NO: 4; an HVR-L1 sequence comprising the amino acid sequence SEQ ID NO: 6; an HVR-L2 sequence comprising the amino acid sequence SEQ ID NO: 7; and an HVR-L3 sequence comprising the amino acid sequence SEQ ID NO: 8. In some embodiments, the specific anti-Tau antibody comprises VH and VL domains having the amino acid sequences set forth in FIG. 5B. In some embodiments, the anti-Tau antibody comprises a VH domain comprising the amino acid sequence SEQ ID NO: 5 and a VL domain comprising the amino acid sequence SEQ ID NO: 9. In some embodiments, the anti-Tau antibody is an IgG4 antibody. In some embodiments, the IgG4 antibody comprises one or more mutations selected from M252Y, S254T, and T256E, according to EU numbering. In some embodiments, the IgG4 antibody comprises M252Y, S254T, and T256E mutations, according to EU numbering. In some embodiments, the antibody comprises an S228P mutation, according to EU numbering.

The term “therapeutic agent” refers to any agent that is used to treat a disease, including but not limited to an agent that treats a symptom of the disease or that slows disease progression.

As used herein, “treatment” (and grammatical variations thereof such as “treat” or “treating”) refers to clinical intervention in an attempt to alter the natural course of a pathology of the individual being treated, and can be performed either for prophylaxis or during the course of the pathology.

Desirable effects of treatment include, but are not limited to, preventing occurrence or recurrence of disease, alleviation or amelioration of one or more symptoms, diminishing or delaying direct or indirect pathological consequence of the disease, halting or slowing worsening of any direct or indirect pathological consequence of the disease, decreasing the rate of disease progression, amelioration or palliation of the disease state, remission, and/improved prognosis. In some embodiments, an anti-Tau antibody described herein is used to delay development of a disease or to slow the progression of a disease, e.g., to slow clinical decline in mild-to-moderate AD or a related tauopathy (such as slowing cognitive decline by slowing loss in memory function). In some specific embodiments, the anti-Tau antibody is used to intercept, prevent, or slow the spread of extracellular forms of Tau, to intercept, prevent, or slow cell-to-cell spread of Tau and/or propagation of Tau toxicity and pathology, throughout cortical and sub cortical networks in the brain of a patient with mild-to-moderate AD or a related tauopathy.

The term “treatment regimen,” as used herein, refers to a combination of dosage, mode of administration, frequency of administration, and/or duration of treatment, with or without addition of another therapy, e.g., a concomitant medication.

The term “effective treatment regimen,” as used herein, refers to a treatment regimen that will offer beneficial effect to a patient receiving the treatment.

The term “modifying a treatment,” as used herein, refers to changing the treatment regimen including, changing dosage, mode of administration, frequency of administration, or duration of treatment, and/or addition or removal of additional therapy.

The term “treatment emergent” as used herein refers to an event that occurs after a first dose of a therapeutic agent is administered. For example, a “treatment emergent adverse event” is an event that is identified upon or after the first dose of a treatment in a clinical study.

An “adverse event” is any untoward medical occurrence in a patient administered a pharmaceutical product, regardless of causal attribution. An adverse event can therefore be any of the following:
Any unfavorable and unintended sign (including an abnormal laboratory finding), symptom, or disease temporally associated with the use of a pharmaceutical product, whether or not considered related to the product
Any new disease or exacerbation of an existing disease (a worsening in the character, frequency, or severity of a known condition)
Recurrence of an intermittent medical condition (e.g., headache) not present at baseline
Any deterioration in a laboratory value or other clinical test (e.g., ECG, X ray) that is associated with symptoms or leads to a change in study treatment or concomitant treatment or discontinuation from study drug (e.g., semorinemab, [¹⁸F]GTP1 radioligand, or an amyloid radioligand)
Adverse events that are related to a protocol mandated intervention, including those that occur prior to assignment of study treatment (e.g., screening invasive procedures such as biopsies).
   For example, adverse events may include one or more of an abnormal laboratory value (e.g., hyperkalemia, elevated potassium), abnormal vital sign (e.g., hypertension), abnormal liver function tests, more frequent headache, and hospitalization.

A “serious adverse event” is an adverse event that meets any of the following criteria: is fatal (i.e., the adverse event actually causes or leads to death); is life threatening (i.e., the adverse event, in the view of the investigator, places the patient at immediate risk of death) (but does not include any adverse event that, had it occurred in a more severe form or was allowed to continue, might have caused death); requires or prolongs inpatient hospitalization; results in persistent or significant disability/incapacity (i.e., the adverse event results in substantial disruption of the patient's ability to conduct normal life functions); is a congenital anomaly/birth defect in a neonate/infant born to a mother exposed to study drug (e.g., [¹⁸F]GTP1 radioligand or an amyloid radioligand); is a significant medical event in the investigator's judgment (e.g., may jeopardize the patient or may require medical/surgical intervention to prevent one of the outcomes listed above).

The terms “severe” and “serious” are not synonymous. Severity refers to the intensity of an adverse event (e.g., rated as mild, moderate, or severe, or according to National Cancer Institute Common Terminology Criteria for Adverse Events); the event itself may be of relatively minor medical significance (such as severe headache without any further findings).

“Adverse events of special interest” as used herein include: cases of potential drug induced liver injury that include an elevated ALT or AST in combination with either an elevated bilirubin or clinical jaundice, as defined by Hy's Law; suspected transmission of an infectious agent by the study drug or [¹⁸F]GTP1 radioligand, such as any organism, virus, or infectious particle (e.g., prion protein transmitting transmissible spongiform encephalopathy), pathogenic or non-pathogenic; treatment emergent clinically significant MRI abnormalities; and/or a severe (Grade 3 or more) infusion related reactions.

A “persistent adverse event” is one that extends continuously, without resolution, between patient evaluation time-points.

A “recurrent adverse event” is one that resolves between patient evaluation time-points and subsequently recurs.

The term “baseline,” as used herein, refers to a point or points in time at which a patient is evaluated before the administration of a first dose of anti-Tau antibody in the treatment methods herein. A “baseline score” or a “baseline assessment” refers to a score assessed prior to treatment, before the first/initial dose of antibody. In some embodiments, a baseline assessment occurs during a pre-treatment screening period.

As used herein, “lifetime” of a patient refers to the remainder of the life of the patient after starting treatment.

The term “progression” as used herein refers to the worsening of a disease over time. The “progression rate” or “rate of progression” of a disease refers to how fast or slow a disease develops over time in a patient diagnosed with the disease. The progression rate of a disease can be represented by measurable changes over time of particular characteristics of the disease. A patient carrying particular genetic trait is said to have, or more likely to have, “increased progression rate” if her disease state progresses faster than those patients without such genetic trait. On the other hand, a patient responding to a therapy is said to have, or more likely to have, “decreased progression rate” if her disease progression slows down following administration of the therapy, when compared to her disease state prior to the treatment or to other patients not receiving the therapy.

The terms “slowed clinical decline,” “slowing the clinical decline,” “reduction in cognitive decline” and other grammatical variations, refer to a reduction in progression rate of a given disease, e.g., following a treatment. In some embodiments, the clinical decline is compared to a patient also diagnosed with the same disease, in a similar stage (e.g., mild-to-moderate AD), but who has received a placebo rather than an anti-Tau antibody, as described herein. Comparisons to placebo (e.g., to a comparable placebo arm of a clinical study) may represent comparisons to disease progression expected without administration of the anti-Tau antibody. Slowing of clinical decline may be measured, e.g., by a reduction in the functional decline and/or cognitive decline, e.g., as described herein, or in a particular cognitive domain, such as memory.

The terms “sample” and “test sample” are used interchangeably herein and refer to a composition that is obtained or derived from a subject of interest that contains a cellular and/or other molecular entity that is to be characterized and/or identified, for example based on physical, biochemical, chemical and/or physiological characteristics. In some embodiments, the definition encompasses blood and other liquid samples of biological origin and tissue samples such as a biopsy specimen or tissue cultures or cells derived therefrom. The source of the tissue sample may be solid tissue as from a fresh, frozen and/or preserved organ or tissue sample or biopsy or aspirate; blood or any blood constituents; bodily fluids; and cells from any time in gestation or development of the subject or plasma.

The term “biological sample” as used herein includes, but is not limited to, whole blood, blood-derived cells, serum, plasma, sputum, tissue biopsies (e.g., lung samples), nasal samples including nasal swabs or nasal polyps, lymph fluid, synovial fluid, cellular extracts, and combinations thereof. In some embodiments, the sample is a clinical sample. In some embodiments, the sample is used in a diagnostic assay. In some embodiments, the sample is a CSF sample or a plasma sample, e.g., a CSF or plasma sample used for assessing Tau levels in a patient. In some embodiments, the sample is a CSF sample taken from a patient suffering from a tau pathology, such as AD. In some embodiments, the sample is a plasma sample taken from a patient suffering from a tau pathology, such as AD.

The term “biological sample” may also include biological samples that have been manipulated in any way after their procurement, such as by treatment with reagents, solubilization, or enrichment for certain components, such as proteins or polynucleotides, or embedding in a semi-solid or solid matrix for sectioning purposes. For the purposes herein a “section” of a tissue sample is meant a single part or piece of a tissue sample, e.g., a thin slice of tissue or cells cut from a tissue sample. In some embodiments, a sample is obtained from a subject or patient prior to treatment with an anti-Tau antibody. In some embodiments, a sample is obtained from a subject or patient following at least one treatment with an anti-Tau antibody.

A “reference sample,” as used herein, refers to any sample, standard, or level that is used for comparison purposes. In some embodiments, a reference sample is obtained from a patient before initiation of treatment with an anti-Tau antibody, as described herein, e.g., to provide a baseline. In some embodiments, a reference sample is obtained from a healthy and/or non-diseased part of the body (e.g., tissue or cells) of the same subject or patient. In some embodiments, a reference sample is obtained from an untreated tissue and/or cell of the body of the same subject or patient. In some embodiments, a reference sample is obtained from a healthy and/or non-diseased part of the body (e.g., tissues or cells) of an individual who is not the subject or patient. In some embodiments, a reference sample is obtained from an untreated tissue and/or cell part of the body of an individual who is not the subject or patient.

In certain embodiments, a reference sample is a single sample or combined multiple samples from the same subject or patient that are obtained at one or more different time points from the time the test sample is obtained. For example, a reference sample may be obtained at an earlier time point from the same subject or patient compared to when the test sample is obtained. In certain embodiments, a reference sample is obtained from one or more individuals with a Tau pathology, e.g., AD, who is not the subject or patient.

In certain embodiments, a reference sample is combined multiple samples from one or more healthy individuals who are not the subject or patient. In certain embodiments, a reference sample is combined multiple samples from one or more individuals with a disease or disorder (e.g., tauopathy, such as, for example, AD or mild-to-moderate AD) who are not the subject or patient. In certain embodiments, a reference sample is pooled CSF, pooled plasma, or serum samples from one or more individuals who are not the subject or patient.

As used herein, the terms “patient” “subject” and “individual” are used interchangeably herein and refer to any single subject for whom treatment is desired. In certain embodiments, the patient is a human. A subject is typically a human. In certain embodiments, a subject is a non-human mammal. Exemplary non-human mammals include laboratory, domestic, pet, sport, and stock animals, e.g., mice, cats, dogs, horses, and cows. Typically, the subject is eligible for treatment, e.g., displays one or more indicia of disease. Generally, such subject or patient is eligible for treatment for a tau pathology, e.g., mild-to-moderate AD. In some embodiments, such eligible subject or patient is one that is experiencing or has experienced one or more signs, symptoms, or other indicators of mild-to-moderate AD or has been diagnosed with mild-to-moderate AD, as described above. In some embodiments, an eligible subject or patient is one that is experiencing or has experienced one or more signs, symptoms, or other indicators of moderate AD or has been diagnosed with moderate AD, as described above.

Intended to be included as a subject are individuals involved in clinical research trials or in epidemiological studies, or subjects used as controls. The subject may have been previously treated with an anti-Abeta or anti-Tau antibody, or antigen-binding fragment thereof, or another drug, or not so treated. The subject may be naive to an additional drug(s) being used when the treatment herein is started, i.e., the subject may not have been previously treated with, for example, a therapy other than anti-Tau antibody at “baseline.” Such “naïve” subjects are generally considered to be candidates for treatment with such additional drug(s).

Patients include people of different ethnic origins. For example, in some embodiments, patients are of non-European descent, e.g., patients who are Black, Hispanic, and/or Asian. In some embodiments, the patient is Black or Hispanic or has a non-European ethnic origin. In some embodiments, the patient is Black. In some embodiments, the patient is Hispanic. In some embodiments, the patient is Asian. In some embodiments, the patient is of non-European descent.

The term “more likely to respond,” as used herein, refers to patients that are most likely to demonstrate beneficial effect following administration of an anti-Tau antibody, including slowing down, delaying, or halting disease progression; and/or slowing, delaying, or halting cell-to-cell spread of Tau toxicity and pathology throughout cortical/sub-cortical networks in the patient's brain. With regard to mild-to-moderate AD, “more likely to respond” also refers to patients that are more likely to demonstrate a reduction in loss of function or cognition following anti-Tau antibody treatment. The phrase “responsive to” in the context of the present disclosure indicates that a patient suffering from or diagnosed with a disorder as described herein, shows a response to anti-Tau antibody treatment.

The terms “Apolipoprotein ε4 carrier” or “Apoε4 carrier” are used interchangeably herein with “apolipoprotein ε4 positive” or “Apoε4 positive” and refer to an individual having at least one apolipoprotein ε4 (or “Apoε4”) allele. An individual with zero Apoε4 alleles is referred to herein as being “Apoε4 negative” or an “Apoε4 non-carrier” (Prekumar, et al., 1996, Am. J Pathol. 148:2083-95).

The terms “pharmaceutical formulation” and “pharmaceutical composition” are used interchangeably herein and refer to a preparation which is in such form as to permit the biological activity of an active ingredient contained therein to be effective, and which contains no additional components which are unacceptably toxic to a subject to which the formulation would be administered.

The terms “therapeutically effective amount,” “effective amount,” and “effective dose” of an agent e.g., an anti-Tau antibody in a pharmaceutical composition, are used interchangeably herein and refer to an amount effective, for periods of time necessary, to achieve a desired therapeutic or prophylactic result. For example, a therapeutically effective amount is an amount effective, for periods of time necessary, to treat the indicated disease, clinical pathology, or symptom, such as to modify the progression of AD, particularly mild-to-moderate AD, and/or to alleviate and/or prevent one or more symptoms of AD. In particular embodiments, an effective amount is used to reduce the rate of memory decline.

A “fixed” or “flat” dose of a therapeutic agent refers to a dose that is administered to a human patient without regard for the weight (WT) or body surface area (BSA) of the patient. The fixed or flat dose is therefore not provided as a mg/kg dose or a mg/m² dose, but rather as an absolute amount of the therapeutic agent or active ingredient.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

The terms “wild type” or “WT” are used interchangeably herein and refer to an amino acid sequence or a nucleotide sequence that is found in nature, including allelic variations. A WT protein has an amino acid sequence or is encoded by a nucleotide sequence that has not been intentionally modified.

The sequences referenced herein are provided in Table 3, infra. It is known in the art that during the processing and expression of Fc-containing proteins that the C-terminal lysine may be cleaved (also known in the art as C-terminal lysine clipping). Accordingly, for each sequence disclosed herein that contains a C-terminal lysine, the corresponding sequence without the C-terminal lysine (i.e. the C-terminal lysine cleavage product) is also contemplated. In some embodiments, the first monomer comprises a C-terminal lysine. In some embodiments, the monomer comprises a C-terminal lysine. In some embodiments, the monomer lacks a C-terminal lysine. In some embodiments, the heavy chain of the anti-Tau antibody comprises a C-terminal lysine. In some embodiments, the heavy chain of the anti-Tau antibody lacks a C-terminal lysine.

It is also known in the art that the C-terminal cleavage process is imprecise and that additional C-terminal residues may be cleaved. Accordingly, for each sequence disclosed herein that contains a C-terminal lysine, the corresponding sequence without the two C-terminal residues is also contemplated. In some embodiments, for each sequence disclosed herein that contains a C-terminal lysine, the corresponding sequence without the three C-terminal residues is also contemplated. In some embodiments, for each sequence disclosed herein that contains a C-terminal lysine, the corresponding sequence without the four C-terminal residues is also contemplated. In some embodiments, for each sequence disclosed herein that contains a C-terminal lysine, the corresponding sequence without the five C-terminal residues is also contemplated. In some embodiments, for each sequence disclosed herein that contains a C-terminal lysine, the corresponding sequence without the six C-terminal residues is also contemplated. In some embodiments, for each sequence disclosed herein that contains a C-terminal lysine, the corresponding sequence without the seven C-terminal residues is also contemplated. In some embodiments, for each sequence disclosed herein that contains a C-terminal lysine, the corresponding sequence without the eight C-terminal residues is also contemplated. In some embodiments, for each sequence disclosed herein that contains a C-terminal lysine, the corresponding sequence without the nine C-terminal residues is also contemplated. In some embodiments, for each sequence disclosed herein that contains a C-terminal lysine, the corresponding sequence without the ten C-terminal residues is also contemplated. In some embodiments, for each sequence disclosed herein that contains a C-terminal lysine, the corresponding sequence without the eleven C-terminal residues is also contemplated. In some embodiments, for each sequence disclosed herein that contains a C-terminal lysine, the corresponding sequence without the twelve C-terminal residues is also contemplated. In some embodiments, for each sequence disclosed herein that contains a C-terminal lysine, the corresponding sequence without the thirteen C-terminal residues is also contemplated. In some embodiments, for each sequence disclosed herein that contains a C-terminal lysine, the corresponding sequence without the fourteen C-terminal residues is also contemplated. In some embodiments, for each sequence disclosed herein that contains a C-terminal lysine, the corresponding sequence without the fifteen C-terminal residues is also contemplated. In some embodiments, the missing C-terminal residues are the result of engineering (e.g., expressing a polynucleotide missing the nucleotide sequences encoding one or more of the C-terminal residues).

An “imaging agent” is a compound that has one or more properties that permit its presence and/or location to be detected directly or indirectly. Examples of imaging agents include proteins and small molecule compounds incorporating a labeled moiety that permits detection.

The term “small molecule” refers to an organic molecule having a molecular weight between 50 Daltons to 2500 Daltons.

A “label” is a marker coupled with a molecule to be used for detection or imaging. Examples of such labels include: a radiolabel, a fluorophore, a chromophore, or an affinity tag. In some embodiments, the label is a radiolabel used for medical imaging, for example Tc⁹⁹, or ¹²³1, or a spin label for nuclear magnetic resonance (NMR) imaging (also known as magnetic resonance imaging, MRI), such as iodine-123, iodine-131, fluorine-19, carbon-13, nitrogen-15, oxygen-17, gadolinium, manganese, iron, etc.

The term “package insert” is used to refer to instructions customarily included in commercial packages of therapeutic products, that contain information about the indications, usage, dosage, administration, combination therapy, contraindications, and/or warnings concerning the use of such therapeutic products. The term “package insert” is also used to refer to instructions customarily included in commercial packages of diagnostic products that contain information about the intended use, test principle, preparation and handling of reagents, specimen collection and preparation, calibration of the assay and the assay procedure, performance and precision data such as sensitivity and specificity of the assay.

### Compositions and Methods

The present disclosure provides compositions and methods for the treatment, prognosis, selection and/or identification of patients for treatment with anti-Tau antibodies. In one aspect, the disclosure is based, in part, on improved methods of treating Tau pathologies, particularly mild-to-moderate or moderate AD.

### A. Exemplary Antibodies

Methods of treating tauopathies with antibodies that bind Tau are provided. In some embodiments, the antibody binds monomeric Tau, oligomeric Tau, non-phosphorylated Tau, and phosphorylated Tau. In some embodiments, the antibody binds to an epitope within amino acids 2 to 24 of mature human Tau. In some embodiments, the antibody binds to an epitope within Tau amino acids 2 to 24 and binds monomeric Tau, oligomeric Tau, non-phosphorylated Tau, and phosphorylated Tau. In some embodiments, the antibody binds to an epitope within, or spanning, amino acids 6 to 23 of mature human Tau. In some embodiments, the antibody binds to an epitope within, or spanning, Tau amino acids 6 to 23 and binds monomeric Tau, oligomeric Tau, non-phosphorylated Tau, and phosphorylated Tau. In some embodiments, the antibody binds an epitope of human Tau having, or consisting of, the sequence AEPRQEFEVMEDHAGTYGLGDRK (SEQ ID NO: 1). In some embodiments, the antibody binds an epitope of cynomolgus monkey Tau having, or consisting of, the sequence AEPRQEFDVMEDHAGTYGLGDRK (SEQ ID NO: 10).

In some embodiments, the humanized monoclonal anti-Tau antibody of this disclosure binds within residues 2 and 24, or within residues 6 and 23, of human Tau (SEQ ID NO: 1) in monomeric Tau, oligomeric Tau, non-phosphorylated Tau, and phosphorylated Tau. In some embodiments, the humanized monoclonal anti-Tau antibody of this disclosure binds within residues 2 and 24 of human Tau (SEQ ID NO: 1) in monomeric Tau, oligomeric Tau, non-phosphorylated Tau, and phosphorylated Tau. In some embodiments, the humanized monoclonal anti-Tau antibody of this disclosure binds within residues 6 and 23 of human Tau (SEQ ID NO: 1) in monomeric Tau, oligomeric Tau, non-phosphorylated Tau, and phosphorylated Tau.

In some embodiments, the humanized monoclonal anti-Tau antibody of this disclosure binds each of monomeric Tau, phosphorylated Tau, non-phosphorylated Tau, and oligomeric Tau with a K_{D} of less than 100 nM, less than 75 nM, or less than 50 nM. In some embodiments, the humanized monoclonal anti-Tau antibody of this disclosure binds each of monomeric Tau, phosphorylated Tau, non-phosphorylated Tau, and oligomeric Tau with a K_{D} of less than 100 nM. In some embodiments, the humanized monoclonal anti-Tau antibody of this disclosure binds each of monomeric Tau, phosphorylated Tau, non-phosphorylated Tau, and oligomeric Tau with a K_{D} of less than 75 nM. In some embodiments, the humanized monoclonal anti-Tau antibody of this disclosure binds each of monomeric Tau, phosphorylated Tau, non-phosphorylated Tau, and oligomeric Tau with a K_{D} of less than 50 nM.

In some embodiments, the humanized monoclonal anti-Tau antibody of this disclosure binds to cynomolgus monkey Tau (SEQ ID NO: 10).

In some embodiments, an anti-Tau antibody comprises at least one, two, or three HVRs selected from (a) HVR-H1 comprising the amino acid sequence of SEQ ID NO: 2; (b) HVR-H2 comprising the amino acid sequence of SEQ ID NO: 3; and (c) HVR-H3 comprising the amino acid sequence of SEQ ID NO: 4. In some embodiments, an anti-Tau antibody comprises (a) HVR-H1 comprising the amino acid sequence of SEQ ID NO: 2; (b) HVR-H2 comprising the amino acid sequence of SEQ ID NO: 3; and (c) HVR-H3 comprising the amino acid sequence of SEQ ID NO: 4. In some embodiments, an anti-Tau antibody comprises (a) HVR-H1 comprising the amino acid sequence of SEQ ID NO: 2; (b) HVR-H2 comprising the amino acid sequence of SEQ ID NO: 3; (c) HVR-H3 comprising the amino acid sequence of SEQ ID NO: 4; (d) HVR-L1 comprising the amino acid sequence of SEQ ID NO: 6; (e) HVR-L2 comprising the amino acid sequence of SEQ ID NO: 7; and (f) HVR-L3 comprising the amino acid sequence of SEQ ID NO: 8.

In some embodiments, an anti-Tau antibody comprises at least one, two, or three HVRs selected from (a) HVR-H1 comprising the amino acid sequence of SEQ ID NO: 16; (b) HVR-H2 comprising the amino acid sequence of SEQ ID NO: 17; and (c) HVR-H3 comprising the amino acid sequence of SEQ ID NO: 18. In some embodiments, an anti-Tau antibody comprises (a) HVR-H1 comprising the amino acid sequence of SEQ ID NO: 16; (b) HVR-H2 comprising the amino acid sequence of SEQ ID NO: 17; and (c) HVR-H3 comprising the amino acid sequence of SEQ ID NO: 18. In some embodiments, an anti-Tau antibody comprises (a) HVR-H1 comprising the amino acid sequence of SEQ ID NO: 16; (b) HVR-H2 comprising the amino acid sequence of SEQ ID NO: 17; (c) HVR-H3 comprising the amino acid sequence of SEQ ID NO: 18; (d) HVR-L1 comprising the amino acid sequence of SEQ ID NO: 19; (e) HVR-L2 comprising the amino acid sequence of SEQ ID NO: 20; and (f) HVR-L3 comprising the amino acid sequence of SEQ ID NO: 21.

In some embodiments, the anti-Tau antibody is humanized. In some embodiments, the anti-Tau antibody comprises HVRs, e.g., as recited above, and further comprises an acceptor human framework, e.g., a human immunoglobulin framework or a human consensus framework.

In some embodiments, the antibody comprises a VH comprising the amino acid sequence of SEQ ID NO: 5. In some embodiments, the antibody comprises a VL comprising the amino acid sequence of SEQ ID NO: 9. In some embodiments, the antibody comprises a VH and a VL comprising the amino acid sequences of SEQ ID NO: 5 and SEQ ID NO: 9, respectively, including post-translational modifications of those sequences, if any.

In some embodiments, the anti-Tau antibody of this disclosure comprises a VH comprising an amino acid sequence that is at least 95% identical to the sequence of SEQ ID NO: 5. In some embodiments, the anti-Tau antibody of this disclosure comprises a VL comprising an amino acid sequence that is at least 95% identical to the sequence of SEQ ID NO: 9. In some embodiments, the anti-Tau antibody of this disclosure comprises a VH comprising an amino acid sequence that is at least 95% identical to the sequence of SEQ ID NO: 5; and a VL comprising an amino acid sequence that is at least 95% identical to the sequence of SEQ ID NO: 9. In some embodiments, the anti-Tau antibody of this disclosure comprises a VH having the amino acid sequence of SEQ ID NO: 5 and a VL having the amino acid sequence of SEQ ID NO:9.

In some embodiments, the antibody is an IgG4 antibody. In some embodiments, the IgG4 antibody comprises one or more mutations selected from M252Y, S254T, and T256E, according to EU numbering. In some embodiments, the IgG4 antibody comprises M252Y, S254T, and T256E mutations, according to EU numbering. In some embodiments, the antibody comprises an S228P mutation, according to EU numbering.

In some embodiments, the anti-Tau antibody comprises a heavy chain comprising an amino acid sequence that is at least 95% identical to the sequence of SEQ ID NO: 11. In some embodiments, the anti-Tau antibody comprises a heavy chain comprising an amino acid sequence that is at least 95% identical to the sequence of SEQ ID NO: 12. In some embodiments, the anti-Tau antibody comprises a light chain comprising an amino acid sequence that is at least 95% identical to the sequence of SEQ ID NO: 13. In some embodiments, the anti-Tau antibody comprises a heavy chain comprising the amino acid sequence of SEQ ID NO: 11. In some embodiments, the anti-Tau antibody comprises a heavy chain comprising the amino acid sequence of SEQ ID NO: 12. In some embodiments, the anti-Tau antibody comprises a light chain comprising the amino acid sequence of SEQ ID NO: 13. In some embodiments, the anti-Tau antibody comprises a heavy chain comprising the amino acid sequence of SEQ ID NO: 11 or SEQ ID NO: 12 and a light chain comprising the amino acid sequence of SEQ ID NO: 13. In some embodiments, the anti-Tau antibody comprises a heavy chain consisting of the amino acid sequence of SEQ ID NO: 11 or SEQ ID NO: 12 and a light chain consisting of the amino acid sequence of SEQ ID NO: 13. In some embodiments, the anti-Tau antibody comprises a heavy chain comprising the amino acid sequence of SEQ ID NO: 12 and a light chain comprising the amino acid sequence of SEQ ID NO: 13. In some embodiments, the anti-Tau antibody comprises of a heavy chain consisting of the amino acid sequence of SEQ ID NO: 12 and a light chain consisting of the amino acid sequence of SEQ ID NO: 13. In some embodiments, the anti-Tau antibody comprises a heavy chain comprising the amino acid sequence of SEQ ID NO: 11 and a light chain comprising the amino acid sequence of SEQ ID NO: 13. In some embodiments, the anti-Tau antibody comprises a heavy chain consisting of the amino acid sequence of SEQ ID NO: 11 and a light chain consisting of the amino acid sequence of SEQ ID NO: 13.

Other N-terminal binding anti-Tau antibodies may be used in the methods of the disclosure described herein. Examples of such antibodies include, without limitation, N-terminal binding anti-Tau antibodies disclosed in PCT/US2016/035409 and/or PCT/US2018/024300.

In some embodiments, the anti-Tau antibody according to any of the above embodiments is a monoclonal antibody, including a chimeric, humanized or human antibody. In some embodiments, the anti-Tau antibody is an antibody fragment, e.g., a Fv, Fab, Fab′, scFv, diabody, or F(ab′)₂ fragment. In some embodiments, the antibody is a full-length antibody, e.g., an intact IgG1 or IgG4 antibody or other antibody class or isotype as defined herein. In some embodiments, the anti-Tau antibody is semorinemab. In some embodiments, the anti-Tau antibody according to any of the above embodiments may incorporate any of the features, singly or in combination, as described in Sections 1-5 below:

### 1. Antibody Affinity

In certain embodiments, an antibody provided herein has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M).

In some embodiments, K_{D} is measured by a radiolabeled antigen-binding assay (RIA). In some embodiments, an RIA is performed with the Fab version of an antibody of interest and its antigen. For example, solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J Mol. Biol. 293:865-881(1999)). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [¹²⁵]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed, and the plate washed eight times with 0.1% polysorbate 20 (TWEEN-20®) in PBS. When the plates have dried, 150 μl/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to other embodiments, K_{D} is measured using a BIACORE® surface plasmon resonance assay. For example, an assay using a BIACORE®-2000 or a BIACORE®-3000 (BIAcore, Inc., Piscataway, NJ) is performed at 25° C. with immobilized antigen CM5 chips at ˜10 resonance units (RU). In some embodiments, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 pM) before injection at a flow rate of 5 μl/minute to achieve approximately 10 resonance units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 μl/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (K_{D}) is calculated as the ratio k_{off}/kₒₙ (Chen et al., J. Mol. Biol. 293:865-881 (1999)). If the on-rate exceeds 10⁶ M⁻¹s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

### 2. Antibody Fragments

In certain embodiments, the antibody provided herein is an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see, e.g., Pluckthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003). Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, MA; see, e.g., U.S. Pat. No. 6,248,516 B1).

Antibody fragments can be made by various techniques, including but not limited to proteolytic digestion of an intact antibody, as well as production by recombinant host cells (e.g., E. coli or phage), as described herein.

### 3. Chimeric and Humanized Antibodies

In certain embodiments, an antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); US Patent Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J Cancer, 83:252-260 (2000) (describing the “guided selection” approach to FR shuffling).

Human framework regions that may be used for humanization include but are not limited to: framework regions selected using the “best-fit” method (see, e.g., Sims et al. J Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

### 4. Multispecific Antibodies

In certain embodiments, an antibody provided herein is a multispecific antibody, e.g., a bispecific antibody. Multispecific antibodies are monoclonal antibodies that have binding specificities for at least two different sites. In certain embodiments, one of the binding specificities is for Tau and the other is for any other antigen. In certain embodiments, one of the binding specificities is for Tau and the other is for amyloid beta. In certain embodiments, bispecific antibodies may bind to two different epitopes of Tau. Bispecific antibodies may also be used to localize cytotoxic agents to cells that express Tau. Bispecific antibodies can be prepared as full-length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992)); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g., Gruber et al., J. Immunol., 152:5368 (1994)); and preparing trispecific antibodies as described, e.g., in Tutt et al. J Immunol. 147: 60 (1991).

Engineered antibodies with three or more functional antigen binding sites, including “Octopus antibodies,” are also included herein (see, e.g., US 2006/0025576A1). The antibody or fragment herein also includes a “Dual Acting FAb” or “DAF” comprising an antigen binding site that binds to Tau as well as another, different antigen (see, US 2008/0069820, for example).

### 5. Antibody Variants

In certain embodiments, amino acid sequence variants of the antibodies provided herein are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

a. Substitution, Insertion, and Deletion Variants

In certain embodiments, antibody variants having one or more amino acid substitutions are provided. Sites of interest for substitutional mutagenesis include the HVRs and FRs. Conservative substitutions are shown in Table 2 under the heading of “preferred substitutions.” More substantial changes are provided in Table 2 under the heading of “exemplary substitutions,” and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

TABLE 2

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity-matured antibody, which may be conveniently generated, e.g., using phage display-based affinity maturation techniques such as those described herein. Briefly, one or more HVR residues are mutated and the variant antibodies displayed on phage and screened for a particular biological activity (e.g., binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g. Chowdhury, Methods Mol. Biol. 207:179-196 (2008)), and/or residues that contact antigen, with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, NJ, (2001)). In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may, for example, be outside of antigen contacting residues in the HVRs. In certain embodiments of the variant VH and VL sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

In some embodiments, the antibody is an IgG4 antibody. IgG4 antibodies are known to possess reduced effector function. IgG4 antibodies are dynamic molecules that are able to undergo a process known as Fab arm exchange. Certain amino acid substitutions, e.g., S228P can prevent Fab arm exchange in IgG4 antibodies. In some embodiments, the IgG4 antibody comprises a S228P substitution.

b. Glycosylation Variants

In certain embodiments, an antibody provided herein is altered to increase or decrease the extent to which the antibody is glycosylated. Addition or deletion of glycosylation sites to an antibody may be accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region (Wright et al. TIBTECH 15:26-32 (1997)). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the disclosure may be made in order to create antibody variants with certain improved properties.

In some embodiments, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g., complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues); however, Asn297 mgy also be located about ±3 amino acids upstream or downstream of position 297, i.e., between positions 294 and 300, due to minor sequence variations in antibodies. Such fucosylation variants may have improved ADCC function (see, e.g., US Patent Publication Nos. US 2003/0157108 (Presta, L.); US 2004/0093621 (Kyowa Hakko Kogyo Co., Ltd)). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Antibody variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

c. Fc Region Variants

In certain embodiments, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g., a substitution) at one or more amino acid positions.

In certain embodiments, the disclosure contemplates an antibody variant that possesses some but not all effector functions, which make it a desirable candidate for applications in which the half-life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity) but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g., Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (Cell Technology, Inc. Mountain View, CA; and CytoTox 96® non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA95:652-656 (1998). Clq binding assays may also be carried out to confirm that the antibody is unable to bind Clq and hence lacks CDC activity. See, e.g., Clq and C3c binding ELISA in WO 2006/029879 and WO 2005/100402. To assess complement activation, a CDC assay may be performed (Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half-life determinations can also be performed using methods known in the art (see, e.g., Petkova, S. B. et al., Int'l. Immunol. 18(12):1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fe mutant with substitution of residues 265 and 297 to alanine (U.S. Pat. No. 7,332,581).

Certain antibody variants with improved or diminished binding to FcRs are described (U.S. Pat. No. 6,737,056; WO 2004/056312, Shields et al., J. Biol. Chem. 9(2): 6591-6604 (2001)).

In certain embodiments, an antibody variant comprises an Fc region with one or more amino acid substitutions which improve ADCC, e.g., substitutions at positions 298, 333, and/or 334 of the Fc region (EU numbering of residues). In some embodiments, alterations are made in the Fc region that result in altered (i.e., either improved or diminished) Clq binding and/or Complement Dependent Cytotoxicity (CDC), e.g., as described in U.S. Pat. No. 6,194,551, WO 99/51642, and Idusogie et al. J Immunol. 164: 4178-4184 (2000).

Antibodies with increased half-lives and improved binding to the neonatal Fc receptor (FcRn), which is responsible for the transfer of maternal IgGs to the fetus (Guyer et al., J Immunol. 117:587 (1976); Kim et al., J. Immunol. 24:249 (1994)), are described in US 2005/0014934A1 (Hinton et al.). Those antibodies comprise an Fc region with one or more substitutions therein which improve binding of the Fc region to FcRn. Such Fc variants include those with substitutions at one or more of Fc region residues: 238, 252, 254, 256, 265, 272, 286, 303, 305, 307, 311, 312, 317, 340, 356, 360, 362, 376, 378, 380, 382, 413, 424 or 434, e.g., substitution of Fc region residue 434 (e.g., U.S. Pat. No. 7,371,826). See also Duncan & Winter, Nature 322:738-40 (1988); U.S. Pat. Nos. 5,648,260; 5,624,821; and WO 94/29351 concerning other examples of Fc region variants. In some embodiments, the antibody is an IgG4 antibody. In some embodiments, the IgG4 antibody comprises one or more mutations selected from M252Y, S254T, and T256E, according to EU numbering. In some embodiments, the IgG4 antibody comprises M252Y, S254T, and T256E mutations, according to EU numbering.

d. Cysteine Engineered Antibody Variants

In certain embodiments, it may be desirable to create cysteine-engineered antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies may be generated as described, e.g., in U.S. Pat. No. 7,521,541.

e. Antibody Derivatives

In certain embodiments, an antibody provided herein may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, polypropylene glycol homopolymers, polypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In some embodiments, conjugates of an antibody and nonproteinaceous moiety that may be selectively heated by exposure to radiation are provided. In some embodiments, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

### A. Recombinant Methods and Compositions

Antibodies may be produced using recombinant methods and compositions, e.g., as described in U.S. Pat. No. 4,816,567. In some embodiments, isolated nucleic acid encoding an anti-Tau antibody described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising the nucleic acid are provided. In a further embodiment, a host cell comprising the nucleic acid is provided. In some embodiments, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In some embodiments, the host cell is eukaryotic, e.g., a Chinese Hamster Ovary (CHO) cell or lymphoid cell (e.g., Y0, NS0, Sp20 cell). In some embodiments, a method of making an anti-Tau antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an anti-Tau antibody, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523 (see also Charlton, Methods in Molecular Biology, Vol. 248 (B.K.C. Lo, ed., Humana Press, Totowa, NJ, 2003), pp. 245-254, describing expression of antibody fragments in E. coli.). After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern (Gerngross, Nat. Biotech. 22:1409-1414 (2004), and Li et al., Nat. Biotech. 24:210-215 (2006)).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts (e.g., U.S. Pat. Nos. 5,959,177, 6,040,498, 6,420,548, 7,125,978, and 6,417,429 (describing PLANTIBODIES™ technology for producing antibodies in transgenic plants)).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK); buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR⁻ CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Mol. Biology, Vol. 248 (B.K.C. Lo, ed., Humana Press, Totowa, NJ), pp. 255-268 (2003).

### B. Assays

Anti-Tau antibodies provided herein may be identified, screened for, or characterized for their physical/chemical properties and/or biological activities by various assays known in the art.

### 1. Binding Assays and Other Assays

An antibody of the disclosure may be tested for its antigen binding activity, e.g., by known methods such as ELISA, Western blot, etc.

Competition assays may also be used to identify an antibody that competes with an antibody described herein for binding to Tau. In certain embodiments, such a competing antibody binds to the same epitope (e.g., a linear or a conformational epitope) that is bound by semorinemab. Detailed exemplary methods for mapping an epitope to which an antibody binds are provided in Morris (1996) “Epitope Mapping Protocols,” in Methods in Molecular Biology vol. 66 (Humana Press, Totowa, NJ).

In an exemplary competition assay, immobilized Tau (such as monomeric Tau) is incubated in a solution comprising a first labeled antibody that binds to Tau (e.g., any antibody described herein, such as semorinemab), and a second unlabeled antibody that is being tested for its ability to compete with the first antibody for binding to Tau. The second antibody may be present in a hybridoma supernatant. As a control, immobilized Tau is incubated in a solution comprising the first labeled antibody but not the second unlabeled antibody. After incubation under conditions permissive for binding of the first antibody to Tau, excess unbound antibody is removed, and the amount of label associated with immobilized Tau is measured. If the amount of label associated with immobilized Tau is substantially reduced in the test sample relative to the control sample, then that indicates that the second antibody is competing with the first antibody for binding to Tau (Harlow and Lane (1988) Antibodies: A Laboratory Manual ch. 14 (Cold Spring Harbor Laboratory, Cold Spring Harbor, NY)).

### 2. Activity Assays

The present disclosure also provides assays for identifying anti-Tau (e.g., pan-Tau) antibodies thereof having biological activity. Biological activity may include, e.g., binding of such antibodies to multiple forms of Tau (e.g., monomeric Tau, oligomeric Tau, non-phosphorylated Tau, and phosphorylated Tau) and reducing the level of Tau protein (e.g., total Tau, total soluble Tau, soluble non-phosphorylated Tau, soluble phosphorylated Tau, total insoluble Tau, insoluble non-phosphorylated Tau, insoluble phosphorylated Tau, hyperphosphorylated Tau, or paired helical filaments containing hyperphosphorylated Tau, in the brain, e.g., in the brain cortex and/or hippocampus). Antibodies having such biological activity in vivo and/or in vitro are also provided.

In certain embodiments, an antibody of the disclosure is tested for such biological activity. For example, an animal model of tauopathy, such as Tau transgenic mice (e.g., P301L), can be used to detect binding of anti-Tau antibodies to brain sections, and for example, to neurofibrillary tangles in the brains of the transgenic mice. Further, an animal model of tauopathy, such as a Tau transgenic mice (e.g., P301L), can be treated with anti-Tau antibodies and experimental techniques known in the art can be used to assess whether such treatment reduces the level of Tau protein (e.g., total Tau, total soluble Tau, soluble phosphorylated Tau, soluble non-phosphorylated Tau, total insoluble Tau, insoluble phosphorylated Tau, insoluble non-phosphorylated Tau, hyperphosphorylated Tau, or paired helical filaments containing hyperphosphorylated Tau) in the mouse brain (e.g., in the brain cortex and/or hippocampus).

### 3. Assays for Diagnosis and Detection

In certain embodiments, any of the anti-Tau antibodies provided herein is useful for detecting the presence of Tau in a biological sample. The term “detecting” as used herein encompasses quantitative or qualitative detection. In certain embodiments, a biological sample comprises a cell or tissue, such as serum, plasma, nasal swabs, sputum, cerebrospinal fluid, aqueous humor of the eye and the like, or tissue or cell samples obtained from an organism such as samples containing neural or brain tissue.

In some embodiments, an anti-Tau antibody for use in a method of diagnosis or detection is provided. In a further aspect, a method of detecting the presence of Tau in a biological sample is provided. In certain embodiments, the method comprises contacting the biological sample with an anti-Tau antibody as described herein under conditions permissive for binding of the anti-Tau antibody to Tau and detecting whether a complex is formed between the anti-Tau antibody and Tau. Such method may be an in vitro or in vivo method.

Exemplary disorders that may be diagnosed using an antibody described herein are tau pathologies, in particular, those characterized by aggregate Tau in the brain, or by Tau load in the extracellular space substantially similar to that found in mild-to-moderate AD. Non-limiting examples of disorders for diagnosis with an antibody described herein include mild-to-moderate or moderate AD, amyotrophic lateral sclerosis, Parkinson's disease, Creutzfeldt-Jacob disease, dementia pugilistica, Down's syndrome, Gerstmann-Straussler-Scheinker disease, inclusion-body myositis, prion protein cerebral amyloid angiopathy, traumatic brain injury, amyotrophic lateral sclerosis/parkinsonism-dementia complex of Guam, non-Guamanian motor neuron disease with neurofibrillary tangles, argyrophilic grain dementia, corticobasal degeneration, diffuse neurofibrillary tangles with calcification, frontotemporal dementia, frontotemporal dementia with parkinsonism linked to chromosome 17, Hallevorden-Spatz disease, multiple system atrophy, Niemann-Pick disease type C, pallido-ponto-nigral degeneration, Pick's disease, progressive subcortical gliosis, progressive supranuclear palsy, subacute sclerosing panencephalitis, tangle only dementia, postencephalitic parkinsonism, and myotonic dystrophy. In some embodiments, the tauopathy is progressive supranuclear palsy.

In certain embodiments, labeled anti-Tau antibodies are provided. Labels include, but are not limited to, labels or moieties that are detected directly (such as fluorescent, chromophoric, electron-dense, chemiluminescent, and radioactive labels), as well as moieties, such as enzymes or ligands, that are detected indirectly, e.g., through an enzymatic reaction or molecular interaction. Exemplary labels include, but are not limited to, the radioisotopes ³³P, ¹⁴C, ¹²⁵I, ³H, and 131I, fluorophores such as rare earth chelates or fluorescein and its derivatives, rhodamine and its derivatives, dansyl, umbelliferone, luceriferases, e.g., firefly luciferase and bacterial luciferase (U.S. Pat. No. 4,737,456), luciferin, 2,3-dihydrophthalazinediones, horseradish peroxidase (HRP), alkaline phosphatase, β-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase, or microperoxidase, biotin/avidin, spin labels, bacteriophage labels, stable free radicals, and the like.

### 4. Immunoconjugates

In some embodiments, any of the anti-Tau antibodies provided herein is useful in forming an immunoconjugate comprising the anti-Tau antibody conjugated to one or more other therapeutic agents or radioactive isotopes.

In some embodiments, the immunoconjugate comprises an antibody as described herein conjugated to a radioactive atom to form a radioconjugate. A variety of radioactive isotopes are available for the production of radioconjugates. Examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. When the radioconjugate is used for detection, it may comprise a radioactive atom for scintigraphic studies, for example Tc-99m or ¹²³I, or a spin label for nuclear magnetic resonance (NMR) imaging (also known as magnetic resonance imaging, MRI), such as iodine-123, iodine-131, indium-111, fluorine-19, carbon-13, nitrogen-15, oxygen-17, gadolinium, manganese or iron.

Conjugates of an antibody may be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody (WO94/11026). The linker may be a “cleavable linker” facilitating release of a cytotoxic drug in the cell. For example, an acid-labile linker, peptidase-sensitive linker, photolabile linker, dimethyl linker or disulfide-containing linker (Chari et al., Cancer Res. 52:127-131 (1992); U.S. Pat. No. 5,208,020) may be used.

The immunoconjugates or ADCs herein expressly contemplate, but are not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, IL., U.S.A).

### C. Pharmaceutical Formulations

In a further aspect, the disclosure provides pharmaceutical formulations comprising any of the anti-Tau antibodies provided herein, e.g., for use in any of the therapeutic methods described. In some embodiments, a pharmaceutical formulation comprises the anti-Tau antibody and a pharmaceutically acceptable carrier. In certain embodiments, the pharmaceutical formulation further comprises at least one additional therapeutic agent, e.g., as described herein.

Pharmaceutical formulations of an anti-Tau antibody as described herein can be prepared by mixing such antibody having the desired degree of purity with one or more optional pharmaceutically acceptable carriers, diluents, and/or excipients (Remington's Pharmaceutical Sciences 16th edition, Osol, A. Ed. (1980)), in the form of lyophilized formulations or aqueous solutions. Exemplary lyophilized antibody formulations are described, e.g., in U.S. Pat. No. 6,267,958. Aqueous antibody formulations include those described in U.S. Pat. No. 6,171,586 and WO2006/044908, the latter formulations including a histidine-acetate buffer.

Pharmaceutically acceptable carriers, diluents, and excipients are generally nontoxic to recipients at the doses and concentrations used, and include, but are not limited to: sterile water, buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

In any of the foregoing embodiments and aspects, the antibody may be semorinemab.

The formulation herein may also contain more than one therapeutic agent or active ingredient, as necessary for the particular indication being treated, preferably those with complementary activities that do not adversely affect each other. For example, it may be desirable to further provide one or more compounds to prevent or treat symptoms of AD. Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, e.g., films, or microcapsules.

The formulations to be used for in vivo administration are generally sterile. Sterility may be readily accomplished, e.g., by filtration through sterile filtration membranes.

### Therapeutic Methods and Compositions for Use Therein

As discussed above, the present disclosure provides the first clinical data showing slowed clinical decline and reduced disease progression in mild-to-moderate and moderate AD using an anti-Tau approach. Specifically, patients with mild-to-moderate or moderate AD showed a reduction in the rate of cognitive decline when treated with semorinemab as compared to a placebo. Furthermore, the dose used did not increase the incidence of the adverse events. Therefore, in one aspect, the disclosure provides use of any of the anti-Tau antibodies in the treatment of mild-to-moderate or moderate AD and related tauopathies. Such anti-Tau antibody is provided for use as a medicament. In some embodiments, the anti-Tau antibodies are for the treatment of mild-to-moderate AD. In some embodiments, the anti-Tau antibodies are for the treatment of moderate AD.

In some embodiments, the tauopathy is as a neurodegenerative tauopathy. In some embodiments, the tau pathology is characterized by Tau pathology in the extracellular space of the patient's brain substantially similar to that found in mild-to-moderate AD. Exemplary Tau pathologies that can be treated with an anti-Tau antibody disclosed herein include, without limitation, mild-to-moderate AD, amyotrophic lateral sclerosis, Parkinson's disease, Creutzfeldt-Jacob disease, dementia pugilistica, Down's syndrome, Gerstmann-Straussler-Scheinker disease, inclusion-body myositis, prion protein cerebral amyloid angiopathy, traumatic brain injury, amyotrophic lateral sclerosis/parkinsonism-dementia complex of Guam, non-Guamanian motor neuron disease with neurofibrillary tangles, argyrophilic grain dementia, corticobasal degeneration, diffuse neurofibrillary tangles with calcification, frontotemporal dementia, frontotemporal dementia with parkinsonism linked to chromosome 17, Hallevorden-Spatz disease, multiple system atrophy, Niemann-Pick disease type C, pallido-ponto-nigral degeneration, Pick's disease, progressive subcortical gliosis, progressive supranuclear palsy, subacute sclerosing panencephalitis, tangle only dementia, postencephalitic parkinsonism, and myotonic dystrophy. In some embodiments, an anti-Tau antibody for use in treating mild-to-moderate AD is provided. In some embodiments, an anti-Tau antibody for use in treating progressive supranuclear palsy is provided. In some embodiments, an anti-Tau antibody for use in treating a tau pathology is provided, wherein the tau pathology is characterized by Tau pathology in the extracellular space of the patient's brain substantially similar to that found in mild-to-moderate AD.

Further, Tau pathologies that can be treated with an anti-Tau antibody include diseases or disorders that show an impairment or loss of a cognitive function, such as reasoning, situational judgement, memory capacity, learning, and/or special navigation. In certain embodiments, the disclosure provides an anti-Tau antibody for use in a method of treating an individual, having any one of the Tau pathologies described above, comprising administering to the individual an effective amount of the anti-Tau antibody. In one such embodiment, the method further comprises administering to the individual an effective amount of at least one additional therapeutic agent, e.g., concomitant symptomatic medications.

In any of the foregoing embodiments and aspects, the antibody of the disclosure may be semorinemab.

A patient is typically first assessed for the presence of one or more tauopathies prior to determining the suitability of treatment. As one nonlimiting example, mild-to-moderate AD may be diagnosed in a patient using the NINCDS-ADRDA (Neurological and Communicative Disorders and Stroke-Alzheimer's Disease Related Disorders Assessment) criteria (McKhann, et al., 1984, Neurology 34:939-44). A potential patient to be administered one or more antibodies described herein may also be tested for the presence or absence of one or more genetic markers that predispose the patient either to (i) a higher or lower likelihood of the patient experiencing one or more tauopathies, (ii) a higher or lower likelihood of the patient benefiting from treatment, or (iii) a higher or lower likelihood of the patient experiencing one or more adverse events or side effects during the course of administration of the antibody. As one nonlimiting example, it is known that patients carrying the Apoε4 allele have a substantially higher risk of developing AD than those lacking the allele (Saunders et al, Neurology 1993; 43: 1467-72; Prekumar et al., Am. J. Pathol. 1996; 148:2083-95).

In some embodiments, the antibody disclosed herein is used to treat mild-to-moderate AD in a patient. The patient can be Apoε4 positive or Apoε4 negative. In some embodiments, the antibody is used to treat an Apoε4 positive patient with mild-to-moderate AD. In some embodiments, the antibody is used to treat an Apoε4 positive patient with moderate AD. In some embodiments, the patient is Apoε4 positive. In some embodiments, the patient is Apoε4 negative.

In some embodiments, the antibody disclosed herein is used to treat a patient having an MMSE score of between 16 and 21, between 16 and 18, or between 19 and 21. In some embodiments, the antibody is used to treat a patient having CDR GS of 1 or 2.

In some embodiments, an antibody disclosed herein is used to treat a patient having an MMSE score of between 16 and 20, between 16 and 19, between 16 and 18, between 16 and 17, between 17 and 21, between 17 and 20, between 17 and 19, between 17 and 18, between 18 and 21, between 18 and 20, between 18 and 19, between 19 and 21, or between 19 and 20. As used herein, an MMSE score between two numbers includes the numbers at each end of the range. For example, an MMSE score between 16 and 21 includes MMSE scores of 16 and 21.

In some embodiments, a patient of this disclosure has an MMSE score of 16-19, inclusive, before administration of a humanized monoclonal anti-Tau antibody, optionally MMSE score of 16-18, inclusive, before administration of the antibody. In some embodiments, a patient of this disclosure has an MMSE score of 16-19, inclusive, before administration of a humanized monoclonal anti-Tau antibody. In some embodiments, a patient of this disclosure has an MMSE score of 16-18, inclusive, before administration of a humanized monoclonal anti-Tau antibody.

In some embodiments, the antibodies disclosed herein are used to treat a patient who is Tau positive. In particular embodiments, the antibody is used to treat a patient having Tau load or Tau pathology typical of that seen in patients diagnosed with mild-to-moderate AD. In particular embodiments, the antibody is used to treat a patient having Tau load or Tau pathology typical of that seen in patients diagnosed with moderate AD.

In some embodiments, the patient of this disclosure is Tau positive and/or amyloid beta (Abeta) positive. In some embodiments, the patient is determined to be Tau positive by administering to the patient a positron emission tomography (PET) tracer that binds to Tau. In some embodiments, the patient is determined to be Abeta positive by administering to the patient a PET tracer that binds to Abeta. In some embodiments, the patient of this disclosure is Tau positive, optionally wherein the patient is determined to be Tau positive by administering to the patient a positron emission tomography (PET) tracer that binds to Tau. In some embodiments, the patient of this disclosure is Abeta positive, optionally wherein the patient is determined to be Abeta positive by administering to the patient a positron emission tomography (PET) tracer that binds to Abeta. In some embodiments, the patient of this disclosure is Tau positive and/or Abeta positive. In some embodiments, the patient of this disclosure is Tau positive. In some embodiments, the patient of this disclosure is Abeta positive. In some embodiments, the patient is determined to be Tau positive by administering to the patient a positron emission tomography (PET) tracer that binds to Tau. In some embodiments, the patient is determined to be Abeta positive by administering to the patient a positron emission tomography (PET) tracer that binds to Abeta.

In further embodiments, the disclosure provides an anti-Tau antibody for use in slowing Tau accumulation (e.g., total Tau, total soluble Tau, soluble phosphorylated Tau, total insoluble Tau, aggregated Tau, insoluble phosphorylated Tau, hyperphosphorylated Tau, or paired helical filaments containing hyperphosphorylated Tau) in a patient. For example, the antibody may be used to slow aggregate Tau in the extracellular space between neuronal cells in the patient's brain. In some embodiments, the disclosure provides an anti-Tau antibody for use in slowing the accumulation of Tau protein in a patient as measured by Tau PET scan. For example, such slowed accumulation can occur in the brain (e.g., in the brain cortex and/or hippocampus). In some embodiments, the disclosure provides an anti-Tau antibody for use in slowing accumulation of phosphorylated Tau, including soluble phosphorylated Tau. In some embodiments, the disclosure provides an anti-Tau antibody for use in slowing accumulation of aggregated Tau. In some embodiments, the disclosure provides an anti-Tau antibody for use in slowing accumulation of insoluble Tau (e.g., insoluble phosphorylated Tau). In some embodiments, the disclosure provides an anti-Tau antibody for use in slowing accumulation of hyperphosphorylated Tau. In some embodiments, the disclosure provides an anti-Tau antibody for use in slowing accumulation of paired helical filaments (e.g., paired helical filaments containing hyperphosphorylated Tau) in a brain tissue (e.g., in the brain cortex and/or hippocampus). In certain embodiments, the disclosure provides an anti-Tau antibody for use in a method of slowing accumulation of Tau protein (e.g., total Tau, total soluble Tau, soluble phosphorylated Tau, total insoluble Tau, aggregated Tau, insoluble phosphorylated Tau, hyperphosphorylated Tau, or paired helical filaments containing hyperphosphorylated Tau) in the brain (e.g., in the brain cortex and/or hippocampus) in a patient comprising administering to the patient an effective amount (e.g., 4500 mg) of the anti-Tau antibody to slow accumulation of Tau. In some embodiments, the antibody binds an epitope within the N-terminal region of Tau (an N-terminal binding anti-Tau antibody), e.g., binding to an epitope within amino acid residues 2 to 24 of mature human Tau, e.g., binding to an epitope within/spanning amino acid residues 6 to 23 of mature human Tau. In some embodiments, the antibody is semorinemab. Without being limited to a hypothesis or theory, the anti-tau antibody described herein may be used to intercept tau extracellularly to block seeding/spreading of further tau deposits to heretofore unaffected neurons.

In further embodiments, the disclosure provides a method of slowing Tau accumulation by administering to a patient a therapeutically effective amount of an anti-Tau antibody (e.g., total Tau, total soluble Tau, soluble phosphorylated Tau, total insoluble Tau, aggregated Tau, insoluble phosphorylated Tau, hyperphosphorylated Tau, or paired helical filaments containing hyperphosphorylated Tau). For example, the antibody may be used to slow aggregate Tau in the extracellular space between neuronal cells in the patient's brain. In some embodiments, the disclosure provides a method of slowing the accumulation of Tau protein in a patient as measured by Tau PET scan by administering to the patient a therapeutically effective amount of an anti-Tau antibody. For example, such slowed accumulation can occur in the brain (e.g., in the brain cortex and/or hippocampus). In some embodiments, the disclosure provides a method of slowing accumulation of phosphorylated Tau, including soluble phosphorylated Tau, by administering to a subject a therapeutically effective amount of an anti-Tau antibody. In some embodiments, the disclosure provides a method of slowing accumulation of aggregated Tau by administering to a subject a therapeutically effective amount of an anti-Tau antibody. In some embodiments, the disclosure provides a method of slowing accumulation of insoluble Tau (e.g. insoluble phosphorylated Tau) by administering to a subject a therapeutically effective amount of an anti-Tau antibody. In some embodiments, the disclosure provides a method of slowing accumulation of hyperphosphorylated Tau by administering to a subject a therapeutically effective amount of an anti-Tau antibody. In some embodiments, the disclosure provides a method of slowing accumulation of paired helical filaments (e.g., paired helical filaments containing hyperphosphorylated Tau) in a brain tissue (e.g., in the brain cortex and/or hippocampus) by administering to a subject a therapeutically effective amount of an anti-Tau antibody. In certain embodiments, the disclosure provides a method of slowing accumulation of Tau protein (e.g., total Tau, total soluble Tau, soluble phosphorylated Tau, total insoluble Tau, aggregated Tau, insoluble phosphorylated Tau, hyperphosphorylated Tau, or paired helical filaments containing hyperphosphorylated Tau) in the brain (e.g., in the brain cortex and/or hippocampus) in a patient comprising administering to the patient a therapeutically effective amount (e.g., 4500 mg) of the anti-Tau antibody to slow accumulation of Tau. In some embodiments, the antibody binds an epitope within the N-terminal region of Tau (an N-terminal binding anti-Tau antibody), e.g., binding to an epitope within amino acid residues 2 to 24 of mature human Tau, e.g., binding to an epitope within/spanning amino acid residues 6 to 23 of mature human Tau. In some embodiments, the antibody is semorinemab.

In some embodiments, a reduction in the level of Tau is determined by measuring the density and/or extent of Tau pathology and/or aggregated Tau. As such, reduced density or extent of Tau pathology and/or aggregated Tau (measured, e.g., by positron emission tomography imaging) is considered indicative of a reduction in the level of Tau. The level of Tau, non-phosphorylated Tau, phosphorylated Tau, or hyperphosphorylated Tau can be measured by positron emission tomography (PET) or by analysis of cerebrospinal fluid, such as cerebrospinal fluid obtained via lumbar puncture. In some embodiments, a reduction in the level of Tau protein is determined by measuring the level of a Tau fragment.

In some embodiments, a level of Tau is measured by a standardized uptake value ratio (SUVR) measurement of a scan that shows distribution of the PET tracer in a patient's brain of this disclosure. In some embodiments, the patient of this disclosure has a high level of Tau, wherein the high level of Tau corresponds to one or more of: (i) an intracerebral Tau level above or equal to median Genentech Tau Probe 1 (GTP1) whole cortical gray (WCG) (top medium split); (ii) an SUVR measurement from the temporal region that is equal to or greater than 1.325; and (iii) an SUVR measurement from the whole cortical gray (WCG) region that is equal to or greater than 1.245. In some embodiments, the patient of this disclosure has a high level of Tau, wherein the high level of Tau corresponds to an intracerebral Tau level above or equal to median Genentech Tau Probe 1 (GTP1) whole cortical gray (WCG) (top medium split). In some embodiments, the patient of this disclosure has a high level of Tau, wherein the high level of Tau corresponds to an SUVR measurement from the temporal region that is equal to or greater than 1.325. In some embodiments, the patient of this disclosure has a high level of Tau, wherein the high level of Tau corresponds to an SUVR measurement from the whole cortical gray (WCG) region that is equal to or greater than 1.245. In some embodiments, the patient has a low level of Tau, wherein the low level of Tau corresponds to one or more of (i) an intracerebral Tau level below median GTP1 WCG (bottom medium split); (ii) an SUVR measurement from the temporal region that is less than 1.325; and (iii) an SUVR measurement from the WCG that is less than 1.245. In some embodiments, the patient has a low level of Tau, wherein the low level of Tau corresponds to an intracerebral Tau level below median GTP1 WCG (bottom medium split). In some embodiments, the patient has a low level of Tau, wherein the low level of Tau corresponds to an SUVR measurement from the temporal region that is less than 1.325. In some embodiments, the patient has a low level of Tau, wherein the low level of Tau corresponds to an SUVR measurement from the WCG that is less than 1.245.

In some embodiments, the disclosure provides an anti-Tau antibody for use in modulating the Tau load (e.g., total Tau, total soluble Tau, soluble phosphorylated Tau, total insoluble Tau, aggregated Tau, insoluble phosphorylated Tau, hyperphosphorylated Tau, or paired helical filaments containing hyperphosphorylated Tau), for example, in the brain (e.g., in the brain cortex and/or hippocampus) of a patient. In some embodiments, the disclosure provides a method of modulating the Tau load (e.g., total Tau, total soluble Tau, soluble phosphorylated Tau, total insoluble Tau, aggregated Tau, insoluble phosphorylated Tau, hyperphosphorylated Tau, or paired helical filaments containing hyperphosphorylated Tau), for example, in the brain (e.g., in the brain cortex and/or hippocampus) of a patient by administering to the patient a therapeutically effective amount of an anti-Tau antibody. In some embodiments, the antibody is semorinemab. In some embodiments, the dose is 4500 mg semorinemab.

In a further aspect, the disclosure provides for the use of an anti-Tau antibody in the manufacture or preparation of a medicament. The medicament may be for treatment of any of the tau pathologies discussed above. In some embodiments, the medicament is for treatment of mild-to-moderate or moderate AD. In some embodiments, the medicament is for the treatment of mild-to-moderate AD. In some embodiments, the medicament is for treatment of moderate AD. In some embodiments, the medicament is for reducing the Tau pathology (e.g., total Tau, total soluble Tau, soluble phosphorylated Tau, total insoluble Tau, aggregated Tau, insoluble phosphorylated Tau, hyperphosphorylated Tau, or paired helical filaments containing hyperphosphorylated Tau) in the brain of a patient.

In some aspects, the disclosure provides a method for alleviating one or more symptoms of a Tau pathology; or provides an anti-Tau antibody, or a medicament comprising anti-Tau antibody, for alleviating one or more symptoms of a Tau pathology (such as any of the diseases or disorders described herein, for example, mild-to-moderate AD or moderate AD). In some aspects, the disclosure provides a method for reducing the number of symptoms or the severity of one or more symptoms of a Tau pathology; or provides an anti-Tau antibody or a medicament comprising anti-Tau antibody for reducing the number of symptoms or the severity of a Tau pathology (such as any of the diseases or disorders described herein, for example, AD). In a particular embodiment, the symptom of the Tau pathology is an impairment in cognition or reduced cognitive capacity, such as progressive decline in cognitive capacity. In some embodiments, the symptom of the Tau pathology is an impairment in praxis, such as progressive decline in praxis. In some embodiments, the symptom of the Tau pathology is an impairment in learning, such as progressive decline in learning. In some embodiments, the symptom of the Tau pathology is an impairment in memory, such as progressive decline in memory capabilities. In some embodiments, the symptom of the Tau pathology is a long-term memory loss. In a specific embodiment, the symptom of the Tau pathology is dementia. In some embodiments, the symptom of the Tau protein pathology is confusion, irritability, aggression, mood swings, or a language impairment. In some embodiments, the symptom of the Tau protein pathology is an impairment or loss of one or more cognitive functions such as reasoning, situational judgment, memory capacity, praxis, and/or learning. In an additional aspect, the disclosure provides methods of treating mild-to-moderate or moderate AD and related tauopathies in a subject in need thereof by administering to the subject a therapeutically effective amount of an anti-Tau antibody. In some embodiments, the method is a method of treating mild-to-moderate AD. In some embodiments, the method is a method of treating moderate AD. The methods provided herein comprise administration of an amount (e.g., therapeutically effective amount such as 4500 mg) of an anti-Tau antibody to a patient (e.g., who displays one or more symptoms of a Tau pathology). In a particular embodiment, the antibody is semorinemab.

In specific aspects, the disclosure provides a method for retaining or increasing memory capacity, memory function, or cognitive function, or for slowing down memory loss or loss of cognitive capacity associated with a Tau pathology. Accordingly, in some aspects, the disclosure provides an anti-Tau antibody, or a medicament comprising anti-Tau antibody, for retaining or increasing memory capacity, memory function, or cognitive function, or for slowing down memory loss or loss of cognitive capacity associated with a Tau pathology (such as any of the diseases or disorders described herein, for example, AD, mild-to-moderate AD, or moderate AD, (where loss of memory generally is a core feature of the disease progression)). Memory may be assessed, e.g., as one of the three domains making up ADAS-Cog11 scores, e.g., as described herein (ADAS-Cog11 memory domain scores). In particular embodiments, memory is assessed in terms of word recognition and/or word recall, e.g., as assessed using an ADAS-Cog11 test. The methods provided herein comprise administration of an amount (e.g., therapeutically effective amount such as 4500 mg) of an anti-Tau antibody to a patient (e.g., who displays one or more symptoms of memory loss or a decrease of memory capacity). In a particular embodiment, the antibody is semorinemab.

In specific aspects, the disclosure provides a method for slowing decline in word recognition capability associated with a Tau pathology. Accordingly, in some aspects, the disclosure provides an anti-Tau antibody, or a medicament comprising anti-Tau antibody, for slowing down loss in word recognition and/or word recall associated with a Tau pathology (such as any of the diseases or disorders described herein, for example, mild-to-moderate AD or moderate AD). Word recognition/recall may be assessed, e.g., as a component of the memory domain making up ADAS-Cog11 scores, e.g., as described herein (ADAS-Cog11 word recognition scores). The methods provided herein comprise administration of an amount (e.g., therapeutically effective amount such as 4500 mg) of an anti-Tau antibody to a patient (e.g., who displays one or more symptoms of decreased word recognition capacity). In a particular embodiment, the antibody is semorinemab.

In some aspects, the disclosure provides a method for slowing decline in language and/or praxis capability associated with a Tau pathology. Accordingly, in some aspects, the disclosure provides an anti-Tau antibody, or a medicament comprising anti-Tau antibody, for slowing down loss in language and/or praxis capacity associated with a Tau pathology (such as any of the diseases or disorders described herein, for example, mild-to-moderate AD or moderate AD). Language and praxis may be assessed, e.g., as domains making up ADAS-Cog11 scores, e.g., as described herein (ADAS-Cog11 language domain scores or ADAS-Cog11 praxis domain scores). The methods provided herein comprise administration of an amount (e.g., therapeutically effective amount such as 4500 mg) of an anti-Tau antibody to a patient (e.g., who displays one or more symptoms of decreased language and/or praxis capacity). In a particular embodiment, the antibody is semorinemab.

In some aspects, the disclosure provides a method for decreasing the rate of progression of a Tau pathology; or provides an anti-Tau antibody, or a medicament comprising anti-Tau antibody, for decreasing the rate of progression of a Tau pathology (such as any of the diseases or disorders described herein, for example, mild-to-moderate AD or moderate AD). The methods provided herein comprise administration of an amount (e.g., therapeutically effective amount such as 4500 mg) of an anti-Tau antibody to a patient (e.g., who displays one or more symptoms of a Tau pathology). In a particular embodiment, the antibody is semorinemab.

Furthermore, the antibodies of the disclosure are useful for treating mild-to-moderate AD or moderate AD without increasing the incidence of adverse events. In some embodiments, the patient is Apoε4 positive. In some embodiments, the patient has an MMSE score of 16-18 or an MMSE score of 16-19.

The anti-Tau antibody will be formulated, dosed, and administered in a fashion consistent with good medical practice. Factors for consideration in this context include the particular disorder being treated, the particular mammal being treated, the clinical condition of the individual subject, the cause of the disorder, the site of delivery of the agent, the method of administration, the scheduling of administration, and other factors known to medical practitioners.

### C. Routes of Administration

An antibody described herein (and any additional therapeutic agent) can be administered by any suitable means, including parenterally. In some embodiments, the antibody is administered intravenously or subcutaneously In some embodiments, the antibody is administered intravenously. In some embodiments, the antibody is administered subcutaneously.

Dosing can be by any suitable route, e.g., by injections, such as intravenous injections, depending in part on whether the administration is brief or chronic. In some embodiments, the antibody is injected subcutaneously. In some embodiments, the antibody is injected intravenously. In some embodiments, semorinemab is administered subcutaneously. In some embodiments, semorinemab is administered intravenously. In some embodiments, the antibody is administered using a syringe (e.g., prefilled or not) or an autoinjector. Various dosing schedules including but not limited to single or multiple administrations over various time-points, bolus administration, and pulse infusion are contemplated herein.

In particular embodiments, the antibody is administered intravenously by infusion. In some embodiments, the infusion rate is 0.1-5.0 mL/min, for example, 0.2 mL/min, 0.3 mL/min, 0.4 mL/min, 0.5 mL/min, 0.6 mL/min, 0.7 mL/min, 0.8 mL/min, 1.0 mL/min, 1.5 mL/min, 2.0 mL/min, 2.5 mL/min, 2.8 mL/min, 3.0 mL/min, 3.2 mL/min, 3.5 mL/min, 4.0 mL/min, or 4.5 mL/min. In some embodiments, the infusion rate is 0.5-3.0 mL/min. The infusion rate may be constant throughout administration or may be increased following an initial period or following the first infusion for a patient receiving treatment. In some embodiments, the infusion rate is 0.5-1 mL/min, for 10-120 minutes of a first infusion, followed by 2-4 mL/minute thereafter, 2.8 to 3.2 mL/minute thereafter, or 3 mL/minute thereafter. In some embodiments, the infusion rate is 0.5-1 mL/min, for 30-60 minutes of a first infusion, followed by 2-4 mL/minute thereafter, 2.8 to 3.2 mL/minute thereafter, or 3 mL/minute thereafter. In some embodiments, the infusion rate is 0.5 for the first 30 minutes of a first infusion, 1 mL/min for a subsequent 30 minutes, and 3 mL/minute thereafter during the infusion, and optionally during subsequent infusions.

In some embodiments, the humanized monoclonal anti-Tau antibody is administered at an infusion rate of 0.5 mL/minute to 3.0 mL/minute. In some embodiments, the humanized monoclonal anti-Tau antibody is administered at an infusion rate of 0.5 mL/minute to 3.0 mL/minute, every four weeks (or monthly). In some embodiments, the humanized monoclonal anti-Tau antibody is administered at an infusion rate of 0.5 mL/min to 1 mL/min, optionally for 10-120 minutes of a first infusion; and 3 mL/minute thereafter. In some embodiments, the humanized monoclonal anti-Tau antibody is administered at an infusion rate of 0.5 mL/min to 1 mL/min.

In some embodiments, semorinemab is administered at an infusion rate of 0.5 mL/minute to 3.0 mL/minute. In some embodiments, semorinemab anti-Tau antibody of this disclosure is administered at an infusion rate of 0.5 mL/minute to 3.0 mL/minute, every four weeks (or monthly). In some embodiments, semorinemab is administered at an infusion rate of 0.5 mL/min to 1 mL/min, optionally for 10-120 minutes of a first infusion; and 3 mL/minute thereafter. In some embodiments, semorinemab is administered at an infusion rate of 0.5 mL/min to 1 mL/min.

In some embodiments the method, the antibody for use and the use of this disclosure further comprises intravenously administering to the patient a 4500 mg dose of semorinemab at a frequency of Q4W for 96 weeks.

### 1 Dosing and Frequency

For treatment of a tauopathy, the appropriate dosage of an antibody described herein (when used alone or in combination with one or more other additional therapeutic agents) will depend on the specific type of disease to be treated the type of antibody, the severity and course of the disease, previous therapy, the patient's clinical history and response to the antibody, and the discretion of the attending physician.

The antibody is suitably administered to the patient at one time or over a series of points in tire. Various dosing schedules including, but not limited to, single or multiple administrations over various time-points, bolus administration, and pulse infusion are contemplated herein.

Depending on the type and severity of the disease, about 10 mg/kg to 100 mg/k of antibody can be used, e.g., in one or more separate administrations, or by continuous infusion. The dosage can be administered in a single dose or a divided dose (e.g., two doses of 25 mg/kg for a total dose of 50 mg/kg). For repeated administrations over several weeks or longer, depending on the condition, the treatment would generally be sustained until a desired suppression of disease symptoms occurs.

One exemplary dosage of the antibody is in the range from about 20 mg/kg to about 80 mg/kg. Thus, one or more doses of about 0.5 mg/kg, 1.0 mg/kg, 1.5 mg/kg, 2.0 mg/kg, 3.0 mg/kg. 4.0 mg/kg, 5.0 mg/kg, 10 mg/kg, 15 mg/kg, 20 mg/kg, 25 mg/kg, 30 mg/kg, 35 mg/kg, 40 mg/kg, 45 mg/kg 50 mg/kg, 55 mg/kg, 60 mg/kg, 70 mg/kg, or 80 mg/kg (or any combination thereof) may be administered to the patient in some embodiments, one or more doses of about 0.5 mg/kg are administered to the patient. In some embodiments, one or more doses of about 1.0 mg/kg are administered to the patient. In some embodiments, one or more doses of about 1.5 mg/kg are administered to the patient. In some embodiments, one or more doses of about 2.0 mg/kg are administered to the patient. In some embodiments, one or more doses of about 3.0 mg/kg are administered to the patient. In some embodiments, one or more doses of about 4.0 mg/kg are administered to the patient. In some embodiments, one or more doses of about 5.0 mg/kg are administered to the patient. In some embodiments, one or more doses of about 10 mg/kg are administered to the patient. In some embodiments, one or more doses of about 15 mg/kg are administered to the patient. In some embodiments, one or more doses of about 20 mg/kg are administered to the patient. In some embodiments, one or more doses of about 25 mg/kg are administered to the patient. In some embodiments, one or more doses of about 30 mg/kg are administered to the patient. In some embodiments, one or more doses of about 35 mg/kg are administered to the patient. In some embodiments, one or more doses of about 40 mg/kg are administered to the patient. In some embodiments, one or more doses of about 45 mg/kg are adminstered to the patient. In some embodiments, one or more doses of about 50 mg/kg are administered to the patient. In some embodiments, one or more doses of about 55 mg/kg are administered to the patient. In some embodiments, one or more doses of about 60 mg/kg are administered to the patient. In some embodiments, one or more doses of about 70 mg/kg are administered to the patient. In some embodiments, one or more doses of about 80 mg/kg are administered to the patient.

In some embodiments, the dosage of the antibody is in the range from 20 mg/kg to 80 mg/kg. Thus, one or more doses of 0.5 mg/kg, 1.0 mg/kg, 1.5 mg/kg, 2.0 mg/kg, 3.0 mg/kg, 4.0 mg/kg, 5.0 mg/kg. 10 mg/kg, 15 mg/kg. 20 mg/kg, 25 mg/kg, 30 mg/kg, 35 mg/kg, 40 mg/kg 45 mg/kg, 50 mg/kg, 55 mg/kg, 60 mg/kg, 70 mg/kg, or 80 mg/kg (or any combination thereof) may be administered to the patient. In some embodiments, one or more doses of 0.5 mg/kg are administered to the patient. In some embodiments, one or more doses of 1.0 mg/kg are administered to the patient. In some embodiments, one or more doses of 1.5 mg/kg are administered to the patient. In some embodiments, one or more doses of 2.0 mg/kg are administered to the patient. In some embodiments, one or more doses of 3.0 mg/kg are administered to the patient. In some embodiments, one or more doses of 4.0 mg/kg are administered to the patient. In some embodiments, one or more doses of 50 mg/kg are administered to the patient. In some embodiments, one or more doses of 10 mg/kg are administered to the patient. In some embodiments, one or more doses of 15 mg/kg are administered to the patient. In some embodiments, one or more doses of 20 mg/kg are administered to the patient. In some embodiments, one or more doses of 25 mg/kg are administered to the patient. In some embodiments, one or more doses of 30 mg/kg are administered to the patient. In some embodiments, one or more doses of 35 mg/kg are administered to the patient. In some embodiments, one or more doses of 40 mg/kg are administered to the patient. In some embodiments, one or more doses of 45 mg/kg are administered to the patient. In some embodiments, one or more doses of 50 mg/kg are administered to the patient. In some embodiments, one or more doses of 55 mg/kg are administered to the patient. In some embodiments, one or more doses of 60 mg/kg are administered to the patient. In some embodiments, one or more doses of 70 mg/kg are administered to the patient. In some embodiments, one or more doses of 80 mg/kg are administered to the patient.

In some embodiments, the total dose administered is in the range of 1000 mg to 10,000 mg. An exemplary dose of about 1500 mg, about 2000 mg, about 2500 mg, about 3000 mg, about 3500 mg, about 4000 mg, about 4500 mg, about 5000 mg, about 5500 mg, about 6000 mg, about 6500 mg, about 7000 mg, about 7500 mg, about 8000 mg, about 8500 mg, about 9000 mg, about 9500 mg, or about 10,000 mg (or any combination thereof) maybe administered to the patient. In some embodiments, one or more doses of about 1500 mg are administered to the patient. In some embodiments, one or more doses of about 2000 mg are administered to the patient. In some embodiments, one or more doses of about 2500 mg are administered to the patient. In some embodiments, one or more doses of about 3000 mg are administered to the patient. In some embodiments, one or more doses of about 3500 mg are administered to the patient. In some embodiments, one or more doses of about 4000 mg are administered to the patient. In some embodiments, one or more doses of about 4500 mg are administered to the patient. In some embodiments, one or more doses of about 5000 mg are administered to the patient. In some embodiments, one or more doses of about 5500 mg are administered to the patient. In some embodiments, one or more doses of about 6000 mg are administered to the patient. In some embodiments, one or more doses of about 6500 mg are administered to the patient. In some embodiments, one or more doses of about 7000 mg are administered to the patient. In some embodiments, one or more doses of about 7500 mg are administered to the patient. In some embodiments, one or more doses of about 8000 mg are administered to the patient. In some embodiments, one or more doses of about 8500 mg are administered to the patient. In some embodiments, one or more doses of about 9000 mg are administered to the patient. In some embodiments, one or more doses of about 9500 mg are administered to the patient. In some embodiments, one or more doses of about 10,000 mg are administered to the patient. In some embodiments, one or more doses of about 4000 mg to about 4500 mg are administered to the patient. In some embodiments, one or more doses of about 4300 mg to about 4700 mg are administered to the patient. In some embodiments, one or more doses of about 4400 mg to about 4600 mg are administered to the patient. In some embodiments, a dose of 1500 mg, 2000 mg 2500 mg, 3000 mg 35 mg, 4000 mg, 4500 mg 5000 mg, 5500 mg, 6000 mg, 6500 mg, 7000 mg, 7500 mg, 8000 mg, 8500 mg, 9000 mg, 9500 mg, or 10,000 mg (or any combination thereof) maybe administered to the patient. In some embodiments, one or more doses of 1500 mg are administered to the patient. In some embodiments, one or more doses of 2000 mg are administered to the patient. In some embodiments, one or more doses of 2500 mg are administered to the patient. In some embodiments, one or more doses of 3000 mg are administered to the patient. In some embodiments, one or more doses of 3500 mg are administered to the patient. In some embodiments, one or more doses of 4000 mg are administered to the patient. In some embodiments, one or more doses of 4500 mg are administered to the patient. In some embodiments, one or more doses of 5000 mg are administered to the patient. In some embodiments, one or more doses of 5500 mg are administered to the patient. In some embodiments, one or more doses of 6000 mg are administered to the patient. In some embodiments, one or more doses of 6500 mg are administered to the patient. In some embodiments, one or more doses of 7000 mg are administered to the patient. In some embodiments, one or more doses of 7500 mg are administered to the patient. In some embodiments, one or more doses of 8000 mg are administered to the patient. In some embodiments, one or more doses of 8500 mg are administered to the patient. In some embodiments, one or more doses of 9000 mg are administered to the patient. In some embodiments, one or more doses of 9500 mg are administered to the patient. In some embodiments, one or more doses of 10,000 mg are administered to the patient. In some embodiments, one or more doses of 4000 mg to 4500 mg are administered to the patient. In some embodiments, one or more doses of 4300 mg to 4700 mg are administered to the patient. In some embodiments, one or more doses of 4400 mg to 4600 mg are administered to the patient. Such doses may be administered intermittently, e.g., every week, every two weeks, every three weeks, every four weeks, ever month, every two months, every three months, or every six months. The progress of this therapy can be monitored by conventional techniques and assays. In a particular embodiment, the antibody is semorinemab.

In some embodiments, the initial dose is the same, or substantially the same, as later doses, and 5 to 50 doses are administered to achieve one or more therapeutic effects, e.g., as described herein. For example, 8 doses, 10 doses, 12 doses, 13 doses, 14 doses, 15 doses, 16 doses, 17 doses, 18 doses, 19 doses, 20 doses. 25 doses, 30 doses, 35 doses, 36 doses, 37 doses, 38 doses. 40 doses, 45 doses, or 50 doses may be administered to achieve one or more therapeutic effects, e.g., as described herein. In particular embodiments, 5-10 doses, 7-9 doses, or 8 doses are administered (e.g., by week 25) to achieve one or more therapeutic effects, e.g., as described herein. In particular embodiments, 9-13 doses, 10-12 doses, or 11 doses are administered (e.g., by week 37) to achieve one or more therapeutic effects, e.g., as described herein. In one particular embodiment, 12-16 doses, 13-15 doses, or 14 doses are administered (e.g., by week 49) to achieve one or more therapeutic effects, e.g., as described herein. In particular embodiments, 5-15 doses are administered to achieve statistically significant reduction in cognitive decline, e.g., measured by ADAS-Cog11, and/or to achieve statistically significant reduction in memory decline, e.g., measured by ADAS-Cog11 memory domain. In some embodiments, 10-15 doses, 12-15 doses, 13-15 doses, 13-14 doses, 14-15 doses, or 14 doses of the antibody are administered (e.g., by week 49) to achieve statistically significant reduction in cognitive decline and/or to achieve statistically significant reduction in memory decline, e.g., measured by ADAS-Cog11. In a particular embodiment, the antibody is semorinemab.

In certain embodiments, an antibody of the disclosure is administered at a dose of 15 ng/kg, 30 mg/kg, 40 mg/kg, 45 mg/kg, 50 mg/kg, 60 mg/kg or a flat dose, e.g., 3500 mg, 4000 mg, 4500 mg, 5000 mg or 5500 mg. In some embodiments, the dose is, administered by intravenous injection, or infusion, every 2 weeks or every 4 weeks for a period of time. In some embodiments, the dose is adminstered intravenously (e.g., by infusion) every 2 weeks for an initial period of time, in certain embodiments, the initial period of time continues for an initial two doses, initial three doses, initial four doses, or an initial five doses. In some embodiments, the dose is administered intravenously (e.g., by infusion) every 4 weeks for subsequent period of time, following the initial dose(s). In certain embodiments, the subsequent period of time (following the initial time period) continues for 4 weeks. 8 weeks, 12 weeks, 24 weeks, 36 weeks, 48 weeks, 49 weeks, 50 weeks, 52 weeks, 55 weeks, 60 weeks, 61 weeks, 62 weeks, 65 weeks, 70 weeks. 72 weeks, 73 weeks, 74 weeks, 75 weeks. 80 weeks, 90 weeks, 100 weeks, 104 weeks, or longer. In certain embodiments, the subsequent period of time (following the initial time period) continues for one month, two months, three months, 6 months, 9 months, 12 months (or one year), eighteen months (or a year and a half), two years, five years, 10 years, 15 years, 20 years, or the lifetime of the patient. In a particular embodiment, the antibody is semorinemab.

In some embodiments, a dose of the humanized monoclonal anti-Tau antibody is administered at least 5 times, at least 8 times, or at least 10 times, or the dose is administered for 5-17 doses, 10-17 doses, or 12-17 doses. In some embodiments, a dose of the humanized monoclonal anti-Tau antibody is administered at least 5 times. In some embodiments, the dose of the humanized monoclonal anti-Tau antibody is administered at least 8 times. In some embodiments, a dose of the humanized monoclonal anti-Tau antibody is administered at least 10 times. In some embodiments, a dose of the humanized monoclonal anti-Tau antibody is administered for 5-17 doses. In some embodiments, a dose of the humanized monoclonal anti-Tau antibody is administered for 10-17 doses. In some embodiments, a dose of the humanized monoclonal anti-Tau antibody is administered for 12-17 doses. In some embodiments, a dose of humanized monoclonal the anti-Tau antibody is administered for 13-15 doses, 13-14 doses, 14-15 doses, or 14 doses. In some embodiments, a dose of the humanized monoclonal anti-Tau antibody is administered for 13-15 doses. In some embodiments, a dose of the humanized monoclonal anti-Tau antibody is administered for 13-14 doses. In some embodiments, a dose of the humanized monoclonal anti-Tau antibody is administered for 14-15 doses. In some embodiments, a dose of the humanized monoclonal anti-Tau antibody is administered for 14 doses. In some embodiments, a dose of the humanized monoclonal anti-Tau antibody is administered for 12-16 doses. In some embodiments, a dose of the humanized monoclonal anti-Tau antibody is administered for 14-17 doses.

In some embodiments, the humanized monoclonal anti-Tau antibody is administered for at least 24 weeks, optionally at least once every 4 weeks (or monthly). In some embodiments, the humanized monoclonal anti-Tau antibody is administered for at least 36 weeks, optionally at least once every 4 weeks (or monthly). In some embodiments, the humanized monoclonal anti-Tau antibody is administered for at least 40, 44, 48, 52, 56, 60, 64, 68, 72, 76, 80, 84, 88, 92, 96, 100, 104, 108, 112, 116, 120, 124, 128, 132, 136, 140, 144, 148, 152, 156, 160, 164, or 168 weeks, optionally at least once every 4 weeks (or monthly). In some embodiments, the humanized monoclonal anti-Tau antibody is administered for at least 40, 44, 48, 52, 56, or 60 weeks, optionally at least once every 4 weeks (or monthly). In some embodiments, the humanized monoclonal anti-Tau antibody is administered for at least 40, 44, or 48 weeks, optionally at least once every 4 weeks (or monthly). In some embodiments, the humanized monoclonal anti-Tau antibody is administered for at least 52, 56, or 60 weeks, optionally at least once every 4 weeks (or monthly). In some embodiments, the humanized monoclonal anti-Tau antibody is administered for at least 48 weeks, optionally at least once every 4 weeks (or monthly). In some embodiments, the humanized monoclonal anti-Tau antibody is administered for at least 60 weeks, optionally at least once every 4 weeks (or monthly). In some embodiments, the humanized monoclonal anti-Tau antibody is administered for at least 144 weeks, optionally at least once every 4 weeks (or monthly). In some embodiments, the humanized monoclonal anti-Tau antibody is administered for at least 156 weeks, optionally at least once every 4 weeks (or monthly).

In some embodiments, the humanized monoclonal anti-Tau antibody is administered once every two weeks, every three weeks, or every four weeks. In some embodiments, the humanized monoclonal anti-Tau antibody is administered once every two weeks (Q2W). In some embodiments, the humanized monoclonal anti-Tau antibody is administered once every three weeks (Q3W). In some embodiments, the humanized monoclonal anti-Tau antibody is administered every four weeks (Q4W). In some embodiments, semorinemab is administered once every two weeks, every three weeks, or every four weeks. In some embodiments, semorinemab is administered once every two weeks (Q2W). In some embodiments, semorinemab is administered once every three weeks (Q3W). In some embodiments, semorinemab is administered every four weeks (Q4W).

In some embodiments, the humanized monoclonal anti-Tau antibody is administered once every two weeks for one to five doses, and then once every four weeks (or once monthly). In some embodiments, the humanized monoclonal anti-Tau antibody is administered once every two weeks for one to three doses, and then once every four weeks (or once monthly). In some embodiments, the humanized monoclonal anti-Tau antibody is administered once every two weeks for one to five doses, and then once every four weeks. In some embodiments, the humanized monoclonal anti-Tau antibody is administered once every two weeks for one to three doses, and then once every four weeks. In some embodiments, the humanized monoclonal anti-Tau antibody is administered once every two weeks for one to five doses, and then once monthly. In some embodiments, the humanized monoclonal anti-Tau antibody is administered once every two weeks for one to three doses, and then once monthly. In some embodiments, the humanized monoclonal anti-Tau antibody is administered Q2W for one to five doses, and then Q4W. In some embodiments, the humanized monoclonal anti-Tau antibody is administered Q2W for one to three doses, and then Q4W. In some embodiments, the humanized monoclonal anti-Tau antibody is administered Q2W for five doses, and then Q4W. In some embodiments, the humanized monoclonal anti-Tau antibody is administered Q2W for three doses, and then Q4W.

In some embodiments, semorinemab is administered at a frequency of Q2W for at least 1 dose. In some embodiments, semorinemab is administered at a frequency of Q2W for at least 2 doses. In some embodiments, semorinemab is administered at a frequency of Q2W for at least 3 doses.

In some embodiments, semorinemab is administered at a frequency of Q4W for at least 10 doses. In some embodiments, semorinemab is administered at a frequency of Q4W for at least 13 doses. In some embodiments, semorinemab is administered at a frequency of Q4W for at least 16 doses. In some embodiments, semorinemab is administered at a frequency of Q4W for at least 34 doses. In some embodiments, semorinemab is administered at a frequency of Q4W for at least 37 doses. In some embodiments, semorinemab is administered Q2W for one to five doses, and then Q4W. In some embodiments, semorinemab is administered Q2W for one to three doses, and then Q4W. In some embodiments, semorinemab is administered Q2W for five doses, and then Q4W. In some embodiments, semorinemab is administered Q2W for three doses, and then Q4W.

In some embodiments, the humanized monoclonal anti-Tau antibody (e.g., semorinemab) is administered at any of the frequencies in this disclosure in one or more cycles. In some embodiments, the humanized monoclonal anti-Tau antibody (e.g., semorinemab) is administered at any of the above frequencies in 1, 2, 3, 4, 5, 6, 7, 8, 9, 10, 11, 12, 13, 14, 15, 16, 17, 18, 19 or 20 cycles. In some embodiments, the humanized monoclonal anti-Tau antibody (e.g., semorinemab) is administered at a frequency of Q2W in one or more cycles. In some embodiments, the humanized monoclonal anti-Tau antibody (e.g., semorinemab) is administered at a frequency of Q2W in 1, 2, 3, 4, 5, 6, 7, 9, 10, 11, 12, 13, 14, 15, 16, 17, 18, 19 or 20 cycles. In some embodiments, the humanized monoclonal anti-Tau antibody (e.g. semorinemab) is administered at a frequency of Q2W for one cycle. In some embodiments, the humanized monoclonal anti-Tau antibody (e.g., semorinemab) is administered at a frequency of Q2W for two cycles. In some embodiments, the humanized monoclonal anti-Tau antibody (e.g., semorinemab) is administered at a frequency of Q2W for three cycles. In some embodiments, the humanized monoclonal anti-Tau antibody (e.g., semorinemab) is administered at a frequency of Q4W in one or more cycles. In some embodiments, the humanized monoclonal anti-Tau antibody (e.g., semorinemab) is administered at a frequency of Q4W in 1, 2, 3, 4, 5, 6, 7, 8, 9, 10, 11, 12, 13, 14, 15, 16, 17, 18, 19 or 20 cycles. In some embodiments, the humanized monoclonal anti-Tau antibody (e.g., semorinemab) is administered at a frequency of Q4W for eleven cycles. In some embodiments, the humanized monoclonal anti-Tau antibody (e.g., semorinemab) is administered at a frequency of Q4W for fourteen cycles. In some embodiments, the humanized monoclonal anti-Tau antibody (e.g., semorinemab) is administered at a frequency of Q4W for thirty-five cycles. In some embodiments, the humanized monoclonal anti-Tau antibody (e.g., semorinemab) is administered at a frequency of Q4W for thirty-eight cycles. In some embodiments, the humanized monoclonal anti-Tau anybody (e.g. semorinemab) is administered at a frequency of Q2W for two four-week cycles (i.e., Cycles 1-2) and thereafter (i.e., Cycle 3 and all subsequent four-week cycles) at a frequency of Q4W. In some embodiments, the humanized monoclonal anti-Tau antibody (e.g., semorinemab) is administered at a frequency of Q2W for the first cycle four-week cycle (i.e., Cycle 1), and thereafter (i.e., Cycle 2 and all subsequent four-week cycles) at a frequency of Q4W.

### 2. Monitoring Assessing Response to Therapeutic Treatment

As used in methods of the present disclosure, the antibody provides therapeutic effect or benefit to the patient. In some embodiments, a patient being treated with an antibody disclosed herein is monitored or assessed to determine if the patient is benefiting from the treatment. In certain embodiments, the therapeutic benefit is a delay in, or inhibition of, progression of AD or a reduction in clinical, functional, or cognitive decline. In some embodiments, therapeutic effect or benefit is reflected in a “patient response” or “response” (and grammatical variations thereof).

Patient response can be assessed using an endpoint indicating a benefit to the patient, including, without limitation, (1) inhibition, to some extent, of disease progression, including stabilizing or slowing down and complete arrest; (2) reduction in amount of plaque or reduction in brain amyloid accumulation and/or reduction in neurofibrillary tangles: (3) reduction in brain tau level and/or reduction in cell to cell spread of Tau toxicity/pathology in the patient's brain; (4) improvement (or reduced deterioration rate) in one or more assessment metrics, including but not limited to ADAS-Cog11 (including ADAS-Cog11 memory domain), ADCS-ADL. CDR-SB, MMSE NPI, and CaGI-Alz scales; (5) improvement in daily functioning of the patient; (6) decrease in one or more biomarkers indicative of the presence of AD, e.g., decrease in Abeta or Tau in cerebrospinal fluid; and (7) an increase in a biomarker indicative of improvement of AD. An assessment of patient response may also include an assessment of any adverse events that may occur and that may be correlated with the treatment.

In some embodiments, the cognitive ability and daily functioning of the patient is assessed prior to, during, and/or after a course of therapy with an antibody described herein, A number of cognitive and functional assessment tools have been developed for use in assessing, diagnosing, and scoring mental function, cognition, and neurological deficit. Exemplary these tools include, but are not limited to, the ADAS-Cog, including the 11 item ADAS-Cog (ADAS-Cog11), the 12 item ADAS-Cog (ADAS-Cog12), the 13-item ADAS-Cog (ADAS-Cog13), and the 14-item ADAS-Cog (ADAS-Cog14); the ADCS-ADL, the CDR-SOB, including CDR Judgment and Problem solving and CDR Memory components: RBANS, and the MMSE.

### ADAS-Cog Scores

In some embodiments, a patient treated with an antibody described herein demonstrates an improvement (i.e., a decrease) in the patient's ADAS-Cog score (such as ADAS-Cog11 or ADAS-Cog11 memory domain) compared to an earlier ADAS-Cog score for the patient from an earlier time point. In some embodiments, the earlier score is from a time-point prior to treatment, that is, before administration of an anti-Tau antibody to the patient. In some embodiments, the methods of treatment herein maintain a patient's cognitive capacity within a range from an earlier ADAS-Cog11 score of the patient, e.g., assessed before administration of the antibody. In particular embodiments, the methods of treatment herein maintain a patient's memory capacity/faculty within a range from an earlier ADAS-Cog11 memory domain score of the patient, e.g., assessed before administration of the antibody. For patients with mild-to-moderate AD, an average cognitive decline of 6 points would be expected without administration of the anti-Tau antibody over the course of about 49 weeks (that is, an average decline of 6 points was expected for placebo-treated patients of Example 1). Also, for patients with mild-to-moderate AD, an average memory decline of 3-4 points would be expected without administration of the anti-Tau antibody over the course of about 49 weeks (that is, an average decline of 3-4 points was expected for placebo-treated patients of Example 1). In certain embodiments, the patient to be treated has mild-to-moderate AD, with an MMSE score of 16-21 and/or a CDR-GS of 1 or 2. In certain embodiments, the patient to be treated has an MMSE score of 16-18 and/or a CDR-GS of 2. In certain embodiments, the patient to be treated has an MMSE score of 16-19 and/or a CDR-GS of 2. In some embodiments, the patient to be treated has an MMSE score of 19-21 and/or a CDR-GS of 1. In some embodiments, the patient to be treated is at a later stage of disease than mild AD and/or has moderate AD.

In some embodiments, the subject has a Mini-Mental State Exam (MMSE) score of 16-19, inclusive, before administration of the anti-Tau antibody. In some embodiments, the subject has a Mini-Mental State Exam (MMSE) score of 16-19, inclusive, before administration of an anti-Tau antibody, optionally an MMSE of 16-18, inclusive, before administration of an anti-Tau antibody. In some embodiments, the subject has a Mini-Mental State Exam (MMSE) score of 16-18, inclusive, before administration of an anti-Tau antibody.

In some embodiments, the patient has a Clinical Dementia Rating Global Score (CDR-GS) of 1 or 2 before administration of an anti-Tau antibody. In some embodiments, the patient has a Clinical Dementia Rating Global Score (CDR-GS) of 1 before administration of an anti-Tau antibody. In some embodiments, the patient has a Clinical Dementia Rating Global Score (CDR-GS) of 2 before administration of an anti-Tau antibody.

In certain embodiments, the disclosure provides a method of maintaining cognitive capacity within 5 points of (i.e. no more than 5 points higher than) an ADAS-Cog11 score of a patient diagnosed with mild to moderate AD or moderate AD, for example, where ADAS-Cog11 scores are assessed for the patient before and after administration of the antibody, e.g., after repeated administration of a certain number of doses of said antibody. In some embodiments, the patient's ADAS-Cog11 score after administration of the antibody is no more than 2, no more than 2.3, no more than 2.5, no more than 2.8, no more than 3, no more than 3.3, no more than 3.5, no more than 3.8, no more than 4, no more than 4.3, no more than 4.5, no more than 4.8, or no more than 5 points higher than that before administration of the antibody. In some embodiments, the patient's ADAS-Cog11 score after administration of the antibody is 2-3, 2-4, 2-5, 3-4, 3-4, or 4-5 points higher than that before administration of the antibody. In some embodiments, the patient's ADAS-Cog11 score after administration of the antibody is within 2, 3, 4, or 5 points higher than that before administration of the antibody. In some embodiments, the patient's ADAS-Cog11 score after administration of the antibody is 2, 3, 4, or 5 points higher than that before administration of the antibody. In certain embodiments, the disclosure provides a method of maintaining cognitive capacity within 5 points of (i.e. no more than 5 points higher than) an ADAS-Cog11 score of a patient diagnosed with mild to moderate AD or moderate AD, for example, where ADAS-Cog11 scores are assessed for the patient before and after multiple administrations of the antibody. In some embodiments, the patient's ADAS-Cog11 score after multiple administrations of the antibody is no more than 2, no more than 2.3, no more than 2.5, no more than 2.8, no more than 3, no more than 3.3, no more than 3.5, no more than 3.8, no more than 4, no more than 4.3, no more than 4.5, no more than 4.8, or no more than 5 points higher than that before administration of the antibody. In some embodiments, the patient's ADAS-Cog11 score after multiple administrations of the antibody is 2-3, 2-4, 2-5, 3-4, 3-4, or 4-5 points higher than that before administration of the antibody. In some embodiments, the patient's ADAS-Cog11 score after multiple administrations of the antibody is within 2, 3, 4, or 5 points higher than that before administration of the antibody. In some embodiments, the patient's ADAS-Cog11 score after multiple administrations of the antibody is 2, 3, 4, or 5 points higher than that before administration of the antibody. In some embodiments, the multiple administrations comprise at least 13 doses. In some embodiments, the multiple administrations comprise at least 16 doses. In some embodiments, the multiple administrations comprise at least 37 doses. In some embodiments, the multiple administrations comprise at least 40 doses. In some embodiments, the antibody is semorinemab. In particular embodiments, the antibody is administered at least once every 4 weeks (or monthly) for at least 49 weeks. In some embodiments, the humanized monoclonal anti-Tau antibody is administered at least once every 4 weeks (or monthly) for at least 48 weeks.

In some embodiments, an ADAS-Cog11 score of the subject assessed after administration of the humanized monoclonal anti-Tau antibody is no more than 2, no more than 2.3, no more than 2.5, no more than 2.8, no more than 3, no more than 3.3, no more than 3.5, no more than 3.8, no more than 4, no more than 4.3, no more than 4.5, no more than 4.8, or no more than 5 points higher than an ADAS-Cog11 score of the subject assessed before administration of said antibody. In some embodiments, an ADAS-Cog11 score of the subject assessed after administration of the humanized monoclonal anti-Tau antibody is no more than 2.5, no more than 3, no more than 3.5, no more than 4, no more than 4.5, or no more than 5 points higher than an ADAS-Cog11 score of the subject assessed before administration of said antibody. In some embodiments, an ADAS-Cog11 score of the subject assessed after administration of the humanized monoclonal anti-Tau antibody is no more than 2 points higher than an ADAS-Cog11 score of the subject assessed before administration of said antibody. In some embodiments, an ADAS-Cog11 score of the subject assessed after administration of the humanized monoclonal anti-Tau antibody is no more than 2.3 points higher than an ADAS-Cog11 score of the subject assessed before administration of said antibody. In some embodiments, an ADAS-Cog11 score of the subject assessed after administration of the humanized monoclonal anti-Tau antibody is no more than 2.5 points higher than an ADAS-Cog11 score of the subject assessed before administration of said antibody. In some embodiments, an ADAS-Cog11 score of the subject assessed after administration of the humanized monoclonal anti-Tau antibody is no more than 2.8 points higher than an ADAS-Cog11 score of the subject assessed before administration of said antibody. In some embodiments, an ADAS-Cog11 score of the subject assessed after administration of the humanized monoclonal anti-Tau antibody is no more than 3 points higher than an ADAS-Cog11 score of the subject assessed before administration of said antibody. In some embodiments, an ADAS-Cog11 score of the subject assessed after administration of the humanized monoclonal anti-Tau antibody is no more than 3.3 points higher than an ADAS-Cog11 score of the subject assessed before administration of said antibody. In some embodiments, an ADAS-Cog11 score of the subject assessed after administration of the humanized monoclonal anti-Tau antibody is no more than 3.5 points higher than an ADAS-Cog11 score of the subject assessed before administration of said antibody. In some embodiments, an ADAS-Cog11 score of the subject assessed after administration of the humanized monoclonal anti-Tau antibody is no more than 3.8 points higher than an ADAS-Cog11 score of the subject assessed before administration of said antibody. In some embodiments, an ADAS-Cog11 score of the subject assessed after administration of the humanized monoclonal anti-Tau antibody is no more than 4 points higher than an ADAS-Cog11 score of the subject assessed before administration of said antibody. In some embodiments, an ADAS-Cog11 score of the subject assessed after administration of the humanized monoclonal anti-Tau antibody is no more than 4.3 points higher than an ADAS-Cog11 score of the subject assessed before administration of said antibody. In some embodiments, an ADAS-Cog11 score of the subject assessed after administration of the humanized monoclonal anti-Tau antibody is no more than 4.5 points higher than an ADAS-Cog11 score of the subject assessed before administration of said antibody. In some embodiments, an ADAS-Cog11 score of the subject assessed after administration of the humanized monoclonal anti-Tau antibody is no more than 4.8 points higher than an ADAS-Cog11 score of the subject assessed before administration of said antibody. In some embodiments, an ADAS-Cog11 score of the subject assessed after administration of the humanized monoclonal anti-Tau antibody is no more than 5 points higher than an ADAS-Cog11 score of the subject assessed before administration of said antibody. In some embodiments, an ADAS-Cog11 score of the subject assessed after administration of the humanized monoclonal anti-Tau antibody is 2-4 points higher than an ADAS-Cog11 score of the subject assessed before administration of said antibody. In some embodiments, an ADAS-Cog11 score of the subject assessed after administration of the humanized monoclonal anti-Tau antibody is 3-4 points higher than an ADAS-Cog11 score of the subject assessed before administration of said antibody. In some embodiments, an ADAS-Cog11 score of the subject assessed after administration of the humanized monoclonal anti-Tau antibody is 2 points higher than an ADAS-Cog11 score of the subject assessed before administration of said antibody. In some embodiments, an ADAS-Cog11 score of the subject assessed after administration of the humanized monoclonal anti-Tau antibody is 3 points higher than an ADAS-Cog11 score of the subject assessed before administration of said antibody. In some embodiments, an ADAS-Cog11 score of the subject assessed after administration of the humanized monoclonal anti-Tau antibody is 4 points higher than an ADAS-Cog11 score of the subject assessed before administration of said antibody.

In some embodiments, this maintenance in cognitive capacity manifests, after at least 20 weeks, at least 25 weeks, at least 30 weeks, at least 35 weeks, at least 37 weeks, at least 40 weeks, at least 45 weeks, at least 47 weeks, at least 49 weeks, at least 51 weeks, or at least 53 weeks, optionally by 49 weeks. In some embodiments, the ADAS-Cog1 score after at least 45 weeks, at least 47 weeks, at least 49 weeks, at least 51 weeks, or at least 53 weeks, optionally by 49 weeks, of administering the antibody is no more 2, 3, 4, or 5 points higher than an earlier ADAS-Cog11 score from the patient before administration of the antibody (baseline score). In some embodiments, a stable ADAS-Cog11 score compared to baseline, or compared to that expected without administering the anti-Tau antibody, indicates a slowing, delay, or halt of the progression of AD, or a slowing, delay, or halt in clinical cognitive decline, e.g., in mild-to-moderate AD, and/or a lack of new clinical cognitive symptoms or impairments, or an overall stabilization of disease progression. In particular embodiments, the antibody is semorinemab (e.g., 4500 mg semorinemab).

In some embodiments, the methods of treatment herein reduce the decline in a patient's cognitive capacity compared to that expected without administration of the antibody (e.g., as indicated by comparison to placebo, such as by comparison to patients in a control arm of a clinical trial). In certain embodiments, the patient to be treated has mild-to-moderate AD, with an MMSE score of 16-21 and/or a CDR-GS of 1 or 2. In certain embodiments, the patient to be treated has a MMSE score of 16-18 and/or a CDR-GS of 2. In certain embodiments, the patient to be treated has a MMSE; score of 16-19 and/or a CDR-GS of 2. In some embodiments, the patient to be treated is at a later stage of disease than mild AD.

In some embodiments, the patient's ADAS-Cog11 score after administration of the antibody is reduced by at least 20%, at least 23%, at least 25%, at least 28%, at least 30%, at least 33%, at least 35%, at least 38%, at least 40%, at least 41%, at least 42%, at least 43%, at least 44%, at least 45%, at least 46%, at least 48%, at least 50%, at least 53%, or at least 55%, or at least 60% compared to that expected without administration of the antibody (e.g., compared to a comparable placebo arm of a clinical study). In some embodiments, the patient's ADAS-Cog11 score after administration of the antibody is reduced by at least 43.2%, 43.4%, 43.6%, 43.8%, or 44% compared to that expected without administration of the antibody (e.g., compared to a comparable placebo arm of a clinical study). In some embodiments, the patient's ADAS-Cog11 score after administration of the antibody is reduced by at least 41%, 41.5%, 41.8%, 42%, 42.2%, 42.4,%, 42.6%, 42.8%, or 43% compared to that expected without administration of the antibody (e.g., compared to a comparable placebo arm of a clinical study). In certain embodiments, the patient's ADAS-Cog11 score after administration of the antibody is reduced by 20-60%, 25-55%, 25-50%, 30-50%, 35-45%, 40-50%, 35-40%, or 40-45% compared to that expected without administration of said antibody (e.g., compared to a comparable placebo arm of a clinical study). In certain embodiments, the patient's ADAS-Cog11 score after administration of the antibody is reduced by 42-44%, 43-44%, 43.4-43.8%, 43.5-43.7% or 43.6% compared to that expected without administration of said antibody (e.g., compared to a comparable placebo arm of a clinical study). In certain embodiments, the patient's ADAS-Cog11 score after administration of the antibody is reduced by 40-45%, 41-43%, 42-42.5% or 42.2% compared to that expected without administration of said antibody (e.g., compared to a comparable placebo arm of a clinical study). In certain embodiments, the patient's ADAS-Cog11 score after administration of the antibody is reduced by 20%, 25%, 30%, 35%, 38%, 40%, 42%, 43% or 45% compared to that expected without administration of said antibody (e.g., compared to a comparable placebo arm of a clinical study). In some embodiments, the antibody is semorinemab. In particular embodiments, 12-16 doses, 12-15 doses, 13-15 doses, 13-14 doses, 14-15 doses, or 14 doses of the antibody are administered (e.g., by week 49) to achieve the therapeutic effect described. In particular embodiments, the antibody is administered at least once every 4 weeks (or monthly) for at least 49 weeks. In some embodiments, the humanized monoclonal anti-Tau antibody is administered at least once every 4 weeks (or monthly) for at least 48 weeks.

In some embodiments, this reduction in cognitive decline occurs after at least 20 weeks, at least 25 weeks, at least 30 weeks, at least 35 weeks, at least 37 weeks, at least 40 weeks, at least 45 weeks, at least 47 weeks, at least 49 weeks, at least 51 weeks, or at least. 53 weeks, optionally by 49 weeks. In some embodiments, the ADAS-Cog11 score after at least 45 weeks, at least 47 weeks, at least 49 weeks, at least 51 weeks, or at least 53 weeks, optionally by 49 weeks, of administering the antibody is no more than 30%, 35%, 40%, 45%, or 50% higher than an earlier ADAS-Cog11 score from the patient before administration of the antibody (baseline score) In some embodiments, a stable ADAS-Cog11 score compared to baseline, or compared to that expected without administering the anti-Tau antibody, indicates a slowing, delay, or halt of the progression of AD, or a slowing, delay, or halt in clinical cognitive decline, e.g., in Mild-to-moderate AD, and/or a lack of new clinical cognitive symptoms or impairments, or an overall stabilization of disease progression. In one particular embodiment, the antibody is semorinemab (e.g., 4500 mg semorinemab).

In particular embodiments, the disclosure provides a method of slowing rate of decline in memory, e.g., slowing loss in memory capacity or memory faculty and/or reducing progressive memory loss. In certain embodiments, the disclosure provides a method of maintaining memory capacity within 2.5 points of an ADAS-Cog11 memory domain score of a patient diagnosed with mild to moderate AD or with moderate AD, for example, where ADAS-Cog11 memory domain scores are assessed for the patient before and after administration of the antibody. In some embodiments, the patient's ADAS-Cog11 memory domain score after administration of the antibody is no more than 1, no more than 1.4, no more than 1.5, no more than 1.6, no more than 1.7, no more than 1.8, no more than 2, no more than 2.2, no more than 2.3, no more than 2.4, or no more than 2.5 points higher than that before administration of the antibody. In some embodiments, the patient's ADAS-Cog11 memory domain score after administration of the antibody is 1-2, 1.5-2, 1-2.5, or 1.5-2.5 points higher than that before administration of the antibody. In some embodiments, the patient's ADAS-Cog11 memory domain score after administration of the antibody is within 1, 1.5, 2, or 2.5 points higher than that before administration of the antibody. In some embodiments, the patient's ADAS-Cog11 memory domain score after administration of the antibody is 1, 1.5, 2, or 2.5 points higher than that before administration of the antibody. In some embodiments, the antibody is semorinemab. In particular embodiments, the antibody is administered at least once every 4 weeks (or monthly) for at least 49 weeks. In particular embodiments, semorinemab is administered at least once every 4 weeks (or monthly) for at least 48 weeks.

In some embodiments, an ADAS-Cog11 memory domain score of the subject assessed after administration of the humanized monoclonal anti-Tau antibody is no more than 1, no more than 1.5, no more than 1.7, no more than 2, no more than 2.3, or no more than 2.5 points higher than an ADAS-Cog11 memory domain score of the patient assessed before administration of said antibody. In some embodiments, an ADAS-Cog11 memory domain score of the subject assessed after administration of the humanized monoclonal anti-Tau antibody is no more than 1 points higher than an ADAS-Cog11 memory domain score of the patient assessed before administration of said antibody. In some embodiments, an ADAS-Cog11 memory domain score of the subject assessed after administration of the humanized monoclonal anti-Tau antibody no more than 1.5 points higher than an ADAS-Cog11 memory domain score of the patient assessed before administration of said antibody. In some embodiments, an ADAS-Cog11 memory domain score of the subject assessed after administration of the humanized monoclonal anti-Tau antibody is no more than 1.7 points higher than an ADAS-Cog11 memory domain score of the patient assessed before administration of said antibody. In some embodiments, an ADAS-Cog11 memory domain score of the subject assessed after administration of the humanized monoclonal anti-Tau antibody is no more than 2 points higher than an ADAS-Cog11 memory domain score of the patient assessed before administration of said antibody. In some embodiments, an ADAS-Cog11 memory domain score of the subject assessed after administration of the humanized monoclonal anti-Tau antibody is no more than 2.3 points higher than an ADAS-Cog11 memory domain score of the patient assessed before administration of said antibody. In some embodiments, an ADAS-Cog11 memory domain score of the subject assessed after administration of the humanized monoclonal anti-Tau antibody is no more than 2.5 points higher than an ADAS-Cog11 memory domain score of the patient assessed before administration of said antibody. In some embodiments, an ADAS-Cog11 memory domain score of the subject assessed after administration of the humanized monoclonal anti-Tau antibody is 1-2 points higher than an ADAS-Cog11 memory domain score of the patient assessed before administration of said antibody. In some embodiments, an ADAS-Cog11 memory domain score of the subject assessed after administration of the humanized monoclonal anti-Tau antibody is 1.5-2.5 points higher than an ADAS-Cog11 memory domain score of the patient assessed before administration of said antibody. In some embodiments, an ADAS-Cog11 memory domain score of the subject assessed after administration of the humanized monoclonal anti-Tau antibody is 1 point higher than an ADAS-Cog11 memory domain score of the patient assessed before administration of said antibody. In some embodiments, an ADAS-Cog11 memory domain score of the subject assessed after administration of the humanized monoclonal anti-Tau antibody is 1.5 points higher than an ADAS-Cog11 memory domain score of the patient assessed before administration of said antibody. In some embodiments, an ADAS-Cog11 memory domain score of the subject assessed after administration of the humanized monoclonal anti-Tau antibody is 2 points higher than an ADAS-Cog11 memory domain score of the patient assessed before administration of said antibody. In some embodiments, an ADAS-Cog11 memory domain score of the subject assessed after administration of the humanized monoclonal anti-Tau antibody is 2.5 points higher than an ADAS-Cog11 memory domain score of the patient assessed before administration of said antibody.

In some embodiments, this maintenance in memory manifests after at least 20 weeks, at least 25 weeks, at least 30 weeks, at least 35 weeks, at least 37 weeks, at least 40 weeks, at least 45 weeks, at least 47 weeks, at least 49 weeks, at least 51 weeks, or at least 53 weeks, optionally by 49 weeks. In some embodiments, the ADAS-Cog11 memory domain score after at least 45 weeks, at least 47 weeks, at least 49 weeks, at least 51 weeks, or at least 53 weeks, optionally by 49 weeks, of administering the antibody is no more 1, 1.5, 2, or 2.5 points higher than an earlier ADAS-Cog11 memory domain score from the patient before administration of the antibody (baseline score). In some embodiments, a stable ADAS-Cog11 memory domain score compared to baseline, or compared to that expected without administering the anti-Tau antibody, indicates a slowing, delay, or halt of the progression of AD, or a slowing, delay, or halt in clinical cognitive decline, e.g., in mild-to-moderate AD (or in moderate AD), and/or a lack of new clinical cognitive symptoms or impairments, or an overall stabilization of disease progression. In particular embodiments, the antibody is semorinemab (e.g., 4500 mg semorinemab).

In some embodiments, the methods of treatment herein reduce the decline in a patient's memory compared to that expected without administration of the antibody (e.g., as indicated by comparison to placebo, such as by comparison to patients in a control arm of a clinical trial). In certain embodiments, the patient to be treated has mild-to-moderate AD (or moderate AD), with an MMSE score of 16-21 and/or a CDR-GS of 1 or 2. In certain embodiments, the patient to be treated has a MMSE score of 16-18 and/or a CDR-GS of 2. In certain embodiments, the patient to be treated has a MMSE score of 16-19 and/or a CDR-GS of 2. In some embodiments, the patient to be treated is at a later stage of disease than mild AD.

In some embodiments, the patient's ADAS-Cog11 memory domain score after administration of the antibody is reduced by at least 20%, at least 23%, at least 25%, at least 28%, at least 30%, at least 33%, at least 35%, at least 38%, at least 40%, at least 41%, at least 42%, at least 43%, at least 44%, at least 45%, at least 46%, at least 48%, at least 50%, at least 53%, or at least 55%, or at least 60% compared to that expected without administration of the antibody (e.g., compared to a comparable placebo arm of a clinical study). In some embodiments, the patient's ADAS-Cog11 memory domain score after administration of the antibody is reduced by at least 43.2%, 43.4%, 43.6%, 43.8%, or 44% compared to that expected without administration of the antibody (e.g., compared to a comparable placebo arm of a clinical study). In some embodiments, the patient's ADAS-Cog11 memory domain score after administration of the antibody is reduced by at least 41%, 41.5%, 41.8%, 42%, 42.2%, 42.4,%, 42.6%, 42.8%, or 43% compared to that expected without administration of the antibody (e.g., compared to a comparable placebo arm of a clinical study). In certain embodiments, the patient's ADAS-Cog11 memory domain score after administration of the antibody is reduced by 20-60%, 25-55%, 25-50%, 30-50%, 35-45%, 40-50%, 35-40%, or 40-45% compared to that expected without administration of said antibody (e.g., compared to a comparable placebo arm of a clinical study). In certain embodiments, the patient's ADAS-Cog11 memory domain score after administration of the antibody is reduced by 42-44%, 43-44%, 43.4-43.8%, 43.5-43.7% or 43.6% compared to that expected without administration of said antibody (e.g., compared to a comparable placebo arm of a clinical study). In certain embodiments, the patient's ADAS-Cog11 memory domain score after administration of the antibody is reduced by 40-45%, 41-43%, 42-42.5% or 42.2% compared to that expected without administration of said antibody (e.g., compared to a comparable placebo arm of a clinical study). In certain embodiments, the patient's ADAS-Cog11 memory domain score after administration of the antibody is reduced by 20%, 25%, 30%, 35%, 38%, 40%, 42%, 43% or 45% compared to that expected without administration of said antibody (e.g., compared to a comparable placebo arm of a clinical study). In some embodiments, the antibody is semorinemab. In particular embodiments, 12-16 doses, 12-15 doses, 13-15 doses, 13-14 doses, 14-15 doses, or 14 doses of the antibody are administered (e.g., by week 49) to achieve the therapeutic effect described. In a particular embodiment, the antibody is administered at least once every 4 weeks (or monthly) for at least 49 weeks.

In some embodiments, this reduction in memory decline occurs after at least 20 weeks, at least 25 weeks, at least 30 weeks, at least 35 weeks, at least 37 weeks, at least 40 weeks, at least 45 weeks, at least 47 weeks, at least 49 weeks, at least 51 weeks, or at least 53 weeks, optionally by 49 weeks. In some embodiments, the ADAS-Cog11 memory domain score after at least 45 weeks, at least 47 weeks, at least 49 weeks, at least 51 weeks, or at least 53 weeks, optionally by 49 weeks, of administering the antibody is no more than 30%, 35%, 40%, 45%, or 50% higher than an earlier ADAS-Cog11 memory domain score from the patient before administration of the antibody (baseline score). In some embodiments, a stable ADAS-Cog11 memory domain score compared to baseline, or compared to that expected without administering the anti-Tau antibody, indicates a slowing, delay, or halt of the progression of AD, or a slowing, delay, or halt in clinical cognitive decline, e.g., in mild-to-moderate AD (or in moderate AD), and/or a lack of new clinical cognitive symptoms or impairments, or an overall stabilization of disease progression. In particular embodiments, the antibody is semorinemab (e.g., 4500 mg semorinemab).

### a) Tau Levels

Tau levels, or Tau load, in a patient, can be assessed using neurological imaging techniques and tools, for example using PET (positron emission tomography) scanning. In such methods, a tracer molecule known to bind to Tau is radiolabeled with a PET-sensitive radioisotope and introduced into the patient. In conjunction with scans of the patient's brain, the location and quantity (i.e., distribution) of Tau can be imaged based on the assumption that the tracer binds to the Tau molecules. Serial PET scans of a patient taken over time, e.g., before and after administration of a treatment (or at one or more intervals throughout the course of a treatment regimen), can permit detection of increased, decreased, or unchanged Tau load in the brain. Tau levels in a patient also can be assessed by measuring Tau in a blood, serum, or CSF sample from the patient. One or more of these techniques can be used to determine whether total Tau is increasing or decreasing, or whether a given isoform of Tau (e.g., aggregated Tau) is increasing or decreasing, at various time points over the course of treatment.

In some embodiments, the Tau tracer is [¹⁸F] Genentech Tau Probe 1 ([¹⁸F]GTP1), as described in U.S. Pat. No. 10,076,581. In some embodiments, other Tau probes can be used. Examples of such tracer molecules include but are not limited to RO-948 (F. Hoffmann-La Roche A G); AV-1451 (“Flortaucipir”, Avid, Inc.); PI-2014, and PI-2620 (AC Immune); MK-6240 (Merck Sharp & Dohme); and T-808 (Eli Lilly & Co.). Methods of quantifying the Tau distribution in a patient's brain, based on imaging a radio-labeled tracer, include “Standardized Uptake Value Ratio” (SUVR) (see, e.g., J Nucl. Med., S. Sanabria Bohorquez et al., 58(1), (2017), incorporated herein by reference).

Tau imaging using a radioligand, such as [¹⁸F]GTP1, provides advantages over conventional CSF biomarker evaluation in that it allows for the relationship between the distribution of Tau pathology and response to anti-Tau therapy to be evaluated. For example, in the clinical study disclosed herein (Example 1), longitudinal [¹⁸F]GTP1 PET imaging data was collected to assess the response of this biomarker to semorinemab, as it has the potential to inform the relationship between spatial distribution of Tau pathology, cognitive function, and disease progression.

Without being bound by theory, at different stages of AD, tau pathology may manifest in different primary configurations, which may play different roles in both the further spread of tau pathology and the relative contribution of tau pathology to clinical decline. While earlier clinical studies investigated treatment in patients with early (prodromal-to-mild) AD, results provided herein investigated treatment in people with mild-to-moderate AD. Accordingly, the earlier clinical study and the clinical study disclosed herein represent different patient populations (in this regard).

### b) Amyloid Levels

Brain amyloid load or burden can be determined using neurological imaging techniques and tools, for example using PET (positron emission tomography) scanning Serial PET scans of a patient taken over time, e.g., before and after administration of a treatment (or at one or more intervals throughout the course of a treatment regimen), can permit detection of increased, decreased, or unchanged amyloid burden in the brain. This technique can further be used to determine tether amyloid accumulation is increasing or decreasing. In some embodiments, detection of amyloid deposits in the brain is performed by measuring CSF Abetal-42 levels (using a pre-specified cutoff point and the Roche Diagnostics Elecsys@ β Amyloid [1-42] immunoassay (below cutoff; ≤1,000 μg/mL)) or a centralized visual assessment of the brain by amyloid PET imaging. In some embodiments, detection of amyloid deposits in the brain is performed using florbetapir, florbetaben, and/or flutemetamol. In some embodiments, a florbetapir PET scan is considered positive if, based on a centralized visual read of the scan, it establishes the presence of moderate-to-frequent neuritic plaques.

Biomarker evidence of Abeta deposition can be assessed by decreased CSF Abetal-42 levels (e.g., using a pre-specified cutoff point and the Roche Diagnostics Elecsys@ β Amyloid [1-42] immunoassay (below cutoff; ≤1,000 μg/mL)) and/or a centralized visual assessment of the brain by amyloid PET imaging. Both approaches have been shown to correlate with the “gold standard” of Aβ pathology at autopsy (Shaw et al. Ann Neurol. 2009:65; 403-13; Clark et al. JAMA 2011; 305:275-83; Le Bastard et al. J Alzheimer's Dis 2013; 33:117-31) (see, e.g., Example 1).

### c) Assessment Schedules

Typically, a patient is assessed at various times before and after administration of the anti-Tau antibody. In some embodiments, the patient is assessed “prior to treatment,” meaning, before initiation of treatment with the antibody, such as before an initial administration of the antibody, that is, before administration of the first/initial dose of antibody to that patient. A “baseline” score refers to a score assessed prior to treatment, before the first/initial dose of antibody.

“Prior to treatment” typically means at any time from diagnosis of the disease (such as AD, or mild-to-moderate AD) up to administration of the treatment provided herein. In some embodiments, prior to treatment is within 12 months, 6 months, 3 months, 2 months, 1 month, 3 weeks, 2 weeks, or 1 week prior to treatment. In some embodiments, baseline assessment occurs on the same day but before the beginning of treatment. The baseline period may last up to 15 days or more. In some embodiments, baseline refers to the average of screening and baseline observations. In some embodiments, baseline is assessed 5-10 minutes before, 15-30 minutes before, 30-60 minutes before, 1-2 hours, before 2-12 hours before, 12-18 hours before, 12-24 hours before, one day before, 2 days before, 3 days before, one week, 10 days, 14 day, or 15 days before the initiation of treatment with the antibody.

In some embodiments, the patient is assessed after initiation of treatment with the antibody, such as after the first, second, third, etc., administration of the antibody to that patient. The patient may be assessed on the same day as the dose is administered, e.g., during the same clinic visit, or on a different day.

In some embodiments, baseline and the time point at which the patient is assessed for therapeutic benefit, and/or safety (adverse effects), are at least 1 week, at least 3 weeks, at least 5 weeks, at least 9 weeks, at least 13 weeks, at least 17 weeks, at least 21 weeks, at least 25 weeks, at least 29 weeks, at least 33 weeks, at least 37 weeks, at least 41 weeks, at least 45 weeks, at least 49 weeks, and/or at least 57 weeks apart. In some embodiments, the patient is assessed at 1 week, 3 weeks (±3 days), 5 weeks (±5 days), 9 weeks (±5 days), 13 weeks (±5 days), 17 weeks (±5 days), 21 weeks (±5 days), 25 weeks (±5 days), 29 weeks (±5 days), 33 weeks (±5 days), 37 weeks (5 days), 41 weeks (±5 days), 45 weeks (±5 days), 49 weeks (±5 days), and/or 57 weeks (7 days) from baseline or an earlier treatment. See, e.g., Example 1, Cohort 1 (double blind treatment period). In some embodiments, baseline and the time point at which the patient is assessed for therapeutic benefit, and/or safety (adverse effects), are at least 53 weeks, at least 57 weeks, at least 61 weeks, at least 65 weeks, at least 69 weeks, at least 73 weeks, at least 77 weeks, at least 81 weeks, at least 85 weeks, at least 89 weeks, at least 93 weeks, at least 97 weeks, at least 101 weeks, at least 105 weeks, at least 109 weeks, at least 113 weeks, at least 117 weeks, at least 121 weeks, at least 125 weeks, at least 129 weeks, at least 133 weeks, at least 137 weeks, at least 141 weeks, at least 145 weeks, and/or at least 157 weeks apart. In some embodiments, the patient is assessed at 53 weeks (±5 days), 57 weeks (±5 days), 61 weeks (±5 days), 65 weeks (±5 days), 69 weeks (±5 days), 73 weeks (±5 days), 77 weeks (±5 days), 81 weeks (±5 days), 85 weeks (±5 days), 89 weeks (±5 days), 93 weeks (±5 days), 97 weeks (±5 days), 101 weeks (±5 days), 105 weeks (±5 days), 109 weeks (±5 days), 113 weeks (±5 days), 117 weeks (±5 days), 121 weeks (±5 days), 125 weeks (±5 days), 129 weeks (±5 days), 133 weeks (5 days), 137 weeks (±5 days), 141 weeks (±5 days), 145 weeks (±5 days), and/or 157 weeks (5 days) from baseline or an earlier treatment. See, e.g., Example 1, Cohort 1 (open label extension period).

In some embodiments, baseline and the time point at which the patient is assessed for therapeutic benefit, and/or safety (adverse effects), are at least 1 week, at least 3 weeks, at least 5 weeks, at least 9 weeks, at least 13 weeks, at least 17 weeks, at least 21 weeks, at least 25 weeks, at least 29 weeks, at least 33 weeks, at least 37 weeks, at least 41 weeks, at least 45 weeks, at least 49 weeks, at least 53 weeks, at least 57 weeks, at least 61 weeks, and/or at least 69 weeks apart. In some embodiments, the patient is assessed at 1 week, 3 weeks (±3 days), 5 weeks (±5 days), 9 weeks (±5 days), 13 weeks (±5 days), 17 weeks (±5 days), 21 weeks (±5 days), 25 weeks (±5 days), 29 weeks (±5 days), 33 weeks (±5 days), 37 weeks (±5 days), 41 weeks (±5 days), 45 weeks (±5 days), 49 weeks (±5 days), 53 weeks (±5 days), 57 weeks (±5 days), 61 weeks (±5 days), and/or 69 weeks (±7 days) from baseline or an earlier treatment. See, e.g., Example 1, Cohort 2 (double blind treatment period).

In some embodiments, the patient is assessed at least 65 weeks, at least 69 weeks, at least 73 weeks, at least 77 weeks, at least 81 weeks, at least 85 weeks, at least 89 weeks, at least 93 weeks, at least 97 weeks, at least 101 weeks, at least 105 weeks, at least 109 weeks, at least 113 weeks, at least 117 weeks, at least 121 weeks, at least 125 weeks, at least 129 weeks, at least 133 weeks, at least 137 weeks, at least 141 weeks, at least 145 weeks, at least 149 weeks, at least 153 weeks, at least 157 weeks, and/or at least 169 weeks after initial administration of the antibody (that is, after the beginning of treatment with the antibody). In some embodiments, the patient is assessed at 53 weeks (±5 days), 57 weeks (±5 days), 61 weeks (±5 days), 65 weeks (±5 days), 69 weeks (±5 days), 73 weeks (±5 days), 77 weeks (±5 days), 81 weeks (±5 days), 85 weeks (±5 days), 89 weeks (±5 days), 93 weeks (±5 days), 97 weeks (±5 days), 101 weeks (±5 days), 105 weeks (±5 days), 109 weeks (±5 days), 113 weeks (±5 days), 117 weeks (±5 days), 121 weeks (±5 days), 125 weeks (±5 days), 129 weeks (±5 days), 133 weeks (±5 days), 137 weeks (±5 days), 141 weeks (±5 days), 145 weeks (±5 days), 149 weeks (±5 days), 153 weeks (±5 days), 157 weeks (±5 days), and/or 169 weeks (±5 days) from baseline or an earlier treatment. See, e.g., Example 1, Cohort 2 (open label extension period).

In some embodiments, baseline and the time point at which the patient is assessed for therapeutic benefit, and/or safety (adverse effects), are at least 1 week, at least 3 weeks, at least 5 weeks, at least 9 weeks, at least 13 weeks, at least 17 weeks, at least 21 weeks, at least 25 weeks, at least 29 weeks, at least 33 weeks, at least 37 weeks, at least 41 weeks, at least 45 weeks, at least 49 weeks, at least 53 weeks, at least 57 weeks, at least 61 weeks, at least 65 weeks, at least 69 weeks, at least 73 weeks, and/or at least 81 weeks apart. In some embodiments, the patient is assessed at 1 week, 3 weeks (±3 days), 5 weeks (±5 days), 9 weeks (±5 days), 13 weeks (±5 days), 17 weeks (±5 days), 21 weeks (±5 days), 25 weeks (±5 days), 29 weeks (±5 days), 33 weeks (±5 days), 37 weeks (±5 days), 41 weeks (±5 days), 45 weeks (±5 days), 49 weeks (±5 days), 53 weeks (±5 days), 57 weeks (±5 days), 61 weeks (±5 days), 65 weeks (±5 days), 69 weeks (±5 days), 71 weeks (±5 days), and/or 81 weeks (±7 days) from baseline or an earlier treatment. See, e.g., Example 1, Cohort 3 (double blind treatment period). In some embodiments, baseline and the time point at which the patient is assessed for therapeutic benefit, and/or safety (adverse effects), are at least 77 weeks, at least 81 weeks, at least 85 weeks, at least 89 weeks, at least 93 weeks, at least 97 weeks, at least 101 weeks, at least 105 weeks, at least 109 weeks, at least 113 weeks, at least 117 weeks, at least 121 weeks, at least 125 weeks, at least 129 weeks, at least 133 weeks, at least 137 weeks, at least 141 weeks, at least 145 weeks, at least 149 weeks, at least 153 weeks, at least 157 weeks, at least 161 weeks, at least 165 weeks, at least 169 weeks, and/or at least 181 weeks apart. In some embodiments, the patient is assessed at 77 weeks (±5 days), 81 weeks (±5 days), 85 weeks (±5 days), 89 weeks (±5 days), 93 weeks (±5 days), 97 weeks (±5 days), 101 weeks (±5 days), 105 weeks (±5 days), 109 weeks (±5 days), 113 weeks (±5 days), 117 weeks (±5 days), 121 weeks (±5 days), 125 weeks (±5 days), 129 weeks (±5 days), 133 weeks (±5 days), 137 weeks (±5 days), 141 weeks (±5 days), 145 weeks (±5 days), 149 weeks (±5 days), 153 weeks (±5 days), 157 weeks (5 days), 161 weeks (±5 days), 165 weeks (±5 days), 169 weeks (±5 days), and/or 181 weeks (5 days) from baseline or an earlier treatment. See, e.g., Example 1, Cohort 3 (open label extension period).

### 3. Co-Administration

Antibodies of the disclosure can be used either alone or in combination with other agents or other therapy. For instance, the anti-Tau antibody may be “co-administered” or with at least one additional therapeutic agent. The additional therapy may comprise any medication (e.g., prescription drugs, over the counter drugs, vaccines, herbal or homeopathic remedies, nutritional supplements) used by a patient in addition to the anti-Tau antibody. Examples include any agent that targets human Tau. In some cases, the additional therapy comprises opiates or opioids, benzodiazepines, barbiturates or hypnotics, medications with clinically significant central antihistaminic or anticholinergic activity, antipsychotic, and/or neuroleptic medication. In particular cases, the additional therapy is a concomitant medication, such as a symptomatic medication (used to ameliorate symptoms of AD), including without limitation one or more cholinesterase inhibitors (e.g., galantamine, rivastigmine, donepezil, and the like), an N-methyl-D-aspartate receptor antagonist (e.g., memantine), and/or a food supplement (such as the food supplement Souvenaid®). In some embodiments, the one or more additional agents of this disclosure are selected from the group consisting of: a symptomatic medication, a neurological drug, a corticosteroid, an antibiotic, an antiviral agent, an additional anti-Tau antibody, a Tau inhibitor, an anti-amyloid beta antibody, a beta-amyloid aggregation inhibitor, an anti-BACE1 antibody, a BACE1 inhibitor; a cholinesterase inhibitor; an NMDA receptor antagonist; a monoamine depletor; an ergoloid mesylate; an anticholinergic antiparkinsonism agent; a dopaminergic antiparkinsonism agent; a tetrabenazine; an anti-inflammatory agent; a hormone; a vitamin; a dimebolin; a homoTaurine; a serotonin receptor activity modulator; an interferon, and a glucocorticoid. In some embodiments, the one or more additional agent is a symptomatic medication. In some embodiments, the one or more additional agent is a neurological drug. In some embodiments, the one or more additional agent is a corticosteroid. In some embodiments, the one or more additional agent is an antibiotic. In some embodiments, the one or more additional agent is an antiviral agent. In some embodiments, the one or more additional agent is an additional anti-Tau antibody. In some embodiments, the one or more additional agent is a Tau inhibitor. In some embodiments, the one or more additional agent is an anti-amyloid beta antibody. In some embodiments, the one or more additional agent is a beta-amyloid aggregation inhibitor. In some embodiments, the one or more additional agent is an anti-BACE1 antibody. In some embodiments, the one or more additional agent is a BACE1 inhibitor. In some embodiments, the one or more additional agent is a cholinesterase inhibitor. In some embodiments, the one or more additional agent is an NMDA receptor antagonist. In some embodiments, the one or more additional agent is a monoamine depletory. In some embodiments, the one or more additional agent is an ergoloid mesylate. In some embodiments, the one or more additional agent is an anticholinergic antiparkinsonism agent. In some embodiments, the one or more additional agent is a dopaminergic antiparkinsonism agent. In some embodiments, the one or more additional agent is a tetrabenazine. In some embodiments, the one or more additional agent is an anti-inflammatory agent. In some embodiments, the one or more additional agent is a hormone. In some embodiments, the one or more additional agent is a vitamin. In some embodiments, the one or more additional agent is a dimebolin. In some embodiments, the one or more additional agent is a homotaurine. In some embodiments, the one or more additional agent is a serotonin receptor activity modulator. In some embodiments, the one or more additional agent is an interferon. In some embodiments, the one or more additional agent is a glucocorticoid.

Accordingly, the anti-Tau antibody may be administered in combination with an additional therapeutic agent, such as a biologically active substance or compound, for example, a known compound used in the medication of tauopathies and/or amyloidoses.

Generally, the other therapeutic agent may include neuron-transmission enhancers, psychotherapeutic drugs, acetylcholine esterase inhibitors, calcium-channel blockers, biogenic amines, benzodiazepine tranquillizers, acetylcholine synthesis, storage or release enhancers, acetylcholine postsynaptic receptor agonists, monoamine oxidase-A or -B inhibitors, N-methyl-D-aspartate glutamate receptor antagonists, non-steroidal anti-inflammatory drugs, antioxidants, serotonergic receptor antagonists, or other therapeutic agents. In particular, the additional therapeutic agent may comprise at least one compound selected from compounds against oxidative stress, anti-apoptotic compounds, metal chelators, inhibitors of DNA repair such as pirenzepine and metabolites, 3-amino-1-propanesulfonic acid (3APS), 1,3-propanedisulfonate (1,3PDS), secretase activators, beta- and gamma-secretase inhibitors, tau proteins, other anti-Tau antibodies (including, but not limited to, antibodies disclosed in WO2012049570, WO2014028777, WO2014165271, WO2014100600, WO2015200806, U.S. Pat. Nos. 8,980,270, and 8,980,271), neurotransmitter, beta-sheet breakers, anti-inflammatory molecules, “atypical antipsychotics” such as, for example clozapine, ziprasidone, risperidone, aripiprazole or olanzapine or cholinesterase inhibitors (ChEIs) such as tacrine, rivastigmine, donepezil, and/or galantamine and other drugs and nutritive supplements such as, for example, vitamin B 12, cysteine, a precursor of acetylcholine, lecithin, choline, Ginkgo biloba, acetyl-L-carnitine, idebenone, propentofylline, and/or a xanthine derivative.

In some embodiments, the anti-Tau antibody is administered in combination with a neurological drug. Neurological drugs include, but are not limited to, an antibody or other binding molecule (including, but not limited to a small molecule, a peptide, an aptamer, or other protein binder) that specifically binds to a target selected from: beta secretase, presenilin, amyloid precursor protein or portions thereof, amyloid beta peptide or oligomers or fibrils thereof, death receptor 6 (DR6), receptor for advanced glycation end-products (RAGE), parkin, and huntingtin: an NMDA receptor antagonist (i.e., memantine), a monoamine depletor (i.e., tetrabenazine); an ergoloid mesylate; an anticholinergic antiparkinsonism agent (i.e., procyclidine, diphenhydramine, trihexylphenidyl, benztropine, biperiden and trihexyphenidyl); a dopaminergic antiparkinsonism agent (i.e., entacapone, selegiline, pramipexole, bromocriptine, rotigotine, selegiline, ropinirole, rasagiline, apomorphine, carbidopa, levodopa, pergolide, tolcapone and amantadine); a tetrabenazine; an anti-inflammatory (including, but not limited to, a nonsteroidal anti-inflammatory drug (i.e., indomethicin and other compounds listed above); a hormone (i.e., estrogen, progesterone and leuprolide); a vitamin (i.e., folate and nicotinamide); a dimebolin; a homoTaurine (i.e., 3-aminopropanesulfonic acid; 3APS); a serotonin receptor activity modulator (i.e., xaliproden); an, an interferon, and a glucocorticoid or corticosteroid. The term “corticosteroid” includes, but is not limited to, fluticasone (including fluticasone propionate (FP)), beclometasone, budesonide, ciclesonide, mometasone, flunisolide, betamethasone and triamcinolone. “Inhalable corticosteroid” means a corticosteroid that is suitable for delivery by inhalation. Exemplary inhalable corticosteroids are fluticasone, beclomethasone dipropionate, budenoside, mometasone furoate, ciclesonide, flunisolide, and triamcinolone acetonide. In some embodiments, the one or more additional agents of this disclosure comprises a therapeutic agent that specifically binds to a target selected from the group consisting of beta secretase, Tau, presenilin, amyloid precursor protein or portions thereof, amyloid beta peptide or oligomers or fibrils thereof, death receptor 6 (DR6), receptor for advanced glycation end-products (RAGE), parkin, and huntingtin. In some embodiments, the one or more additional agent is a therapeutic agent that specifically binds to a target that is beta secretase. In some embodiments, the one or more additional agent is a therapeutic agent that specifically binds to a target that is Tau. In some embodiments, the one or more additional agent is a therapeutic agent that specifically binds to a target that is presenilin. In some embodiments, the one or more additional agent is a therapeutic agent that specifically binds to a target that is amyloid precursor protein or portions thereof. In some embodiments, the one or more additional agent is a therapeutic agent that specifically binds to a target that is amyloid beta peptide or oligomers or fibrils thereof. In some embodiments, the one or more additional agent is a therapeutic agent that specifically binds to a target that is death receptor 6 (DR6). In some embodiments, the one or more additional agent is a therapeutic agent that specifically binds to a target that is receptor for advanced glycation end-products (RAGE). In some embodiments, the one or more additional agent is a therapeutic agent that specifically binds to a target that is parkin. In some embodiments, the one or more additional agent is a therapeutic agent that specifically binds to a target that is huntingtin.

In some embodiments, the monoamine depletor is tetrabenazine. In some embodiments, the anticholinergic antiparkinsonism agent is selected from the group consisting of procyclidine, diphenhydramine, trihexylphenidyl, benztropine, biperiden and trihexyphenidyl. In some embodiments, the anticholinergic antiparkinsonism agent is procyclidine. In some embodiments, the anticholinergic antiparkinsonism agent is diphenhydramine. In some embodiments, the anticholinergic antiparkinsonism agent is trihexylphenidyl. In some embodiments, the anticholinergic antiparkinsonism agent is benztropine. In some embodiments, the anticholinergic antiparkinsonism agent is biperiden. In some embodiments, the anticholinergic antiparkinsonism agent is trihexyphenidyl. In some embodiments, the dopaminergic antiparkinsonism agent is selected from the group consisting of: entacapone, selegiline, pramipexole, bromocriptine, rotigotine, selegiline, ropinirole, rasagiline, apomorphine, carbidopa, levodopa, pergolide, tolcapone and amantadine. In some embodiments, the dopaminergic antiparkinsonism agent is entacapone. In some embodiments, the dopaminergic antiparkinsonism agent is selegiline. In some embodiments, the dopaminergic antiparkinsonism agent is pramipexole. In some embodiments, the dopaminergic antiparkinsonism agent is bromocriptine. In some embodiments, the dopaminergic antiparkinsonism agent is rotigotine. In some embodiments, the dopaminergic antiparkinsonism agent is selegiline. In some embodiments, the dopaminergic antiparkinsonism agent is ropinirole. In some embodiments, the dopaminergic antiparkinsonism agent is rasagiline. In some embodiments, the dopaminergic antiparkinsonism agent is apomorphine. In some embodiments, the dopaminergic antiparkinsonism agent is carbidopa. In some embodiments, the dopaminergic antiparkinsonism agent is levodopa. In some embodiments, the dopaminergic antiparkinsonism agent is pergolide. In some embodiments, the dopaminergic antiparkinsonism agent is tolcapone. In some embodiments, the dopaminergic antiparkinsonism agent is amantadine. In some embodiments, the anti-inflammatory agent is selected from the group consisting of a nonsteroidal anti-inflammatory drug and indomethacin. In some embodiments, the anti-inflammatory agent is a nonsteroidal anti-inflammatory drug. In some embodiments, the anti-inflammatory agent is indomethacin. In some embodiments, the hormone is selected from the group consisting of estrogen, progesterone, and leuprolide. In some embodiments, the hormone is estrogen. In some embodiments, the hormone is progesterone. In some embodiments, the hormone is leuprolide. In some embodiments, the vitamin is selected from the group consisting of folate and nicotinamide. In some embodiments, the vitamin is folate. In some embodiments, the vitamin is nicotinamide. In some embodiments, the homoTaurine is 3-aminopropanesulfonic acid or 3APS. In some embodiments, the serotonin receptor activity modulator is xaliproden.

In certain particular embodiments, the co-administered agent is one or more selected from the group of a corticosteroid, an antibiotic, an antiviral agent, a different anti-Tau antibody, a Tau inhibitor, an anti-amyloid beta antibody, an beta-amyloid aggregation inhibitor, an anti-BACE1 antibody, a BACE1 inhibitor; a therapeutic agent that specifically binds a target; a cholinesterase inhibitor; an NMDA receptor antagonist; a monoamine depletor; an ergoloid mesylate; an anticholinergic antiparkinsonism agent; a dopaminergic antiparkinsonism agent; a tetrabenazine; an anti-inflammatory agent; a hormone; a vitamin; a dimebolin; a homoTaurine; a serotonin receptor activity modulator; an interferon, and a glucocorticoid.

In some embodiments, one or more anti-amyloid beta (anti-Abeta) antibodies may be administered with an anti-Tau antibody discussed herein. Non-limiting examples of such anti-Abeta antibodies include crenezumab, solanezumab, bapineuzumab, aducanumab, gantenerumab, donanemab (Lilly) and lecanemab (BAN-2401; Biogen, Eisai Co., Ltd.). In some embodiments, one or more beta-amyloid aggregation inhibitors may be administered with an anti-Tau antibody discussed herein. Non-limiting exemplary beta-amyloid aggregation inhibitors include ELND-005 (also referred to as AZD-103 or scyllo-inositol), tramiprosate, and PTI-80 (Exebryl-1®; ProteoTech). In some embodiments, one or more BACE inhibitors may be administered with the anti-Tau antibody. Non-limiting examples of BACE inhibitors include E-2609 (Biogen, Eisai Co., Ltd.), AZD3293 (also known as LY3314814; AstraZeneca, Eli Lilly & Co.), MK-8931 (verubecestat), and JNJ-54861911 (Janssen, Shionogi Pharma).

In some embodiments, one or more Tau inhibitors may be administered with an anti-Tau antibody discussed herein. Non-limiting examples of Tau inhibitors include methylthioninium, LMTX (also known as leuco-methylthioninium or Trx-0237; TauRx Therapeutics Ltd.), Rember™ (methylene blue or methylthioninium chloride [MTC]; Trx-0014; TauRx Therapeutics Ltd), PBT2 (Prana Biotechnology), and PTI-51-CH3 (TauPro™; ProteoTech). In some embodiments, one or more anti-Tau antibodies other than semorinemab are co-administered. In some embodiments, the different anti-Tau antibody is one or more selected from the group consisting of a different N-terminal binder, a mid-domain binder, and a fibrillar Tau binder. In some embodiments, the additional anti-Tau antibody is a different N-terminal binder. In some embodiments, the additional anti-Tau antibody is a mid-domain binder. In some embodiments, the additional anti-Tau antibody is a fibrillar Tau binder. In some embodiments, the additional anti-Tau antibody is selected from the group consisting of Gosuranemab, Tilavonemab, Bepranemab, and Zagotenemab. In some embodiments, the additional anti-Tau antibody is Gosuranemab (also referred to as BMS-986168). In some embodiments, the additional anti-Tau antibody is Tilavonemab (also referred to as C2N-8E12). In some embodiments, the additional anti-Tau antibody is Bepranemab. In some embodiments, the anti-Tau antibody is Zagotenemab. Non-limiting examples of other anti-Tau antibodies include BIIB092 or BMS-986168 (Biogen, Bristol-Myers Squibb) and ABBV-8E12 or C2N-8E12 (AbbVie, C2N Diagnostics, LLC). In some embodiments, a general misfolding inhibitor, such as NPT088 (NeuroPhage Pharmaceuticals), is administered with the anti-Tau antibody.

In some embodiments, one or more anti-Abeta antibodies are co-administered. Non-limiting examples of such anti-Abeta antibodies include crenezumab, solanezumab, bapineuzumab, aducanumab, donanemab, lecanemab and gantenerumab. In some embodiments, the anti-amyloid beta antibody of this disclosure is aducanemab, lecanemab, or donanemab. In some embodiments, the anti-amyloid beta antibody of this disclosure is crenezumab or gantenerumab. In some embodiments, the anti-amyloid beta antibody of this disclosure is aducanemab. In some embodiments, the anti-amyloid beta antibody of this disclosure is lecanemab. In some embodiments, the anti-amyloid beta antibody of this disclosure is donanemab. In some embodiments, the anti-amyloid beta antibody of this disclosure is crenezumab. In some embodiments, the anti-amyloid beta antibody of this disclosure is gantenerumab.

In some embodiments, an “atypical antipsychotic” is co-administered, such as, e.g., clozapine, ziprasidone, risperidone, aripiprazole or olanzapine for the treatment of positive and negative psychotic symptoms including hallucinations, delusions, thought disorders (manifested by marked incoherence, derailment, tangentiality), and bizarre or disorganized behavior, as well as anhedonia, flattened affect, apathy, and social withdrawal.

Other compounds that can be co-administered, in some embodiments, include, e.g., therapeutic agents discussed in WO 2004/058258 (see especially pages 16 and 17), including therapeutic drug targets (page 36-39), alkanesulfonic acids and alkanolsulfuric acid (pages 39-51), cholinesterase inhibitors (pages 51-56), NMDA receptor antagonists (pages 56-58), estrogens (pages 58-59), non-steroidal anti-inflammatory drugs (pages 60-61), antioxidants (pages 61-62), peroxisome proliferators-activated receptors (PPAR) agonists (pages 63-67), cholesterol-lowering agents (pages 68-75); amyloid inhibitors (pages 75-77), amyloid formation inhibitors (pages 77-78), metal chelators (pages 78-79), anti-psychotics and anti-depressants (pages 80-82), nutritional supplements (pages 83-89) and compounds increasing the availability of biologically active substances in the brain (see pages 89-93) and prodrugs (pages 93 and 94), which document is incorporated herein by reference, but especially the compounds mentioned on the pages indicated above.

Such combination therapies noted above encompass combined administration (where two or more therapeutic agents are included in the same or separate formulations), and separate administration, in which case, administration of the anti-Tau antibody (e.g., semorinemab) can occur prior to, simultaneously, and/or following, administration of the additional therapeutic agent or agents. In some embodiments, the anti-Tau antibody (e.g., semorinemab) is administered prior to administration of the additional therapeutic agent or agents. In some embodiments, the anti-Tau antibody (e.g., semorinemab) is administered simultaneously with administration of the additional therapeutic agent or agents. In some embodiments, the anti-Tau antibody (e.g., semorinemab) is administered subsequent to administration of the additional therapeutic agent or agents. In some embodiments, administration of the anti-Tau antibody and administration of an additional therapeutic agent occur within about one month, or within about one, two or three weeks, or within about one, two, three, four, five, or six days, of each other. In some embodiments, the anti-Tau antibody and the additional therapeutic agent are administered within a few minutes apart, such as 5 minutes, 10 minutes, 15 minutes, 20 minutes, 30 minutes, 45 minutes, 1 hours, 2 hours, 3 hours, or 6 hours apart.

Thus, the antibodies disclosed herein may be optionally formulated with one or more agents currently used to prevent or treat the disorder in question or one or more of its symptoms. The effective amount of such other agents depends on the amount of antibody present in the formulation, the type of disorder or treatment, and other factors discussed above. These are generally used in the same dosages and with administration routes as described herein, or about from 1 to 99% of the dosages described herein or in any dosage and by any route that is empirically/clinically determined to be appropriate. It will be understood by one of ordinary skill in the art that an anti-Tau antibody disclosed herein may be co-administered simultaneously with any of the foregoing therapeutic agents or may be administered prior to or subsequent to administration of any of the foregoing therapeutic agents.

It is understood that any of the above formulations or therapeutic methods may be carried out using an immunoconjugate of the disclosure in place of or in addition to an anti-Tau antibody.

### D. Articles of Manufacture

In another aspect, the disclosure provides an article of manufacture containing materials useful for the treatment, prevention, monitoring and/or diagnosis of tau pathologies. The article of manufacture comprises a container and a label or package insert on, or associated with, the container. Suitable containers include, for example, bottles, vials, syringes, IV solution bags, etc. The containers may be formed from a variety of materials such as glass or plastic. The container holds a composition, which is by itself or combined with another composition effective for treating, preventing, monitoring and/or diagnosing the condition and may have a sterile access port (for example, the container may be an intravenous solution bag or a vial having a stopper pierceable by a hypodermic injection needle).

At least one active agent in the composition is an anti-Tau antibody described herein. The label or package insert indicates that the composition is used for treating the condition of choice. Moreover, the article of manufacture ma comprise (a) a first container with a composition contained therein, wherein the composition comprises the anti-Tau antibody; and (b) a second container with a composition contained therein, wherein the composition comprises an additional therapeutic agent. The article of manufacture in this embodiment of the disclosure may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI) or infusion phosphate-buffered saline. Ringer's solution, and dextrose solution. It may further include other materials desirable from a commercial and user standpoint, including other buffers, diluents, filters, needles, and syringes.

### E. Methods of Maintaining or Slowing Decline in Cognitive Capacity

The present disclosure relates to methods of slowing decline in cognitive capacity in a subject diagnosed with mild-to-moderate Alzheimer's disease (AD) or moderate AD. The present disclosure also relates to methods of maintaining cognitive capacity within 5 points of an Alzheimer's disease Assessment Scale, Cognitive Subscale, 11-item version (ADAS-Cog11) score of a patient diagnosed with mild-to-moderate AD or moderate AD, wherein the ADAS-Cog11 score of the patient assessed after administration of 12 to 17 doses of said antibody is no more than 2.5, no more than 3, no more than 3.5, no more than 4, no more than 4.5, or no more than 5 points higher than an ADAS-Cog11 score of the patient assessed before administration of said antibody, thereby maintaining cognitive capacity within 5 points of the ADAS-Cog11 score of the patient. In some embodiments, the method of this disclosure comprises administering to the subject diagnosed with mild-to-moderate AD or moderate AD a 4500 mg dose of humanized monoclonal anti-Tau antibody, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO: 8.

One aspect of the present disclosure provides a method of slowing decline in cognitive capacity in a patient diagnosed with mild-to-moderate Alzheimer's disease (AD), comprising administering to said patient a 4500 mg dose of humanized monoclonal anti-Tau antibody, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

In another aspect, the present disclosure provides a humanized monoclonal anti-Tau antibody provided at a dose of 4500 mg for use in slowing decline in cognitive capacity in a patient diagnosed with mild-to-moderate AD, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

In another aspect, the present disclosure provides use of a humanized monoclonal anti-Tau antibody for the manufacture of a medicament for slowing decline in cognitive capacity in a patient diagnosed with mild-to-moderate AD, wherein the anti-Tau antibody is formulated to be provided at a dose of 4500 mg and comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

A further aspect of the present disclosure provides a method of slowing decline in cognitive capacity in a patient diagnosed with moderate AD, comprising administering to said patient a 4500 mg dose of humanized monoclonal anti-Tau antibody, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

In another aspect, the present disclosure provides a humanized monoclonal anti-Tau antibody provided at a dose of 4500 mg for use in slowing decline in cognitive capacity in a patient diagnosed with moderate AD, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

In another aspect, the present disclosure provides use of a humanized monoclonal anti-Tau antibody for the manufacture of a medicament for slowing decline in cognitive capacity in a patient diagnosed with moderate AD, wherein the anti-Tau antibody is formulated to be provided at a dose of 4500 mg and comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

A further aspect of the present disclosure provides a method of maintaining cognitive capacity within 5 points of an Alzheimer's disease Assessment Scale, Cognitive Subscale, 11-item version (ADAS-Cog11) score of a patient diagnosed with mild-to-moderate AD, comprising administering to said patient a 4500 mg dose of humanized monoclonal anti-Tau antibody, wherein the ADAS-Cog11 score of the patient assessed after administration of 12 to 17 doses of said antibody is no more than 2.5, no more than 3, no more than 3.5, no more than 4, no more than 4.5, or no more than 5 points higher than an ADAS-Cog11 score of the patient assessed before administration of said antibody, thereby maintaining cognitive capacity within 5 points of the ADAS-Cog11 score of the patient, and wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

In another aspect, the present disclosure provides a humanized monoclonal anti-Tau antibody provided at a dose of 4500 mg for use in maintaining cognitive capacity no more than 5 points higher than an Alzheimer's disease Assessment Scale, Cognitive Subscale, 11-item version (ADAS-Cog11) score of a patient diagnosed with mild-to-moderate AD, after administration of 12 to 17 doses, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

In another aspect, the present disclosure provides use of a humanized monoclonal anti-Tau antibody for the manufacture of a medicament for maintaining cognitive capacity no more than 5 points higher than an Alzheimer's disease Assessment Scale, Cognitive Subscale, 11-item version (ADAS-Cog11) score of a patient diagnosed with mild-to-moderate AD, following administration of 12 to 17 doses, wherein the anti-Tau antibody is formulated to be provided at a dose of 4500 mg and comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

A further aspect of the present disclosure provides a method of maintaining cognitive capacity within 5 points of an Alzheimer's disease Assessment Scale, Cognitive Subscale, 11-item version (ADAS-Cog11) score of a patient diagnosed with moderate AD, comprising administering to said patient a 4500 mg dose of humanized monoclonal anti-Tau antibody, wherein the ADAS-Cog11 score of the patient assessed after administration of 12 to 17 doses of said antibody is no more than 2.5, no more than 3, no more than 3.5, no more than 4, no more than 4.5, or no more than 5 points higher than an ADAS-Cog11 score of the patient assessed before administration of said antibody, thereby maintaining cognitive capacity within 5 points of the ADAS-Cog11 score of the patient, and wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

In another aspect, the present disclosure provides a humanized monoclonal anti-Tau antibody provided at a dose of 4500 mg for use in maintaining cognitive capacity no more than 5 points higher than an Alzheimer's disease Assessment Scale, Cognitive Subscale, 11-item version (ADAS-Cog11) score of a patient diagnosed with moderate AD, after administration of 12 to 17 doses, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

In another aspect, the present disclosure provides use of a humanized monoclonal anti-Tau antibody for the manufacture of a medicament for maintaining cognitive capacity no more than 5 points higher than an Alzheimer's disease Assessment Scale, Cognitive Subscale, 11-item version (ADAS-Cog11) score of a patient diagnosed with moderate AD, following administration of 12 to 17 doses, wherein the anti-Tau antibody is formulated to be provided at a dose of 4500 mg and comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

An additional aspect of the present disclosure provides a method of slowing decline in cognitive capacity in a patient diagnosed with mild-to-moderate AD, the method comprising intravenously administering to the patient a 4500 mg dose of semorinemab at a frequency of Q2W for 3 doses and subsequently at a frequency of Q4W.

In another aspect, the present disclosure provides semorinemab at a dose of 4500 mg for use in slowing decline in cognitive capacity in a patient diagnosed with mild-to-moderate AD, wherein the semorinemab is intravenously administered at a frequency of Q2W for 3 doses and subsequently at a frequency of Q4W.

In another aspect, the present disclosure provides use of semorinemab for the manufacture of a medicament for slowing decline in cognitive capacity in a patient diagnosed with mild-to-moderate AD, wherein the semorinemab is formulated for intravenous administration at a dose of 4500 mg, wherein the medicament is administered at a frequency of Q2W for 3 doses and subsequently at a frequency of Q4W.

A further aspect of the present disclosure provides a method of maintaining cognitive capacity within 5 points of an ADAS-Cog11 score of a patient diagnosed with mild-to-moderate AD, the method comprising intravenously administering to the patient a 4500 mg dose of semorinemab at a frequency of Q2W for 3 doses and subsequently at a frequency of Q4W.

In another aspect, the present disclosure provides semorinemab at a dose of 4500 mg for use in maintaining cognitive capacity no more than 5 points higher than an ADAS-Cog11 score of a patient diagnosed with mild-to-moderate AD, wherein the semorinemab is intravenously administered at a frequency of Q2W for 3 doses and subsequently at a frequency of Q4W.

In another aspect, the present disclosure provides use of semorinemab for the manufacture of a medicament for maintaining cognitive capacity no more than 5 points higher than an ADAS-Cog11 score of a patient diagnosed with mild-to-moderate AD, wherein the semorinemab is formulated for intravenous administration at a dose of 4500 mg, wherein the medicament is formulated to be administered at a frequency of Q2W for 3 doses and subsequently at a frequency of Q4W.

An additional aspect of the present disclosure provides a method of maintaining cognitive capacity within 2.5 points of an ADAS-Cog11 score of a patient diagnosed with mild-to-moderate AD, the method comprising intravenously administering to the patient a 4500 mg dose of semorinemab at a frequency of Q2W for 3 doses and subsequently at a frequency of Q4W.

In another aspect, the present disclosure provides semorinemab at a dose of 4500 mg for use in maintaining cognitive capacity no more than 2.5 points higher than an ADAS-Cog11 score of a patient diagnosed with mild-to-moderate AD, wherein the semorinemab is intravenously administered at a frequency of Q2W for 3 doses and subsequently at a frequency of Q4W.

In another aspect, the present disclosure provides use of semorinemab for the manufacture of a medicament for maintaining cognitive capacity no more than 2.5 points higher than an ADAS-Cog11 score of a patient diagnosed with mild-to-moderate AD, wherein the semorinemab is formulated for intravenous administration at a dose of 4500 mg, wherein the medicament is formulated to be administered at a frequency of Q2W for 3 doses and subsequently at a frequency of Q4W.

An additional aspect of the present disclosure provides a method of slowing decline in cognitive capacity in a patient diagnosed with moderate AD, the method comprising intravenously administering to the patient a 4500 mg dose of semorinemab at a frequency of Q2W for 3 doses and subsequently at a frequency of Q4W.

In another aspect, the present disclosure provides semorinemab at a dose of 4500 mg for use in slowing decline in cognitive capacity in a patient diagnosed with moderate AD, wherein the semorinemab is intravenously administered at a frequency of Q2W for 3 doses and subsequently at a frequency of Q4W.

In another aspect, the present disclosure provides use of semorinemab for the manufacture of a medicament for slowing decline in cognitive capacity in a patient diagnosed with moderate AD, wherein the semorinemab is formulated for intravenous administration at a dose of 4500 mg, wherein the medicament is administered at a frequency of Q2W for 3 doses and subsequently at a frequency of Q4W.

A further aspect of the present disclosure provides a method of maintaining cognitive capacity within 5 points of an ADAS-Cog11 score of a patient diagnosed with moderate AD, the method comprising intravenously administering to the patient a 4500 mg dose of semorinemab at a frequency of Q2W for 3 doses and subsequently at a frequency of Q4.

In another aspect, the present disclosure provides semorinemab at a dose of 4500 mg for use in maintaining cognitive capacity no more than 5 points higher than an ADAS-Cog11 score of a patient diagnosed with moderate AD, wherein the semorinemab is intravenously administered at a frequency of Q2W for 3 doses and subsequently at a frequency of Q4W.

In another aspect, the present disclosure provides use of semorinemab for the manufacture of a medicament for maintaining cognitive capacity no more than 5 points higher than an ADAS-Cog11 score of a patient diagnosed with moderate AD, wherein the semorinemab is formulated for intravenous administration at a dose of 4500 mg, wherein the medicament is formulated to be administered at a frequency of Q2W for 3 doses and subsequently at a frequency of Q4W.

A further aspect of the present disclosure provides a method of maintaining cognitive capacity within 2.5 points of an ADAS-Cog11 score of a patient diagnosed with moderate AD, the method comprising intravenously administering to the patient a 4500 mg dose of semorinemab at a frequency of Q2W for 3 doses and subsequently at a frequency of Q4W

In another aspect, the present disclosure provides semorinemab at a dose of 4500 mg for use in maintaining cognitive capacity no more than 2.5 points higher than an ADAS-Cog11 score of a patient diagnosed with moderate AD, wherein the semorinemab is intravenously administered at a frequency of Q2W for 3 doses and subsequently at a frequency of Q4W.

In another aspect, the present disclosure provides use of semorinemab for the manufacture of a medicament for maintaining cognitive capacity no more than 2.5 points higher than an ADAS-Cog11 score of a patient diagnosed with moderate AD, wherein the semorinemab is formulated for intravenous administration at a dose of 4500 mg, wherein the medicament is formulated to be administered at a frequency of Q2W for 3 doses and subsequently at a frequency of Q4W.

Unless otherwise indicated, any of the above aspects of methods, antibodies-for-use or uses for maintaining or slowing decline in cognitive capacity can comprises any of the anti-Tau antibodies, pharmaceutical compositions, routes of administration, doses, dosing regimens, or co-administrations disclosed herein.

### F. Methods of Maintaining Memory or Slowing Memory Decline

The present disclosure relates to methods of slowing memory decline in a subject with mild-to-moderate Alzheimer's Disease (AD) or moderate AD. The present disclosure also relates to maintaining memory within 2.5 points of an Alzheimer's disease Assessment Scale, Cognitive Subscale, 11-item version (ADAS-Cog11) memory domain score of a subject diagnosed with mild-to-moderate AD or moderate AD, wherein the ADAS-Cog11 memory domain score of the subject assessed after administration of 12 to 17 doses of said antibody is no more than 1, no more than 1.5, no more than 1.7, no more than 2, no more than 2.3, or no more than 2.5 points higher than an ADAS-Cog11 memory domain score of the subject assessed before administration of said antibody, thereby maintaining memory within 2.5 points of the ADAS-Cog11 memory domain score of the subject. In some embodiments, the method of this disclosure comprises administering to the subject diagnosed with mild-to-moderate AD or moderate AD a 4500 mg dose of humanized monoclonal anti-Tau antibody, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

An aspect of the present disclosure provides a method of slowing memory decline in a patient diagnosed with mild-to-moderate AD, comprising administering to said subject a 4500 mg dose of humanized monoclonal anti-Tau antibody, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

In another aspect, the present disclosure provides a humanized monoclonal anti-Tau antibody provided at a dose of 4500 mg for use in slowing memory decline in a subject diagnosed with mild-to-moderate AD, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

In another aspect, the present disclosure provides use of a humanized monoclonal anti-Tau antibody for the manufacture of a medicament for slowing memory decline in a subject diagnosed with mild-to-moderate AD, wherein the antibody is formulated to be provided at a dose of 4500 mg and comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

A further aspect of the present disclosure provides a method of slowing memory decline in a patient diagnosed with moderate AD, comprising administering to said patient a 4500 mg dose of humanized monoclonal anti-Tau antibody to slow the decline in memory in the patient, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

In another aspect, the present disclosure provides a humanized monoclonal anti-Tau antibody provided at a dose of 4500 mg for use in slowing memory decline in a patient diagnosed with moderate AD, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

In another aspect, the present disclosure provides use of a humanized monoclonal anti-Tau antibody for the manufacture of a medicament for slowing memory decline in a patient diagnosed with moderate AD, wherein the antibody is formulated to be provided at a dose of 4500 mg and comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

A further aspect of the present disclosure provides a method of maintaining memory within 2.5 points of an Alzheimer's disease Assessment Scale, Cognitive Subscale, 11-item version (ADAS-Cog11) memory domain score of a patient diagnosed with mild-to-moderate AD, comprising administering to said patient a 4500 mg dose of humanized monoclonal anti-Tau antibody, wherein the ADAS-Cog11 memory domain score of the patient assessed after administration of 12 to 17 doses of said antibody is no more than 1, no more than 1.5, no more than 1.7, no more than 2, no more than 2.3, or no more than 2.5 points higher than an ADAS-Cog11 memory domain score of the patient assessed before administration of said antibody, thereby maintaining memory within 2.5 points of the ADAS-Cog11 memory domain score of the patient, and wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

In another aspect, the present disclosure provides a humanized monoclonal anti-Tau antibody provided at a dose of 4500 mg for use in maintaining memory within 2.5 points of an Alzheimer's disease Assessment Scale, Cognitive Subscale, 11-item version (ADAS-Cog11) memory domain score of a patient diagnosed with mild-to-moderate AD, after administration of 12 to 17 doses, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

In another aspect, the present disclosure provides use of a humanized monoclonal anti-Tau antibody for the manufacture of a medicament for maintaining memory within 2.5 points of an Alzheimer's disease Assessment Scale, Cognitive Subscale, 11-item version (ADAS-Cog11) memory domain score of a patient diagnosed with mild-to-moderate AD, following administration of 12 to 17 doses, wherein the anti-Tau antibody is formulated to be provided at a dose of 4500 mg and comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

An additional aspect of the present disclosure provides a method of maintaining memory within 2.5 points of an Alzheimer's disease Assessment Scale, Cognitive Subscale, 11-item version (ADAS-Cog11) memory domain score of a patient diagnosed with moderate AD, comprising administering to said patient a 4500 mg dose of humanized monoclonal anti-Tau antibody, wherein the ADAS-Cog11 memory domain score of the patient assessed after administration of 12 to 17 doses of said antibody is no more than 1, no more than 1.5, no more than 1.7, no more than 2, no more than 2.3, or no more than 2.5 points higher than an ADAS-Cog11 memory domain score of the patient assessed before administration of said antibody, thereby maintaining memory within 2.5 points of the ADAS-Cog11 memory domain score of the patient, and wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

In another aspect, the present disclosure provides a humanized monoclonal anti-Tau antibody provided at a dose of 4500 mg for use in maintaining memory within 2.5 points of an Alzheimer's disease Assessment Scale, Cognitive Subscale, 11-item version (ADAS-Cog11) memory domain score of a patient diagnosed with moderate AD, after administration of 12 to 17 doses, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

In another aspect, the present disclosure provides use of a humanized monoclonal anti-Tau antibody for the manufacture of a medicament for maintaining memory within 2.5 points of an Alzheimer's disease Assessment Scale, Cognitive Subscale, 11-item version (ADAS-Cog11) memory domain score of a patient diagnosed with moderate AD, following administration of 12 to 17 doses, wherein the anti-Tau antibody is formulated to be provided at a dose of 4500 mg and comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

A further aspect of the present disclosure provides a method of slowing memory decline in a patient diagnosed with mild-to-moderate AD, the method comprising intravenously administering to the patient a 4500 mg dose of semorinemab at a frequency of Q2W for 3 doses and subsequently at a frequency of Q4W.

In another aspect, the present disclosure provides semorinemab at a dose of 4500 mg for use in slowing memory decline in a patient diagnosed with mild-to-moderate AD, wherein the semorinemab is intravenously administered at a frequency of Q2W for 3 doses and subsequently at a frequency of Q4W.

In another aspect, the present disclosure provides use of semorinemab for the manufacture of a medicament for slowing memory decline in a patient diagnosed with mild-to-moderate AD, wherein the semorinemab is formulated for intravenous administration at a dose of 4500 mg, wherein the medicament is formulated to be administered at a frequency of Q2W for 3 doses and subsequently at a frequency of Q4W.

A further aspect of the present disclosure provides a method of slowing memory decline in a patient diagnosed with moderate AD, the method comprising intravenously administering to the patient a 4500 mg dose of semorinemab at a frequency of Q2W for 3 doses and subsequently at a frequency of Q4W.

In another aspect, the present disclosure provides semorinemab at a dose of 4500 mg for use in slowing memory decline in a patient diagnosed with moderate AD, wherein the semorinemab is intravenously administered at a frequency of Q2W for 3 doses and subsequently at a frequency of Q4W.

In another aspect, the present disclosure provides use of semorinemab for the manufacture of a medicament for slowing memory decline in a patient diagnosed with moderate AD, wherein the semorinemab is formulated for intravenous administration at a dose of 4500 mg, wherein the medicament is formulated to be administered at a frequency of Q2W for 3 doses and subsequently at a frequency of Q4W.

Unless otherwise indicated, any of the above aspects of methods, antibodies-for-use or uses for maintaining or slowing memory decline can comprises any of the anti-Tau antibodies, pharmaceutical compositions, routes of administration, doses, dosing regimens, or co-administrations disclosed herein.

### G. Methods of Slowing Decline in Language Capacity or Praxis Capacity

The present disclosure relates to methods of slowing decline in language capacity in a subject diagnosed with mild-to-moderate Alzheimer's disease (AD) or moderate AD. The present disclosure also relates to methods of slowing decline in praxis capacity in a subject diagnosed with mild-to-moderate AD or moderate AD. In some embodiments, the methods of this disclosure comprises administering to the subject diagnosed with mild-to-moderate AD or moderate AD a. 4500 mg dose of humanized monoclonal anti-Tau antibody, wherein the anti-Tau antibody comprises an HVR-H1 comprising the amino acid sequence set forth in SEQ ID N0:2; an HVR-H2 comprising the amino acid sequence set forth in SEQ U) NO:3; an HVR-H3 comprising the amino acid sequence set forth in SEQ ID NO:4 an HVR-L1 comprising the amino acid sequence set forth in SEQ ID NO:6; an HVR-L2 comprising the amino acid sequence set forth in SEQ ID N0:7; and an HVR-L3 comprising the amino acid sequence set forth in SEQ ID NO:8.

An aspect of the present disclosure provides a method of slowing decline in language capacity in a patient diagnosed with mild-to-moderate AD, comprising administering to said patient a 4500 mg dose of humanized monoclonal anti-Tau antibody, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

In another aspect, the present disclosure provides a humanized monoclonal anti-Tau antibody provided at a dose of 4500 mg for use in slowing decline in language capacity in a patient diagnosed with mild-to-moderate AD, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

In another aspect, the present disclosure provides use of a humanized monoclonal anti-Tau antibody for the manufacture of a medicament for slowing decline in language capacity in a patient diagnosed with mild-to-moderate AD, wherein the antibody is formulated to be provided at a dose of 4500 mg and comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

An additional aspect of the present disclosure provides a method of slowing decline in language capacity in a patient diagnosed with moderate AD, comprising administering to said patient a 4500 mg dose of humanized monoclonal anti-Tau antibody, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

In another aspect, the present disclosure provides a humanized monoclonal anti-Tau antibody provided at a dose of 4500 mg for use in slowing decline in language capacity in a patient diagnosed with moderate AD, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

In another aspect, the present disclosure provides use of a humanized monoclonal anti-Tau antibody for the manufacture of a medicament for slowing decline in language capacity in a patient diagnosed with moderate AD, wherein the antibody is formulated to be provided at a dose of 4500 mg and comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

A further aspect of the present disclosure provides a method of slowing decline in praxis capacity in a patient diagnosed with mild-to-moderate AD, comprising administering to said patient a 4500 mg dose of humanized monoclonal anti-Tau antibody, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

In another aspect, the present disclosure provides a humanized monoclonal anti-Tau antibody provided at a dose of 4500 mg for use in slowing decline in praxis capacity in a patient diagnosed with mild-to-moderate AD, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

In another aspect, the present disclosure provides use of a humanized monoclonal anti-Tau antibody for the manufacture of a medicament for slowing decline in praxis capacity in a patient diagnosed with mild-to-moderate AD, wherein the antibody is formulated to be provided at a dose of 4500 mg and comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

A further aspect of the present disclosure provides a method of slowing decline in praxis capacity in a patient diagnosed with moderate AD, comprising administering to said patient a 4500 mg dose of humanized monoclonal anti-Tau antibody, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

In another aspect, the present disclosure provides a humanized monoclonal anti-Tau antibody provided at a dose of 4500 mg for use in slowing decline in praxis capacity in a patient diagnosed with moderate AD, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

In another aspect, the present disclosure provides use of a humanized monoclonal anti-Tau antibody for the manufacture of a medicament for slowing decline in praxis capacity in a patient diagnosed with moderate AD, wherein the antibody is formulated to be provided at a dose of 4500 mg and comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

An additional aspect of the present disclosure provides a method of slowing decline in language capacity in a patient diagnosed with mild-to-moderate AD, the method comprising intravenously administering to the patient a 4500 mg dose of semorinemab at a frequency of Q2W for 3 doses and subsequently at a frequency of Q4W.

In another aspect, the present disclosure provides semorinemab at a dose of 4500 mg for use in slowing decline in language capacity in a patient diagnosed with mild-to-moderate AD, wherein the semorinemab is intravenously administered at a frequency of Q2W for 3 doses and subsequently at a frequency of Q4W.

In another aspect, the present disclosure provides use of semorinemab for the manufacture of a medicament for slowing decline in language capacity in a patient diagnosed with mild-to-moderate AD, wherein the semorinemab is formulated for intravenous administration at a dose of 4500 mg, wherein the medicament is formulated to be administered at a frequency of Q2W for 3 doses and subsequently at a frequency of Q4W.

A further aspect of the present disclosure provides a method of slowing decline in praxis capacity in a patient diagnosed with mild-to-moderate AD, the method comprising intravenously administering to the patient a 4500 mg dose of semorinemab at a frequency of Q2W for 3 doses and subsequently at a frequency of Q4W.

In another aspect, the present disclosure provides semorinemab at a dose of 4500 mg for use in slowing decline in praxis capacity in a patient diagnosed with mild-to-moderate AD, wherein the semorinemab is intravenously administered at a frequency of Q2W for 3 doses and subsequently at a frequency of Q4W.

In another aspect, the present disclosure provides use of semorinemab for the manufacture of a medicament for slowing decline in praxis capacity in a patient diagnosed with mild-to-moderate AD, wherein the semorinemab is formulated for intravenous administration at a dose of 4500 mg, wherein the medicament is formulated to be administered at a frequency of Q2W for 3 doses and subsequently at a frequency of Q4W.

An additional aspect of the present disclosure provides a method of slowing decline in language capacity in a patient diagnosed with moderate AD, the method comprising intravenously administering to the patient a 4500 mg dose of semorinemab at a frequency of Q2W for 3 doses and subsequently at a frequency of Q4W.

In another aspect, the present disclosure provides semorinemab at a dose of 4500 mg for use in slowing decline in language capacity in a patient diagnosed with moderate AD, wherein the semorinemab is intravenously administered at a frequency of Q2W for 3 doses and subsequently at a frequency of Q4W.

In another aspect, the present disclosure provides use of semorinemab for the manufacture of a medicament for slowing decline in language capacity in a patient diagnosed with moderate AD, wherein the semorinemab is formulated for intravenous administration at a dose of 4500 mg, wherein the medicament is formulated to be administered at a frequency of Q2W for 3 doses and subsequently at a frequency of Q4W.

A further aspect of the present disclosure provides a method of slowing decline in praxis capacity in a patient diagnosed with moderate AD, the method comprising intravenously administering to the patient a 4500 mg dose of semorinemab at a frequency of Q2W for 3 doses and subsequently at a frequency of Q4W.

In another aspect, the present disclosure provides at a dose of 4500 mg for use in slowing decline in praxis capacity in a patient diagnosed with moderate AD, wherein the semorinemab is intravenously administered at a frequency of Q2W for 3 doses and subsequently at a frequency of Q4W.

In another aspect, the present disclosure provides use of semorinemab for the manufacture of a medicament for slowing decline in praxis capacity in a patient diagnosed with moderate AD, wherein the semorinemab is formulated for intravenous administration at a dose of 4500 mg, wherein the medicament is formulated to be administered at a frequency of Q2W for 3 doses and subsequently at a frequency of Q4W.

Unless otherwise indicated, any of the above aspects of methods, antibodies-for-use or uses for slowing decline in language capacity or praxis capacity can comprises any of the anti-Tau antibodies, pharmaceutical compositions, routes of administration, doses, dosing regimens, or co-administrations disclosed herein.

H. Methods of Treating without Increased Risk of an Adverse Event

The present disclosure relates to methods of treating a patient diagnosed with mild-to-moderate Alzheimer's disease (AD) or moderate AD without increased risk of an adverse event. In some embodiments, the methods of this disclosure comprises administering to the subject a 4500 mg dose of humanized monoclonal anti-Tau antibody, without increasing (or without significantly increasing) the risk of a treatment emergent adverse event, optionally wherein the dose is repeated for 12 to 17 doses, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

An aspect of the present disclosure provides a method of treating a subject diagnosed with mild-to-moderate AD without increased risk of an adverse event, comprising administering to said patient a 4500 mg dose of humanized monoclonal anti-Tau antibody, without increasing (or without significantly increasing) the risk of a treatment emergent adverse event, optionally wherein the dose is repeated for 12 to 17 doses, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

In another aspect, the present disclosure provides a humanized monoclonal anti-Tau antibody provided at a dose of 4500 mg for use in treating a patient diagnosed with mild-to-moderate AD without increased risk of an adverse event, optionally wherein the dose is repeated for 12 to 17 doses, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

In another aspect, the present disclosure provides use of a humanized monoclonal anti-Tau antibody for the manufacture of a medicament for treating a patient diagnosed with mild-to-moderate AD without increased risk of an adverse event, wherein the anti-Tau antibody is formulated to be provided at a dose of 4500 mg and comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO: 8.

A further aspect of the present disclosure provides a method of treating a patient diagnosed with moderate AD without increased risk of an adverse event, comprising administering to said patient a 4500 mg dose of humanized monoclonal anti-Tau antibody, without increasing (or without significantly increasing) the risk of a treatment emergent adverse event, optionally wherein the dose is repeated for 12 to 17 doses, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

In another aspect, the present disclosure provides a humanized monoclonal anti-Tau antibody provided at a dose of 4500 mg for use in treating a patient diagnosed with moderate AD without increased risk of an adverse event, optionally wherein the dose is repeated for 12 to 17 doses, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

In another aspect, the present disclosure provides use of a humanized monoclonal anti-Tau antibody for the manufacture of a medicament for treating a patient diagnosed with moderate AD without increased risk of an adverse event, wherein the anti-Tau antibody is formulated to be provided at a dose of 4500 mg and comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

In some embodiments, the adverse event is at least one or more selected from the group consisting of: an infusion-related reaction, a neuroimaging abnormality, immunogenicity; suicide ideation, headache, worsening cognitive function, alteration of consciousness, seizures, unsteadiness, vomiting, a fall, urinary tract infection, anxiety, headache, agitation, depression, dizziness, diarrhea, hypertension, nasopharyngitis, arthralgia, constipation, COVID-19, insomnia, upper respiratory tract infection, abdominal pain, back pain, cough, hematuria, nausea, extremity pain, anemia, confused state, and hallucination. In some embodiments, the adverse event is an infusion-related reaction. In some embodiments, the adverse event is a neuroimaging abnormality. In some embodiments, the adverse event is immunogenicity. In some embodiments, the adverse event is suicide ideation. In some embodiments, the adverse event is a headache. In some embodiments, the adverse event is worsening cognitive function. In some embodiments, the adverse event is alteration of consciousness. In some embodiments, the adverse event is seizures. In some embodiments, the adverse event is unsteadiness. In some embodiments, the adverse event is vomiting. In some embodiments, the adverse event is a fall. In some embodiments, the adverse event is a urinary tract infection. In some embodiments, the adverse event is anxiety. In some embodiments, the adverse event is a headache. In some embodiments, the adverse event is agitation. In some embodiments, the adverse event is depression. In some embodiments, the adverse event is dizziness. In some embodiments, the adverse event is diarrhea. In some embodiments, the adverse event is hypertension. In some embodiments, the adverse event is nasopharyngitis. In some embodiments, the adverse event is arthralgia. In some embodiments, the adverse event is constipation. In some embodiments, the adverse event is COVID-19. In some embodiments, the adverse event is insomnia. In some embodiments, the adverse event is an upper respiratory tract infection. In some embodiments, the adverse event is abdominal pain. In some embodiments, the adverse event is back pain. In some embodiments, the adverse event is cough. In some embodiments, the adverse event is hematuria. In some embodiments, the adverse event is nausea. In some embodiments, the adverse event is extremity pain. In some embodiments, the adverse event is anemia. In some embodiments, the adverse event is confused state. In some embodiments, the adverse event is hallucination.

In some embodiments, a Tau PET tracer of this disclosure, administered to the patient before and/or after administration of the anti-Tau antibody, does not increase the risk of an adverse event.

In some embodiments, administration of the humanized monoclonal anti-Tau antibody of this disclosure does not increase the risk of an adverse event.

A further aspect of this disclosure provides a method of treating a patient diagnosed with mild-to-moderate AD without increased risk of an adverse event, the method comprising intravenously administering to the patient a 4500 mg dose of semorinemab at a frequency of Q2W for 3 doses and subsequently at a frequency of Q4W.

In another aspect, the present disclosure provides semorinemab at a dose of 4500 mg for use in treating a patient diagnosed with mild-to-moderate AD without increased (or without significantly increased) risk of an adverse event, wherein the semorinemab is intravenously administered at a frequency of Q2W for 3 doses and subsequently at a frequency of Q4W.

In another aspect, the present disclosure provides use of semorinemab for the manufacture of a medicament for treating a patient diagnosed with mild-to-moderate AD without increased (or without significantly increased) risk of an adverse event, wherein the semorinemab is formulated for intravenous administration at a dose of 4500 mg, wherein the medicament is formulated to be administered at a frequency of Q2W for 3 doses and subsequently at a frequency of Q4W.

An additional aspect of this disclosure provides a method of treating a patient diagnosed with moderate AD without increased risk of an adverse event, the method comprising intravenously administering to the patient a 4500 mg dose of semorinemab at a frequency of Q2W for 3 doses and subsequently at a frequency of Q4W.

In another aspect, the present disclosure provides semorinemab at a dose of 4500 mg for use in treating a patient diagnosed with moderate AD without increased (or without significantly increased) risk of an adverse event, wherein the semorinemab is intravenously administered at a frequency of Q2W for 3 doses and subsequently at a frequency of Q4W.

In another aspect, the present disclosure provides use of semorinemab for the manufacture of a medicament for treating a patient diagnosed with moderate AD without increased (or without significantly increased) risk of an adverse event, wherein the semorinemab is formulated for intravenous administration at a dose of 4500 mg, wherein the medicament is formulated to be administered at a frequency of Q2W for 3 doses and subsequently at a frequency of Q4W.

Unless otherwise indicated, any of the above aspects of methods, antibodies-for-use or uses for maintaining or slowing memory decline can comprises any of the anti-Tau antibodies, pharmaceutical compositions, routes of administration, doses, dosing regimens, or co-administrations disclosed herein.

Although the foregoing disclosure has been described in some detail by way of illustration and example for purposes of clarity of understanding, the descriptions and examples should not be construed as limiting the scope of the disclosure. The disclosures of all patent applications and publications and scientific literature cited herein are expressly incorporated in their entirety by reference for any purpose.

TABLE 3
TABLE OF AMINO ACID SEQUENCES REFERRED TO
IN THE DISCLOSURE:

| SEQ | | |
|---|---|---|
| ID | | |
| NO | Description | Sequence |
| 1 | Human Tau | AEPRQEFEVMEDHAGTYGLGDRK |
| | epitope (2-24) | |
| 2 | hMTAU HVR-H1 | SYGMS |
| 3 | hMTAU HVR-H2 | TINSGGTYTYYPDSVKG |
| 4 | hMTAU HVR-H3 | SYSGAMDY |
| 5 | hMTAU heavy | EVQLVESGGG LVQPGGSLRL SCAASGLIFR SYGMSWVRQA |
| | chain variable | PGKGLEWVAT INSGGTYTYY PDSVKGRFTI SRDNSKNTLY |
| | region (VH) | LQMNSLRAED TAVYYCANSY SGAMDYWGQG TLVTVSS |
| 6 | hMTAU HVR-L1 | RSSQSIVHSNGNTYLE |
| 7 | hMTAU HVR-L2 | KVSNRFS |
| 8 | hMTAU HVR-L3 | FQGSLVPWT |
| 9 | hMTAU light | DDVLTQTPLS LPVTPGQPAS ISCRSSQSIV HSNGNTYLEW |
| | chain variable | YLQKPGQSPQ LLIYKVSNRF SGVPDRESGS GSGTDETLKI |
| | region (VL) | SRVEAEDVGV YYCFQGSLVP WTFGQGTKVE IK |
| 10 | Cynomolgus | AEPRQEFDVMEDHAGTYGLGDRK |
| | monkey Tau | |
| | epitope (2-24) | |
| 11 | hMTAU heavy | EVQLVESGGG LVQPGGSLRL SCAASGLIFR SYGMSWVRQA |
| | chain version | PGKGLEWVAT INSGGTYTYY PDSVKGRFTI SRDNSKNTLY |
| | 1 | LQMNSLRAED TAVYYCANSY SGAMDYWGQG TLVTVSSAST |
| | | KGPSVFPLAP CSRSTSESTA ALGCLVKDYF PEPVTVSWNS |
| | | GALTSGVHTF PAVLQSSGLY SLSSVVTVPS SSLGTKTYTC |
| | | NVDHKPSNTK VDKRVESKYG PPCPPCPAPE FLGGPSVFLF |
| | | PPKPKDTLYI TREPEVTCVV VDVSQEDPEV QFNWYVDGVE |
| | | VHNAKTKPRE EQFNSTYRVV SVLTVLHQDW LNGKEYKCKV |
| | | SNKGLPSSIE KTISKAKGQP REPQVYTLPP SQEEMTKNQV |
| | | SLTCLVKGFY PSDIAVEWES NGQPENNYKT TPPVLDSDGS |
| | | FFLYSRLTVD KSRWQEGNVF SCSVMHEALH NHYTQKSLSL |
| | | SLGK |
| 12 | hMTAU heavy | EVQLVESGGG LVQPGGSLRL SCAASGLIFR SYGMSWVRQA |
| | chain version | PGKGLEWVAT INSGGTYTYY PDSVKGRFTI SRDNSKNTLY |
| | 2 | LQMNSLRAED TAVYYCANSY SGAMDYWGQG TLVTVSSAST |
| | | KGPSVFPLAP CSRSTSESTA ALGCLVKDYF PEPVTVSWNS |
| | | GALTSGVHTF PAVLQSSGLY SLSSVVTVPS SSLGTKTYTC |
| | | NVDHKPSNTK VDKRVESKYG PPCPPCPAPE FLGGPSVFLF |
| | | PPKPKDTLYI TREPEVTCVV VDVSQEDPEV QFNWYVDGVE |
| | | VHNAKTKPRE EQFNSTYRVV SVLTVLHQDW LNGKEYKCKV |
| | | SNKGLPSSIE KTISKAKGQP REPQVYTLPP SQEEMTKNQV |
| | | SLTCLVKGFY PSDIAVEWES NGQPENNYKT TPPVLDSDGS |
| | | FFLYSRLTVD KSRWQEGNVF SCSVMHEALH NHYTQKSLSL |
| | | SLG |
| 13 | hMTAU light | DDVLTQTPLS LPVTPGQPAS ISCRSSQSIV HSNGNTYLEW |
| | chain | YLQKPGQSPQ LLIYKVSNRF SGVPDRFSGS GSGTDFTLKI |
| | | SRVEAEDVGV YYCFQGSLVP WTFGQGTKVE IKRTVAAPSV |
| | | FIFPPSDEQL KSGTASVVCL LNNFYPREAK VQWKVDNALQ |
| | | SGNSQESVTE QDSKDSTYSL SSTLTLSKAD YEKHKVYACE |
| | | VTHQGLSSPV TKSFNRGEC |
| 14 | MTAU heavy | EVQLVESGGD LAKPGGSLKL SCTASGLIFR SYGMSWVRQT |
| | chain variable | PDKRLEWVAT INSGGTYTYY PDSVKGRFTI SRDNAKNTLY |
| | region (VH) | LQMSSLKSED TAMYYCANSY SGAMDYWGQG TSVTVSS |
| 15 | MTAU light | DDLLTQTPLS LPVSLGDPAS ISCRSSQSIV HSNGNTYFEW |
| | chain variable | YLQKPGQSPK LLIYKVSNRF SGVPDRESGS GSGTDFTLKI |
| | region (VL) | SRVEAEDLGV YYCFQGSLVP WTFGGGTKLE IK |
| 16 | MTAU HVR-H1 | SYGMS |
| 17 | MTAU HVR-H2 | TINSGGTYTYYPDSVKG |
| 18 | MTAU HVR-H3 | SYSGAMDY |
| 19 | MTAU HVR-L1 | RSSQSIVHSNGNTYFE |
| 20 | MTAU HVR-L2 | KVSNRFS |
| 21 | MTAU HVR-L3 | FQGSLVPWT |

### Numbered Embodiments

Particular embodiments of the disclosure are set forth in the following numbered paragraphs:

1. A method of slowing decline in cognitive capacity in a patient diagnosed with mild-to-moderate Alzheimer's disease (AD), comprising
administering to said patient a 4500 mg dose of humanized monoclonal anti-Tau antibody, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.
   2. A method of maintaining cognitive capacity within 5 points of an Alzheimer's disease Assessment Scale, Cognitive Subscale, 11-item version (ADAS-Cog11) score of a patient diagnosed with mild-to-moderate AD, comprising
administering to said patient a 4500 mg dose of humanized monoclonal anti-Tau antibody,
wherein the ADAS-Cog11 score of the patient assessed after administration of 12 to 17 doses of said antibody is no more than 2.5, no more than 3, no more than 3.5, no more than 4, no more than 4.5, or no more than 5 points higher than an ADAS-Cog11 score of the patient assessed before administration of said antibody,
thereby maintaining cognitive capacity within 5 points of the ADAS-Cog11 score of the patient, and
wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.
   3. A method of slowing decline in cognitive capacity in a patient diagnosed with moderate AD, comprising
administering to said patient a 4500 mg dose of humanized monoclonal anti-Tau antibody, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.
   4. A method of maintaining cognitive capacity within 5 points of an Alzheimer's disease Assessment Scale, Cognitive Subscale, 11-item version (ADAS-Cog11) score of a patient diagnosed with moderate AD, comprising
administering to said patient a 4500 mg dose of humanized monoclonal anti-Tau antibody,
wherein the ADAS-Cog11 score of the patient assessed after administration of 12 to 17 doses of said antibody is no more than 2.5, no more than 3, no more than 3.5, no more than 4, no more than 4.5, or no more than 5 points higher than an ADAS-Cog11 score of the patient assessed before administration of said antibody,
thereby maintaining cognitive capacity within 5 points of the ADAS-Cog11 score of the patient, and
wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.
   5. A method of slowing memory decline in a patient diagnosed with mild-to-moderate AD, comprising
administering to said patient a 4500 mg dose of humanized monoclonal anti-Tau antibody, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.
   6. A method of maintaining memory within 2.5 points of an Alzheimer's disease Assessment Scale, Cognitive Subscale, 11-item version (ADAS-Cog11) memory domain score of a patient diagnosed with mild-to-moderate AD, comprising
administering to said patient a 4500 mg dose of humanized monoclonal anti-Tau antibody,
wherein the ADAS-Cog11 memory domain score of the patient assessed after administration of 12 to 17 doses of said antibody is no more than 1, no more than 1.5, no more than 1.7, no more than 2, no more than 2.3, or no more than 2.5 points higher than an ADAS-Cog11 memory domain score of the patient assessed before administration of said antibody,
thereby maintaining memory within 2.5 points of the ADAS-Cog11 memory domain score of the patient, and
wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.
   7. A method of slowing memory decline in a patient diagnosed with moderate AD, comprising
administering to said patient a 4500 mg dose of humanized monoclonal anti-Tau antibody to slow the decline in memory in the patient, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.
   8. A method of maintaining memory within 2.5 points of an Alzheimer's disease Assessment Scale, Cognitive Subscale, 11-item version (ADAS-Cog11) memory domain score of a patient diagnosed with moderate AD, comprising
administering to said patient a 4500 mg dose of humanized monoclonal anti-Tau antibody,
wherein the ADAS-Cog11 memory domain score of the patient assessed after administration of 12 to 17 doses of said antibody is no more than 1, no more than 1.5, no more than 1.7, no more than 2, no more than 2.3, or no more than 2.5 points higher than an ADAS-Cog11 memory domain score of the patient assessed before administration of said antibody,
thereby maintaining memory within 2.5 points of the ADAS-Cog11 memory domain score of the patient, and
wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.
   9. A method of slowing decline in language capacity in a patient diagnosed with mild-to-moderate AD, comprising
administering to said patient a 4500 mg dose of humanized monoclonal anti-Tau antibody, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.
   10. A method of slowing decline in language capacity in a patient diagnosed with moderate AD, comprising
administering to said patient a 4500 mg dose of humanized monoclonal anti-Tau antibody, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.
   11. A method of slowing decline in praxis capacity in a patient diagnosed with mild-to-moderate AD, comprising
administering to said patient a 4500 mg dose of humanized monoclonal anti-Tau antibody, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.
   12. A method of slowing decline in praxis capacity in a patient diagnosed with moderate AD, comprising
administering to said patient a 4500 mg dose of humanized monoclonal anti-Tau antibody, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.
   13. A method of treating a patient diagnosed with mild-to-moderate AD without increased risk of an adverse event, comprising administering to said patient a 4500 mg dose of humanized monoclonal anti-Tau antibody, without increasing (or without significantly increasing) the risk of a treatment emergent adverse event, optionally wherein the dose is repeated for 12 to 17 doses, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.
   14. A method of treating a patient diagnosed with moderate AD without increased risk of an adverse event, comprising administering to said patient a 4500 mg dose of humanized monoclonal anti-Tau antibody, without increasing (or without significantly increasing) the risk of a treatment emergent adverse event, optionally wherein the dose is repeated for 12 to 17 doses, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.
   15. A humanized monoclonal anti-Tau antibody provided at a dose of 4500 mg for use in slowing decline in cognitive capacity in a patient diagnosed with mild-to-moderate AD, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.
   16. A humanized monoclonal anti-Tau antibody provided at a dose of 4500 mg for use in maintaining cognitive capacity no more than 5 points higher than an Alzheimer's disease Assessment Scale, Cognitive Subscale, 11-item version (ADAS-Cog11) score of a patient diagnosed with mild-to-moderate AD, after administration of 12 to 17 doses, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.
   17. A humanized monoclonal anti-Tau antibody provided at a dose of 4500 mg for use in slowing decline in cognitive capacity in a patient diagnosed with moderate AD, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.
   18. A humanized monoclonal anti-Tau antibody provided at a dose of 4500 mg for use in maintaining cognitive capacity no more than 5 points higher than an Alzheimer's disease Assessment Scale, Cognitive Subscale, 11-item version (ADAS-Cog11) score of a patient diagnosed with moderate AD, after administration of 12 to 17 doses, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.
   19. A humanized monoclonal anti-Tau antibody provided at a dose of 4500 mg for use in slowing memory decline in a patient diagnosed with mild-to-moderate AD, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.
   20. A humanized monoclonal anti-Tau antibody provided at a dose of 4500 mg for use in maintaining memory within 2.5 points of an Alzheimer's disease Assessment Scale, Cognitive Subscale, 11-item version (ADAS-Cog11) memory domain score of a patient diagnosed with mild-to-moderate AD, after administration of 12 to 17 doses, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.
   21. A humanized monoclonal anti-Tau antibody provided at a dose of 4500 mg for use in slowing memory decline in a patient diagnosed with moderate AD, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.
   22. A humanized monoclonal anti-Tau antibody provided at a dose of 4500 mg for use in maintaining memory within 2.5 points of an Alzheimer's disease Assessment Scale, Cognitive Subscale, 11-item version (ADAS-Cog11) memory domain score of a patient diagnosed with moderate AD, after administration of 12 to 17 doses, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.
   23. A humanized monoclonal anti-Tau antibody provided at a dose of 4500 mg for use in slowing decline in language capacity in a patient diagnosed with mild-to-moderate AD, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.
   24. A humanized monoclonal anti-Tau antibody provided at a dose of 4500 mg for use in slowing decline in language capacity in a patient diagnosed with moderate AD, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.
   25. A humanized monoclonal anti-Tau antibody provided at a dose of 4500 mg for use in slowing decline in praxis capacity in a patient diagnosed with mild-to-moderate AD, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.
   26. A humanized monoclonal anti-Tau antibody provided at a dose of 4500 mg for use in slowing decline in praxis capacity in a patient diagnosed with moderate AD, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.
   27. A humanized monoclonal anti-Tau antibody provided at a dose of 4500 mg for use in treating a patient diagnosed with mild-to-moderate AD without increased risk of an adverse event, optionally wherein the dose is repeated for 12 to 17 doses, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.
   28. A humanized monoclonal anti-Tau antibody provided at a dose of 4500 mg for use in treating a patient diagnosed with moderate AD without increased risk of an adverse event, optionally wherein the dose is repeated for 12 to 17 doses, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.
   29. Use of a humanized monoclonal anti-Tau antibody for the manufacture of a medicament for slowing decline in cognitive capacity in a patient diagnosed with mild-to-moderate AD, wherein the anti-Tau antibody is formulated to be provided at a dose of 4500 mg and comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.
   30. Use of a humanized monoclonal anti-Tau antibody for the manufacture of a medicament for maintaining cognitive capacity no more than 5 points higher than an Alzheimer's disease Assessment Scale, Cognitive Subscale, 11-item version (ADAS-Cog11) score of a patient diagnosed with mild-to-moderate AD, following administration of 12 to 17 doses, wherein the anti-Tau antibody is formulated to be provided at a dose of 4500 mg and comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.
   31. Use of a humanized monoclonal anti-Tau antibody for the manufacture of a medicament for slowing decline in cognitive capacity in a patient diagnosed with moderate AD, wherein the anti-Tau antibody is formulated to be provided at a dose of 4500 mg and comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.
   32. Use of a humanized monoclonal anti-Tau antibody for the manufacture of a medicament for maintaining cognitive capacity no more than 5 points higher than an Alzheimer's disease Assessment Scale, Cognitive Subscale, 11-item version (ADAS-Cog11) score of a patient diagnosed with moderate AD, following administration of 12 to 17 doses, wherein the anti-Tau antibody is formulated to be provided at a dose of 4500 mg and comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.
   33. Use of a humanized monoclonal anti-Tau antibody for the manufacture of a medicament for slowing memory decline in a patient diagnosed with mild-to-moderate AD, wherein the antibody is formulated to be provided at a dose of 4500 mg and comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.
   34. Use of a humanized monoclonal anti-Tau antibody for the manufacture of a medicament for maintaining memory within 2.5 points of an Alzheimer's disease Assessment Scale, Cognitive Subscale, 11-item version (ADAS-Cog11) memory domain score of a patient diagnosed with mild-to-moderate AD, following administration of 12 to 17 doses, wherein the anti-Tau antibody is formulated to be provided at a dose of 4500 mg and comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.
   35. Use of a humanized monoclonal anti-Tau antibody for the manufacture of a medicament for slowing memory decline in a patient diagnosed with moderate AD, wherein the antibody is formulated to be provided at a dose of 4500 mg and comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.
   36. Use of a humanized monoclonal anti-Tau antibody for the manufacture of a medicament for maintaining memory within 2.5 points of an Alzheimer's disease Assessment Scale, Cognitive Subscale, 11-item version (ADAS-Cog11) memory domain score of a patient diagnosed with moderate AD, following administration of 12 to 17 doses, wherein the anti-Tau antibody is formulated to be provided at a dose of 4500 mg and comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.
   37. Use of a humanized monoclonal anti-Tau antibody for the manufacture of a medicament for slowing decline in language capacity in a patient diagnosed with mild-to-moderate AD, wherein the antibody is formulated to be provided at a dose of 4500 mg and comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.
   38. Use of a humanized monoclonal anti-Tau antibody for the manufacture of a medicament for slowing decline in language capacity in a patient diagnosed with moderate AD, wherein the antibody is formulated to be provided at a dose of 4500 mg and comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.
   39. Use of a humanized monoclonal anti-Tau antibody for the manufacture of a medicament for slowing decline in praxis capacity in a patient diagnosed with mild-to-moderate AD, wherein the antibody is formulated to be provided at a dose of 4500 mg and comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.
   40. Use of a humanized monoclonal anti-Tau antibody for the manufacture of a medicament for slowing decline in praxis capacity in a patient diagnosed with moderate AD, wherein the antibody is formulated to be provided at a dose of 4500 mg and comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.
   41. Use of a humanized monoclonal anti-Tau antibody for the manufacture of a medicament for treating a patient diagnosed with mild-to-moderate AD without increased risk of an adverse event, wherein the anti-Tau antibody is formulated to be provided at a dose of 4500 mg and comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.
   42. Use of a humanized monoclonal anti-Tau antibody for the manufacture of a medicament for treating a patient diagnosed with moderate AD without increased risk of an adverse event, wherein the anti-Tau antibody is formulated to be provided at a dose of 4500 mg and comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.
   43. The method of paragraph 13 or 14, the anti-Tau antibody for use of paragraph 27 or 28, or the use of paragraph 41 or 42, wherein the adverse event is at least one or more selected from the group consisting of: an infusion-related reaction, a neuroimaging abnormality, immunogenicity; suicide ideation, headache, worsening cognitive function, alteration of consciousness, seizures, unsteadiness, vomiting, a fall, urinary tract infection, anxiety, headache, agitation, depression, dizziness, diarrhea, hypertension, nasopharyngitis, arthralgia, constipation, COVID-19, insomnia, upper respiratory tract infection, abdominal pain, back pain, cough, hematuria, nausea, extremity pain, anemia, confused state, and hallucination.
   44. The method of any one of paragraphs 13-14 and 43, the anti-Tau antibody for use of any one of paragraphs paragraph 27-28 and 43, or the use of any one of paragraphs 41-43, wherein a Tau PET tracer, administered to the patient before and/or after administration of the antibody, does not increase the risk of an adverse event.
   45. The method of any one of paragraphs 1-14 and 43-44, the anti-Tau antibody for use of any one of paragraphs 15-28 and 43-44, or the use of any one of paragraphs 29-44, wherein the patient has a Mini-Mental State Exam (MMSE) score of 16-19, inclusive, before administration of said antibody, optionally an MMSE of 16-18, inclusive, before administration of said antibody.
   46. The method of any one of paragraphs 1-14 and 43-45, the anti-Tau antibody for use of any one of paragraphs 15-28 and 43-45, or the use of any one paragraphs 29-45, wherein the patient has a Clinical Dementia Rating Global Score (CDR-GS) of 1 or 2 before administration of said antibody.
   47. The method of any one of paragraphs 1-14 and 43-46, the anti-Tau antibody for use of any one of paragraphs 15-28 and 43-46, or the use of any one of paragraphs 29-46, wherein the dose is repeated at least 5 times, at least 8 times, or at least 10 times, or the dose is repeated for 5-17 doses, 10-17 doses, or 12-17 doses.
   48. The method, the anti-Tau antibody for use, or the use of paragraph 47, wherein the dose is repeated for 13-15 doses, 13-14 doses, 14-15 doses, or 14 doses.
   49. The method, the anti-Tau antibody for use, or the use of paragraph 47, wherein the dose is repeated for 12-16 doses.
   50. The method, the anti-Tau antibody for use, or the use of paragraph 47, wherein the dose is repeated for 14-17 doses.
   51. The method of any one of paragraphs 1-14 and 43-50, the anti-Tau antibody for use of any one of paragraphs 15-28 and 43-50, or the use of any one of paragraphs 29-50, wherein the antibody is administered for at least 24 weeks, optionally at least once every 4 weeks (or monthly).
   52. The method of any one of paragraphs 1-14 and 43-50, the anti-Tau antibody for use of any one of paragraphs 15-28 and 43-50, or the use of any one of paragraphs 29-50, wherein the antibody is administered for at least 36 weeks, optionally at least once every 4 weeks (or monthly).
   53. The method of any one of paragraphs 1-14 and 43-52, the anti-Tau antibody for use of any one of paragraphs 15-28 and 43-52, or the use of any one of paragraphs 29-52, wherein the antibody is administered for at least 40, 44, 48, 52, 56, 60, 64, 68, 72, 76, 80, 84, 88, 92, 96, 100, 104, 108, 112, 116, 120, 124, 128, 132, 136, 140, 144, 148, 152, 156, 160, 164, or 168 weeks, optionally at least once every 4 weeks (or monthly).
   54. The method of any one of paragraphs 1-14 and 43-52, the anti-Tau antibody for use of any one of paragraphs 15-28 and 43-53, or the use of any one of paragraphs 29-53, wherein the antibody is administered for at least 40, 44, 48, 52, 56, or 60 weeks, optionally at least once every 4 weeks (or monthly).
   55. The method, the anti-Tau antibody for use, or the use of paragraph 54, wherein the antibody is administered at least once every 4 weeks (or monthly) for at least 48 weeks.
   56. The method of any one of paragraphs 1-5, 7, 9-14 and 43-55, the anti-Tau antibody for use of any one of paragraphs 15-19, 21, 23-27 and 43-55, or the use of any one of paragraphs 29-33, 35, and 37-55, wherein an ADAS-Cog11 score of the patient assessed after administration of said antibody is no more than 2.5, no more than 3, no more than 3.5, no more than 4, no more than 4.5, or no more than 5 points higher than an ADAS-Cog11 score of the patient assessed before administration of said antibody.
   57. The method of any one of paragraphs 1-5, 7, 9-14 and 43-56, the anti-Tau antibody for use of any one of paragraphs 15-19, 21, 23-27 and 43-55, or the use of any one of paragraphs 29-33, 35, and 37-55, wherein an ADAS-Cog11 score of the patient assessed after administration of said antibody is no more than 4 points higher than an ADAS-Cog11 score of the patient assessed before administration of said antibody.
   58. The method of any one of paragraphs 1-5, 7, 9-14 and 43-56, the anti-Tau antibody for use of any one of paragraphs 15-19, 21, 23-27 and 43-56, or the use of any one of paragraphs 29-33, 35, and 37-56, wherein an ADAS-Cog11 score of the patient assessed after administration of said antibody is 2-4 points higher than an ADAS-Cog11 score of the patient assessed before administration of said antibody.
   59. The method of any one of paragraphs 1-5, 7, 9-14 and 43-58, the anti-Tau antibody for use of any one of paragraphs 15-19, 21, 23-27 and 43-58, or the use of any one of paragraphs 29-33, 35, and 37-58, wherein an ADAS-Cog11 score of the patient assessed after administration of said antibody is 3-4 points higher than an ADAS-Cog11 score of the patient assessed before administration of said antibody.
   60. The method of any one of paragraphs 1-14 and 43-55, the anti-Tau antibody for use of any one of paragraphs 15-28 and 43-55, or the use of any one of paragraphs 29-55, wherein an ADAS-Cog11 memory domain score of the patient assessed after administration of said antibody is no more than 1, no more than 1.5, no more than 1.7, no more than 2, no more than 2.3, or no more than 2.5 points higher than an ADAS-Cog11 memory domain score of the patient assessed before administration of said antibody.
   61. The method of any one of paragraphs 1-14, 43-55 and 60, the anti-Tau antibody for use of any one of paragraphs 15-28, 43-55 and 60, or the use of any one of paragraphs 29-55 and 60, wherein an ADAS-Cog11 memory domain score of the patient assessed after administration of said antibody is no more than 2 points higher than an ADAS-Cog11 memory domain score of the patient assessed before administration of said antibody.
   62. The method of any one of paragraphs 1-14, 43-55 and 60-61, the anti-Tau antibody for use of any one of paragraphs 15-28, 43-55 and 60-61, or the use of any one of paragraphs 29-55 and 60-61, wherein an ADAS-Cog11 memory domain score of the patient assessed after administration of said antibody is 1-2 points higher than an ADAS-Cog11 memory domain score of the patient assessed before administration of said antibody.
   63. The method of any one of paragraphs 1-14, 43-55 and 60, the anti-Tau antibody for use of any one of paragraphs 15-28, 43-55 and 60, or the use of any one of paragraphs 29-55 and 60, wherein an ADAS-Cog11 memory domain score of the patient assessed after administration of said antibody is 1.5-2.5 points higher than an ADAS-Cog11 memory domain score of the patient assessed before administration of said antibody.
   64. The method, the anti-Tau antibody for use, or the use of paragraph 59 or 63, wherein the antibody is administered at least once every 4 weeks (or monthly) for at least 48 weeks.
   65. The method of any one of paragraphs 1-14 and 43-64, the anti-Tau antibody for use of any one of paragraphs 15-28 and 43-64, or the use of any one of paragraphs 29-64, wherein an ADAS-Cog11 score of the patient assessed after administration of said antibody is reduced by at least 25%, at least 30%, at least 35%, at least 40%, at least 45%, or at least 50% compared to that expected without administration of the antibody.
   66. The method of any one of paragraphs 1-14 and 43-65, the anti-Tau antibody for use of any one of paragraphs 15-28 and 43-65, or the use any one of paragraphs 29-65, wherein an ADAS-Cog11 score of the patient assessed after administration of said antibody is reduced by at least 40% compared to that expected without administration of said antibody.
   67. The method of any one of paragraphs 1-14 and 43-66, the anti-Tau antibody for use of any one of paragraphs 15-28 and 43-66, or the use of any one of paragraphs 29-66, wherein an ADAS-Cog11 score of the patient assessed after administration of said antibody is reduced by 25-50% compared to that expected without administration of said antibody.
   68. The method of any one of paragraphs 1-14 and 43-67, the anti-Tau antibody for use of any one of paragraphs 15-28 and 43-67, or the use of any one of paragraphs 29-67, wherein an ADAS-Cog11 score of the patient assessed after administration of said antibody is reduced by 40-50% compared to that expected without administration of said antibody.
   69. The method, the anti-Tau antibody for use, or the use of paragraph 68, wherein the antibody is administered at least once every 4 weeks (or monthly) for at least 48 weeks.
   70. The method of any one of paragraphs 1-14 and 43-69, the anti-Tau antibody for use of any one of paragraphs 15-28 and 43-69, or the use of any one of paragraphs 29-69, wherein the method, the anti-Tau antibody for use or the use comprises administering the antibody once every two weeks, every three weeks, or every four weeks.
   71. The method of any one of paragraphs 1-14 and 43-70, the anti-Tau antibody for use of any one of paragraphs 15-28 and 43-70, or the use of any one of paragraphs 29-70, wherein the method, the anti-Tau antibody for use or the use comprises administering the antibody once every two weeks for one to five doses, and then once every four weeks (or once monthly).
   72. The method of any one of paragraphs 1-14 and 43-71, the anti-Tau antibody for use of any one of paragraphs 15-28 and 43-71, or the use of any one of paragraphs 29-71, wherein the method, the anti-Tau antibody for use or the use comprises administering the antibody once every two weeks for three doses, and then once every four weeks (or once monthly).
   73. The method of any one of paragraphs 1-14 and 43-72, the anti-Tau antibody for use of any one of paragraphs 15-28 and 43-72, or the use of any one of paragraphs 29-72, wherein the method, the anti-Tau antibody for use or the use comprises administering the antibody intravenously.
   74. The method, the anti-Tau antibody for use, or the use of paragraph 73, wherein the administration occurs at an infusion rate of 0.5 to 3.0 mL/minute.
   75. The method, the anti-Tau antibody for use, or the use of paragraph 74, wherein the administration rate occurs at an infusion rate of 0.5-3.0 mL/minute, every four weeks (or monthly).
   76. The method, the anti-Tau antibody for use, or the use of paragraph 75, wherein the infusion rate is 0.5-1 mL/min, optionally for 10-120 minutes of a first infusion; and 3 mL/minute thereafter.
   77. The method of any one of paragraphs 1-14 and 43-76, the anti-Tau antibody for use of any one of paragraphs 15-28 and 43-76, or the use of any one of paragraphs 29-76, wherein the antibody is an IgG4 antibody.
   78. The method, the anti-Tau antibody for use, or the use of paragraph 77, wherein the antibody comprises M252Y, S254T, and T256E mutations, according to EU numbering.
   79. The method, the anti-Tau antibody for use, or the use of paragraph 75, wherein the antibody comprises an S228P mutation, according to EU numbering.
   80. The method of any one of paragraphs 1-14 and 43-79, the anti-Tau antibody for use of any one of paragraphs 15-28 and 43-79, or the use of any one of paragraphs 29-79, wherein the antibody comprises a heavy chain variable region comprising an amino acid sequence that is at least 95% identical to the sequence of SEQ ID NO: 5; and/or a light chain variable region comprising an amino acid sequence that is at least 95% identical to the sequence of SEQ ID NO: 9.
   81. The method of any one of paragraphs 1-14 and 43-80, the anti-Tau antibody for use of any one of paragraphs 15-28 and 43-80, or the use of any one of paragraphs 29-80, wherein the antibody comprises a heavy chain variable region having the amino acid sequence of SEQ ID NO: 5 and/or a light chain variable region having the amino acid sequence of SEQ ID NO:9.
   82. The method of any one of paragraphs 1-14 and 43-81, the anti-Tau antibody for use of any one of paragraphs 15-28 and 43-81, or the use of any one of paragraphs 29-81, wherein the antibody is semorinemab.
   83. The method of any one of paragraphs 1-14 and 43-82, the anti-Tau antibody for use of any one of paragraphs 15-28 and 43-82, or the use of any one of paragraphs 29-82, wherein the patient is Apoε4 positive.
   84. The method of any one of paragraphs 1-14 and 43-82, the anti-Tau antibody for use of any one of paragraphs 15-28 and 43-82, or the use of any one of paragraphs 29-82, wherein the patient is Apoε4 negative.
   85. The method of any one of paragraphs 1-14 and 43-84, the anti-Tau antibody for use of any one of paragraphs 15-28 and 43-84, or the use of any one of paragraphs 29-84, wherein the patient has an MMSE score of 19-21 before administration of the antibody.
   86. The method of any one of paragraphs 1-14 and 43-84, the anti-Tau antibody for use of any one of paragraphs 15-28 and 43-84, or the use of one of paragraphs 29-84, wherein the patient has an MMSE score of 16-19, inclusive, before administration of the antibody, optionally MMSE score of 16-18, inclusive, before administration of the antibody.
   87. The method of any one of paragraphs 1-14 and 43-86, the anti-Tau antibody for use of any one of paragraphs 15-28 and 43-86, or the use of any one of paragraphs 29-86, wherein the patient is Tau positive and/or amyloid beta (Abeta) positive, optionally wherein the patient is determined to be Tau positive by administering to the patient a positron emission tomography (PET) tracer that binds to Tau and optionally wherein the patient is determined to be Abeta positive by administering to the patient a PET tracer that binds to Abeta.
   88. The method, the anti-Tau antibody for use or the use of paragraph 87, wherein a level of Tau is measured by a standardized uptake value ratio (SUVR) measurement of a scan that shows distribution of the PET tracer in the patient's brain.
   89. The method, the anti-Tau antibody for use or the use of paragraph 88, wherein the patient has a high level of Tau, wherein the high level of Tau corresponds to one or more of:
(i) an intracerebral Tau level above or equal to median Genentech Tau Probe 1 (GTP1) whole cortical gray (WCG) (top medium split);
(ii) an SUVR measurement from the temporal region that is equal to or greater than 1.325; and
(iii) an SUVR measurement from the whole cortical gray (WCG) region that is equal to or greater than 1.245.
   90. The method, the anti-Tau antibody for use or the use of paragraph 88, wherein the patient has a low level of Tau, wherein the low level of Tau corresponds to one or more of:
(i) an intracerebral Tau level below median GTP1 WCG (bottom medium split);
(ii) an SUVR measurement from the temporal region that is less than 1.325; and
(iii) an SUVR measurement from the WCG that is less than 1.245.
   91. The method, the anti-Tau antibody for use, or the use of any one of paragraphs 87-90, wherein the PET tracer is at least one selected from the group consisting of [¹⁸F] Genentech Tau Probe 1 ([¹⁸F]GTP1), RO-948, AV-1451 (Flortaucipir), PI-2014, PI-2620, MK-6240, and T-808 and the PET tracer that binds to Abeta is at least one selected from the group consisting of florbetapir, florebetaben, and flutemetamol.
   92. The method, the anti-Tau antibody for use, or the use of any one of paragraphs 87-91, wherein the Tau is measured in a CSF sample or a plasma sample taken from the patient.
   93. The method of any one of paragraphs 1-14 and 43-92, the anti-Tau antibody for use of any one of paragraphs 15-28 and 43-92, or the use of any one of paragraphs 29-92, wherein the patient is co-administered one or more additional agents.
   94. The method, the anti-Tau antibody for use, or the use of paragraph 93, wherein the one or more additional agents are selected from the group consisting of: a symptomatic medication, a neurological drug, a corticosteroid, an antibiotic, an antiviral agent, an additional anti-Tau antibody, a Tau inhibitor, an anti-amyloid beta antibody, a beta-amyloid aggregation inhibitor, an anti-BACE1 antibody, a BACE1 inhibitor; a cholinesterase inhibitor; an NMDA receptor antagonist; a monoamine depletor; an ergoloid mesylate; an anticholinergic antiparkinsonism agent; a dopaminergic antiparkinsonism agent; a tetrabenazine; an anti-inflammatory agent; a hormone; a vitamin; a dimebolin; a homoTaurine; a serotonin receptor activity modulator; an interferon, and a glucocorticoid.
   95. The method, the anti-Tau antibody for use, or the use of paragraph 94, wherein the symptomatic medication is selected from the group consisting of a cholinesterase inhibitor, galantamine, rivastigmine, donepezil, an N-methyl-D-aspartate receptor antagonist, memantine, and a food supplement (optionally wherein the food supplement is Souvenaid®).
   96. The method, the anti-Tau antibody for use, or the use of paragraph 94 or 95, wherein the anti-amyloid beta antibody is aducanemab, lecanemab, or donanemab.
   97. The method, the anti-Tau antibody for use, or the use of any one of paragraphs 94-96, wherein the anti-amyloid beta antibody is crenezumab or gantenerumab.
   98. The method, the anti-Tau antibody for use, or the use of paragraph 94, wherein the additional anti-Tau antibody is selected from the group consisting of a different N-terminal binder, a mid-domain binder, and a fibrillar Tau binder.
   99. The method, the anti-Tau antibody for use, or the use of paragraph 94 or 98, wherein the additional anti-Tau antibody is selected from the group consisting of Gosuranemab, Tilavonemab, Bepranemab, and Zagotenemab.
   100. The method, the anti-Tau antibody for use, or the use of paragraph 93, wherein the one or more additional agents comprises a therapeutic agent that specifically binds to a target selected from the group consisting of beta secretase, Tau, presenilin, amyloid precursor protein or portions thereof, amyloid beta peptide or oligomers or fibrils thereof, death receptor 6 (DR6), receptor for advanced glycation end-products (RAGE), parkin, and huntingtin.
   101. The method, the anti-Tau antibody for use, or the use of paragraph 94, wherein the monoamine depletor is tetrabenazine.
   102. The method, the anti-Tau antibody for use, or the use of paragraph 94, wherein the anticholinergic antiparkinsonism agent is selected from the group consisting of procyclidine, diphenhydramine, trihexylphenidyl, benztropine, biperiden and trihexyphenidyl.
   103. The method, the anti-Tau antibody for use, or the use of paragraph 94, wherein the dopaminergic antiparkinsonism agent is selected from the group consisting of: entacapone, selegiline, pramipexole, bromocriptine, rotigotine, selegiline, ropinirole, rasagiline, apomorphine, carbidopa, levodopa, pergolide, tolcapone and amantadine.
   104. The method, the anti-Tau antibody for use, or the use of paragraph 94, wherein the anti-inflammatory agent is selected from the group consisting of a nonsteroidal anti-inflammatory drug and indomethacin.
   105. The method, the anti-Tau antibody for use, or the use of paragraph 94, wherein the hormone is selected from the group consisting of estrogen, progesterone, and leuprolide.
   106. The method, the anti-Tau antibody for use, or the use of paragraph 94, wherein the vitamin selected from the group consisting of folate and nicotinamide.
   107. The method, the anti-Tau antibody for use, or the use of paragraph 94, wherein the homoTaurine is 3-aminopropanesulfonic acid or 3APS.
   108. The method, the anti-Tau antibody for use, or the use of paragraph 94, wherein the serotonin receptor activity modulator is xaliproden.
   109. The method of any one of paragraphs 1-12 and 45-108, the anti-Tau antibody for use of any one of paragraphs 15-26 and 45-108, or the use of any one of paragraphs 29-40 and 45-108, wherein administration of said antibody does not increase the risk of an adverse event.
   110. The method, the anti-Tau antibody for use, or the use of paragraph 109, wherein the adverse event is at least one selected from the group consisting of: an infusion-related reaction, a neuroimaging abnormality, immunogenicity; suicide ideation, headache, worsening cognitive function, alteration of consciousness, seizures, unsteadiness, vomiting, a fall, urinary tract infection, anxiety, headache, agitation, depression, dizziness, diarrhea, hypertension, nasopharyngitis, arthralgia, constipation, COVID-19, insomnia, upper respiratory tract infection, abdominal pain, back pain, cough, hematuria, nausea, extremity pain, anemia, confused state, and hallucination.
   111. The method of any one of paragraphs 1-14 and 43-110, the anti-Tau antibody for use of any one of paragraphs 15-28 and 43-110, or the use of any one of paragraphs 29-110, wherein the patient is Black or Hispanic or has a non-European ethnic origin.
   112. A method of slowing decline in cognitive capacity in a patient diagnosed with mild-to-moderate AD, the method comprising intravenously administering to the patient a 4500 mg dose of semorinemab at a frequency of Q2W for 3 doses and subsequently at a frequency of Q4W.
   113. A method of maintaining cognitive capacity within 5 points of an ADAS-Cog11 score of a patient diagnosed with mild-to-moderate AD, the method comprising intravenously administering to the patient a 4500 mg dose of semorinemab at a frequency of Q2W for 3 doses and subsequently at a frequency of Q4W.
   114. A method of treating a patient diagnosed with mild-to-moderate AD without increased risk of an adverse event, the method comprising intravenously administering to the patient a 4500 mg dose of semorinemab at a frequency of Q2W for 3 doses and subsequently at a frequency of Q4W.
   115. A method of slowing memory decline in a patient diagnosed with mild-to-moderate AD, the method comprising intravenously administering to the patient a 4500 mg dose of semorinemab at a frequency of Q2W for 3 doses and subsequently at a frequency of Q4W.
   116. A method of maintaining cognitive capacity within 2.5 points of an ADAS-Cog11 score of a patient diagnosed with mild-to-moderate AD, the method comprising intravenously administering to the patient a 4500 mg dose of semorinemab at a frequency of Q2W for 3 doses and subsequently at a frequency of Q4W.
   117. A method of slowing decline in language capacity in a patient diagnosed with mild-to-moderate AD, the method comprising intravenously administering to the patient a 4500 mg dose of semorinemab at a frequency of Q2W for 3 doses and subsequently at a frequency of Q4W.
   118. A method of slowing decline in praxis capacity in a patient diagnosed with mild-to-moderate AD, the method comprising intravenously administering to the patient a 4500 mg dose of semorinemab at a frequency of Q2W for 3 doses and subsequently at a frequency of Q4W.
   119. A method of slowing decline in cognitive capacity in a patient diagnosed with moderate AD, the method comprising intravenously administering to the patient a 4500 mg dose of semorinemab at a frequency of Q2W for 3 doses and subsequently at a frequency of Q4W.
   120. A method of maintaining cognitive capacity within 5 points of an ADAS-Cog11 score of a patient diagnosed with moderate AD, the method comprising intravenously administering to the patient a 4500 mg dose of semorinemab at a frequency of Q2W for 3 doses and subsequently at a frequency of Q4W.
   121. A method of treating a patient diagnosed with moderate AD without increased risk of an adverse event, the method comprising intravenously administering to the patient a 4500 mg dose of semorinemab at a frequency of Q2W for 3 doses and subsequently at a frequency of Q4W.
   122. A method of slowing memory decline in a patient diagnosed with moderate AD, the method comprising intravenously administering to the patient a 4500 mg dose of semorinemab at a frequency of Q2W for 3 doses and subsequently at a frequency of Q4W.
   123. A method of maintaining cognitive capacity within 2.5 points of an ADAS-Cog11 score of a patient diagnosed with moderate AD, the method comprising intravenously administering to the patient a 4500 mg dose of semorinemab at a frequency of Q2W for 3 doses and subsequently at a frequency of Q4W.
   124. A method of slowing decline in language capacity in a patient diagnosed with moderate AD, the method comprising intravenously administering to the patient a 4500 mg dose of semorinemab at a frequency of Q2W for 3 doses and subsequently at a frequency of Q4W.
   125. A method of slowing decline in praxis capacity in a patient diagnosed with moderate AD, the method comprising intravenously administering to the patient a 4500 mg dose of semorinemab at a frequency of Q2W for 3 doses and subsequently at a frequency of Q4W.
   126. Semorinemab at a dose of 4500 mg for use in slowing decline in cognitive capacity in a patient diagnosed with mild-to-moderate AD, wherein the semorinemab is intravenously administered at a frequency of Q2W for 3 doses and subsequently at a frequency of Q4W.
   127. Semorinemab at a dose of 4500 mg for use in maintaining cognitive capacity no more than 5 points higher than an ADAS-Cog11 score of a patient diagnosed with mild-to-moderate AD, wherein the semorinemab is intravenously administered at a frequency of Q2W for 3 doses and subsequently at a frequency of Q4W.
   128. Semorinemab at a dose of 4500 mg for use in treating a patient diagnosed with mild-to-moderate AD without increased (or without significantly increased) risk of an adverse event, wherein the semorinemab is intravenously administered at a frequency of Q2W for 3 doses and subsequently at a frequency of Q4W.
   129. Semorinemab at a dose of 4500 mg for use in slowing memory decline in a patient diagnosed with mild-to-moderate AD, wherein the semorinemab is intravenously administered at a frequency of Q2W for 3 doses and subsequently at a frequency of Q4W.
   130. Semorinemab at a dose of 4500 mg for use in maintaining cognitive capacity no more than 2.5 points higher than an ADAS-Cog11 score of a patient diagnosed with mild-to-moderate AD, wherein the semorinemab is intravenously administered at a frequency of Q2W for 3 doses and subsequently at a frequency of Q4W.
   131. Semorinemab at a dose of 4500 mg for use in slowing decline in language capacity in a patient diagnosed with mild-to-moderate AD, wherein the semorinemab is intravenously administered at a frequency of Q2W for 3 doses and subsequently at a frequency of Q4W.
   132. Semorinemab at a dose of 4500 mg for use in slowing decline in praxis capacity in a patient diagnosed with mild-to-moderate AD, wherein the semorinemab is intravenously administered at a frequency of Q2W for 3 doses and subsequently at a frequency of Q4W.
   133. Semorinemab at a dose of 4500 mg for use in slowing decline in cognitive capacity in a patient diagnosed with moderate AD, wherein the semorinemab is intravenously administered at a frequency of Q2W for 3 doses and subsequently at a frequency of Q4W.
   134. Semorinemab at a dose of 4500 mg for use in maintaining cognitive capacity no more than 5 points higher than an ADAS-Cog11 score of a patient diagnosed with moderate AD, wherein the semorinemab is intravenously administered at a frequency of Q2W for 3 doses and subsequently at a frequency of Q4W.
   135. Semorinemab at a dose of 4500 mg for use in treating a patient diagnosed with moderate AD without increased (or without significantly increased) risk of an adverse event, wherein the semorinemab is intravenously administered at a frequency of Q2W for 3 doses and subsequently at a frequency of Q4W.
   136. Semorinemab at a dose of 4500 mg for use in slowing memory decline in a patient diagnosed with moderate AD, wherein the semorinemab is intravenously administered at a frequency of Q2W for 3 doses and subsequently at a frequency of Q4W.
   137. Semorinemab at a dose of 4500 mg for use in maintaining cognitive capacity no more than 2.5 points higher than an ADAS-Cog11 score of a patient diagnosed with moderate AD, wherein the semorinemab is intravenously administered at a frequency of Q2W for 3 doses and subsequently at a frequency of Q4W.
   138. Semorinemab at a dose of 4500 mg for use in slowing decline in language capacity in a patient diagnosed with moderate AD, wherein the semorinemab is intravenously administered at a frequency of Q2W for 3 doses and subsequently at a frequency of Q4W.
   139. Semorinemab at a dose of 4500 mg for use in slowing decline in praxis capacity in a patient diagnosed with moderate AD, wherein the semorinemab is intravenously administered at a frequency of Q2W for 3 doses and subsequently at a frequency of Q4W.
   140. Use of semorinemab for the manufacture of a medicament for slowing decline in cognitive capacity in a patient diagnosed with mild-to-moderate AD, wherein the semorinemab is formulated for intravenous administration at a dose of 4500 mg, wherein the medicament is administered at a frequency of Q2W for 3 doses and subsequently at a frequency of Q4W.
   141. Use of semorinemab for the manufacture of a medicament for maintaining cognitive capacity no more than 5 points higher than an ADAS-Cog11 score of a patient diagnosed with mild-to-moderate AD, wherein the semorinemab is formulated for intravenous administration at a dose of 4500 mg, wherein the medicament is formulated to be administered at a frequency of Q2W for 3 doses and subsequently at a frequency of Q4W.
   142. Use of semorinemab for the manufacture of a medicament for treating a patient diagnosed with mild-to-moderate AD without increased (or without significantly increased) risk of an adverse event, wherein the semorinemab is formulated for intravenous administration at a dose of 4500 mg, wherein the medicament is formulated to be administered at a frequency of Q2W for 3 doses and subsequently at a frequency of Q4W.
   143. Use of semorinemab for the manufacture of a medicament for slowing memory decline in a patient diagnosed with mild-to-moderate AD, wherein the semorinemab is formulated for intravenous administration at a dose of 4500 mg, wherein the medicament is formulated to be administered at a frequency of Q2W for 3 doses and subsequently at a frequency of Q4W.
   144. Use of semorinemab for the manufacture of a medicament for maintaining cognitive capacity no more than 2.5 points higher than an ADAS-Cog11 score of a patient diagnosed with mild-to-moderate AD, wherein the semorinemab is formulated for intravenous administration at a dose of 4500 mg, wherein the medicament is formulated to be administered at a frequency of Q2W for 3 doses and subsequently at a frequency of Q4W.
   145. Use of semorinemab for the manufacture of a medicament for slowing decline in language capacity in a patient diagnosed with mild-to-moderate AD, wherein the semorinemab is formulated for intravenous administration at a dose of 4500 mg, wherein the medicament is formulated to be administered at a frequency of Q2W for 3 doses and subsequently at a frequency of Q4W.
   146. Use of semorinemab for the manufacture of a medicament for slowing decline in praxis capacity in a patient diagnosed with mild-to-moderate AD, wherein the semorinemab is formulated for intravenous administration at a dose of 4500 mg, wherein the medicament is formulated to be administered at a frequency of Q2W for 3 doses and subsequently at a frequency of Q4W.
   147. Use of semorinemab for the manufacture of a medicament for slowing decline in cognitive capacity in a patient diagnosed with moderate AD, wherein the semorinemab is formulated for intravenous administration at a dose of 4500 mg, wherein the medicament is administered at a frequency of Q2W for 3 doses and subsequently at a frequency of Q4W.
   148. Use of semorinemab for the manufacture of a medicament for maintaining cognitive capacity no more than 5 points higher than an ADAS-Cog11 score of a patient diagnosed with moderate AD, wherein the semorinemab is formulated for intravenous administration at a dose of 4500 mg, wherein the medicament is formulated to be administered at a frequency of Q2W for 3 doses and subsequently at a frequency of Q4W.
   149. Use of semorinemab for the manufacture of a medicament for treating a patient diagnosed with moderate AD without increased (or without significantly increased) risk of an adverse event, wherein the semorinemab is formulated for intravenous administration at a dose of 4500 mg, wherein the medicament is formulated to be administered at a frequency of Q2W for 3 doses and subsequently at a frequency of Q4W.
   150. Use of semorinemab for the manufacture of a medicament for slowing memory decline in a patient diagnosed with moderate AD, wherein the semorinemab is formulated for intravenous administration at a dose of 4500 mg, wherein the medicament is formulated to be administered at a frequency of Q2W for 3 doses and subsequently at a frequency of Q4W.
   151. Use of semorinemab for the manufacture of a medicament for maintaining cognitive capacity no more than 2.5 points higher than an ADAS-Cog11 score of a patient diagnosed with moderate AD, wherein the semorinemab is formulated for intravenous administration at a dose of 4500 mg, wherein the medicament is formulated to be administered at a frequency of Q2W for 3 doses and subsequently at a frequency of Q4W.
   152. Use of semorinemab for the manufacture of a medicament for slowing decline in language capacity in a patient diagnosed with moderate AD, wherein the semorinemab is formulated for intravenous administration at a dose of 4500 mg, wherein the medicament is formulated to be administered at a frequency of Q2W for 3 doses and subsequently at a frequency of Q4W.
   153. Use of semorinemab for the manufacture of a medicament for slowing decline in praxis capacity in a patient diagnosed with moderate AD, wherein the semorinemab is formulated for intravenous administration at a dose of 4500 mg, wherein the medicament is formulated to be administered at a frequency of Q2W for 3 doses and subsequently at a frequency of Q4W.
   154. The method of any one of paragraph 112-125, the semorinemab for use of any one of paragraphs 126-139, or the use of any one of paragraphs 140-153, wherein the semorinemab is administered at a frequency of Q4W for at least 10 doses.
   155. The method, the semorinemab for use, or the use of paragraph 154, wherein the semorinemab is administered at a frequency of Q4W for at least 13 doses.
   156. The method, the semorinemab for use, or the use of paragraph 154, wherein the semorinemab is administered at a frequency of Q4W for at least 16 doses.
   157. The method of any one of paragraphs 112-125 and 154-156, the semorinemab for use of any one of paragraphs 126-139 and 154-156, or the use of any one of paragraphs 140-156, wherein the semorinemab is administered at an infusion rate of 0.5 m/min to 3.0 mL/min.
   158. The method, the semorinemab for use, or the use of paragraph 157, wherein the infusion rate is 0.5 mL/min to 1 mL/min, optionally for 10-120 minutes of a first infusion; and 3 mL/minute thereafter.
   159. The method of any one of paragraphs 112-125 and 154-158, the semorinemab for use of any one of paragraphs 126-139 and 154-158, or the use of any one of paragraphs 140-158, wherein the method, the semorinemab for use and the use further comprises intravenously administering to the patient a 4500 mg dose of semorinemab at a frequency of Q4W for 96 weeks.

### EXAMPLES

The following illustrative examples are representative of embodiments of compositions and methods described herein and are not meant to be limiting in any way.

### Example 1

Study GN40040 was a proof of concept study using clinical outcome assessments (COAs), a novel Tau PET imaging technology, and other biomarkers to test that semorinemab administration to patients with mild-to-moderate AD stops or slows cell-to-cell spread and propagation of Tau pathology in the brain and improves clinical outcomes.

### Drug and Placebo

Semorinemab and placebo were prepared and diluted into 100 mL IV bags according to the pharmacy manual, and infusions were administered per the instructions outlined in Table 4 and the pharmacy manual.

TABLE 4

| First Infusion ^{a} | Subsequent Infusions |
|---|---|
| Begin infusion at an initial rate of | If the participant experienced an |
| 0.5 mL/min (30 mL/hr). | infusion-related or hypersensitivity reaction |
| If no infusion-related or hypersensitivity | during the prior infusion, begin infusion at an |
| reaction occurs during the first 30 minutes, | initial rate of 0.5 mL/min and follow |
| then increase the rate to 1.0 mL/min | instructions for first infusion. |
| (60 mL/hr). | If the participant tolerated the prior infusion |
| If no infusion-related or hypersensitivity | well (defined by an absence of Grade 2 |
| reaction occurs during the subsequent | reactions during a final infusion rate of |
| 30 minutes, then increase the rate to | 3.0 mL/min), begin infusion at a rate of |
| 3.0 mL/min (180 mL/hr). | 3.0 mL/min. |
| If a reaction develops, stop or slow the | If a reaction develops, stop or slow the |
| infusion. Administer medications and | infusion. Administer medications and |
| supportive care in accordance with | supportive care in accordance with |
| institutional guidelines. The patient should | institutional guidelines. The patient should |
| be monitored until all infusion-related | be monitored until all infusion-related |
| adverse events are Grade 1 or are resolved. | adverse events are Grade 1 or are resolved. |
| If the reaction resolves, resume the infusion | If the reaction resolves, resume the infusion |
| at a 50% reduction in rate (i.e., 50% of rate | at a 50% reduction in rate (i.e., 50% of rate |
| being used at the time that the reaction | being used at the time that the reaction |
| occurred). If the reaction recurs after | occurred). If the reaction recurs after |
| resumption of the infusion, then stop the | resumption of the infusion, then stop the |
| infusion. | infusion. |

OLE = open-label extension.
^{a} First infusion instructions apply to the first infusion of semorinemab or placebo during the double-blind treatment period and the first infusion of semorinemab during the OLE period.

### Investigational Medicinal Products

Semorinemab and placebo were the investigational medicinal products (IMPs) in this study. Study drug (semorinemab or placebo) was administered intravenously in the double-blind treatment period, and semorinemab was administered intravenously in the optional OLE period. Study drug administration will occur Q2W for the first three doses of the double-blind treatment period and Q4W thereafter during the double-blind treatment period. semorinemab was administered Q4W in the OLE period.

### Non-Investigational Medicinal Products

Depending on local classification, the [¹⁸F]GTP1 Tau PET radioligand and/or the amyloid PET radioligand(s) may be considered non-investigational medicinal products or IMPs.

### Objectives and Endpoints

The study evaluated the clinical efficacy, safety, pharmacokinetics, and pharmacodynamics of semorinemab in patients with mild-to-moderate AD, ages 50-85, who are amyloid positive by cerebrospinal fluid (CSF) or amyloid positron emission tomography (PET). Specific objectives and corresponding endpoints for the study are outlined below.

### Primary Efficacy Objective

The primary efficacy objective for this study was to evaluate the effects of semorinemab compared with placebo on cognition and function on the basis of the following endpoints:
Change from baseline to the last visit of the double-blind treatment period (Week 49 for Cohort 1, Week 61 for Cohort 2, and Week 73 for Cohort 3) in cognitive function as measured by the Alzheimer's Disease Assessment Scale, Cognitive Subscale, 11 item version (ADAS Cog11)
Change from baseline to the last visit of the double-blind treatment period (Week 49 for Cohort 1, Week 61 for Cohort 2, and Week 73 for Cohort 3) in functional capacities as measured by the Alzheimer's Disease Cooperative Study Activities of Daily Living Inventory (ADCS-ADL) The placebo control group helped establish a baseline safety profile, to identify any adverse events that may be non-study drug related and served as a comparison group for efficacy measures in the double-blind treatment period of the study.

### Secondary Efficacy Objectives

The secondary efficacy objectives for this study along with the corresponding endpoints were as follows:
To evaluate the effect of semorinemab on global cognition and function compared with placebo on the basis of the following endpoint:
   Change from baseline to the last visit of the double-blind treatment period (Week 49 for Cohort 1, Week 61 for Cohort 2, and Week 73 for Cohort 3) on the Clinical Dementia Rating-Sum of Boxes (CDR SB)
To evaluate the effect of semorinemab on cognition compared with placebo on the basis of the following endpoint:
   Change from baseline to the last visit of the double-blind treatment period (Week 49 for Cohort 1, Week 61 for Cohort 2, and Week 73 for Cohort 3) on the Mini Mental State Examination (MMSE)

### Exploratory Efficacy Objectives

The exploratory efficacy objectives for this study were as follows:
To evaluate the effect of semorinemab on behavioral symptoms compared with placebo on the basis of the following endpoint:
   Change from baseline to the last visit of the double-blind treatment period (Week 49 for Cohort 1, Week 61 for Cohort 2, and Week 73 for Cohort 3) on the Neuropsychiatric Inventory (NPI)
To evaluate the effect of semorinemab on caregiver impression of changes in patient cognition and functional abilities compared with placebo on the basis of the following endpoint:
   Ratings on the Caregiver Global Impression Scales for Alzheimer's disease (CaGI-Alz) at the last visit of the double-blind treatment period (Week 49 for Cohort 1, Week 61 for Cohort 2, and Week 73 for Cohort 3) Another exploratory endpoint involved:
WCG [¹⁸F]GTP1 tau PET SUVR

### Safety Objectives

The safety objectives for this study were to evaluate the safety and tolerability of semorinemab compared with placebo on the basis of the following endpoints:
Nature, frequency, severity, and timing of adverse events and serious adverse events, with severity determined according to WHO toxicity grading scale
Changes from baseline in vital signs, physical findings, neurologic findings, ECG, and clinical laboratory results during and following semorinemab administration
Changes from baseline in suicidal ideation and behavior during and following semorinemab administration as assessed by the Columbia Suicide Severity Rating Scale (C SSRS)
Nature, frequency, severity, and timing of neuroimaging abnormalities that could represent cerebral vasogenic edema and/or microhemorrhage, during semorinemab administration

### Pharmacokinetic Objectives

The pharmacokinetic (PK) objective for this study was to characterize the semorinemab PK profile on the basis of the following endpoint:
Serum concentration of semorinemab at specified time-points

The exploratory PK objectives for this study were as follows:
To characterize the semorinemab PK profile in CSF on the basis of the following endpoint: CSF concentration of semorinemab at specified time-points
To evaluate potential relationships between drug exposure and the efficacy and safety of semorinemab on the basis of the following endpoints:
   Relationship between serum (and CSF if available) concentration or PK parameters for semorinemab and safety endpoints
   Relationship between serum (and CSF if available) concentration or PK parameters for semorinemab and efficacy endpoints
To evaluate potential relationships between selected covariates and exposure to semorinemab on the basis of the following endpoint:
   Relationship between selected covariates (including but not limited to age and sex) and serum concentration or PK parameters for semorinemab

### Immunogenicity Objectives

The immunogenicity objectives for this study were as follows:
To evaluate the immune response to semorinemab on the basis of the following endpoint:
   Incidence of anti-drug antibodies (ADAs) during the study relative to the prevalence of ADAs at baseline
To evaluate the potential effects of ADAs on the basis of the following endpoint:
   Relationship between ADA status and efficacy, safety, or PK endpoints

### Biomarker Objective

The exploratory biomarker objective for this study was to evaluate the effect of semorinemab on biomarkers to provide evidence of proof of activity, to aid in defining mechanism of action, to evaluate the relationship between changes in biomarkers and efficacy, and to evaluate if biomarkers, at baseline, identify a subset of patients with more rapid disease progression and/or enhanced clinical benefit to semorinemab on the basis of the following endpoints:
Relationship between biomarkers in blood and CSF (if available) and efficacy, safety, PK, immunogenicity, or other biomarker endpoints
Relationship between change from baseline brain volume and cortical thickness as measured by magnetic resonance imaging (MRI) and efficacy, safety, PK, immunogenicity, or other biomarker endpoints
Relationship between change from baseline intracerebral Tau pathology burden as measured by [¹⁸F] Genentech Tau probe 1 (GTP1) PET and efficacy, safety, PK, immunogenicity, or other biomarker endpoints

Exploratory biomarker research may have included, but was not limited to, analyses of soluble Tau, neurofilament light chain, and genetic markers associated with AD, neurodegeneration, and neuro-inflammation. Research may involve extraction of DNA, to enable whole genome sequencing (WGS), and analysis of single nucleotide polymorphisms (SNPs).

### Study Design

This Phase II, multicenter, randomized, double blind, placebo controlled, parallel group study evaluated the clinical efficacy, safety, pharmacokinetics, and pharmacodynamics of semorinemab in patients with mild-to-moderate AD.

Briefly, participants aged 50-85 years who fulfilled National Institute on Aging-Alzheimer's Association criteria for probable AD dementia and had Mini-Mental Status Examination (MMSE) scores of 16-21 (inclusive), global Clinical Dementia Rating (CDR) scores of 1 or 2 were randomized to receive monthly IV doses of either placebo or semorinemab (4500 mg) over 48 weeks (i.e., Weeks 1-49). During the course of the study, the protocol was amended to mitigate potential COVID-19 associated disruptions by extending the blinded period of the study to 60 weeks (i.e., Weeks 1-61) for participants who missed at least one dose of study medication during the pandemic. Randomization was stratified by MMSE (16-18 vs. 19-21) and APOε status (ε4+ vs. ε4−). The co-primary efficacy endpoints were change from Baseline to Week 49 on the 11-item version of the Alzheimer's Disease Assessment Scale-Cognitive Subscale (ADAS-Cog11) and on the Alzheimer's Disease Cooperative Study-Activities of Daily Living scale (ADCS-ADL). Secondary and exploratory efficacy endpoints included change from baseline on the MMSE, CDR Sum of Boxes (CDR-SB), and whole cortical grey [18F]GTP1 tau PET SUVR. Analyses incorporated a mixed-effect model of repeated measures (MMRM) using an unstructured covariance matrix. Key safety assessments included physical and neurologic examinations, clinical laboratory assessments, brain magnetic resonance imaging (MRI), and adverse event monitoring.

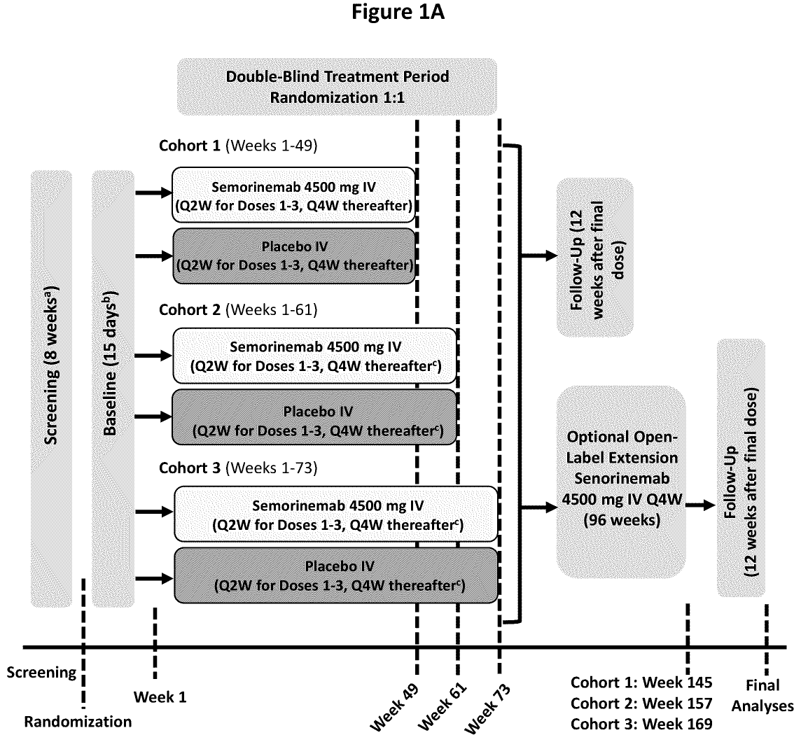

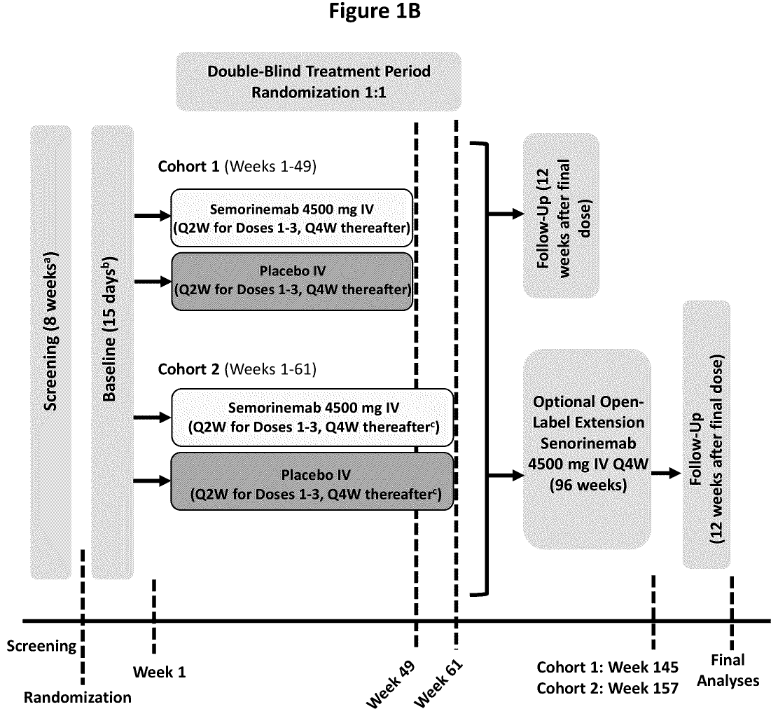

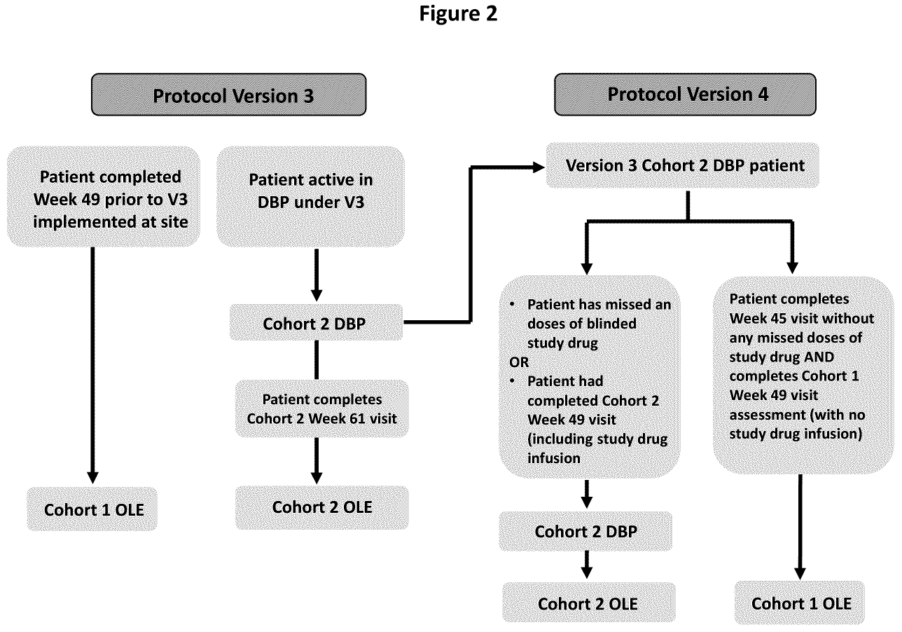

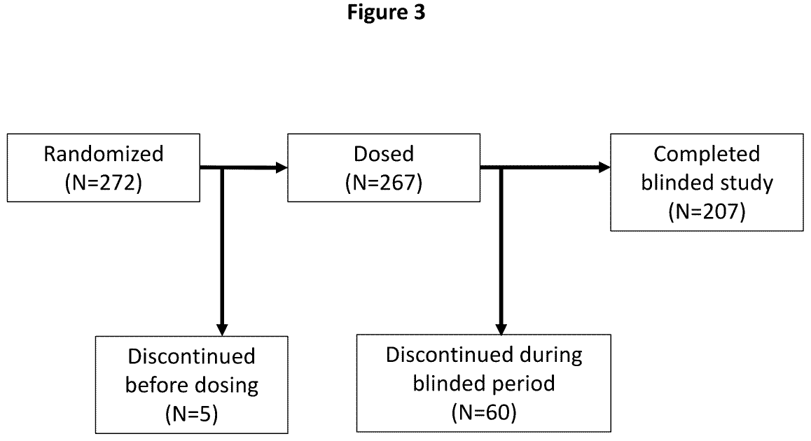

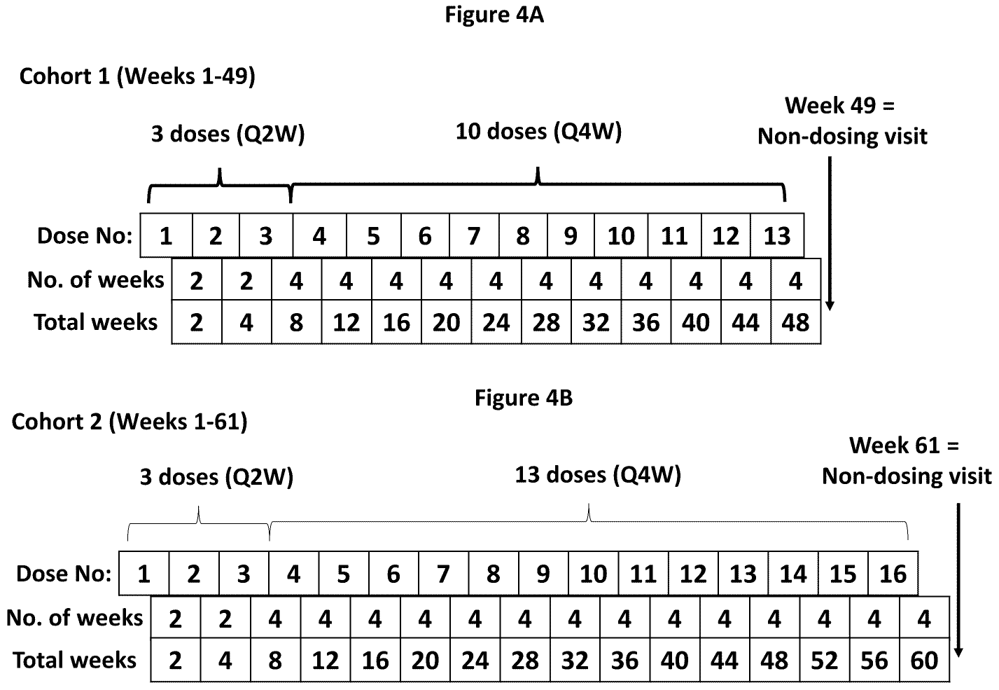

The study consisted of a screening period, a double-blind treatment period, an optional open label extension (OLE) period, and a safety follow up period (see FIGS. 1A and 1B). An extended baseline visit (up to 15 days) was included in the double-blind treatment period, following randomization and prior to the initiation of study drug. Study drug (semorinemab or placebo) was administered intravenously in the double-blind treatment period, and semorinemab was administered intravenously in the optional OLE period. Study drug administration occurred every 2 weeks (Q2W) for the first three doses of the double-blind treatment period and every 4 weeks (Q4W) thereafter during the double-blind treatment period. That is, the dosing frequency was Q4W, except during the first month, when a dose of semorinemab also was administered to patients on Week 3 to rapidly increase the serum concentrations to those achieved at steady state. Because of anticipated disruptions in study drug administration associated with global COVID-19 disruptions, up to three study cohorts were anticipated, as described below:
Cohort 1: Patients who had completed or discontinued from the double-blind treatment period (i.e., completed Week 49 assessments) prior to implementation of the protocol (Protocol Version 3). For Cohort 1, the Week 49 visit represented the end of the double-blind treatment period and patients continued into the OLE or safety follow-up periods per the original schedule. In addition, patients in other cohorts who were active in the double-blind treatment period and had completed blinded study drug treatment through Week 45 without any missed doses were eligible to revert to the Cohort 1 Schedule of Activities at the Week 49 visit and continue on to the OLE after completing the Week 49 visit assessments. See FIG. 2.

If a decision was made to expand the study size, any additional patients recruited into the study were assigned to Cohort 1. If a Cohort 1 patient missed any blinded study drug infusion, the patient was moved into Cohort 2.
Cohort 2: Patients who were active in the double-blind treatment period after implementation of Protocol Version 3 were assigned to Cohort 2. In Cohort 2, the double-blind treatment period was extended to Week 61 unless the criteria above for reverting to the Cohort 1 Schedule of Activities were fulfilled.
Cohort 3: If, by the second half of 2020, COVID-19 disruptions resulted in a significant proportion of patients having missed more than three study drug infusions in the double-blind treatment period, the Sponsor had the option to establish Cohort 3, consisting of patients still active in the double-blind treatment period and who missed more than three doses of blinded study drug at the time of the decision. For this cohort, the double-blind treatment period was extended to Week 73. Cohort 3, however, was never activated.

For patients in Cohort 2 and Cohort 3, if two or more consecutive study drug infusions were missed during the double-blind treatment period, study drug administration was re-initiated with Q2W dosing for the next three doses, followed by Q4W dosing thereafter. This brief course of higher frequency dosing, which was identical to dosing at the start of the double-blind treatment period, was expected to rapidly restore study drug exposure to prior steady-state levels.

For eligible patients in all cohorts, semorinemab was administered Q4W for 96 weeks during the OLE period. Study treatment was defined as study drug plus the [^{18F}]GTP1 radioligand used during PET imaging procedures. [¹⁸F]GTP1 radioligand was supplied as a sterile non pyrogenic solution in sterile borosilicate glass vials with gray butyl septa and aluminum ring seals. The final product bears a label with the following items: total activity (mCi), volume (mL), strength (mCi/mL), calibration date and time, batch number, study identification, and shelf life.

Patients were randomly assigned to receive semorinemab (4500 mg) or placebo administered by IV infusion in a 1:1 ratio. Randomization of patients will be managed by a central interactive voice or web-based response system (IxRS) vendor using stratified permuted block randomization.

All patients participating in the OLE received semorinemab 4500 mg IV infusion. Semorinemab and placebo were supplied as sterile liquids in glass vials. Because both APOε genotype (de Oliviera et al. J. of Neurol. Sci. 2015; 359(1-2):127-132) and baseline cognitive performance (Kennedy et al. Alzheimers Dement (NY) 2015; 1:46-52) may be associated with subsequent rates of clinical progression, randomization was stratified by both APOε status (Apoε4+ vs. Apoε4−) and screening MMSE scores (16-18 vs. 19-21).

Patients were selected on the basis of clinical diagnosis of probable AD dementia, according to the National Institute on Aging-Alzheimer's Association [NIA-AA] Diagnostic Criteria and Guidelines for AD. Clinical diagnosis for each patient was supported by information provided on a Diagnostic Verification Form (DVF), reviewed and approved by the Sponsor or Sponsor delegate.

Eligible patients were 50-85 years old at the beginning of screening, met diagnostic criteria for probable AD dementia (McKhann et al. Alzheimers Dement 2011; 7:263-9), and had evidence of cerebral amyloidosis as indicated by CSF analysis (i.e., CSF enrolled patients) or positive amyloid PET scan by qualitative read (i.e., PET enrolled patients). The choice between CSF versus PET for determination of cerebral amyloidosis was made on the basis of the capability of an individual site and/or the preference of an individual patient. If a patient is amyloid negative based on one of the two modalities (CSF assessment or amyloid PET), then the patient may undergo assessment with the other modality during screening; amyloid positivity by either modality is sufficient for eligibility. At the time of screening, eligible patients had a MMSE score between 16 and 21 (inclusive) and a Clinical Dementia Rating-Global Score (CDR-GS) of 1 or 2.

The MMSE was administered to patients at screening to determine eligibility for the trial, at baseline, and at other post-baseline time-points.

The CDR interview was administered to both the patient with AD and his or her caregiver. The ratings of degree of impairment obtained for each of the six categories of function (the six “boxes”) were synthesized into one global rating of dementia (range, 0-3). A more refined measure of impairment was available by using the CDR SB that is the sum of the six domains and has a range of 0-18. Reliability and validity have been established, as it has high inter-rater reliability. The CDR was used as a global assessment of severity of dementia.

The ADAS Cog11 (11 item version) was used. The ADAS Cog11 was administered to the patient. Equivalent alternate forms of the word recall and word recognition subtests were used in successive test administrations.

The ADCS-ADL was administered to caregivers and covered both basic activities of daily living (ADL) (e.g., eating and toileting) and more complex ADL or instrumental ADL (e.g., using the telephone, managing finances, preparing a meal).

The NPI was administered to caregivers and focused on 12 common behavioral symptoms most often reported in AD.

For CaGI Alz assessment, caregivers were asked to rate the patient's change in memory and ADL since study treatment started and since the previous CaGI Alz assessment (e.g., the prior 6 months). These items were used as anchors to determine meaningful change on other COAs.

Patients had baseline and longitudinal Tau related biomarker evaluation via [¹⁸F]GTP1 PET imaging, with the exception of any additional patients enrolled if the study size was expanded. For those patients, [¹⁸F]GTP1 PET imaging will be optional. For patients undergoing [¹⁸F]GTP1 PET imaging, PET scans were performed at the baseline and Week 49 visits during the double blind treatment period, and for the subset of these patients who continue into the optional OLE period, [¹⁸F]GTP1 PET imaging was performed at the conclusion of the OLE period.

Patients also had the option to have CSF collected via lumbar puncture (LP) at the screening and/or baseline visits and the last study visit during the double-blind treatment period (i.e., at Week 49 for Cohort 1, Week 61 for Cohort 2, or Week 73 for Cohort 3). Patients continuing to the optional OLE period were encouraged to have an LP performed at Week 97 (Cohort 1), Week 109 (Cohort 2), or Week 121 (Cohort 3) and at the conclusion of the OLE period.

[¹⁸F]GTP1 PET and MRI evaluation used a standard protocol. Screening amyloid PET scans and MRIs were read by a central reader to determine eligibility.

### Patients

This study included 272 patients and was conducted at approximately 43 sites in North America and Europe. If a decision was made to expand the study size, up to an additional 100 patients could be enrolled to further mitigate the impact of COVID-19-related disruptions the study. The patients were male and female patients with mild-to-moderate AD, between the ages of 50 and 85 years.

As this study was conducted in multiple geographic regions, patients of different ethnic origins were likely enrolled in the study (including persons of non-European descent). This information was collected (where permitted by local regulations) to evaluate results (e.g., possible differences in PK exposure [concentration of the drug in the blood] or treatment effect) on patients of different ethnic origins.

### Target Population

Inclusion Criteria: Patients meet the following criteria for study entry:
Signed Informed Consent Form by the patient (co-signed by the patient's legally authorized representative, if deemed appropriate by the investigator and/or required by the local regulations, guidelines, and Institutional Review Board or Ethics Committee)
Ability to comply with the study protocol, in the investigator's judgment
Age between 50 and 85 years, inclusive, at time of signing Informed Consent Form
National Institute on Aging/Alzheimer's Association core clinical criteria for probable AD dementia (to ensure that the patients selected are likely to fulfill the clinical diagnostic criteria for mild-to-moderate AD dementia, evidence of prior decline consistent with AD was verified through observations made by clinician or caregiver and recorded on the DVF, subject to adjudication of diagnosis by the Sponsor or Sponsor delegates, to ensure that patients are enrolled on the basis of objectively ascertained and well documented diagnosis of AD (McKhann et al. Alzheimers Dement 2011; 7:263-9).
Evidence of the AD pathological process, by a positive amyloid assessment either on CSF Abetal-42 as measured on Elecsys β Amyloid(1-42) Test System or amyloid PET scan by visual read by the core/central PET vendor.

If a patient is amyloid negative based on CSF assessment, they may undergo an amyloid PET scan during screening to potentially be enrolled. The patient may undergo an LP for CSF assessment or an amyloid PET scan only one time each during screening.

If a patient was amyloid negative based on an amyloid PET scan, they could undergo an LP for CSF assessment during screening for potential eligibility. The patient may undergo an LP for CSF assessment or an amyloid PET scan only one time each during screening.

Under certain circumstances, a previously acquired amyloid PET scan could be used for study inclusion. If the previously acquired amyloid PET scan was considered valid and was read negative by the core/central PET vendor, the patient could undergo CSF assessment for potential eligibility, but not an additional amyloid PET scan for enrollment.
AD dementia of mild-to-moderate severity, as defined by a screening MMSE score of 16-21 points, inclusive, and a CDR GS of 1 or 2

MMSE obtained during the screening period for Study GN39763 could be used provided this was obtained within 8 weeks of the randomization date for Study GN40040 and provided the screen failure was not due to evidence of amyloid negativity.
Currently not receiving non-investigational AD medications except for as defined below:

If the patient is receiving non-investigational AD medications, the dosing regimen must have been stable for 2 months prior to the start of screening. There should be no a priori intent to initiate, discontinue, or alter the dose of any AD therapy for the duration of the study. However, following the initiation of study drug, standard of care symptomatic medications for AD may be initiated, dose adjusted, or discontinued as deemed clinically appropriate. Symptomatic medications included, e.g., cholinesterase inhibitors, memantine, and/or the medical food supplement Souvenaid®).
Inclusion subject to Sponsor review of clinical criteria at screening (via the DVF)
Availability of a person (referred to as the “caregiver” throughout this protocol) who in the investigator's judgment:
   Has frequent and sufficient contact with the patient (i.e., at least 10 hours/week) to be able to provide accurate information regarding the patient's cognitive, behavioral, and functional abilities; agrees to provide information at clinic visits (for items which require caregiver input for scale completion); signs the necessary consent form; and has sufficient cognitive capacity to accurately report upon the patient's behavior and cognitive and functional abilities
   Is in sufficiently good general health to have a high likelihood of maintaining the same level of interaction with the patient and participation in study procedures throughout the study duration

Every effort was made to have the same caregiver participate throughout the duration of the study for completing the designated caregiver clinical outcome assessments (COAs).
Fluency in the language of the tests administered at the study site
Completion of at least 6 years of formal education after the age of 5 years
Willingness and ability to complete all required aspects of the study (including MRI, clinical genotyping, and, if applicable, PET imaging) (the patient should be capable of completing study procedures either alone or with the help of caregiver(s)).
Adequate visual and auditory acuity, in the investigator's judgment, to perform the neuropsychological testing (eyeglasses and hearing aids are permitted)
For women of childbearing potential: agreement to remain abstinent (refrain from heterosexual intercourse) or use contraceptive measures, and agreement to refrain from donating eggs.
For men: agreement to remain abstinent (refrain from heterosexual intercourse) or use a condom, and agreement to refrain from donating sperm.

### Exclusion Criteria

Patients who meet any of the following criteria were excluded from study entry:
Pregnant or breastfeeding, or intending to become pregnant during the study or within 9 weeks after the final dose of study drug or 4 days after the final dose of [¹⁸F]GTP1 radioligand or an amyloid radioligand, whichever is longer
Inability to tolerate MRI procedures or contraindication to MRI
Contraindication to PET imaging
For patients undergoing LP for confirmation of amyloid positivity: contraindication to lumbar dural puncture, including coagulopathy, concomitant anticoagulation (except for a platelet inhibitor such as aspirin or clopidogrel), thrombocytopenia (platelet count less than 50,000), prior lumbar spinal surgery, significant deformity of the lumbosacral region, or other factor that precludes safe LP in the opinion of the investigator
Body mass index greater than 40
Hospitalization during the 4 weeks prior to screening
Planned procedure or surgery during the study that in the investigator's opinion would affect cognitive assessments or otherwise interfere with compliance with the protocol
Residence in a skilled nursing facility (e.g., convalescent home or long-term care facility)
Blood transfusion within 8 weeks prior to screening/planned transfusion during the study
Poor peripheral venous access
Any serious medical condition or abnormality in clinical laboratory tests that remains abnormal on retest and, in the investigator's judgment, precludes the patient's safe participation in and completion of the study, or bias the assessment of the clinical or mental status of the participant to a significant degree. Including, but not limited to:
   Severe chronic kidney disease (Stage 4 or 5, according to National Kidney Foundation guidelines)
   Hypertension not stably controlled by current medication (e.g., sustained systolic blood pressure >160 mmHg or diastolic blood pressure >95 mmHg)
   Diabetes not stably controlled by current medication (e.g., hemoglobin Alc □ 8%, or any history of clinically significant hypoglycemia, hyperosmolar syndrome, ketoacidosis, or other significant complication of diabetes within 2 years before screening)
   Heart failure (e.g., New York Heart Association Class II or higher)
   Clinically significant, abnormal ECG at screening (e.g., evidence of significant conduction blockade, or evidence of prior myocardial infarction, unless associated with a known myocardial infarction more than 2 years before screening)
   History of cancer, except if considered to be cured; there is an appropriately treated carcinoma in situ of the cervix or Stage I uterine cancer; there has been no significant clinical progression during the past 5 years, with no active anti-cancer therapy or radiotherapy and, in the opinion of the investigator, is not likely to progress or require treatment in the ensuing 5 years; or the cancer is prostate cancer or basal cell carcinoma, where there has been no significant progression over the previous 2 years
QT interval corrected through use of Fridericia's formula (QTcF) >470 ms in females and >450 ms in males, demonstrated by at least two ECGs >30 minutes apart
Abnormal screening thyroid function tests or tests that remain abnormal on retest or require a new treatment or an adjustment of current treatment
Screening folic acid or vitamin B12 levels that are sufficiently low or remain low on retest such that deficiency requires initiation or alteration of treatment and/or may be contributing to cognitive impairment

### Cerebrovascular Neurologic Psychiatric Exclusion

Patients who meet any of the following cerebrovascular/neurologic/psychiatric criteria were excluded from study entry:
History of seizures (exception for childhood febrile seizures or other remote, non-recurrent seizure)
History of prior traumatic brain injury graded as mild-to-moderate or severe, defined as a head injury resulting in loss of consciousness lasting 30 minutes or longer, an initial Glasgow Coma Scale of 12 or worse at presentation, posttraumatic amnesia or confusion lasting 24 hours or longer, or any associated abnormal brain imaging finding at presentation
Any evidence of a condition other than AD that may affect cognition, including, but not limited to, dementia with Lewy bodies, vascular dementia, Parkinson's disease, corticobasal degeneration, Creutzfeldt Jakob disease, progressive supranuclear palsy, frontotemporal degeneration, Huntington disease, normal pressure hydrocephalus, hypoxia, severe sleep apnea or other chronic sleep disturbance, or baseline intellectual disability
History of schizophrenia, schizoaffective disorder, major depression, or bipolar disorder (a history of major depression is acceptable if patient has had no episode within the past year, is considered in remission, or depression is controlled by treatment)
At risk of suicide in the opinion of the investigator
Substance abuse, meeting criteria for alcohol, cannabis, phencyclidine, other hallucinogen, inhalant, opioid, sedative, hypnotic, anxiolytic, or stimulant use disorder of any severity (per the Diagnostic and Statistical Manual of Mental Disorders, Version 5) within the past 2 years
History or presence of clinically evident vascular disease potentially affecting the brain (e.g., clinically significant carotid, vertebral stenosis, or plaque; aortic aneurysm; intracranial aneurysm; cerebral or other intracranial hemorrhage; arteriovenous malformation) that, in the opinion of the investigator, has the potential to affect cognitive function
History or presence of any stroke with clinical symptoms within the past 2 years, or documented history within the last 6 months of an acute event consistent, in the opinion of the investigator, with a transient ischemic attack
History of cerebral amyloid angiopathy or MRI evidence of >6 microhemorrhages, any macrohemorrhage, or superficial siderosis comprising more than one region or a single region >1 cm
History or presence of intracranial tumor that is clinically relevant (e.g., glioma, cerebral metastasis) in the opinion of the investigator
Presence of infections that affect brain function or history of infections that resulted in neurologic sequelae (e.g., HIV, syphilis, neuroborreliosis, viral or bacterial meningitis/encephalitis)
History or presence of CNS or systemic autoimmune disorders potentially causing progressive neurologic disease with associated cognitive deficits (e.g., multiple sclerosis, lupus erythematosus, anti-phospholipid antibody syndrome, Behçet disease)
MRI evidence of more than two lacunar infarcts, any territorial infarct >1 cm³, or significant fluid attenuated inversion recovery hyperintense lesions in the cerebral deep white matter corresponding to a Fazekas deep white matter score of 3 or that otherwise may, in the investigator's opinion, contribute to cognitive dysfunction

### Infection and Immune Disorder Exclusion

Patients who meet any of the following infection and immune disorder criteria were excluded from study entry:
Systemically, clinically significantly immunocompromised patients, owing to continuing effects of immunosuppressive medication
Positive for hepatitis C virus antibody at screening
Positive for hepatitis B surface antigen at screening
Positive for HIV antibody at screening
Serious infection requiring oral or IV antibiotics within 30 days prior to screening
Known history of severe allergic, anaphylactic, or other hypersensitivity reactions to chimeric, human, or humanized antibodies or fusion proteins

### Screening

After signing the Informed Consent Form, patients entered a screening period of up to 8 weeks to determine eligibility. Extensions to this 8-week period (e.g., to complete an assessment of cerebral amyloidosis) were granted on a case by case basis by the Medical Monitor.

On the basis of any initial screening procedures, provided that the patient remains eligible, a DVF was completed. The DVF contained results from the MMSE and CDR, along with information supportive of an AD diagnosis. The DVF was reviewed and approved prior to performing MRI, [¹⁸F]GTP1 or amyloid PET scans, or LP.

During COVID-19-related disruptions, extensions to the screening period were granted on a case-by-case basis by the Medical Monitor for patients and clinical sites impacted by COVID-19 and/or any associated restrictions. For screening activities interrupted and later resumed, the Medical Monitor advised, on a case-by-case basis, whether specific screening procedures need to be repeated.

### Study Periods

### Double-Blind Treatment Period

The double-blind treatment period of the study included a baseline period and a double-blind treatment period. The baseline period lasted up to 15 days; however, extensions to the 15-day baseline period, for instance, to complete [¹⁸F]GTP1 PET imaging, were granted on a case-by-case basis. If the baseline period was extended to 2 months or longer because of COVID 19-related disruptions and baseline activities were interrupted and later resumed, the Medical Monitor advised, on a case by-case basis, whether specific baseline procedures needed to be repeated prior to Week 1.

The double-blind treatment periods were Weeks 1-49 for Cohort 1, Weeks 1-61 for Cohort 2, and Weeks 1-73 for Cohort 3. Treatment with study drug was Q2W for the first three doses (i.e., doses at Weeks 1, 3, and 5) and Q4W thereafter, up to and including the last double-blind dose (up to a total of 13 doses for Cohort 1, 16 doses for Cohort 2, and 19 doses for Cohort 3). There was no administration of study drug at the last visit of the double-blind treatment period. For Cohort 2 and Cohort 3, if two or more consecutive study drug infusions were missed during the double-blind treatment period, study drug administration was re-initiated with Q2W dosing for the next three doses, followed by Q4W dosing thereafter. Safety, efficacy, PK, and biomarker assessments were performed prior to the first administration of study drug and at several postbaseline visits, including the last visit of the double-blind treatment period. Patients who missed study drug infusions during the double-blind treatment period for reasons related to COVID-19-related disruptions may have additional COAs administered remotely (by telephone or video call) at the time of the first missed infusion and at the time when in-clinic infusions resumed.

### Open Label Extension Period

An optional 96-week OLE period was available to subjects who completed the double-blind treatment period and who, in the judgment of the investigator, would potentially benefit from open-label Semorinemab treatment. For the OLE period, all subjects received Semorinemab 4500 mg IV beginning at Week 53 (Cohort 1), Week 65 (Cohort 2), or Week 77 (Cohort 3).

199 subjects were randomized into a placebo (N=95) or Semorinemab (N=104) treatment group (non-blinded). 149 subjects (Placebo, N=72 and Semorinemab, N=77) are still continuing in the OLE study. 50 subjects from both groups did not continue based on the reasons in Table 5.

TABLE 5

| | Placebo | | Semorinemab | | |
|---|---|---|---|---|---|
| | (N (%)) | | (N (%)) | | Total N |
| Adverse event | 4 | (4.2%) | 3 | (2.9%) | 7 |
| Death | 2 | (2.1%) | 0 | | 2 |
| Lost to follow-up | 0 | | 4 | (3.8%) | 4 |
| Withdrawal by subject | 16 | (16.8%) | 19 | (18.3%) | 35 |
| Other various reasons | 1 | (1.1%) | 1 | (1.0%) | 2 |

### Safety Follow Up Period

All patients were followed for safety after their final dose of study drug. Patients not entering the OLE period had a safety follow up visit 12 weeks after the final dose of study drug (i.e., safety follow up at Week 57 for Cohort 1, Week 69 for Cohort 2, or Week 81 for Cohort 3). Patients entering the OLE period had a safety follow up visit 12 weeks after the final dose of open label treatment (i.e., safety follow up at Week 157 for Cohort 1, Week 169 for Cohort 2, or Week 181 for Cohort 3). Patients who discontinued from treatment early, in either the double-blind treatment period or in the OLE period, had a treatment discontinuation visit 12 weeks following their final treatment dose.

### Assessments

### Clinical Outcome Assessment (COA)

COAs provided an understanding of the effect a treatment has on a patient. A variety of observer (caregiver) and clinician reported outcomes were collected to characterize the efficacy and clinical profile of study treatment. The primary outcome measures, the ADAS Cog11 and the ADCS-ADL, are validated instruments that have been widely used in assessing AD. Additional observer (caregiver) and clinician reported outcomes were used to evaluate patient cognition, function, and behavior. Site staff were provided with a standardized rater training program to certify them to administer the COAs identified in this protocol.

To minimize the contribution of potential confounding factors to these cognitive assessments, any patient with a current untreated depressive episode (i.e., presence of clinically significant depressive symptoms) were excluded from the study.

The COAs listed in Table 6 were administered to all patients and/or caregivers enrolled in this study. The C-SSRS was used to monitor safety; all other COAs were used as assessments of treatment efficacy.

TABLE 6

| Clinical Outcome Assessments | Patient | Caregiver | Concept |
|---|---|---|---|
| Mini-Mental State Examination (MMSE) | x | | Cognition |
| Alzheimer's Disease Assessment Scale-Cognitive | x | | Cognition |
| Subscale, 11-item version (ADAS-Cog11) | | | |
| Clinical Dementia Rating (CDR) Scale | x | x | Cognition and |
| | | | Function |
| Alzheimer's Disease Cooperative Study-Activities of | | x | Function |
| Daily Living Inventory (ADCS-ADL) | | | |
| Neuropsychiatric Inventory (NPI) | | x | Behavioral |
| | | | Symptoms |
| Caregiver Global Impression Scales for Alzheimer's | | x | Cognition and |
| disease (CaGI-Alz) | | | Function |
| Columbia-Suicide Severity Rating Scale (C-SSRS) | x | | Suicidality |

### Assessment of Severity of Adverse Events

The WHO toxicity grading scale was used for assessing adverse event severity. Table 7 was used for assessing severity for adverse events that are not specifically listed in the WHO toxicity grading scale.

TABLE 7

| Grade | Severity |
|---|---|
| 1 | Mild; transient or mild discomfort (<48 hours); no medical intervention |
| | or therapy require |
| 2 | Moderate; mild to moderate limitation in activity; some assistance may |
| | be needed; no or minimal medical intervention or therapy required |
| 3 | Severe; marked limitation in activity; some assistance usually required; |
| | medical intervention or therapy required; hospitalization possible |
| 4 | Life-threatening; extreme limitation in activity; significant assistance |
| | required; significant medical intervention or therapy required, |
| | hospitalization or hospice care probable |

### Infusion Related Reactions or Hypersensitivity

Monoclonal antibodies such as semorinemab may be associated with a potential immune response in clinical trials, such as hypersensitivity or hypersensitivity like reactions, including severe, anaphylactic reactions. All participants were monitored for infusion related reactions, hypersensitivity, or hypersensitivity like reactions during the infusion and immediately afterward.

### Neuroimaging Abnormalities

The following were performed to monitor for potential neuroimaging abnormalities:
All patients had an MRI at screening and at several post-baseline time-points. MRI was performed prior to any dosing on the corresponding study visit and was read locally in real time for the evaluation of any new, clinically significant abnormality prior to the dose being given. However, if no such abnormalities were identified in the local MRI interpretation, the dose may be given prior to the receipt of the central MRI report. In case of an abnormality identified by the central MRI reading, the investigator assessed clinical significance of it and proceed as follows:
During COVID-19-related disruptions, if study-associated MRIs could not be obtained, the patient was monitored for any signs or symptoms suggestive of new, clinically significant neurologic abnormalities. If no new, clinically significant abnormalities were observed, study drug could be administered without an MRI, but a subsequent unscheduled study associated MRI was obtained as local conditions allowed. If new, clinically significant neurologic abnormalities were observed, study drug was withheld and the patient referred for appropriate follow-up and medical treatment, including, if appropriate, an MRI. Preliminary analyses of blinded safety data from Studies GN39763 and GN40040 suggested that rates of MRI abnormalities in these studies were consistent with background rates reported in this patient population (Carlson et al. Alzheimers Dement 2011; 7:396-401; Caunca et al. J Am Heart Assoc [serial on the Internet]. September 2016 [cited 14 Apr. 2020]. Available from:
   https://www.ncbi.nlm.nih.gov/pmc/articles/PMC5079015/).
All patients regularly underwent neurologic examinations to evaluate for any neurologic signs or symptoms. In case of findings suggestive of a new, clinically significant CNS disturbance or lesion, patients underwent an MRI examination as soon as possible.
All MRIs were read locally in real time for the evaluation of any clinically significant new or worsening abnormality. Study drug was withheld at the corresponding study visit if, in the investigator's judgment, any clinically significant new or worsening MRI abnormality (e.g., symptomatic or asymptomatic intracranial tumor, cerebral infarct [lacunar or territorial], cerebral hemorrhage [macrohemorrhage, microhemorrhage, superficial siderosis], vasogenic edema, sulcal effusion) was observed. Restarting study drug treatment could occur only after discussion with the Medical Monitor.
Regardless of severity, all events of a clinically significant new or worsening MRI abnormality that occurred at any time after receiving study drug were considered to be adverse events of special interest to be reported in an expedited manner.

### Immunogenicity

Semorinemab is a pan Tau IgG4 monoclonal antibody engineered to contain Fc mutations (YTE) that enhance binding to FcRn and has been shown to slow peripheral antibody clearance in humans. ADAs to semorinemab in humans may be associated with changes in semorinemab exposure, reductions in treatment efficacy, or safety findings such as hypersensitivity reactions. In the Phase I study (GN39058), there was no evidence of treatment emergent ADAs. Immunogenicity in this Phase II study (GN40040) were evaluated using validated immunoassays and by assessing the incidence of ADAs after treatment relative to their prevalence at baseline.

### Columbia Suicide Severity Rating Scale

The C-SSRS is an interview-based instrument used to assess baseline incidence of suicidal ideation and behavior and to prospectively assess suicidal ideation and behavior at post-baseline visits. Post-baseline assessments assessed suicidal ideation and behavior since the previous visit. The C-SSRS was used to monitor safety. It was administered to the patient and measured five subtypes of suicidal ideation and behavior thought by the FDA to be important to capture in a prospective assessment of suicidality (FDA 2012). If any C-SSRS responses were suggestive of an adverse event, the investigator determined whether the criteria for an adverse event had been met and, if so, reported the event on the Adverse Event eCRF.

### Biomarker Assessments

Blood and CSF biomarker assessments were used to verify amyloid positivity, evaluate the relationship between [¹⁸F]GTP1 PET and CSF Tau measures, demonstrate evidence of the biologic activity of semorinemab in patients, identify biomarkers that may be predictive of response to semorinemab, define PK and PD relationships, advance the understanding of the mechanism of action of semorinemab in patients, and increase the knowledge and understanding of disease biology.

### End of Study

The end of this study was defined as the date when the last patient, last visit occurs or the date at which safety follow up is received from the last patient, whichever occurs later. The end of the study was expected to occur up to 45 months after the last patient is enrolled.

### Length of Study

The entire study, from screening of the first patient to the end of the study, was expected to last up to 60 months. If the decision was made to expand the study size and enroll up to an additional 100 patients, the entire study would be expected to last up to 72 months. Statistical Features

### Determination of Sample Size

This study enrolled about 272 subjects from different countries worldwide (Table 8) randomized to either an IV active dose arm or to an IV placebo dose arm in a 1:1 ratio. This sample size provided approximately 80% power to detect a 35% relative reduction in mean ADAS Cog11 change from baseline when semorinemab is compared with placebo assuming an average decline of 6 points for placebo-treated patients, a standard deviation across patients of 7.5, a 10% dropout rate, and a two sided α=0.2 significance level. During the second half of 2020, blinded data was reviewed to inform a decision on potential expansion of the study size. Study expansion, with enrollment of up to 100 additional patients, would be considered if conditions associated with COVID-19 disruptions resulted in study drug treatment interruptions to the degree that they may significantly compromise study integrity and/or the size of the evaluable patient population despite the extension to the double-blind period implemented in the protocol, and if preliminary data continued to suggest that semorinemab exhibits an acceptable and tolerable safety profile in this patient population. The number of additional patients would approximate the number of currently randomized patients that had missed study drug infusions due to COVID-19-related disruptions.

TABLE 8

| Country | Number of Subjects Enrolled |
|---|---|
| France | 22 |
| Poland | 43 |
| Spain | 36 |
| United States | 171 |
| Total worldwide | 272 |

### Statistical Considerations and Analysis Plan

The efficacy analyses were based on the modified intent to treat population, which is defined as all randomized patients who received at least one dose of study drug and had at least one post-baseline ADAS-Cog11 and/or ADCS-ADL measurement. For the efficacy analysis, patients were grouped according to the treatment assigned at randomization.

The safety analysis was based on all randomized patients who received at least one dose of either semorinemab or placebo. Patients were grouped according to semorinemab treatment actually received.
A primary analysis occurred after the last patient completed the assessments for the last visit of the double-blind treatment period (Week 49 for Cohort 1, Week 61 for Cohort 2, or Week 73 for Cohort 3).
A final analysis occurred after the last patient has completed the OLE and completed the assessments for the last visit of the OLE period (Week 145 for Cohort 1, Week 157 for Cohort 2, or Week 169 for Cohort 3).

### Summaries of Conduct of Study

The number of patients who enroll, discontinue (early discontinuation of treatment or early termination from the study), complete the double-blind treatment period (through Week 49 for Cohort 1, Week 61 for Cohort 2, and Week 73 for Cohort 3), and continue into the OLE was tabulated by treatment group. Reasons for early discontinuation of treatment or early termination from the study was listed and summarized by treatment group. Any eligibility criteria exceptions and other major protocol deviations also was summarized by treatment group.

### Summaries of Treatment Group Comparability

Demographic and baseline characteristics such as age, sex, race, Apoε4 status, and baseline MMSE score were summarized with means, standard deviations, medians, and ranges for continuous variables and with frequencies and proportions for categorical variables, as appropriate. Summaries are presented by treatment arm and overall.

### Efficacy Analyses

### Primary Efficacy Endpoints

The co-primary efficacy endpoints are change in ADAS-Cog11 and ADCS-ADL scores from baseline to the last visit of the double-blind treatment period (Week 49 for Cohort 1, Week 61 for Cohort 2, and Week 73 for Cohort 3). The difference in mean change from baseline to the last visit of the double-blind treatment period between semorinemab- and placebo-treated patients was estimated using an analysis of covariance model adjusting for Apoε4 status (Apoε4+ vs. Apoε4−), screening MMSE (16-18 vs. 19-21), baseline [¹⁸F]GTP1 PET standard uptake value ratio, age, baseline ADAS Cog11 or ADCS-ADL scores, and if appropriate, the number of missed study drug infusions during the double-blind treatment period due to COVID-19-related disruptions. Confidence intervals, as well as least squares estimates, were used to aid in interpretation of study results.

### Secondary Efficacy Endpoints

The secondary endpoints were change in CDR SB and MMSE scores from baseline to the last visit of the double-blind treatment period (Week 49 for Cohort 1, Week 61 for Cohort 2, and Week 73 for Cohort 3). Secondary efficacy endpoints were analyzed in the same manner as the primary endpoint.

### Safety Analyses

The safety analysis population included all randomized patients who received at least one dose of either semorinemab or placebo, or GTP1, with patients grouped according to treatment arm. Patients were analyzed according to actual semorinemab treatment received.

All adverse events that occurred after informed consent were summarized by mapped term, appropriate thesaurus level, and toxicity grade. In addition, all serious adverse events, including deaths and events leading to discontinuation, were listed separately and summarized.

Laboratory data were summarized by descriptive statistics by treatment group. In addition, all laboratory abnormalities were summarized by grade using the WHO grading scale.

Adverse events of special interest and neuroimaging abnormalities were listed and summarized by treatment group.

Vital signs (pulse rate, blood pressure, body temperature, and respiratory rate), weight, and other disease-specific data were summarized by descriptive statistics by treatment group. Changes from baseline were summarized by treatment group.

### Pharmacokinetic Analyses

Individual and mean serum semorinemab concentration-time data were tabulated and plotted by treatment group and/or cohort, as appropriate, and Cₘₐₓ and minimal concentration were reported. Estimates for PK parameters were tabulated and summarized by descriptive statistics (e.g., mean, standard deviation, minimum, and maximum) for patients completing the study without missed doses, as appropriate. Individual and mean semorinemab CSF concentration-time data were tabulated by treatment group. Additional PK analyses were conducted as appropriate.

### Immunogenicity Analyses

Baseline prevalence and postbaseline incidence of ADA to semorinemab was summarized. ADA response and potential effect of ADA response to relevant clinical safety and activity endpoints was assessed for evaluable patients.

The immunogenicity analyses included patients with at least one pre-dose and one post-dose ADA assessment, with patients grouped according to treatment arm.

The number and proportion of ADA positive patients and ADA negative patients during both the treatment and follow up period was summarized by treatment group. Patients were considered to have treatment emergent ADAs if they were ADA negative at baseline but developed an ADA response following study drug administration, or if they were ADA positive at baseline and the titer of one or more postbaseline samples is at least 4-fold greater (i.e., equal to greater than 0.60 titer unit) than the titer of the baseline sample. Patients were considered to be ADA negative if they are ADA negative at baseline and all post-baseline samples were negative or treatment unaffected if they were ADA positive at baseline but do not have any post-baseline samples with a titer that is at least 4-fold greater than the titer of the baseline sample.

The relationship between ADA status and safety, efficacy, PK, and biomarker endpoints was analyzed and reported via descriptive statistics.

### Biomarker Analyses

Exploratory analyses were conducted to evaluate the effect of semorinemab on exploratory biomarkers. Exploratory biomarkers may be analyzed before and after dosing with semorinemab to determine the relationship between PK exposure and exploratory biomarker levels. In addition, relationships amongst biomarkers may be assessed.

WGS and SNP data were analyzed in the context of this study and explored in aggregate with data from other studies to increase researchers' understanding of disease pathobiology and guide the development of new therapeutic approaches.

### Results:

### Overview

Briefly, a total of 272 participants were randomized 1:1 in the semorinemab and placebo treatment arms. The modified intent-to-treat population (mITT; n=238) included those who received ≥1 dose(s) of study medication and underwent baseline and at least 1 post-baseline assessment(s). Within the mITT population, the overall distributions of age (mean=72.0, SD=8.2), gender (64.3% female), education (80.5% high school graduate or higher), and APOε status (63.1% ε4+) did not differ between treatment arms. At Week 49, the semorinemab arm demonstrated a 42.2% reduction (−2.89 points, SE=0.85, p=0.0008) in decline on ADAS-Cog11 relative to the placebo arm. Similar rates of clinical decline on ADCS-ADL, MMSE, and CDR-SB and similar rates of tau accumulation per whole cortical grey [¹⁸F]GTP1 tau PET SUVR were seen between treatment arms. Findings remained consistent when other analysis populations were examined, including the pre-specified analysis population (mITT*: mITT participants who missed 1 blinded study drug doses), in which the semorinemab arm demonstrated a 43.6% reduction (−2.96 points, SE=0.97, p=0.0025) in decline on ADAS-Cog11 relative to the placebo arm. Preliminary analyses of safety data indicated that the rates of adverse events, serious adverse events, deaths, and withdrawals due to adverse events were reasonably balanced between treatment arms. Serum and CSF pharmacokinetics of semorinemab were consistent with prior studies. Similar serum semorinemab exposure was observed between the mITT and mITT* populations.

### Populations Analyzed

All subjects (N=272); safety evaluable (N=267) (subjects that enrolled and received at least one dose of semorinemab (N=135) or placebo (N=132)); and completed (N=200; semorinemab (N)=104 and placebo (N)=96). The ethnicity of the safety evaluable subjects is provided in Table 9.

TABLE 9
Demographics of Safety Evaluable Group

| | Placebo | Semorinemab |
|---|---|---|
| Overall number of baseline subjects | 132 | 135 |
| Age, N (SD) | 73.1 (8.0) | 71.6 (8.2) |
| Female (N) | 80 | 93 |
| Male (N) | 52 | 42 |
| Ethnicity | | |
| Hispanic or Latino | 3 | 4 |
| Not Hispanic or Latino | 111 | 116 |
| Unknown or Not Reported | 18 | 15 |
| Race | | |
| Asian | 0 | 1 |
| Black or African American | 5 | 4 |
| White | 115 | 121 |
| Unknown or not reported | 12 | 9 |

A total of 272 subjects were randomized. Study discontinuations were well-balanced between the placebo and semorinemab arms, as provided in Table 10 below (N=267).

TABLE 10

| | |
|---|---|
| Placebo | Semorinemab |
| (N (%)) | (N (%)) |

| Discontinued during | 36 | (27.3%) | 31 | (22.9%) |
|---|---|---|---|---|
| double-blind period | | | | |
| Adverse event | 6 | (4.5%) | 6 | (4.4%) |
| Death | 2 | (1.5%) | 1 | (0.74%) |
| Lost to follow-up | 1 | (0.76%) | 0 | |
| Withdrawal by subject | 21 | (15.9%) | 19 | (14.1%) |
| Physician Decision | 3 | (2.3%) | 0 | |
| Other | 3 | (2.3%) | 5 | (3.7%) |

The well-balanced baseline characteristics of the present clinical trial's 272 patients and subgroup analyses support the trial's top line results (meeting one of its two co-primary endpoints by demonstrating a statistically significant 43.6% improvement in the rate of cognitive decline with semorinemab compared to placebo (p=0.0025), as measured ADAS-Cog11 at week 49 in a prespecified modified intent to treat (mITT) population).

### Analysis Populations:

mITT: 238 participants who received ≥1 dose(s) of study drug and underwent baseline and at least one post-baseline ADAS-Cog11 assessment
Pre-specified analysis population, mITT*: 204 participants in the mITT population who missed ≤1 blinded study drug doses

Serum PK showed similar exposure levels in mITT and mITT* populations and topline results were consistent between mITT and mITT* populations.

Baseline demographics and disease characteristics of the mITT population are summarized in Table 11 below. As shown in Table 11, baseline demographics and disease characteristics were well balanced between the two treatment arms (mITT, N=238)

TABLE 11

| | Placebo | | Semorinemab | | All Subjects | |
|---|---|---|---|---|---|---|
| | (N = 115) | | (N = 123) | | (N = 238) | |
| Adults (18-64 years) | 17 | | 29 | | 46 | |
| Elderly (65-84 years) | 92 | | 89 | | 181 | |
| Elderly (85 years and over) | 6 | | 5 | | 11 | |
| Age, mean (SD) | 72.8 | (8.2) | 71.2 | (8.2) | n/a | |
| Female, N (%) | 70 | | 84 | | 154 | |
| Male, N (%) | 45 | | 39 | | 84 | |
| Ethnicity | | | | | | |
| Hispanic or Latino | 2 | | 4 | | 6 | |
| Not Hispanic or Latino | 97 | | 104 | | 201 | |
| Unknown or Not Reported | 16 | | 15 | | 31 | |
| Race | | | | | | |
| Asian | 0 | | 1 | | 1 | |
| Black or African American | 4 | | 4 | | 8 | |
| White | 101 | | 109 | | 210 | |
| Unknown or not reported | 10 | | 9 | | 19 | |
| APOE4 positive (+) | 70 | | 80 | | 150 | |
| APOE4 negative (−) | 45 | | 43 | | 88 | |
| MMSE 16-18 | 55 | | 61 | | 116 | |
| MMSE 19-21 | 60 | | 62 | | 122 | |
| CDR-GS - 1 | 102 | | 113 | | 215 | |
| CDR-GS - 2 | 13 | | 10 | | 23 | |
| Symptomatic AD therapy, N | 85 | | 100 | | 185 | |
| (%) | | | | | | |
| ADAS-Cog11, mean (SD) | 24.09 | (0.589) | 23.93 | (0.533) | 24.01 | (6.10) |
| ADCS-ADL, mean (SD) | 59.74 | (0.842) | 62.03 | (0.764) | 60.92 | (8.80) |
| CDR-SB, mean (SD) | 6.51 | (0.182) | 6.23 | (0.156) | 6.36 | (1.84) |
| MMSE, mean (SD) | 18.15 | (0.197) | 18.38 | (0.182) | 18.27 | (2.06) |
| GTP1 WCG, mean (SD) | 1.42 | (0.305) | 1.43 | (0.301) | 1.42 | (0.302) |

Symptomatic AD therapy includes: donepezil, galantamine, rivastigmine, memantine, Souvenaid

### Key Efficacy Populations:

1) MITT max 1 missed dose (also referred to herein as “mITT*”) (N=204 for ADAS-Cog11 at baseline) (visit level selection, where the maximum number of missed dose is either 1, or 2 with the supplemental dose up to the visit in question; patients who remain above target exposure (90%), limited sample size at week 61). “mITT*” was a pre-specified population, due to COVID-19 disruptions.
2) MITT max 1 missed dose (or mITT*), week 49 completers (N=165 for ADAS-Cog11 at baseline) (visit level selection, where the maximum number of missed dose is either 1, or 2 with the supplemental dose AND the week 49 ADAS-Cog test was completed; most reliable treatment trend estimate up to week 49, but limited sample size at week 61).
3) MITT (N=238 for ADAS-Cog11 at baseline) (all patients from both cohort 1 and 2, regardless of their missed dose status; largest sample size; recommended if results are consistent with the above 2 analysis).
4) Cohort 1 (N=148 for ADAS-Cog11 at baseline) (MITT patients who followed the protocol schedule assigned to Cohort 1 (i.e., 49-week blinded period)).
5) Cohort 2 (N=93 for ADAS-Cog11 at baseline) (MITT patients who followed the protocol schedule assigned to Cohort 2 (i.e., 61-week blinded period)).

### Power and Testing

This clinical trial was designed for 80% power at α=0.2 to detect a 35% relative reduction (RR) in decline on ADAS-Cog11, for 260 subjects enrolled with 10% expected dropout rate by Week 49.

Expected mean placebo change from baseline of 6 points with SD=7.5 by Week 49.

Signal-to-noise for ADCS-ADL was assumed to be similar to ADAS-Cog11 based on limited available datasets containing both endpoints; a separate power calculation was not performed.

At the time of this analysis, there was 83% power at the α=0.2 level to detect a 35% RR in ADAS-Cog11.

159 subjects (“MITT Max 1 missed dose” or “mITT*”) had a Week 49 ADAS-Cog11 assessment.

Mean (unadjusted) placebo change from baseline is in line with prior expectation, 6.29 points with SD=6.21.

The power for ADCS-ADL with the same parameters is lower, 63% (change from baseline 6.95, SD=9.78).

### Analysis Methods Used:

Data used for primary and secondary analyses includes COAs collected both before and after onset of COVID-19 disruptions; some CDR-SB and ADCS-ADL assessments (but not ADAS-Cog11) were collected remotely after onset of COVID-19 disruptions.

Baseline was defined as the average of screening and baseline observations (all cognitive and functional endpoints). ADAS-Cog11 and ADCS-ADL were also collected at Week 61 for patients in Cohort 2 and are included in the primary analysis. Due to the limited sample size, the Week 61 results were not interpreted on their own when looking the max 1 missed dose analysis population.

Mixed effects repeated measures (MMRM) model on endpoint change from baseline: model was adjusted for baseline age and stratification factors, and AD concomitant medication; the model treated a visit as categorical (no assumption on shape of longitudinal trajectories) and used the missing-at-random (MAR) assumption to handle missing data.

### Executive Summary:

In this clinical study, participants with mild-to-moderate AD who were treated with semorinemab demonstrated a statistically significant and clinically meaningful reduction in decline on co-primary cognitive endpoint (ADAS-Cog11). Safety data was in line with previous data: semorinemab was well tolerated, with an acceptable safety profile, and no unanticipated safety signals.

### Efficacy Summary:

ADAS-Cog11: This clinical trial showed a statistically significant relative reduction (43.6%; p=0.0025) of cognitive decline at Week 49 in the semorinemab arm versus placebo. Semorinemab treatment benefit was also observed at Week 25 and Week 37. Overall, findings were consistent between analysis populations.

ADCS-ADL: There was no evidence of treatment benefit of semorinemab in ADL. The observed treatment effect was expressed as a relative reduction ranges between [−2% to −29%] at week 25, 37 and 49 in the different analysis populations.

Moderate correlations in results are observed between the co-primary endpoints (ADAS-Cog11 and ADCS-ADL) on the subject level. Results are summarized in Tables 12A and 12B, below.

TABLE 12A

| | Placebo | | | | | | | |
|---|---|---|---|---|---|---|---|---|
| | | Least | | | | | | |
| | | Squares (LS) | | | Semorinemab | | | |
| | | Mean | | 95% CI for LS | | | | 95% CI for LS |
| | | Change from | | Mean Change | | Least Squares | | Mean Change |
| | (N) | Baseline (SE) | | from Baseline | (N) | Mean (SE) | | from Baseline |
| ADAS-Cog11 | | | | | | | | |
| Baseline | 115 | 24.09 | (0.589) | | 123 | 23.93 | (0.533) | |
| Week 25 | 107 | 3.57 | (0.576) | (2.43, 4.70) | 112 | 2.36 | (0.600) | (1.18, 3.54) |
| Week 37 | 102 | 5.15 | (0.608) | (3.95, 6.34) | 103 | 3.96 | (0.634) | (2.71, 5.20) |
| Week 49 | 96 | 6.85 | (0.643) | (5.58, 8.11) | 102 | 3.96 | (0.658) | (2.66, 5.26) |
| Week 61 | 30 | 8.47 | (0.965) | (6.55, 10.38) | 38 | 5.71 | (0.907) | (3.92, 7.51) |
| ADCS-ADL | | | | | | | | |
| Baseline | 115 | 59.74 | (0.842) | | 123 | 62.03 | (0.764) | |
| Week 25 | 114 | −3.67 | (0.823) | (−5.29, −2.05) | 122 | −4.82 | (0.862) | (−6.51, −3.12) |
| Week 37 | 109 | −5.35 | (0.913) | (−7.15, −3.55) | 113 | −6.47 | (0.947) | (−8.33, −4.60) |
| Week 49 | 101 | −6.80 | (0.974) | (−8.72, −4.88) | 107 | −7.63 | (1.002) | (−9.61, −5.66) |
| Week 61 | 33 | −7.57 | (1.462) | (−10.47, −4.68) | 40 | −9.29 | (1.343) | (−11.95, −6.63) |
| CDR-SB | | | | | | | | |
| Baseline | 115 | 6.51 | (0.182) | | 123 | 6.23 | (0.156) | |
| Week 25 | 114 | 1.11 | (0.182) | (0.75, 1.47) | 122 | 1.00 | (0.189) | (0.63, 1.38) |
| Week 49 | 101 | 1.54 | (0.214) | (1.12, 1.96) | 109 | 1.80 | (0.217) | (1.37, 2.23) |
| Week 61 | 33 | 2.28 | (0.393) | (1.50, 3.06) | 40 | 2.45 | (0.367) | (1.37, 2.23) |
| MMSE | | | | | | | | |
| Baseline | 115 | 18.15 | (0.197) | | 123 | 18.38 | (0.182) | |
| Week 25 | 106 | −2.30 | (0.287) | (−2.87, −1.74) | 116 | −2.20 | (0.294) | (−2.78, −1.62) |
| Week 49 | 97 | −3.12 | (0.325) | (−3.76, −2.48) | 105 | −2.86 | (0.330) | (−3.51, −2.21) |
| Week 61 | 29 | −4.22 | (0.466) | (−5.14, −3.30) | 39 | −3.14 | (0.429) | (−4.00, −2.29) |

TABLE 12B

| | | | 95% CI for | | |
|---|---|---|---|---|---|
| | Difference | | Difference in | | |
| | in LS Mean | | LS Mean | % Relative | |
| | Change (SE) | | Change | Reduction | P-value |
| ADAS-Cog11 | | | | | |
| Week 25 | −1.21 | (0.754) | (−2.69, 0.28) | 33.8% | 0.1111 |
| Week 37 | −1.19 | (0.804) | (−2.77, 0.39) | 23.1% | 0.1401 |
| Week 49 | −2.89 | (0.849) | (−4.56, −1.21) | 42.2% | 0.0008 |
| Week 61 | −2.75 | (1.287) | (−5.31, −0.20) | 32.5% | 0.0351 |
| ADCS-ADL | | | | | |
| Week 25 | −1.14 | (1.072) | (−3.26, 0.97) | 31.1% | 0.2872 |
| Week 37 | −1.12 | (1.207) | (−3.50, 1.26) | 20.9% | 0.3541 |
| Week 49 | −0.83 | (1.295) | (−3.39, 1.72) | 12.3% | 0.5207 |
| Week 61 | −1.72 | (1.911) | (−5.50, 2.07) | 22.7% | 0.3704 |
| CDR-SB | | | | | |
| Week 25 | −0.11 | (0.237) | (−0.57, 0.36) | 9.6% | 0.6544 |
| Week 49 | 0.26 | (0.282) | (−0.29, 0.82) | −17.2% | 0.3501 |
| Week 61 | 0.17 | (0.525) | (−0.87, 1.22) | −7.6% | 0.7431 |
| MMSE | | | | | |
| Week 25 | 0.10 | (0.372) | (−0.63, 0.83) | −4.3% | 0.7889 |
| Week 49 | 0.27 | (0.429) | (−0.58, 1.11) | −8.5% | 0.5366 |
| Week 61 | 1.08 | (0.618) | (−0.15, 2.30) | −25.5% | 0.0851 |

COVID-19 impact: 45% missed 1 or more doses, the distribution of missed doses was balanced between the arms.

There were no major imbalances in baseline characteristics or discontinuations.

There was similar semorinemab exposure in MITT and “Max. 1 missed dose” populations (based on serum and CSF PK).

There was a directional trend towards slower ADAS-Cog11 progression at higher semorinemab exposure (based on serum and CSF PK).

### Safety Summary:

Overall, there were balanced AEs, SAEs, Deaths, and Withdrawals due to AEs between the arms (with some small numerical differences favoring one or the other treatment arm). AE and SAE rates were as expected for study population; generally balanced across arms. Grade 3 AEs were slightly higher in the semo arm, due to reports of fall and subarachnoid hemorrhage; however, reported event numbers remained small.

Missed doses (due to COVID-19 restrictions or other factors) did not impact the overall interpretation of treatment effects for the efficacy endpoints. There were no major imbalances in baseline characteristics, discontinuations that could have implications for the interpretation of the treatment effects for the efficacy endpoints. Planned sensitivity analyses suggested that the missed doses did not significantly impact topline analyses of the data from the blinded portion of the study.

Infusion Related Reactions, Nasopharyngitis plus Upper Respiratory Tract Infection, Arthralgia, Insomnia, Back Pain and Confusional State AEs were numerically slightly higher in the semo arm; with the exception of 1 event, all of these are non-serious AEs.

Neuroimaging abnormalities were slightly higher in placebo, within expected reporting rates.

There were no imbalances in the NPI score and suicidal ideation/behavior.

There were no treatment-emergent ADAs observed with semorinemab.

### Results by Treatment Groups

### ADAS-Cog11 Treatment Benefit (MITT, Max 1 Missed Dose)

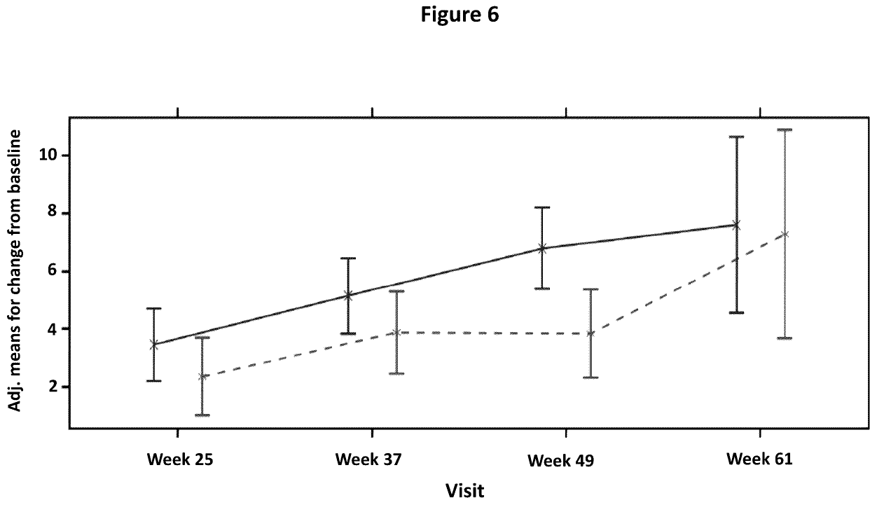

Table 13, below, shows Mixed-effect Model Repeated Measures (MMRM) analysis of change from baseline in ADAS-Cog11, for the Double Blind Period, maximum 1-dose-missed flag, for Modified Intent to Treat patients. See also FIG. 6.

As seen, a statistically significant treatment effect (RR=43.6% p-value=0.003) was observed at week 49 (an increase in ADAS-Cog11 score corresponds to disease worsening). Although statistically not significant, the point estimates favor the semorinemab arm at Week 25 (RR=31.9%) and 37 (RR=24.7%).

TABLE 13
Mixed-effect Model Repeated Measure (MMRM) Analysis of
Change from Baseline, ADAS-Cog11, Double-Blind Period,
Max one missed flag, Modified Intent to Treat Patients

| Visit | Placebo | | Semorinemab 4500 mg IV | |
|---|---|---|---|---|
| Statistics | (N = 115) | | (N = 123) | |
| Baseline | | | | |
| n | 101 | | 103 | |
| Mean (SE) | 24.05 | (0.631) | 24.05 | (0.604) |
| Week 25 | | | | |
| n | 97 | | 100 | |
| Adjusted Mean (SE) | 3.45 | (0.633) | 2.35 | (0.675) |
| 95% CI for Adjusted Mean | (2.20, 4.69) | | (1.01, 3.68) | |
| Difference in Adjusted Means (SE) | | | −1.10 | (0.829) |
| 95% CI for Difference in Adjusted | | | (−2.74, 0.53) | |
| Means | | | 31.9% | |
| Relative Reduction in Adjusted | | | 0.1859 | |
| Mean | | | | |
| p-value | | | | |
| Week 37 | | | | |
| n | 88 | | 84 | |
| Adjusted Mean (SE) | 5.14 | (0.666) | 3.86 | (0.719) |
| 95% CI for Adjusted Mean | (3.82, 6.45) | | (2.44, 5.28) | |
| Difference in Adjusted Means (SE) | | | −1.28 | (0.887) |
| 95% CI for Difference in Adjusted | | | (−3.03, 0.47) | |
| Means | | | 24.8% | |
| Relative Reduction in Adjusted | | | 0.1520 | |
| Mean | | | | |
| p-value | | | | |
| Week 49 | | | | |
| n | 82 | | 77 | |
| Adjusted Mean (SE) | 6.79 | (0.717) | 3.83 | (0.769) |
| 95% CI for Adjusted Mean | (5.38, 8.20) | | (2.32, 5.35) | |
| Difference in Adjusted Means (SE) | | | −2.96 | (0.965) |
| 95% CI for Difference in Adjusted | | | (−4.86, −1.05) | |
| Means | | | 43.6% | |
| Relative Reduction in Adjusted | | | 0.0025 | |
| Mean | | | | |
| p-value | | | | |
| Week 61 | | | | |
| n | 16 | | 14 | |
| Adjusted Mean (SE) | 7.52 | (1.515) | 6.14 | (1.614) |

| 95% CI for Adjusted Mean | (4.45, 10.59) | (2.88, 9.41) | |
|---|---|---|---|
| Difference in Adjusted Mean (SE) | | −1.38 | (2.177) |

| | |
|---|---|
| 95% CI for Difference in Adjusted | (−5.80, 3.05) |
| Means | 18.3% |
| Relative Reduction in Adjusted | 0.5314 |
| Mean | |
| p-value | |

ADAS-Cog11 Treatment Benefit (mITT)

Analysis of the broader mITT population is consistent with success in meeting one of the two co-primary endpoints (ADAS-Cog11). ADAS-Cog11 showed a 42.2% improvement in the rate of cognitive decline vs. placebo at week 49 (treatment difference −2.89; p=0.0008). Semorinemab's treatment effect on ADAS-Cog11 was confirmed in this larger (n=238) mITT population that included all trial participants who had received >1 dose of study drug and had at least one post-baseline ADAS-Cog11 assessment. Data from this population show a 42.2% improvement in the rate of cognitive decline with semorinemab compared to placebo (p=0.0008), as measured by ADAS-Cog11. This treatment effect was observed consistently in prespecified subgroups based on disease severity, baseline Tau load, and Apoε carrier status, as reported in more detail below.

Benefit on cognition was driven primarily by the memory domain subcomponent of ADAS-Cog11, a core feature of AD, also as reported in more detail below.

ADAS-Cog11 Treatment Benefit in Populations Subgroups

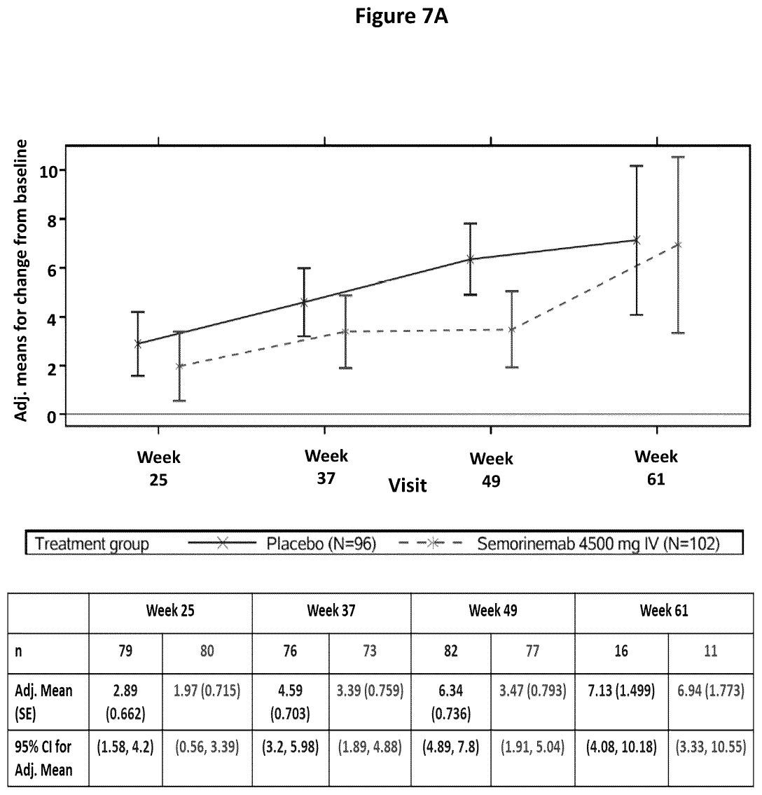

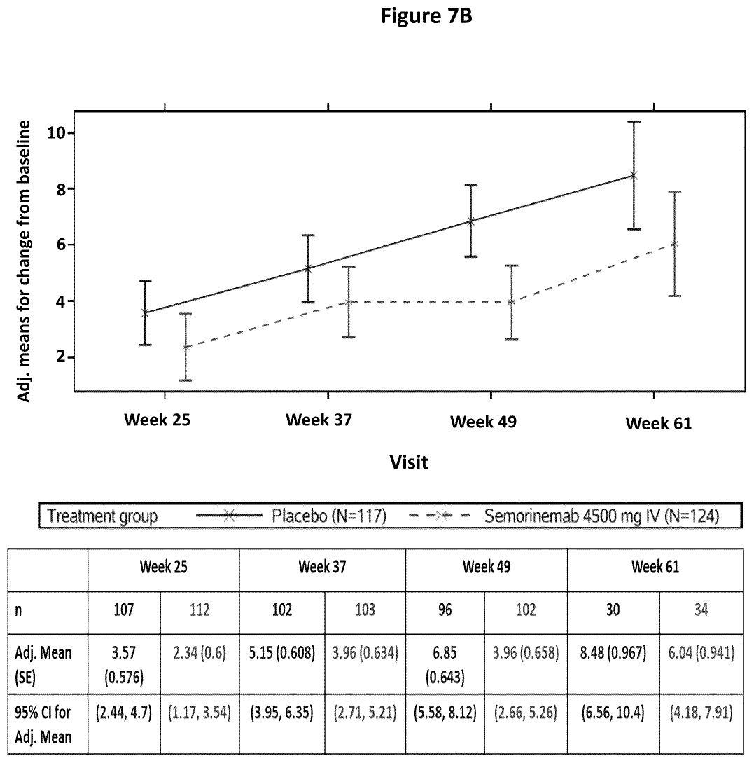

ADAS-Cog11 treatment benefit was consistent in other pre-specified analysis populations at week 25, 37, and 49. See, e.g., FIGS. 7A-7B. As FIG. 7A shows, results were similar to the MITT and Max 1 missed dose population and the semo arm was favored at week 25, 37, and 49. As FIG. 7B shows, results at week 25, 37, and 49 were similar in both the MITT and Max 1 missed dose populations; due to sample size limitations, the week 61 results were not consistent across analysis populations.

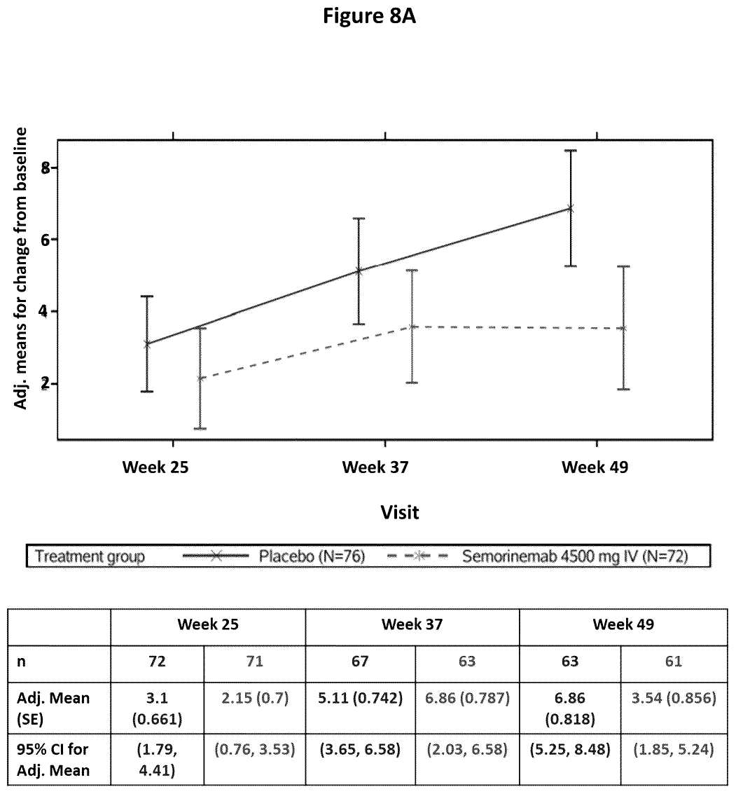

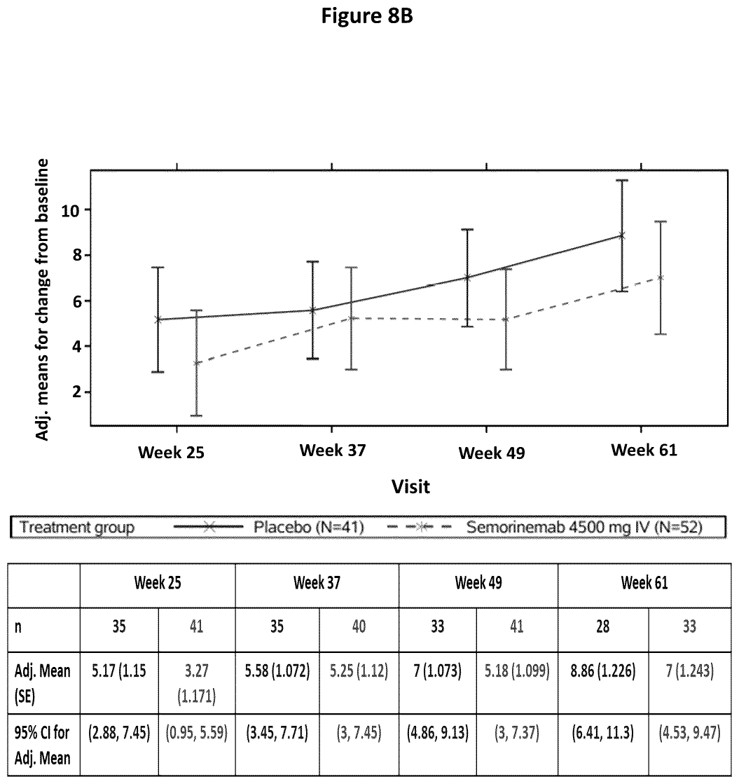

ADAS-Cog11 treatment benefit also was consistent in the Cohort 1 and Cohort 2 subgroups. See, e.g., FIGS. 8A-8B. As FIG. 8A shows, a robust treatment benefit was observed for Cohort 1 patients through all timepoints. As FIG. 8B shows, a consistent treatment effect can be observed for Cohort 2 patients throughout (numerically slightly smaller than for Cohort 1).

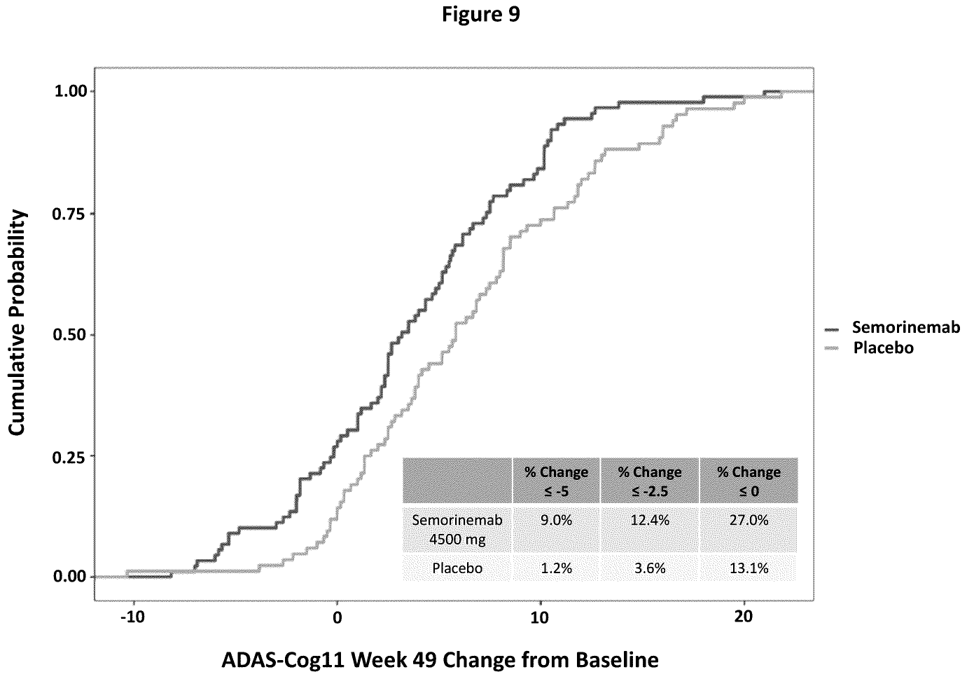

ADAS-Cog11 treatment benefit also was robust across a wide range of disease progression rate. See, e.g., FIG. 9. As FIG. 9 shows, probability curves run parallel and are shifted, indicating that the treatment benefit in terms of % responder was robust over a range of cutoff (about −5 to about 15).

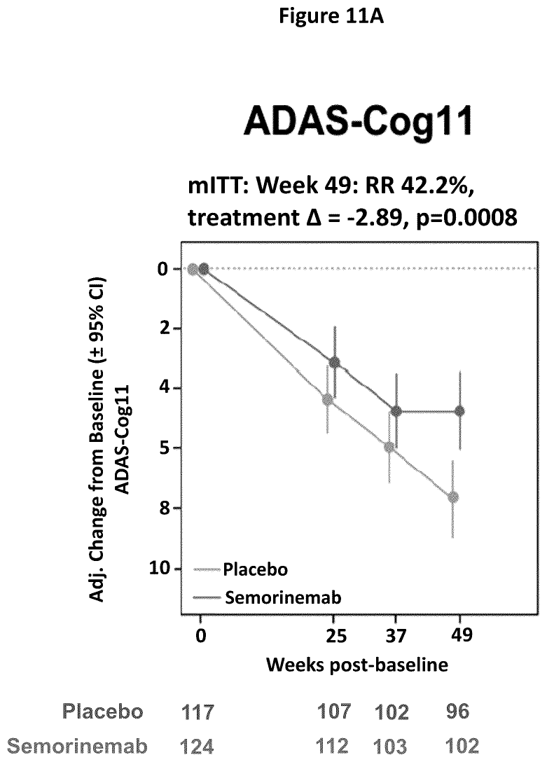

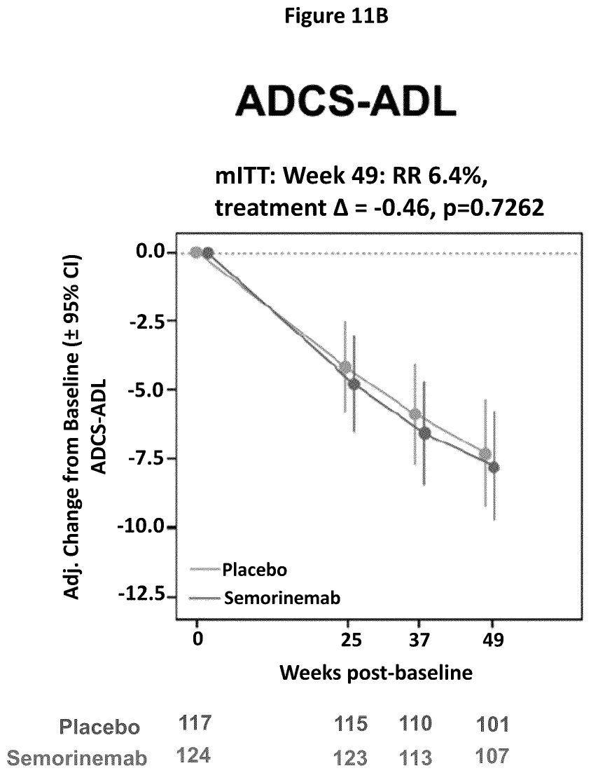

Analysis of the mITT population demonstrated that semorinemab reduced the rate of cognitive decline and had no significant effect on the rate of functional decline in mild-to-moderate AD. See, FIG. 11A and FIG. 11B. FIGS. 11A-11B depict primary end point results for the mITT population. FIG. 11A depicts ADAS-Cog11 adjusted change from baseline over time in placebo (grey circles) and Semorinemab (green circles) arms. FIG. 11B depicts ADCS-ADL adjusted change from baseline over time in placebo (grey circles) and Semorinemab (green circles) arms. Findings were consistent at Week 61, and in other analysis populations including the pre-specified analysis population (mITT*) in which the semorinemab arm demonstrated a 43.6% reduction (−2.96 points, SE=0.97, p=0.0025) in decline on ADAS-Cog11 relative to the placebo arm.

Analysis of Results from Additional Subgroups

### Subgroups Based on Baseline Tau Load, Disease Severity, and APOε4 Carrier Status:

Pre-specified subgroup analyses were consistent with the overall group results, for subgroups based on Tau load, disease severity, and APOε 4 carrier status.

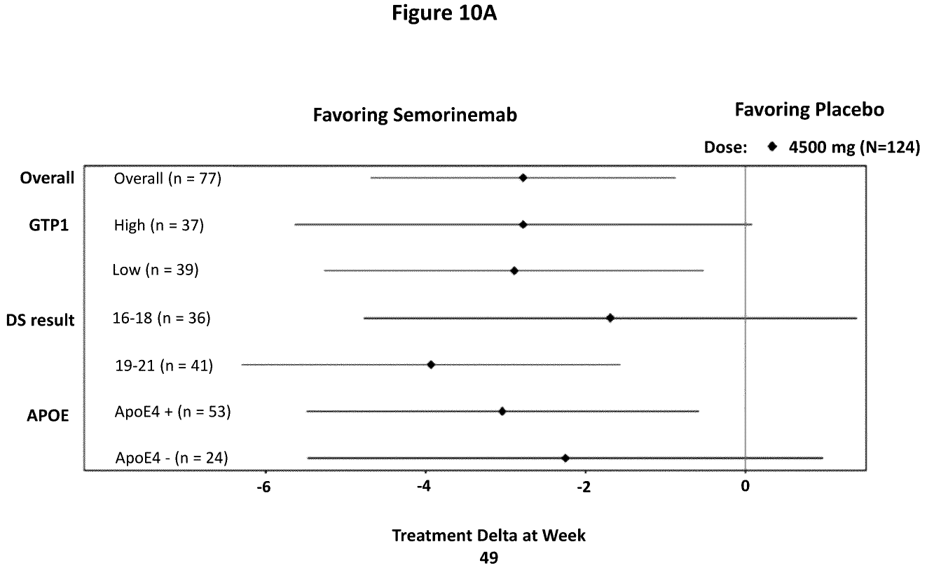

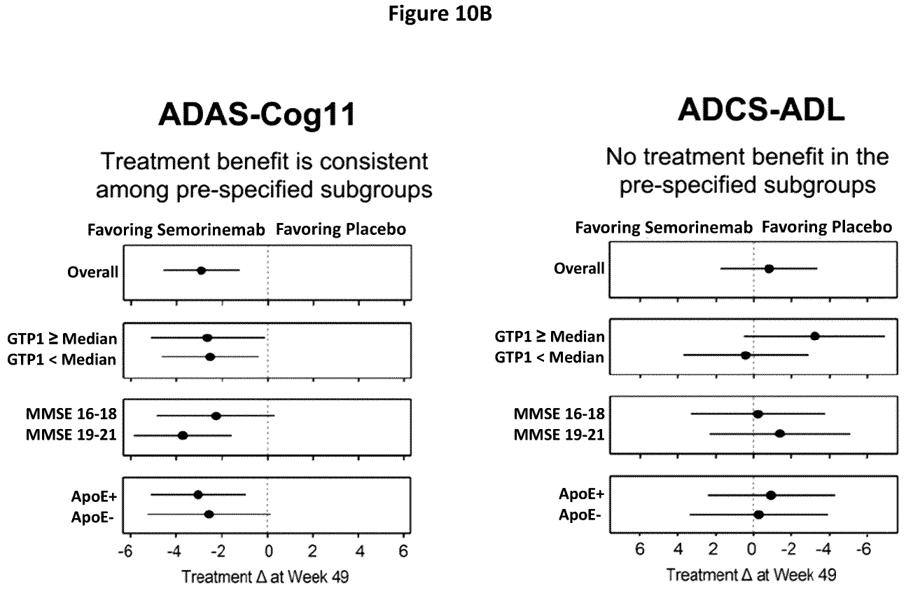

ADAS-Cog11 treatment benefits also were consistent among pre-specified subgroups, including high or low GTP1 (where high GTP1 here is defined as above or equal to median GTP1 WCG and low GTP1 is defined here as below median GTP1 WCG); MMSE 16-18 or MMSE 19-21; and Apoε4 positive or Apoε4 negative. See, e.g., FIG. 10A. As FIG. 10A shows, there were consistent differences in adjusted means versus placebo, using Forest plots from Mixed-Effect repeated measures model analysis of change from baseline, ADAS-Cog11, Double Blind Period, in (MITT, Max 1 missed dose) at Week 49, for different pre-specified subgroups. FIG. 10A depicts a forest plot showing the differences of adjusted means from the Mixed-Effect repeated measures model that analyzes the change from baseline to week 49 of ADAS-Cog11 for MIIT subjects who missed maximum one dose. Pre-specified subgroups included high or low GTP1 (where high GTP1 is defined as above or equal to median GTP1 WCG and low GTP1 is defined as below median GTP1 WCG); MMSE 16-18 or MMSE 19-21; and Apoε4 positive or Apoε4 negative. FIG. 10A depicts a forest plot showing the differences of adjusted means from the Mixed-Effect repeated measures model that analyzes the change from baseline to week 49 of ADAS-Cog11 for MIIT subjects who missed maximum one dose. Pre-specified subgroups included high or low GTP1 (where high GTP1 is defined as above or equal to median GTP1 WCG and low GTP1 is defined as below median GTP1 WCG); MMSE 16-18 or MMSE 19-21; and Apoε4 positive or Apoε4 negative.

FIG. 10B further compares ADAS-Cog11 results with ADCS-ADL results, further depicting consistent differences in adjusted means versus placebo using Forest plots from Mixed-Effect repeated measures model analysis of change from baseline, ADAS-Cog11 or ADCS-ADL, Double Blind Period, in (MITT, Max 1 missed dose) at Week 49, for the different pre-specified subgroups. FIG. 10B compares ADAS-Cog11 results with ADCS-ADL results, further depicting forest plots showing the differences of adjusted means from the Mixed-Effect repeated measures model that analyzes the change from baseline to week 49 of ADAS-Cog11, or ADCS-ADL, for MIIT subjects who missed maximum one dose, for the different pre-specified subgroups.

### Analyses of ADAS-Cog11 Domains:

### Memory Domain:

ADAS-Cog11 domain analyses show that semorinemab's treatment effect was driven predominantly by the memory domain, which is a core feature of AD. The table below provides a “breakdown” of the ADAS-Cog11 scores, based on three cognitive domains: a memory domain, language domain, and praxis domain. ADAS-Cog11 cognitive domains are defined herein according to Verma et al. Alzheimer's Research & Therapy 2015. Results are shown in Table 14, below. A memory domain value of an ADAS-Cog11 score may be referred to herein as a “ADAS-Cog11 memory domain score” or simply “memory domain.”

TABLE 14

| | | | Memory | Language | Praxis |
|---|---|---|---|---|---|
| | | | Domain | Domain | Domain |
| | Week | n | (mean) | (mean) | (mean) |
| semorinemab | 0 | 124 | 0.000000 | 0.0000000 | 0.0000000 |
| 4500 mg | 25 | 115 | 0.949122 | 0.8552632 | 0.7318841 |
| | 37 | 106 | 1.739579 | 1.1148867 | 1.0770440 |
| | 49 | 104 | 1.643546 | 0.9591503 | 1.0865385 |
| Placebo | 0 | 117 | 0.000000 | 0.0000000 | 0.0000000 |
| | 25 | 108 | 1.913629 | 0.7663551 | 0.6884735 |
| | 37 | 104 | 2.813480 | 0.9640523 | 1.1314103 |
| | 49 | 97 | 3.342760 | 1.5910653 | 1.2594502 |

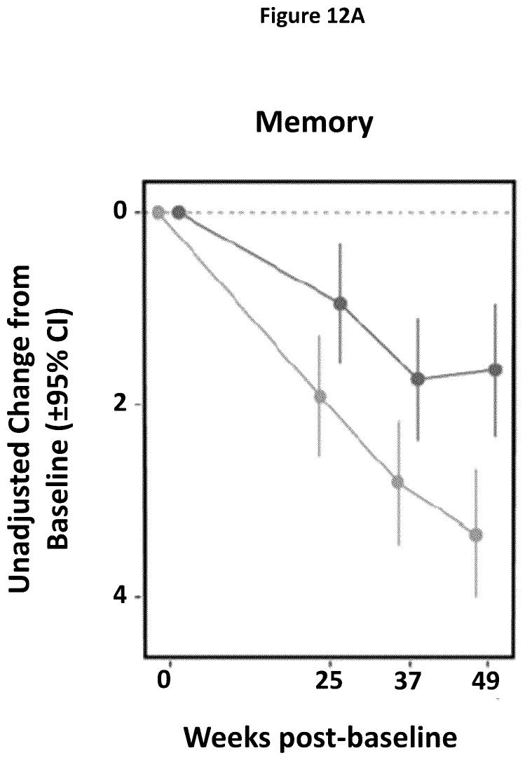

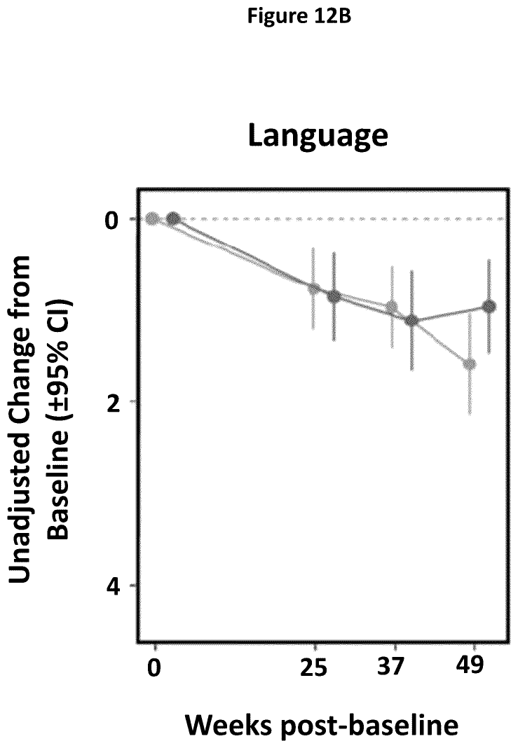

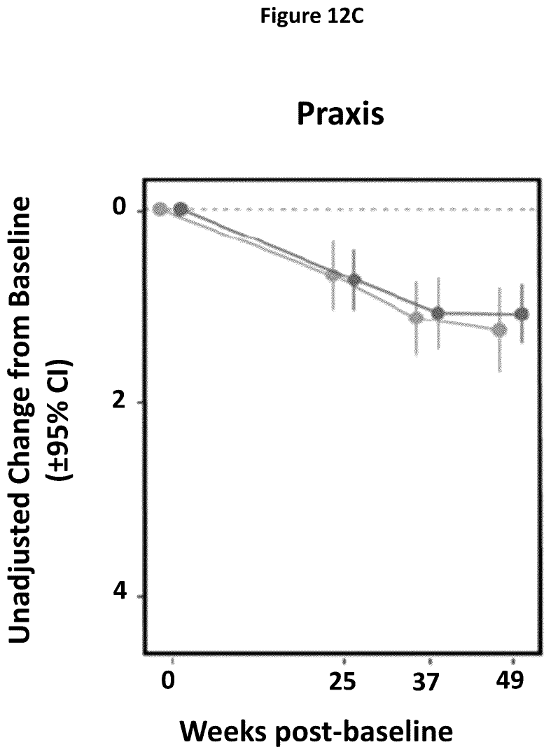

FIGS. 12A-12C depict treatment effects in different cognitive domains (i.e., memory, language, praxis) within the ADAS-Cog11 (as defined as by Verma et al., Alzheimer's Research and Therapy, 2015) for the mITT population over time (weeks post-baseline). FIG. 12A depicts unadjusted change from baseline for the ADAS-Cog11 memory domain scores over time;

FIG. 12B depicts unadjusted change from baseline for the ADAS-Cog11 language domain scores over time; FIG. 12C depicts unadjusted change from baseline for the ADAS-Cog11 praxis domain scores over time.

### Results for Secondary Endpoints:

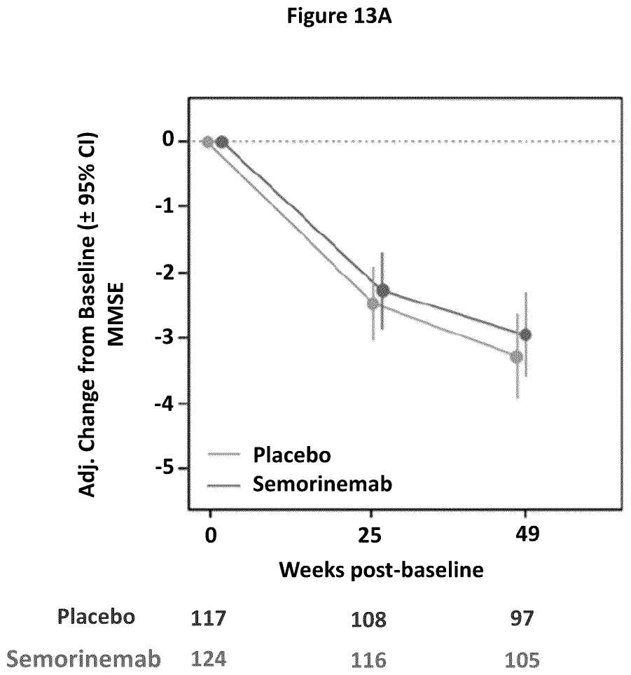

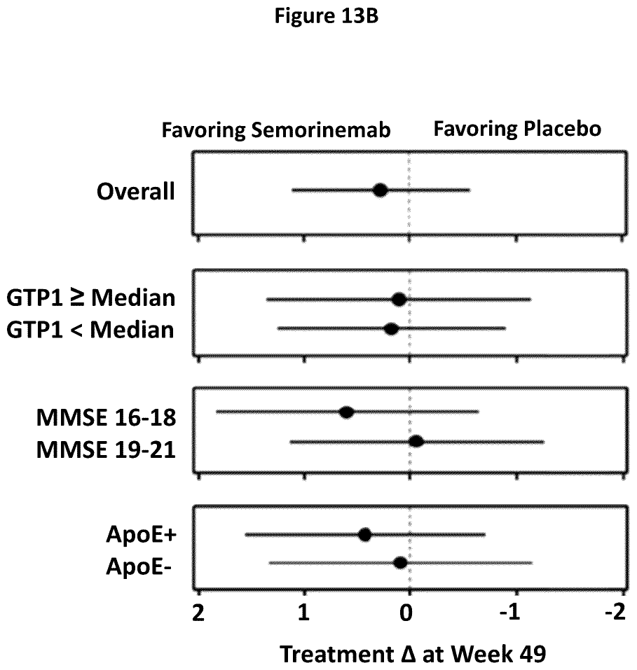

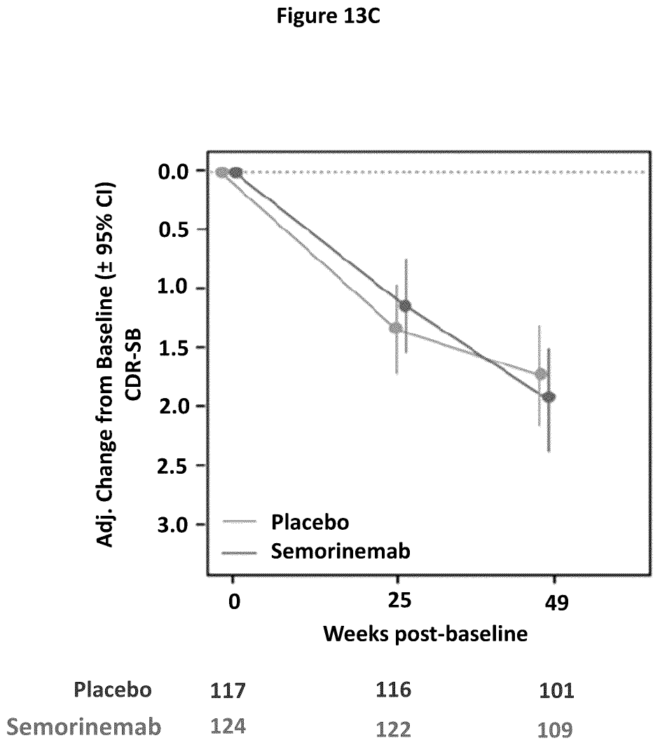

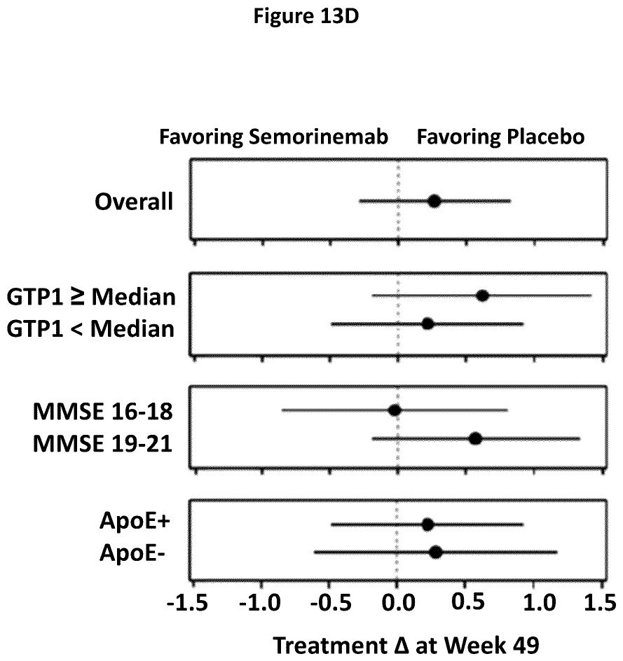

Semorinemab showed no significant effect in mild-to-moderate AD progression measured by secondary endpoints (i.e., MMSE and CDR-SB). See, FIGS. 13A-13D. For FIGS. 13A-13B, MMSE at Week 49 provides RR 10.0% (Treatment Δ=0.33, p=0.4530). FIG. 13A depicts MMSE adjusted change from baseline over time (weeks post-baseline) in the placebo (grey circles) and Semorinemab (green circles) arms. FIG. 13B depicts the Forest plot from the Mixed-Effect repeated measures model that analyzes the change from baseline in MMSE for different pre-specified subgroups (high or low GTP1 (where high GTP1 is defined as above or equal to median GTP1 WCG and low GTP1 is defined as below median GTP1 WCG); MMSE 16-18 or MMSE 19-21; and Apoε4 positive or Apoε4 negative). FIG. 13C depicts CDR-SB (inverse) adjusted change from baseline over time (weeks post-baseline) in the placebo (grey circles) and Semorinemab (green circles) arms. FIG. 13D depicts the Forest plot from the Mixed-Effect repeated measures model that analyzes the change from baseline in CDR-SB for the different pre-specified subgroups (high or low GTP1 (where high GTP1 is defined as above or equal to median GTP1 WCG and low GTP1 is defined as below median GTP1 WCG); MMSE 16-18 or MMSE 19-21; and Apoε4 positive or Apoε4 negative).

### Results Regarding Tau Accumulation:

Biomarker analyses included Tau positron emission tomography (PET) scans and plasma Tau levels. There was no identifiable treatment effect on global or regional Tau distribution as assessed by PET analysis.

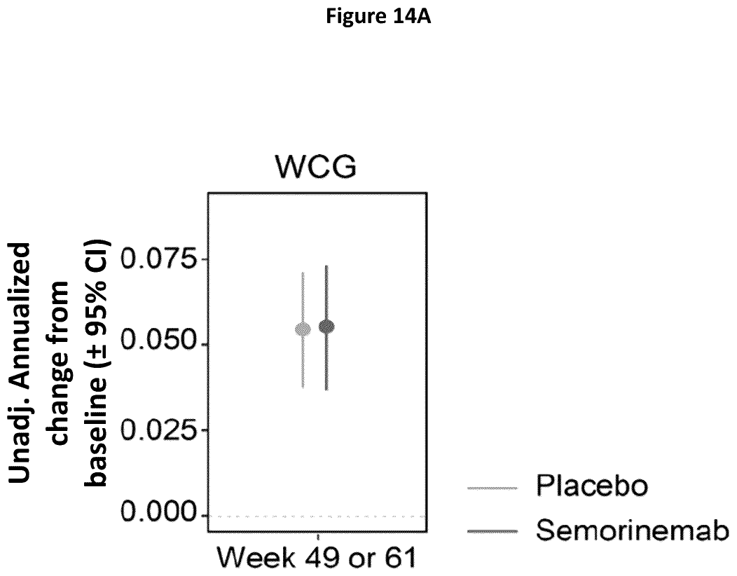

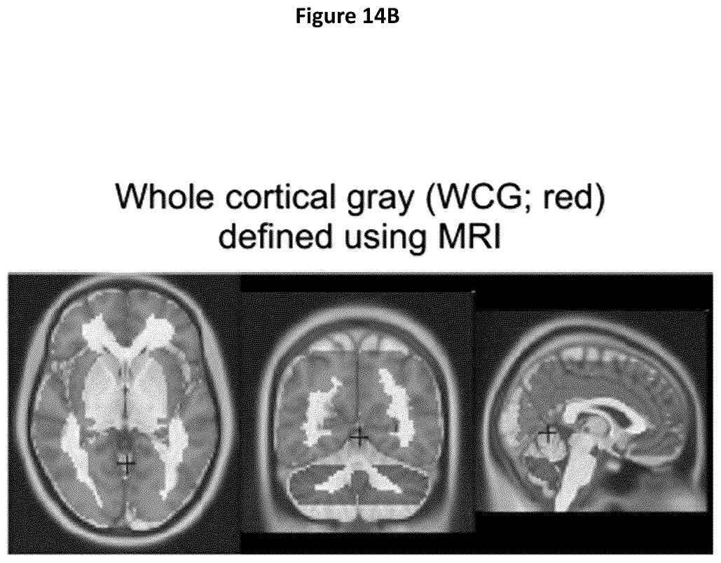

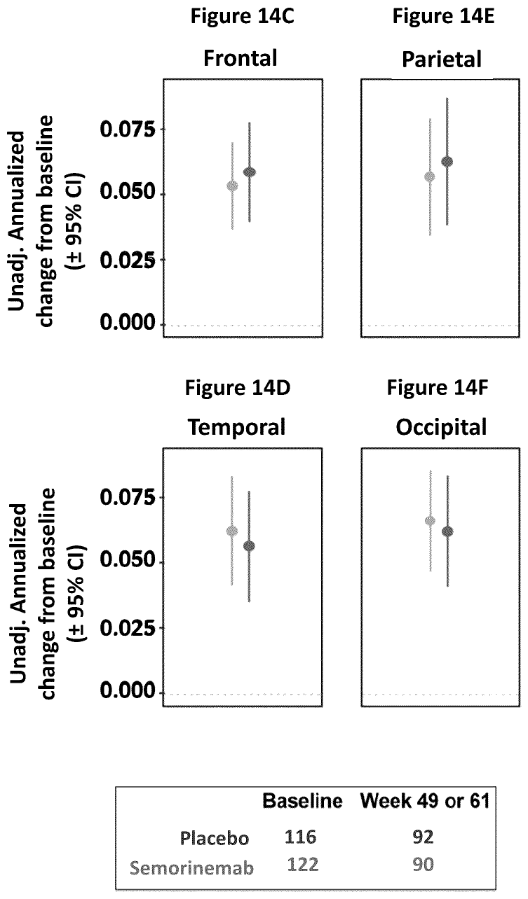

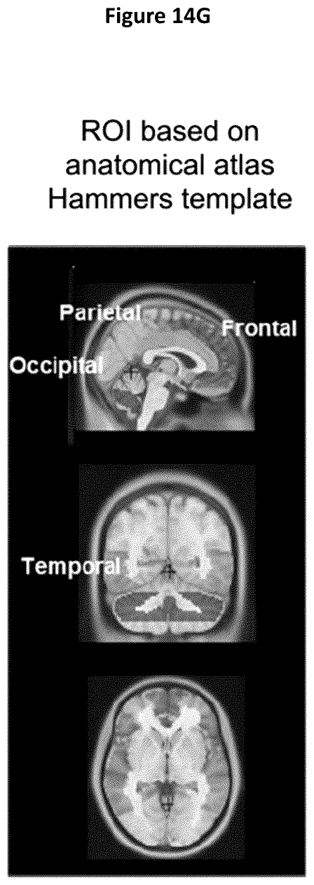

Semorinemab had no significant effect in Tau accumulation in mild-to-moderate AD. See FIGS. 14A-14G. As shown, similar changes in [¹⁸F]GTP1 tau PET signal were observed between the two treatment arms. FIG. 14A depicts unadjusted annualized change from baseline in [¹⁸F]GTP1 tau PET signal of the Whole Cortical Grey region for the placebo (grey) or Semorinemab (green) arms at Week 49 and 61. FIG. 14B shows whole cortical gray region in red, illustrating the location of the whole cortical gray region. FIG. 14C depicts unadjusted annualized change from baseline in [¹⁸F]GTP1 tau PET signal of the frontal region for the placebo (grey) or Semorinemab (green) arms at Week 49 and 61. FIG. 14D depicts unadjusted annualized change from baseline in [¹⁸F]GTP1 tau PET signal of the temporal region for the placebo (grey) or Semorinemab (green) arms at Week 49 and 61. FIG. 14E depicts the unadjusted annualized change from baseline in [¹⁸F]GTP1 tau PET signal of the parietal region for the placebo (grey) or Semorinemab (green) arms at Week 49 and 61. FIG. 14F depicts unadjusted annualized change from baseline in [¹⁸F]GTP1 tau PET signal of the occipital region for the placebo (grey) or Semorinemab (green) arms at Week 49 and 61. FIG. 14G illustrates ROI based on anatomical atlas Hammers template.

In the temporal and occipital regions, however, numerical trends are observed in reducing Tau accumulation.

### PK and PD Results:

Analysis of plasma Tau showed a pronounced increase of plasma Tau levels with semorinemab treatment, which is suggestive of peripheral tau binding. Levels of semorinemab in plasma were in the expected range, as was the ratio of the level of semorinemab in the CSF to that in plasma (mean 0.29%).

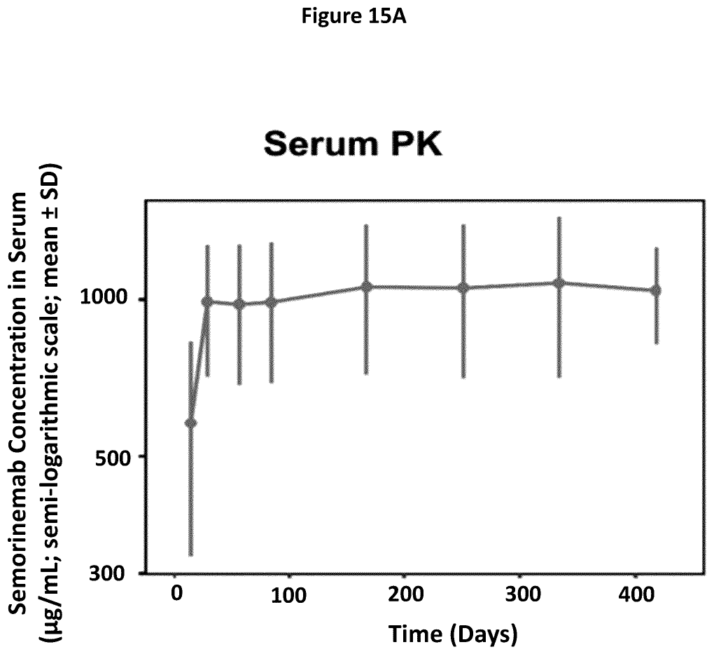

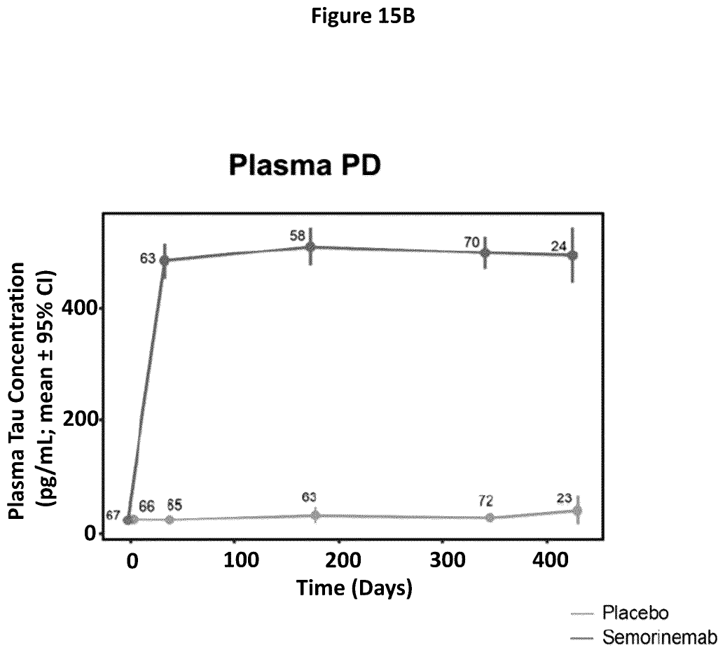

Serum pharmacokinetics profile was similar to previous studies and plasma pharmacodynamics support peripheral tau binding (that is, semorinemab appears to be binding Tau in the plasma). See, FIGS. 15A-14B. FIG. 15A depicts Semorinemab concentration in serum (μg/mL) over time (days), giving a CSF/serum ratio in line with that observed of other monoclonal antibodies (CSF/Serum ratio [%; mean (SD]:0.29 (SD 0.13) (in concordance with observed ratios for other mAbs). FIG. 15B depicts plasma Tau concentration (pg/mL) over time (days) in the placebo (grey circles) and Semorinemab (green circles) arms, showing support for semorinemab engagement with tau peripherally.

### Safety Results:

Safety data from the trial confirmed that semorinemab is well-tolerated with an acceptable safety profile, consistent with previous data. Adverse events and serious adverse events were well-balanced between the two treatment arms, and there were no unanticipated safety signals. See, e.g., Table 15 below.

In the table, and herein: AE, adverse event; IRR, infusion related reaction; SAE, severe adverse event.

### Adverse Events of Double-Blind Period

The Adverse Events Reporting among the safety evaluable group (N=267) is provided in Table 15. The Adverse Events and number of subjects affected per group (placebo or semorinemab) are listed in Table 16 are those that occurred in the double-blind period only. Other Adverse Events are listed in Table 17.

TABLE 15
Adverse Events Reporting of Safety Evaluable Group

| | Placebo | Semorinemab |
|---|---|---|
| Adverse Events Reporting Groups | (N) | (N) |
| Total number of subjects exposed | 132 | 135 |
| Total number of subjects with any adverse event | 105 | 113 |
| Total number of subjects affected by serious adverse events | 22 | 22 |
| Total number of subjects affected by non-serious adverse | 71 | 77 |
| events | | |
| Total number of deaths (all causes) | 2 | 1 |
| Total number of deaths resulting from adverse events | 0 | 0 |
| Subjects with ≥ Grade 3 adverse event | 21 | 30 |
| Subjects with IRR | 5 | 14 |
| Subjects with ≥ Grade 3 IRR | 0 | 0 |

TABLE 16

| | Placebo | Semorinemab |
|---|---|---|
| Adverse Event | (N) | (N) |
| 1. All-Cause Mortality | | |
| Total number affected | 2 | 1 |
| Total number at risk | 132 | 135 |
| 2. Serious Adverse Events | | |
| Total number affected by any Serious Adverse Event | 22 | 22 |
| Total number at risk by any Serious Adverse Event | 132 | 135 |
| 3. Blood and lymphatic system disorders | | |
| Number of subjects affected and number of events | 0 | 1 |
| Number of subjects at risk | 132 | 135 |
| 4. Cardiac disorders | | |
| Atrial fibrillation: Number of subjects affected and number of | 0 | 1 |
| events | | |
| Bradycardia: Number of subjects affected and number of | 0 | 1 |
| events | | |
| Cardiac failure: Number of subjects affected and number of | 1 | 0 |
| events | | |
| Stress cardiomyopathy: Number of subjects affected and | 0 | 1 |
| number of events | | |
| Number of subjects at risk | 132 | 135 |
| 5. Gastrointestinal disorders | | |
| Abdominal pain: Number of subjects affected and number of | 2 | 0 |
| events | | |
| Colitis: Number of subjects affected and number of events | 0 | 1 |
| Hernial eventration: Number of subjects affected and number | 1 | 0 |
| of events | | |
| Inguinal hernia strangulated: Number of subjects affected and | 0 | 1 |
| number of events | | |
| Number of subjects at risk | 132 | 135 |
| 6. General disorders and administration site conditions | | |
| Death: Number of subjects affected and number of events | 1 | 1 |
| Non-cardiac chest pain: Number of subjects affected and | 1 | 0 |
| number of events | | |
| Number of subjects at Risk | 132 | 135 |
| 7. Hepatobiliary disorders | | |
| Cholelithiasis: Number of subjects affected and number of | 1 | 0 |
| events | | |
| Number of subjects at Risk | 132 | 135 |
| 8. Infections and infestations | | |
| COVID-19: Number of subjects affected and number of | 2 | 1 |
| events | | |
| Cystitis: Number of subjects affected and number of events | 0 | 1 |
| Gastroenteritis: Number of subjects affected and number of | 0 | 1 |
| events | | |
| Pneumonia: Number of subjects affected and number of | 2 | 0 |
| events | | |
| Urinary tract infection: Number of subjects affected and | 0 | 1 |
| number of events | | |
| Urosepsis: Number of subjects affected and number of events | 1 | 0 |
| 9. Injury, poisoning and procedural complications | | |
| Craniocerebral injury: Number of subjects affected and | 0 | 1 |
| number of events | | |
| Femoral neck fracture: Number of subjects affected and | 0 | 1 |
| number of events | | |
| Head injury: Number of subjects affected and number of | 1 | 0 |
| events | | |
| Hip fracture: Number of subjects affected and number of | 2 | 0 |
| events | | |
| Joint dislocation: Number of subjects affected and number of | 0 | 1 |
| events | | |
| Patella fracture: Number of subjects affected and number of | 0 | 1 |
| events | | |
| Number of subjects at risk | 132 | 135 |
| 10. Investigations | | |
| SARS-CoV-2 positive: Number of subjects affected and | 0 | 1 |
| number of events | | |
| Number of subjects at risk | 132 | 135 |
| 11. Metabolism and nutrition disorders | | |
| Dehydration: Number of subjects affected and number of | 1 | 1 |
| events | | |
| Diabetic metabolic decompensation: Number of subjects | 0 | 1 |
| affected and number of events | | |
| Number of subjects at risk | 132 | 135 |
| 12. Neoplasms benign, malignant and unspecified (including | | |
| cysts and polyps) | | |
| Adenocarcinoma metastatic: Number of subjects affected and | 0 | 1 |
| number of events | | |
| Invasive ductal breast carcinoma: Number of subjects | 0 | 1 |
| affected and number of events | | |
| Ovarian cancer: Number of subjects affected and number of | 0 | 1 |
| events | | |
| Renal neoplasm: Number of subjects affected and number of | 1 | 0 |
| events | | |
| Squamous cell carcinoma: Number of subjects affected and | 0 | 1 |
| number of events | | |
| Transitional cell carcinoma recurrent: Number of subjects | 0 | 1 |
| affected and number of events | | |
| Number of subjects at risk | 132 | 135 |
| 13. Nervous system disorders | | |
| Cerebrovascular accident: Number of subjects affected and | 1 | 0 |
| number of events | | |
| Dysarthria: Number of subjects affected and number of | 0 | 1 |
| events | | |
| Seizure: Number of subjects affected and number of events | 1 | 1 |
| Subarachnoid hemorrhage: Number of subjects affected and | 0 | 1 |
| number of events | | |
| Syncope: Number of subjects affected and number of events | 1 | 1 |
| Upper motor neuron lesion: Number of subjects affected and | 0 | 1 |
| number of events | | |
| Number of subjects at risk | 132 | 135 |
| 14. Psychiatric disorders | | |
| Agitation: Number of subjects affected and number of events | 3 | 0 |
| Number of subjects at risk | 132 | 135 |
| 15. Renal and urinary disorders | | |
| Hydronephrosis: Number of subjects affected and number of | 1 | 0 |
| events | | |
| Nephrolithiasis: Number of subjects affected and number of | 1 | 0 |
| events | | |
| Renal failure: Number of subjects affected and number of | 0 | 1 |
| events | | |
| Number of subjects at risk | 132 | 135 |
| 16. Respiratory, thoracic and mediastinal disorders | | |
| Pulmonary embolism: Number of subjects affected and | 1 | 0 |
| number of events | | |
| Number of subjects at risk | 132 | 135 |
| 17. Vascular disorders | | |
| Aortic aneurysm: Number of subjects affected and number of | 1 | 0 |
| events | | |
| Peripheral artery thrombosis: Number of subjects affected | 0 | 1 |
| and number of events | | |
| Number of subjects at risk | 132 | 135 |

TABLE 17
Other Adverse Events or Non-Serious Adverse Events

| | Placebo | Semorinemab |
|---|---|---|
| Other Adverse Event | (N) | (N) |
| 1. Other Adverse Events | | |
| Total number affected | 71 | 77 |
| Total number at risk | 132 | 135 |
| 2. Gastrointestinal disorder | | |
| Diarrhea: Number of subjects affected | 5 | 7 |
| Diarrhea: Number of events | 6 | 7 |
| Number of subjects at risk | 132 | 135 |
| 3. Infections and infestations | | |
| Nasopharyngitis: Number of subjects affected | 3 | 9 |
| Nasopharyngitis: Number of events | 3 | 10 |
| Urinary tract infection: Number of subjects affected | 16 | 10 |
| Urinary tract infection: Number of events Number of | 19 | 14 |
| events | | |
| Number of subjects at risk | 132 | 135 |
| 4. Injury, poisoning and procedural complications | | |
| Fall: Number of subjects affected | 19 | 14 |
| Fall: Number of events | 28 | 19 |
| Infusion related reaction: Number of subjects affected | 5 | 14 |
| Infusion related reaction: Number of events | 9 | 30 |
| Number of subjects at risk | 132 | 135 |
| 5. Musculoskeletal and connective tissue disorders | | |
| Arthralgia: Number of subjects affected and number of | 4 | 7 |
| events | | |
| Number of subjects at risk | 132 | 135 |
| 6. Nervous system disorders | | |
| Dizziness: Number of subjects affected | 9 | 8 |
| Dizziness: Number of events | 11 | 8 |
| Headache: Number of subjects affected | 9 | 11 |
| Headache: Number of events | 10 | 14 |
| Number of subjects at risk | 132 | 135 |
| 7. Psychiatric disorders | | |
| Agitation: Number of subjects affected and number of | 5 | 8 |
| events | | |
| Anxiety: Number of subjects affected | 12 | 9 |
| Anxiety: Number of events | 12 | 11 |
| Depression: Number of subjects affected and number of | 6 | 10 |
| events | | |
| Insomnia: Number of subjects affected and number of | 2 | 7 |
| events | | |
| Number of subjects at risk | 132 | 135 |
| 8. Vascular disorders | | |
| Hypertension: Number of subjects affected | 5 | 8 |
| Hypertension: Number of events | 5 | 10 |
| Number of subjects at risk | 132 | 135 |

### Conclusions:

This clinical trial showed a relative reduction (RR=43.6%, CHG=2.96 points, p-value=0.003) of cognitive decline at Week 49 in the semorinemab arm versus placebo, as measured by ADAS-Cog11 and treatment benefit was also observed at Week 25 and 37. Overall, findings were consistent between pre-specified analysis populations.

These results are statistically significant in the extent of reduction in cognitive decline and the extent of maintenance of cognitive capacity (as measured by ADAS-Cog11 scores) over the time period, during which the patient was expected to show a 6 point ADAS-Cog11 score increase (that is, a 6 point decline) without anti-Tau antibody treatment. These results surprisingly robust and are especially surprising, given that patients enrolled in the study (including those in the placebo arm) also were being treated with symptomatic medication. These results provide evidence of an anti-Tau monoclonal antibody approach in treating patient populations at later stages (moderate, mild-to-moderate) of AD, surprisingly reducing cognitive decline to such an extent over a course of treatment.

Preliminary results also support robust tau binding in the periphery (plasma total tau) and, in the small subset of patients with CSF, indicated a reduction in mid-domain tau in CSF (p-tau 181, total Tau, Tau 93-105) with semorinemab treatment.

### Clinical Trial Summary:

This clinical trial provided first positive cognitive results for a Tau-targeting monoclonal antibody in Alzheimer's Disease. Top-line data from this Phase 2 trial of semorinemab in mild-to-moderate AD showed a statistically significant reduction on one of two co-primary endpoints, ADAS-Cog11, and provided the first evidence of clinical activity in tau-targeting monoclonal antibody in a MMSE 16-21 (mild-to-moderate) AD population. This clinical trial, a placebo-controlled Phase 2 study evaluating the safety and efficacy of the anti-tau monoclonal antibody, semorinemab, in mild-to-moderate AD, met one of its co-primary endpoints, ADAS-Cog11. The second coprimary endpoint, ADCS-ADL, was not met. Safety data showed that semorinemab is well tolerated with an acceptable safety profile and no unanticipated safety signals.

Semorinemab demonstrated a statistically significant reduction in cognitive decline from baseline by 43.6% compared to placebo (p=0.0025) as measured by the Alzheimer's Disease Assessment Scale, Cognitive Subscale, 11-item Version (ADAS-Cog11) at week 49 in people with mild-to-moderate AD (i.e., Mini-Mental State Examination (MMSE) 16-21). There was no effect on the other co-primary endpoint of reducing the rate of functional decline from baseline as measured by the Alzheimer's Disease Cooperative Study-Activities of Daily Living (ADCS-ADL) or secondary efficacy endpoints for the Mini-Mental State Examination (MMSE) or the Clinical Dementia Rating-Sum of Boxes (CDR-SB). The safety was consistent with previous clinical data reported.

This was the first time a therapeutic effect by a monoclonal anti-Tau antibody therapy has been seen, and the first time a monoclonal antibody has had a therapeutic impact on cognition in the mild-to-moderate AD patient population.

This clinical trial showed a relative reduction (RR=43.6%, CHG=2.96 points, p-value=0.003) in cognitive decline at Week 49 in the semorinemab arm versus placebo, as measured by ADAS-Cog11 and treatment benefit is also observed at Week 25 and 37. Overall, findings are consistent between pre-specified analysis populations.

After 12 months of treatment, participants with mild-to-moderate AD receiving semorinemab 4500 mg Q4W showed evidence for slowing cognitive decline on ADAS-Cog, primarily on the memory domain.

No significant treatment benefit of semorinemab in ADCS-ADL (RR=−12.6%, CHG=−0.99 points, p-value=0.492) was observed at Week 49. No significant treatment effect was observed at week 25, 37 either and the overall findings were consistent between pre-specified analysis populations.

No significant treatment effect was observed for the secondary endpoints CDR-SB and MMSE.

There was no evidence of treatment effect of semorinemab in Tau accumulation. Global Tau accumulation measured by GTP1 PET was observed and was consistent with projections from a previous GTP1 natural history study in this patient population.

Preliminary results supported robust tau binding in the periphery (plasma total tau), and, in a small subset of patients with CSF, were suggestive of a reduction in mid-domain tau in CSF (p-tau 181, total Tau, Tau 93-105) with semorinemab treatment

Safety data showed that semorinemab was well tolerated, with acceptable safety profile and no unanticipated signals.

Missed doses (due to COVID-19 restrictions or other factors) did not impact the overall interpretation of treatment effects for the efficacy endpoints.

There were no major imbalances in baseline characteristics, discontinuations that could have implications for the interpretation of the treatment effects for the efficacy endpoints

Serum and CSF PK were consistent with the 4500 mg arm of earlier clinical trials in patients with early (prodromal-to-mild) AD and Ph1 results.

Directional trend towards slower ADAS-Cog11 progression at higher semorinemab exposure was observed.

No treatment-emergent ADAs were observed with semorinemab.

1. A method of slowing decline in cognitive capacity in a patient diagnosed with mild-to-moderate Alzheimer's disease (AD), comprising administering to said patient a 4500 mg dose of humanized monoclonal anti-Tau antibody, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

2. A method of maintaining cognitive capacity within 5 points of an Alzheimer's disease Assessment Scale, Cognitive Subscale, 11-item version (ADAS-Cog11) score of a patient diagnosed with mild-to-moderate AD, comprising administering to said patient a 4500 mg dose of humanized monoclonal anti-Tau antibody,
wherein the ADAS-Cog11 score of the patient assessed after administration of 12 to 17 doses of said antibody is no more than 2.5, no more than 3, no more than 3.5, no more than 4, no more than 4.5, or no more than 5 points higher than an ADAS-Cog11 score of the patient assessed before administration of said antibody,
thereby maintaining cognitive capacity within 5 points of the ADAS-Cog11 score of the patient, and
wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

3. A method of slowing decline in cognitive capacity in a patient diagnosed with moderate AD, comprising administering to said patient a 4500 mg dose of humanized monoclonal anti-Tau antibody, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

4. A method of maintaining cognitive capacity within 5 points of an Alzheimer's disease Assessment Scale, Cognitive Subscale, 11-item version (ADAS-Cog11) score of a patient diagnosed with moderate AD, comprising administering to said patient a 4500 mg dose of humanized monoclonal anti-Tau antibody,
wherein the ADAS-Cog11 score of the patient assessed after administration of 12 to 17 doses of said antibody is no more than 2.5, no more than 3, no more than 3.5, no more than 4, no more than 4.5, or no more than 5 points higher than an ADAS-Cog11 score of the patient assessed before administration of said antibody,
thereby maintaining cognitive capacity within 5 points of the ADAS-Cog11 score of the patient, and
wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

5. A method of slowing memory decline in a patient diagnosed with mild-to-moderate AD, comprising administering to said patient a 4500 mg dose of humanized monoclonal anti-Tau antibody, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

6. A method of maintaining memory within 2.5 points of an Alzheimer's disease Assessment Scale, Cognitive Subscale, 11-item version (ADAS-Cog11) memory domain score of a patient diagnosed with mild-to-moderate AD, comprising administering to said patient a 4500 mg dose of humanized monoclonal anti-Tau antibody,
wherein the ADAS-Cog11 memory domain score of the patient assessed after administration of 12 to 17 doses of said antibody is no more than 1, no more than 1.5, no more than 1.7, no more than 2, no more than 2.3, or no more than 2.5 points higher than an ADAS-Cog11 memory domain score of the patient assessed before administration of said antibody,
thereby maintaining memory within 2.5 points of the ADAS-Cog11 memory domain score of the patient, and
wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

7. A method of slowing memory decline in a patient diagnosed with moderate AD, comprising administering to said patient a 4500 mg dose of humanized monoclonal anti-Tau antibody to slow the decline in memory in the patient, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

8. A method of maintaining memory within 2.5 points of an Alzheimer's disease Assessment Scale, Cognitive Subscale, 11-item version (ADAS-Cog11) memory domain score of a patient diagnosed with moderate AD, comprising administering to said patient a 4500 mg dose of humanized monoclonal anti-Tau antibody,
wherein the ADAS-Cog11 memory domain score of the patient assessed after administration of 12 to 17 doses of said antibody is no more than 1, no more than 1.5, no more than 1.7, no more than 2, no more than 2.3, or no more than 2.5 points higher than an ADAS-Cog11 memory domain score of the patient assessed before administration of said antibody,
thereby maintaining memory within 2.5 points of the ADAS-Cog11 memory domain score of the patient, and
wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

9. A method of slowing decline in language capacity in a patient diagnosed with mild-to-moderate AD, comprising administering to said patient a 4500 mg dose of humanized monoclonal anti-Tau antibody, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

10. A method of slowing decline in language capacity in a patient diagnosed with moderate AD, comprising administering to said patient a 4500 mg dose of humanized monoclonal anti-Tau antibody, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

11. A method of slowing decline in praxis capacity in a patient diagnosed with mild-to-moderate AD, comprising administering to said patient a 4500 mg dose of humanized monoclonal anti-Tau antibody, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

12. A method of slowing decline in praxis capacity in a patient diagnosed with moderate AD, comprising administering to said patient a 4500 mg dose of humanized monoclonal anti-Tau antibody, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

13. A method of treating a patient diagnosed with mild-to-moderate AD without increased risk of an adverse event, comprising administering to said patient a 4500 mg dose of humanized monoclonal anti-Tau antibody, without increasing (or without significantly increasing) the risk of a treatment emergent adverse event, optionally wherein the dose is repeated for 12 to 17 doses, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

14. A method of treating a patient diagnosed with moderate AD without increased risk of an adverse event, comprising administering to said patient a 4500 mg dose of humanized monoclonal anti-Tau antibody, without increasing (or without significantly increasing) the risk of a treatment emergent adverse event, optionally wherein the dose is repeated for 12 to 17 doses, wherein the anti-Tau antibody comprises an HVR-H1 comprising the amino acid sequence set forth in SEQ ID NO:2; an HVR-H2 comprising the amino acid sequence set forth in SEQ ID NO:3; an HVR-H3 comprising the amino acid sequence set forth in SEQ ID NO:4; an HVR-L1 comprising the amino acid sequence set forth in SEQ ID NO:6; an HVR-L2 comprising the amino acid sequence set forth in SEQ ID NO:7; and an HVR-L3 comprising the amino acid sequence set forth in SEQ ID NO:8.

15.-76. (canceled)

77. The method of claim 1, wherein the antibody is an IgG4 antibody.

78. The method of claim 77, wherein the antibody comprises M252Y, S254T, and T256E mutations, according to EU numbering.

79. The method of claim 78, wherein the antibody comprises an S228P mutation, according to EU numbering.

80. The method of claim 1, wherein the antibody comprises a heavy chain variable region comprising an amino acid sequence that is at least 95% identical to the sequence of SEQ ID NO: 5; and/or a light chain variable region comprising an amino acid sequence that is at least 95% identical to the sequence of SEQ ID NO: 9.

81. The method of claim 1, wherein the antibody comprises a heavy chain variable region having the amino acid sequence of SEQ ID NO: 5 and/or a light chain variable region having the amino acid sequence of SEQ ID NO:9.

82. The method of claim 1, wherein the antibody is semorinemab.

83-159. (canceled)
